(19) United States
(12) Patent Application Publication
(10) Pub. No.: US 2026/0248702 A1
(43) Pub. Date: Aug. 27, 2026

(54) Composition Comprising a UV-Filter Stabilizer

(71) Applicant: Symrise AG, Holzminden (DE)

(72) Inventors: Jürgen CLAUS, Bevern (DE); Ev SUESS, Bevern (DE); Imke MEYER, Bodenwerder (DE); Nikolas BUGDAHN, Holzminden (DE); Sabine LANGE, Holzminden (DE); Daniel BERNDT, Halle (DE); Lukas SCHAAF, Hameln (DE)

(21) Appl. No.: 18/992,367

(22) PCT Filed: Aug. 7, 2023
(86) PCT No.: PCT/EP2023/071804

§ 371 Date: Jan. 8, 2025

(30) Foreign Application Priority Data
Aug. 5, 2022 (WO) PCT/EP2022/072095
Aug. 4, 2023 (WO) PCT/EP2023/071719

(51) Int. Cl. A61K 8/49; A61K 8/44; A61K 8/46; A61Q 17/04

(52) U.S. Cl. CPC A61K 8/49; A61K 8/445; A61K 8/466; A61Q 17/04; A61K 2800/30

(57) ABSTRACT

The present invention refers to sunscreen products, cosmetic or pharmaceutical preparations or homecare products comprising a photo-unstable UV-filter and as stabilizer therefore, a certain mycosporine-like amino acid compound. Additionally, the present invention relates to the use of such mycosporine-like amino acid compound for improving the photostability of a photo-unstable UV-filter. Finally, the present invention relates to a method of stabilizing a photo-unstable UV-filter against the degradative effects of UV irradiation or of enhancing the UV-absorbing properties of a UV-absorbing compound by said mycosporine-like amino acid compound.

### TECHNICAL FIELD

The present invention refers to sunscreen products, cosmetic or pharmaceutical preparations or homecare products comprising a photo-unstable UV-filter and as stabilizer therefore, a certain mycosporine-like amino acid compound. Additionally, the present invention relates to the use of such mycosporine-like amino acid compound for improving the photostability of a photo-unstable UV-filter. Finally, the present invention relates to a method of stabilizing a photo-unstable UV-filter against the degradative effects of UV irradiation or of enhancing the UV-absorbing properties of a UV-absorbing compound by said mycosporine-like amino acid compound.

### BACKGROUND ART

The negative effects of exposure of UV light are well-known. Prolonged exposure to sunlight causes damage such as sunburn to the skin and dries out hair making it brittle. When skin is exposed to UV light having a wavelength of from 290 nm to 400 nm, long term damage can lead to serious conditions such as skin cancer, the type of damage depending on the wavelength of the radiation.

UV light also contributes to aging by causing free radicals to form in the skin. Free radicals include, for example, singlet oxygen, hydroxyl radical, the superoxide anion, nitric oxide and hydrogen radicals. Free radicals attack DNA, membrane lipids and proteins, generating carbon radicals. These in turn react with oxygen to produce a peroxyl radical that can attack adjacent fatty acids to generate new carbon radicals. This cascade leads to a chain reaction producing lipid peroxidation products. Damage to the cell membrane results in loss of cell permeability, increased intercellular ionic concentration, and decreased ability to excrete or detoxify waste products. The end result is a loss of skin elasticity and the appearance of wrinkles. This process is commonly referred to as photo-aging.

Moreover, UVA radiation can trigger phototoxic or photo allergic skin reactions.

UV rays are classified according to their wavelength as UVA rays (320 to 400 nm) or UVB rays (280 to 320 nm).

To attenuate these negative effects of UV-radiation, i.e. for the purpose of protecting the skin and keratin materials against UV-radiation, anti-sun/sunscreen compositions comprising screening agents that are active in the UVA range and in the UVB range, i.e. within the full range of 280 nm to 400 nm, are generally used.

UV-filters are compounds which have a pronounced absorption capacity for ultraviolet radiation. They are used especially in sunscreen products, cosmetic, dermatological and pharmacological preparations or sunscreen products, but also to improve the light fastness of industrial products, such as paints, varnishes, plastics, textiles, polymers such as, for example, polymers and copolymers of mono- and di-olefins, polystyrenes, polyurethanes, polyamides, polyesters, polyureas and polycarbonates, packaging materials and rubbers.

The UV-filters are classified as UVA and UVB filters depending on the location of their absorption maxima; if a UV-filter absorbs both UVA and UVB, it is referred to as a UVA/UVB broadband absorber or UVA/UVB broadband filter.

In order to achieve the desired maximum protection from UV-radiation sunscreen products, cosmetic, especially dermatological, and pharmacological preparations or homecare products are formulated to contain a mixture of UV-filters with varying concentrations and the choice of UV-filters used is determined by the legislation within the country or economic area. For example, UV-filters which can be used for the protection of skin are regulated in the USA by the America FDA via their OTC monograph system and are regulated in the European Union by the Cosmetic Regulation. Regulations covering the use of UV-filters exist in other countries and regions as well. These regulations not only stipulate the filters which can be used but also fix a maximum usage level for each UV-filter. Thus, there are limited UV-filters available to achieve high efficacy with respect to both SPF and UVA or UVB protection.

However, it is known in the art that some of said approved and used UV-filters, such as Ethylhexyl-Methoxycinnamate (Neo Heliopan® AV), Butyl-Methoxydibenzoylmethane (Avobenzone; Neo Heliopan® 357), Isoamyl-p-Methoxycinnamate (Neo Heliopan® E1000), Diethylamino-hydroxy-Benzoylhexylbenzoate (Uvinul A plus), are relatively sensitive to ultraviolet radiation (UVR), i.e. have potential to degrade chemically when exposed to UVR after exposure, in particular prolonged exposure. More specifically, they have an annoying tendency to be degraded more or less rapidly under the action of this UV.

Exposure to UVR dissipates the available concentration of the UV-filters in a formulation, resulting in a product with decreased efficacy, both in terms of level of protection and time of protection. Thus, the labelled SPF of a sunscreen formulation containing said photo-unstable UV-filters is not necessarily effective of the product's UVR protection.

In terms of photophysics, UVR exposure causes organic UV absorbing molecules to leave the ground state and enter to single-excited state. The excited-state filters may be returned to the ground state by singlet quenching and thereby conserved to continue providing UVR protection or transferred from the singlet-state to the triplet-excited state. In the triplet state, the UV-filter molecule may undergo triplet quenching and return to the ground state. If, however, triple quenching or another photophysical pathway does not restore the sunscreen molecule to the ground state, the filter undergoes photodegradation by one or more photochemical reactions. Thus, photodegraded filters lose the ability to filter UVR, thereby severely decreasing the effectiveness, i.e. UV protective capacity, of the overall formulation.

Thus, this substantial lack of photochemical stability of said photo-unstable UV-filters towards ultraviolet radiation, to which they are by nature intended to be subjected, does not make it possible to ensure constant protection during prolonged exposure to the sun, and so the use must make repeated application at regular and frequent time intervals in order to obtain effective protection of the skin against UV rays.

Additionally, it is believed that the photodegradation of photo-unstable UV-filters caused by photoisomerization or photolysation can result in phototoxic or photoallergic degradation products, for example free radicals, with are associated with a risk of adverse health effects when coming into direct contact with the skin.

These UV-light absorbing filters require stabilization by one or more compounds or materials in the formulation.

In order to increase the photostability of said photo-unstable UV-filters the combined use with a photostabilizer is suggested. For example, an increase in photostability of avobenzone can be observed when it is combined with zinc oxide or avobenzone's photostability can be enhanced by addition of octocrylene or phenylbenzimidazole sulfonic acid.

However, the use of some of the photostabilizers is controversial. For example, octocrylene and benzophenones has been recently incriminated to potentially induce adverse effects on the endocrine system in addition to having allergic and/or photoallergic potential and might be harmful to corals.

Thus, there is still an ongoing need to provide further substances which stabilize photo-unstable UV-filters against photodegradation. The sunscreen, cosmetic or pharmaceutical industry or homecare product business is permanently searching for ingredients which can erase this negative aspect.

Another disadvantage is that the vast majority of cosmetic registered organic UV-filters are only miscible or soluble in oil, i.e. they are only suitable to be added to the oil phase of a sunscreen formulation. The solubility of each UV-filter is thereby dependent on the oils used. Hence, the formulation of a sunscreen is limited by the choice of certain oils and thus also limited by choosing exemplary emollients to improve the sensory. This may affect the compliance of the consumer. The oil soluble photo-unstable UV-filters are predominantly stabilized with oil soluble substances in the oil phase.

Hence, there is a need for novel water-soluble compounds that are photostable themselves and provide stabilization of an oil soluble photo-unstable UV-filters against photodegradation, for example in the water phase of an formulation.

When searching for agents for improving photostability of UV-filters it must be taken into account that the substances used in the sunscreen, cosmetic or pharmaceutical or homecare sector must be toxicologically acceptable, well tolerated by the skin, stable, especially in the customary cosmetic and/or pharmaceutical formulations, substantially and preferably odourless and able to be prepared inexpensively, i.e. using standard methods.

It is therefore an object of the present invention to provide photostable sunscreen products, cosmetic or pharmaceutical, in particular dermatological, preparations, or homecare products comprising or consisting of a photo-unstable UV-filter in which the UV-filter is stabilized against photodegradation in order to preserve the UV protective capacity, and, thus, to provide a constant protection during prolonged exposure to the sun.

It is another object of the present invention to provide a substance, which is capable of stabilizing photo-unstable UV-filters against photodegradation or at least to reduce the loss of photodegradation of photo-unstable UV-filters during exposure to UV-radiation and to prevent the formation of reactive intermediates of photo-unstable filter substances behaving as photo-oxidants when coming into direct contact with the skin.

It is still another object of the present invention to provide a method of stabilizing a photo-unstable UV-filter against the degradative effects of UV irradiation or of enhancing the UV-absorbing properties of a photo-unstable UV-absorbing compound.

The problem is solved by the subject matter of the independent patent claims. Further aspects of the present invention are however also apparent from the wording of the dependent patent claims, the following detailed description in conjunction with the accompanying examples and figures.

### SUMMARY OF THE INVENTION

In order to accomplish the above problem, the present invention provides in a first aspect a sunscreen product, a cosmetic or pharmaceutical preparation or homecare product, comprising or consisting of
(a) at least one photo-unstable UV-filter; and
(b) at least one mycosporine-like amino acid compound,
   represented either by the general formula (V)

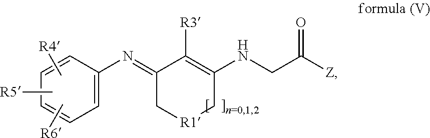

as defined herein, or a tautomer or a stereoisomer or a salt thereof;
or
represented by the general formula (VI)

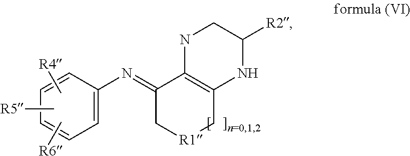

as defined herein, or a tautomer or a stereoisomer or a salt thereof;
or any mixture of the afore-mentioned compounds.

In a second aspect, the present invention provides for the use of at least one mycosporine-like amino acid compound as defined herein or a mixture thereof for stabilizing the photostability of a photo-unstable UV-filter.

Finally, the present invention relates in a further aspect to a method of stabilizing a photo-unstable UV-filter against the degradative effects of UV irradiation or of enhancing the UV-absorbing properties of a photo-unstable UV-absorbing compound comprising admixing the photo-unstable UV-filter with an effective amount of at least one mycosporine-like amino acid or compound as defined herein or a mixture thereof.

The present invention is specified in the appended claims. The invention itself, and its preferred variants, other objects and advantages, are however also apparent from the following detailed description in conjunction with the accompanying examples and figures.

### DESCRIPTION OF FIGURES

FIG. 1 are absorption spectra showing the photostability E1%/cm measurement of MAA-1 (mycosporine-like amino acid compound)

FIG. 2 are absorption spectra showing the photostability E1%/cm measurement of MAA-13 (mycosporine-like amino acid compound)

FIG. 3 are absorption spectra showing the photostability E1%/cm measurement of MAA-20 (mycosporine-like amino acid compound)

FIG. 4 are absorption spectra showing the photostability E1%/cm measurement of MAA-28 (mycosporine-like amino acid compound)

FIG. 5 are absorption spectra showing the photostability E1%/cm measurement of Ethylhexyl Methoxycinnamate (UV-filter)

FIG. 6 are absorption spectra showing the photostability E1%/cm measurement of Butyl Methoxydibenzoylmethane (UV-filter)

FIG. 7 are absorption spectra showing the photostability E1%/cm measurement of Isoamyl p-Methoxycinnamate (UV-filter)

FIG. 8 are absorption spectra showing the photostability E1%/cm measurement of a mixture of MAA-1 and Ethylhexyl Methoxycinnamate

FIG. 9 are absorption spectra showing the photostability E1%/cm measurement of a mixture of MAA-1 and Butyl Methoxydibenzoylmethane

FIG. 10 are absorption spectra showing the photostability E1%/cm measurement of a mixture of MAA-1 and Isoamyl p-Methoxycinnamate

FIG. 11 are absorption spectra showing the photostability E1%/cm measurement of a mixture of MAA-13 and Isoamyl p-Methoxycinnamate

FIG. 12 are absorption spectra showing the photostability E1%/cm measurement of a mixture of MAA-13 and Ethylhexyl Methoxycinnamate

FIG. 13 are absorption spectra showing the photostability E1%/cm measurement of a mixture of MAA-20 and Isoamyl p-Methoxycinnamate

FIG. 14 are absorption spectra showing the photostability E1%/cm measurement of a mixture of MAA-20 and Ethylhexyl Methoxycinnamate

FIG. 15 are absorption spectra showing the photostability E1%/cm measurement of a mixture of MAA-28 and Isoamyl p-Methoxycinnamate

FIG. 16 are absorption spectra showing the photostability E1%/cm measurement of a mixture of MAA-28 and Ethylhexyl Methoxycinnamate

FIG. 17 are absorption spectra showing the photostability E1%/cm measurement of a mixture of MAA-1 and MAA-13

FIG. 18 are absorption spectra showing the photostability E1%/cm measurement of a mixture of MAA-20 and MAA-13

### DETAILED DESCRIPTION OF THE INVENTION

In the context of the present invention, the following general meanings apply:

As used herein, the singular form “a”, “a” and “the” include plural references unless the context clearly dictates otherwise. Thus, for example reference to “cosmetic preparation”, “the cosmetic preparation” or “a cosmetic preparation” also includes a plurality of cosmetic preparations.

The terms “comprising”, “including”, and “containing” are to be understood as open-ended terms and mean that the named components following said term are essential but not “limited to”, and other components may be added and are still embraced by the present invention.

The term “consisting of” as used according to the present invention means that the total amount of components (a) and (b) adds up to 100% by weight, based on the total weight of the sunscreen product or cosmetic or pharmaceutical preparation, and signifies that the subject matter is closed-ended and can only include the limitations that are expressly recited.

Whenever reference is made to “comprising” it is intended to cover both meanings as alternatives, that is the meaning can be either “comprising” or “consisting of” unless the context dictates otherwise.

The term “optionally” means that the subsequently described compound may but need not to be present in the composition, and that the description includes variants, where the compound is included or variants, where the compound is absent.

The term “or” or “and/or” is used as a function word to indicate that two words or phrases are to be taken together or separately.

The endpoints of all ranges directed to the same component or property are inclusive and independently combinable.

The term “compound(s)” or “compound(s) of the present invention” refers to all compounds encompassed by the structural formulae (V) and/or formula (VI) and/or formula (VII) and/or formula (XIII) and/or formula (IX) and/or formula (X) and/or formula (A) and their sub-formulae disclosed herein and includes each subgenus and all specific compounds within the formula whose structure is disclosed herein.

The compounds may be identified by either their chemical structure and/or chemical name. When the chemical structure and chemical name are in conflict, the chemical structure determines the identity of the compound.

The term “at least one . . . compound” means that the food or cosmetic or pharmaceutical preparation, in particular sunscreen product, or homecare product according to the present invention can comprise either one of said subsequently described individual compound or a mixture of two, three, four, five, six or even more different of said subsequently compounds.

The term “effective amount of a compound” means the amount of compound, that is sufficient to achieve the desired effect or improvement.

The term “cosmetic or pharmaceutical preparations” in the context of the present invention are compositions for cosmetic or pharmaceutical purposes which contain a UV absorber in order to protect skin or hair against UV-radiation. In addition to providing photo-protection, the cosmetic or pharmaceutical preparations according to the present invention are useful for providing anti-aging benefits to skin, whitening or preventing darkening of skin, improving appearance of skin, diminishing the visible signs of skin aging and improving skin's radiance and firmness.

The term “sunscreen composition” or “sunscreen” or “skin-care product”, also known as “sunblock”, “sun cream” or “suntan lotion”, refers to any topical product, which reflects and/or absorbs certain parts of UV radiation and thus helps protect against sunburn and most importantly prevent skin cancer. Thus, the term “sunscreen composition” is to be understood as not only including sunscreen compositions, but also any cosmetic compositions that provide UV protection. According to the present invention the sunscreen composition may comprise one or more active agents, e.g., organic UV-filters, as well as other ingredients or additives, e.g., emulsifiers, emollients, viscosity regulators, stabilizers, preservatives, or fragrances. Sunscreens come as lotions, sprays, gels, foams (such as an expanded foam lotion or whipped lotion), sticks, powders and other topical products. UV-absorbing compounds are used not only in sunscreen, but also in other personal care products, such as lipstick, shampoo, hair spray, body wash, toilet soap, and insect repellent.

The term “homecare products” in the context of the present invention are the essentials for daily care and cleaning purpose in households. They are used for maintaining hygiene and a good aura of the homes. The home care products generally include laundry detergents (powder, liquid and tablet), fabric conditioners, dishwashing detergents (liquid and tablet), hard floor and surface cleaners, glass cleaners, carpet cleaners, oven cleaners, air fresheners, disinfectants, stain removers, car wash products. These products are usually manufactured in the form of a liquid, powder, spray, granules and others. The sunscreen containing formulations prevent premature photodamage and photobleaching to surfaces and the homecare formulation itself.

The term “light stabilizers” as used herein are compounds suitable for protecting body-care and household cleaning and treating agents against photolytic degradation.

The term “photostability” refers to the ability of a UV-filter or any other molecule, which is exposed to sunlight, to stay stable upon irradiation. In particular, this means that the compound does not undergo a degradation process upon UV radiation.

The term “sun protection factor (SPF)” as used herein indicates how well the skin is protected by a sunscreen composition. In particular, the factor indicates how much longer the protected skin may be exposed to the sun without getting a sunburn in comparison to untreated skin. For example, if a sunscreen composition with an SPF of 15 is evenly applied to the skin, the sunscreen allows the skilled person to stay in the sun 15 times longer. In other words, SPF 15 means that 1/15 of the burning UV radiation will reach the skin, assuming sunscreen is applied evenly at a thick dosage of 2 milligrams per square centimeter (mg/cm²).

The term “critical wavelength” is defined as the wavelength at which the area under the UV protection curve (% protection versus wavelength) represents 90% of the total area under the curve in the UV region (280-400 nm). For example, a critical wavelength of 370 nm indicates that the protection of the sunscreen composition is not limited to the wavelengths of UV-B, i.e. wavelengths from 280-320 nm, but extends to 370 nm in such a way that 90% of the total area under the protective curve in the UV region are reached at 370 nm. The higher the critical wavelength the better the protection against UV radiation.

According to the present invention, the primary object of the present invention is solved by a sunscreen product or cosmetic, especially dermatological, or pharmaceutical preparation, comprising or consisting of
(a) at least one photo-unstable UV-filter; and
(b) at least one mycosporine-like amino acid compound,
   represented either by the general formula (V)

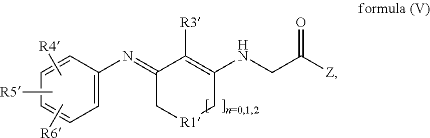

wherein
   Z is selected from the group consisting of O—R2′ or amide′;
   R1′ is selected from the group consisting of CH₂, unsubstituted or substituted CH(alkyl), unsubstituted or substituted C(alkyl)₂, unsubstituted or substituted CH(cycloalkyl), unsubstituted or substituted CH(aryl), unsubstituted or substituted CH(heterocycloalkyl), unsubstituted or substituted CH(heteroaryl), CR7′R8′, C═O, halogen, O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), and unsubstituted or substituted N(heteroaryl), and unsubstituted or substituted C-spirocycles;
   R2′ is selected from the group consisting of H, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted cycloalkyl, unsubstituted or substituted aryl, unsubstituted or substituted heterocycloalkyl, unsubstituted or substituted heteroaryl;
   amide′ is selected from the group consisting of NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), and unsubstituted or substituted NR9′R10′, unsubstituted or substituted —N(O-alkyl)(alkl) (Weinreb amide), —N(OH)(H) (hydroxamate), and unsubstituted or substituted —N(OH)(alkyl) (alkylated hydroxamate);
   R3′ is selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9′R10′, and unsubstituted or substituted C-spirocycles;
   the substituents R4′, R5′ and R6′ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, —S(═O)₂OH, sulfonyl, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), and unsubstituted or substituted NH(heteroaryl), unsubstituted or substituted NR9′R10′, and unsubstituted or substituted C-spirocycles;
   R7′ and R8′ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl) and unsubstituted or substituted NR9′R10′;
   R9′ und R10′ which may be identical or different, are independently from each other selected from the group consisting of H, OH, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, unsubstituted or substituted cycloalkyl, unsubstituted or substituted aryl, unsubstituted or substituted heterocycloalkyl, and unsubstituted or substituted heteroaryl;
   Y is selected from the group consisting of H and unsubstituted or substituted alkyl; and
   R is unsubstituted or substituted alkyl;
or a tautomer or a stereoisomer or a salt of the afore-mentioned compounds;
or represented by the general formula (VI)

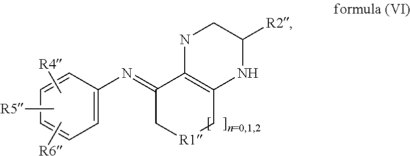

wherein
   R1″ is selected from the group consisting of CH₂, unsubstituted or substituted CH(alkyl), unsubstituted or substituted C(alkyl)₂, unsubstituted or substituted CH(cycloalkyl), unsubstituted or substituted CH(aryl), unsubstituted or substituted CH(heterocycloalkyl), unsubstituted or substituted CH(heteroaryl), —CR7″R8″, C═O, halogen, O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), unsubstituted or substituted N(heteroaryl), and unsubstituted or substituted C-spirocycles;
   R2″ is selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —C(═O)-amide″, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9″R10″, and unsubstituted or substituted C-spirocycles;
   X is selected from the group consisting of CH₂, unsubstituted or substituted CH(alkyl), unsubstituted or substituted CH(alkenyl), unsubstituted or substituted CH(alkynyl), unsubstituted or substituted CH(alkoxy), unsubstituted or substituted CH(alkylthio), unsubstituted or substituted CH(cycloalkyl), unsubstituted or substituted CH(aryl), unsubstituted or substituted CH(heterocycloalkyl), unsubstituted or substituted CH(heteroaryl), —CR7″R8″, C═O, O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), unsubstituted or substituted N(heteroaryl), and unsubstituted or substituted C-spirocycles;
   the substituents R4″, R5″ and R6″ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, —S(═O)₂OH, sulfonyl, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), and unsubstituted or substituted NH(heteroaryl), unsubstituted or substituted NR9″R10″, and unsubstituted or substituted C-spirocycles;
   R7″ and R8″ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl) and unsubstituted or substituted NR9″R10″;
   amide″ is selected from the group consisting of NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9″R10″, unsubstituted or substituted —N(O-alkyl)(alkyl) (Weinreb amide), —N(OH)(H) (hydroxamate), and unsubstituted or substituted —N(OH)(alkyl) (alkylated hydroxamate);
   R9″ und R10″ which may be identical or different, are independently from each other selected from the group consisting of H, OH, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, unsubstituted or substituted cycloalkyl, unsubstituted or substituted aryl, unsubstituted or substituted heterocylcoalkyl, and unsubstituted or substituted heteroaryl;
   Y is selected from the group consisting of H and unsubstituted or substituted alkyl; and
   R is unsubstituted or substituted alkyl;
or a tautomer or a stereoisomer or a salt of the afore-mentioned compounds;
or any mixture of the afore-mentioned compounds.

The component (a) of the sunscreen product or cosmetic or pharmaceutical preparation according to the first aspect of the present invention relates to at least one photo-unstable UV-filter, which is commonly used in sunscreen products or cosmetic or pharmaceutical preparations.

A UV-filter is a compound or a mixture of compounds that block or absorb ultraviolet (UV) light. UV classifications include UVA (320 to 400 nm), UVB (290 to 320 nm) and UVC (200 to 280 nm). UV-absorbing compounds are used not only in sunscreen, but also in other personal care products, such as lipstick, shampoo, hair spray, body wash, toilet soap, and insect repellent. Chemical filters protect against UV-radiation by absorbing, reflecting, or scattering. Reflection and scattering are accomplished by inorganic physical UV-filters, such as titanium dioxide (TiO₂) and zinc oxide (ZnO). Absorption, mainly of UVB, is done by organic UV-filters, which are known as chemical UV-filters. The sunscreen product or cosmetic or pharmaceutical preparations according to the present invention provide broad spectrum photo protection, i.e. protection from both UVA and UVB.

Many different organic compounds can serve as UV-filters. They fall into several structural classes:
(1) Benzophenones
   a. Benzophenone-3 (BP3)
   b. Benzophenone-4 (BP4)
(2) Salicylates
   a. Homosalate (HMS)
   b. 2-ethylhexyl salicylate (EHS)
(3) p-Aminobenzoic acid and derivatives
   a. Ethylhexyl dimethyl PABA (OD-PABA)
   b. 4-p-aminobenzoic acid (PABA)
(4) Benzimidazole derivatives
   a. Phenylbenzimidazole sulfonic acid (PMDSA)
   b. Disodium phenyl dibenzimidazole tetrasulfonate (bisdisulizole disodium)
(5) Triazines
   a. Ethylhexyltriazone (OT)
   b. Diethylhexyl butamido triazone (DBT)
   c. Bis-ethylhexyloxyphenol methoxyphenyl triazine (EMT)
(6) Benzotriazoles
   a. Drometrizole trisiloxane (DRT)
   b. Methylene bis-benzotriazolyl tetramethylbutylphenol (MBP, biscotrizole)
(7) Dibenzoylmethane derivatives
   a. 4-tert-Butyl-4′-methoxydibenzoylmethane (BM-DBM, avobenzone)
(8) Cinnamates
   a. Ethylhexyl methoxycinnamate (OMC)
   b. Isoamyl p-methoxycinnamate (IMC, amiloxate)
(9) Camphor derivatives
   a. Terephtalydene dicamphor sulfonic acid (PDSA)
   b. 3-benzylidene camphor (3BC)
   c. Benzylidene camphor sulfonic acid (BCSA)
   d. 4-methylbenzylidene camphor (4-MBC)
   e. Polyacrylamidomethyl benzylidene camphor (PBC)
(10) Camphor benzalkonium methosulfate (CBM)

The term “photo-unstable UV-filter” in the context of the present invention means a UV-filter which is photo-unstable and does not retain its integrity upon exposure to light. In other words, a photo-unstable UV-filter degrades more or less rapidly upon exposure to light, i.e. loses its essential quality for a sunscreen. This characteristic decreases the UV-filter's ability to protect against UV-radiation. Exposure to UV-radiation dissipates the available concentration of the organic UV-filters in a sunscreen formulation, resulting in a product with decreased efficacy, i.e. UV protective capacity, both in terms of level of protection and time of protection.

The photostability of a UV-filter can be determined in vitro using absorbance measures, by which the loss in absorbance of the UV-filter before and after irradiation, for example with 5 MED, is measured. The loss in absorbance of the UV-filter goes along with the degradation of the UV-filter by one or more photochemical reactions, including photofragmentation, isomerization, tautomerization, photoaddition and the like. UV-filters with an absorbance loss≥20% after irradiation are classified as photo-unstable. Alternatively, the photostability of a UV-filter can be determined by analytical measures, that is by quantitative determination of the amount (i.e. concentration) by which the filter is degraded.

The photo-unstable UV-filter component in the sunscreen product or cosmetic or pharmaceutical preparation according to the first aspect of the present invention is selected from the group consisting of: Ethylhexyl-methoxycinnamate (Neo Heliopan® AV), Butyl-methoxydibenzoylmethane (Avobenzone; Neo Heliopan® 357), Isoamyl-p-Methoxycinnamate (Neo Heliopan® E1000), Diethylamino-hydroxybenzoyl-hexyl-benzoate (Uvinul A plus) and any mixture thereof.

### Ethylhexyl-Methoxycinnamate

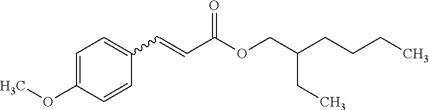 [SMILES: CCCCC(CC)COC(=O)C=Cc1ccc(OC)cc1]

is a UVB filter substance, which provides protection against UVB radiation in a wavelength range of 280 bis 320 nm.

### Butyl-Methoxydibenzoylmethane or Avobenzone

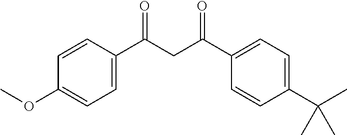 [SMILES: COc1ccc(C(=O)CC(=O)c2ccc(C(C)(C)C)cc2)cc1]

is a Dibenzoylmethane derivative. Avobenzone exists in the ground state as a mixture of the enol and keto forms, favoring the chelated enol. This enol form is stabilized by intramolecular hydrogen-bonding within the P-diketone. Its ability to absorb ultraviolet light over a wider range of wavelengths from 320 to 380 nm than many other sunscreen agents has led to its use in many commercial preparations marketed as broad-spectrum sunscreen. Avobenzone has an absorption maximum of 357 nm. Avobenzone has been shown to degrade significantly in light, resulting in less protection over time. The UVA light in a day of sunlight in a temperate climate is sufficient to break down most of the compound.

### Isoamyl-p-Methoxycinnamate

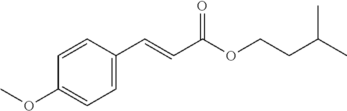 [SMILES: COc1ccc(/C=C/C(=O)OCCC(C)C)cc1]

is a UVB filter substance, which provides protection against UVB radiation in a wavelength range of 280 bis 320 nm.

### Diethylamino-hydroxybenzoyl-hexyl-benzoate

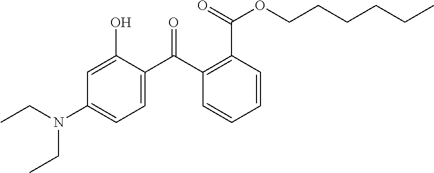 [SMILES: CCCCCCOC(=O)c1ccccc1C(=O)c1ccc(N(CC)CC)cc1O]

is a UV-filter with high absorption in the UVA range in a wavelength range of 320 to 380 nm.

The aforesaid photo-unstable UV-filter is used in the sunscreen, cosmetic or pharmaceutical preparation or homecare product either as a single component or in a mixture with two, three or more further of said photo-unstable UV-filter(s) as specified above.

The component (b) in the sunscreen product or cosmetic or pharmaceutical preparation according to the first aspect of the present invention is at least one mycosporine-like amino acid compound represented either by the general formula (V)

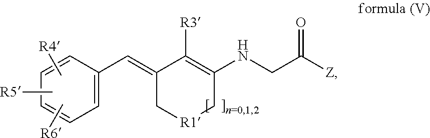

as defined herein;
or represented by the general formula (VI)

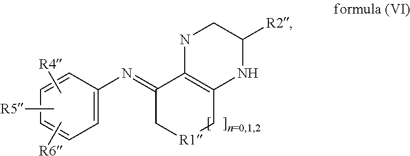

as defined herein.

Mycosporine-like amino acids (MAAs) are small secondary metabolites produced by organisms that live in environments with high volumes of sunlight, usually marine environments. The MAAs are imine derivatives of mycosporines and contain an amino-cyclohexene-imine ring linked to an amino acid, amino alcohol or amino group. The compounds are capable of electron delocalization. In addition, said compounds demonstrate antioxidant qualities.

Unless stated otherwise, in the context of the present invention, especially for the definition of the mycosporine-like amino acid compounds represented by any of the general formulae the following general meanings apply:

The term “halogen” residue/moiety or group alone or as part of another substituent according to the present invention refers to F, Cl, Br or I.

The term “alkyl” alone or as part of another substituent according to the present invention refers to a saturated or mono- or polyunsaturated linear or branched monovalent hydrocarbon radical obtained by removing a hydrogen atom from a single carbon atom of a corresponding parent alkane.

In a preferred variation, the term “alkyl” also includes any alkyl moieties in radicals derived therefrom, such as alkoxy, alkylthio, alkylsulphonyl saturated linear or branched hydrocarbon radicals having 1 to 10, 1 to 8, 1 to 6, or 1 to 4 carbon atoms.

If the alkyl radical is further bonded to another atom, it becomes an alkylene radical or alkylene group. In other words, the term “alkylene” also refers to a divalent linear or branched alkyl. For example, —CH₂CH₃ is an ethyl, while —CH₂CH₂— is an ethylene.

The term “alkylene” alone or as part of another substituent refers to a saturated linear or branched divalent hydrocarbon radical obtained by removing two hydrogen atoms from a single carbon atom or two different carbon atoms of a starting alkane.

In preferred variants according to the present invention, the linear or branched alkyl group or alkylene group comprises 1 to 10 carbon atoms. In other still more preferred variants, the linear or branched alkyl group or alkylene group comprises 1 to 6 carbon atoms.

More preferred according to the invention are saturated linear or branched C1 to C6 alkyl groups or saturated linear or branched C1 to C6 alkylene groups.

Most preferred the linear or branched alkyl groups or alkylene groups with 1 to 4 carbon atoms.

Preferred alkyl radicals/moieties or alkyl groups include, but are not limited to: C1 to C6 alkyl comprising methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

In a preferred variant, the alkyl radical/moiety is methyl, ethyl, propyl, isopropyl, butyl, isobutyl or tert-butyl, more preferred form the group consisting of methyl and ethyl.

The alkyl group or alkylene group as defined above may further be substituted.

The term “alkyl” or “alkylene” further includes radicals or groups having any degree of saturation, i.e., groups having only single carbon-carbon bonds (“alkyl” or “alkylene”), groups having one or more double carbon-carbon bonds (“alkenyl”), radicals having one or more triple carbon-carbon bonds (“alkynyl”), and groups having a mixture of single, double and/or triple carbon-carbon bonds.

The term “alkenyl” alone or as part of another substituent according to the present invention refers to an unsaturated linear or branched monovalent hydrocarbon radical having at least one carbon-carbon double bond (C═C double bond). The radical may be in either the cis or trans conformation around the double bond(s). So that the term “alkenyl” also includes the corresponding cis/trans isomers.

In preferred variants according to the present invention, the linear or branched alkenyl group comprises 2 to 10 carbon atoms. In other preferred variants, the linear or branched alkenyl group comprises 2 to 6 carbon atoms.

In still further preferred variants, the linear or branched alkenyl group comprises 2 to 4 carbon atoms.

Preferred according to the invention are mono- or di-unsaturated linear or branched C1 to C6 alkenyl groups.

Typical alkenyl radicals or alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl, cycloprop-2-en-1-yl; butenyls such as but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl and the like.

The alkenyl group as defined above may further be substituted.

The term “alkynyl” alone or as part of another substituent according to the present invention refers to an unsaturated linear or branched monovalent hydrocarbon radical having at least one carbon-carbon triple bond (C≡C triple bond).

In preferred variants according to the present invention, the linear or branched alkynyl group comprises 2 to 10 carbon atoms. In other preferred variants, the alkynyl group comprises 2 to 6 carbon atoms. In still further preferred variants, the alkynyl group comprises 2 to 4 carbon atoms.

Most preferred according to the invention are mono- or di-unsaturated linear or branched C1 to C6 alkynyl groups.

Typical alkynyl radicals/moieties or alkynyl groups include, but are not limited to, ethynyl; propynyls such as prop-1-yn-1-yl, prop-2-in-1-yl, etc.; butynyls such as but-1-in-1-yl, but-1-in-3-yl, but-3-in-1-yl, and the like.

The alkynyl group as defined above may further be substituted. The alkyl group or alkylene group as defined above may further be substituted.

The term “alkoxy” alone or as part of another substituent according to the present invention refers to a linear or branched radical of the formula —O—R, where R is alkyl or substituted alkyl, as defined herein.

In preferred variants according to the present invention, the linear or branched alkoxy group comprises 2 to 10 carbon atoms. In other preferred variants, the linear or branched alkoxy group comprises 2 to 6 carbon atoms. In still further preferred variants, the linear or branched alkoxy group comprises 2 to 4 carbon atoms.

Most preferred according to the invention are linear or branched C1 to C6 alkoxy groups.

Typical alkoxy radicals/moieties or alkoxy groups include C1 to C6 alkoxy comprising C1 to C4 alkoxy such as. methoxy, ethoxy, n-propoxy, 1-methylethoxy, butoxy, 1-methylpropoxy, 2-methylpropoxy or 1,1-dimethylethoxy; as well as pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy.

In a preferred variant, the alkoxy radical or alkoxy group is methoxy (—O-methyl), ethoxy (—O-ethyl), propoxy (—O-propyl) or butoxy (—O-butyl), more preferred from the group consisting of methoxy (—O-methyl) and ethoxy (—O-ethyl).

The alkoxy group or alkylene group as defined above may further be substituted.

The term “alkylthio” or “thioalkoxy” alone or as part of another substituent according to the present invention refers to a radical of the formula —S—R, wherein R is alkyl or substituted alkyl, as defined herein.

According to the invention, the term “alkyl” or “alkylene” also includes heteroalkyl radicals or heteroalkyl groups. The term “heteroalkyl” by itself or as part of other substituents refers to alkyl groups in which one or more of the carbon atom(s) is/are independently replaced by the same or another heteroatom or by the same or another heteroatomic group(s). Typical heteroatoms or heteroatomic groups that may replace the carbon atoms include, but are not limited to, —O—, —S—, —N—, —Si—, —NH—, —S(O)—, —S(O)₂—, —S(O)NH—, —S(O)₂NH—, and the like, and combinations thereof. The heteroatoms or heteroatomic groups may be located at any internal position of the alkyl group.

Typical heteroatomic groups that may be included in these groups include, but are not limited to, —O—, —S—, —O—O—, —S—S—, —O—S—, —NRR—, =NN=, —N═N—, —N═N—NRR, —PR—, —P(O)₂—, —POR—, —O—P(O)₂—, —SO—, —SO₂—, —SR₂OR—, and the like, wherein R is independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl as defined herein.

The alkyl group or alkylene group as defined above may further be substituted.

The term “acyl” or “alkanoyl” alone or as part of another substituent according to the present invention refers to a radical R—C(═O)—, wherein R is hydrogen, unsubstituted or substituted alkyl, unsubstituted substituted cycloalkyl, unsubstituted or substituted aryl, arylalkyl, unsubstituted or substituted arylalkyl, unsubstituted or substituted heteroalkyl, unsubstituted or substituted heteroarylalkyl, as defined herein.

Representative examples include, but are not limited to, formyl, acetyl, propionyl, butyryl, valeryl, benzoyl, cyclohexylcarbonyl, cyclohexylmethylcarbonyl, benzylcarbonyl, and the like.

The term “cycloalkyl” alone or as part of another substituent according to the present invention refers to a saturated or mono- or polyunsaturated, non-aromatic, cyclic monovalent hydrocarbon radical in which the carbon atoms are ring-linked and which has no heteroatom.

The carbon ring can occur as a monocyclic compound, which has only a single ring, or as a polycyclic compound, which has two or more rings.

In one preferred variation, the term “cycloalkyl” includes a three- to ten-membered monocyclic cycloalkyl radical or cycloalkyl group or a nine- to twelve-membered polycyclic cycloalkyl radical or cycloalkyl group. In other still more preferred variants, the cycloalkyl moiety comprises a five-, six- or seven-membered monocyclic cycloalkyl moiety or a nine- to twelve-membered bicyclic cycloalkyl moiety.

In a preferred embodiment according to the present invention, a cycloalkyl radical or group comprises 3 to 20 carbon atoms. In an even more preferred embodiment, a cycloalkyl radical comprises 6 to 15 carbon atoms. In a most preferred embodiment, a cycloalkyl radical comprises 6 to 10 carbon atoms. Most preferred are monocyclic C3 to C7 cycloalkyl groups.

Typical cycloalkyl radicals or cycloalkyl groups include, but are not limited to, saturated carbocyclic radicals having 3 to 20 carbon atoms, such as C3 to C12 carbocyclyl, comprising cyclopropyl, cyclobutyl, cyclopentyl, cyclohexyl, cycloheptyl, cyclooctyl, cyclononyl, cyclodecyl, cycloundecyl, and cyclododecyl; cyclopentyl, cyclohexyl, cycloheptyl, as well as cyclopropyl-methyl, cyclopropyl-ethyl, cyclobutyl-methyl, cyclobutyl-ethyl, cyclopentyl-methyl, cyclopentyl-ethyl, cyclohexyl-methyl, or C3- to C7-carbocyclyl comprising cyclopropyl, cyclobutyl, cyclopentyl, cyclohexyl, cycloheptyl, cyclopropyl-methyl, cyclopropyl-ethyl, cyclobutyl-methyl, cyclopentyl-ethyl, cyclohexyl-methyl, cyclobut-1-en-1-yl, cyclobut-1-en-3-yl, 3-dien-1-yl and the like.

Polycyclic cycloalkyl radicals or cycloalkyl groups preferred according to the invention include but are not limited to naphthyl, indenyl, groups and the like.

According to the invention, the term “cycloalkyl” also includes cycloalkenyls, i.e. unsaturated cyclic hydrocarbon radicals containing C═C double bonds between two carbon atoms of the ring molecule. In a broader sense, cycloalkenyls are compounds with one, two or more double bond(s), where the number of possible, mostly conjugated double bonds in the molecule depends on the ring size.

Typical cycloalkenyls include, but are not limited to, cyclopropenyl, cyclopentenyl, cyclohexenyl, cyclopentadienyl, and the like.

According to the invention, the term “cycloalkyl” further includes cycloalkynyls, i.e. unsaturated, —C≡C-triple bonds, containing cyclic hydrocarbon radicals between two carbon atoms of the ring molecule, the triple bond depending on the ring size for reasons of ring tension.

Typical cycloalkynyles include cyclooctin.

The cycloalkyl, cycloalkenyl or cycloalkynyl moieties or groups, as defined above, may further be substituted.

The term “aryl” alone or as part of another substituent according to the present invention refers to a monovalent aromatic hydrocarbon radical derived by removing a hydrogen atom from a single carbon atom of an aromatic ring SY-stem.

In one preferred variation, the term “aryl” includes a three- to ten-membered monocyclic aryl radical or aryl group or a nine- to twelve-membered polycyclic aryl radical or aryl group. In other still more preferred variants, the carboaryl radical comprises a five-six- or seven-membered monocyclic carboaryl radical or a nine- to twelve-membered bicyclic carboaryl radical.

In a preferred embodiment according to the present invention, the aryl radical comprises 3 to 20 carbon atoms. In a preferred variation, the aryl moiety comprises 6 to 15 ring atoms. In an even more preferred embodiment, an aryl radical comprises 6 to 10 carbon atoms.

Most preferred according to the invention are monocyclic C3 to C10 aryl groups. Most preferred are monocyclic C3 to C7 aryl groups.

Typical aryl radicals include, without being limited thereto, benzene, phenyl, biphenyl, naphthyl such as 1- or 2-naphthyl, tetrahydronaphthyl, fluorenyl, indenyl, and phenanthrenyl. Typical carboaryl moieties further include, but are not limited to, groups derived from aceanthrylene, acenaphthylene, acephenanthrylene, anthracene, azulene, benzene, chrysene, corone, fluoranthene, fluorene, hexacene, hexaphene, hexalene, as-indacene, S-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

Aromatic polycyclic aryl radicals or aryl groups preferred according to the invention include, but are not limited to, naphthalene, biphenyl and the like.

The aryl moiety or group, as defined above, may further be substituted.

The term “arylalkyl” alone or as part of another substituent according to the present invention refers to an acyclic alkyl group in which one of the hydrogen atoms attached to a carbon atom, typically a terminal or sp carbon atom, is replaced by an aryl group as defined herein. In other words, arylalkyl may also be considered as alkyl substituted by aryl. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like.

The term “heteroarylalkyl” alone or as part of another substituent refers to a cyclic alkyl group in which one of the hydrogen atoms attached to a carbon atom is replaced by a heteroaryl group. In a preferred embodiment according to the present invention, the heteroarylalkyl group is a 6- to 20-membered heteroarylalkyl, e.g., the alkyl, alkenyl, or alkynyl group of the heteroarylalkyl is a C1- to C6-alkyl and the heteroaryl group is a 5- to 15-membered heteroaryl group. In other embodiments, the heteroarylalkyl is a 6- to 13-membered heteroarylalkyl, e.g., the alkyl, alkenyl, or alkynyl group is a C1- to C3-alkyl and the heteroaryl group is a 5 to 10-membered heteroaryl.

The term “heterocycloalkyl” alone or as part of another substituent according to the present invention refers to a saturated or mono- or polyunsaturated, non-aromatic, cyclic monovalent hydrocarbon radical in which one or more carbon atom(s) is/are independently replaced by the same or a different heteroatom. Typical heteroatoms to replace the carbon atom(s) include, but are not limited to, N, P, O, S, Si, etc. Typical heterocycloalkyl groups include, without being limited thereto, groups derived from epoxides, azirines, thiiranes, imidazolidine, morpholine, piperazine, piperidine, pyrazolidine, pyrrolidone, quinuclidine and the like.

In a preferred embodiment according to the present invention, the heterocycloalkyl moiety or group comprises 3 to 20 ring atoms. In a preferred variation, the heterocycloalkyl moiety comprises 6 to 15 ring atoms. In an even more preferred embodiment, the heterocycloalkyl moiety comprises 6 to 10 carbon atoms.

The heterocycloalkyl moiety can occur as a monocyclic compound, which has only a single ring, or as a polycyclic compound, which has two or more rings, such as bicyclic, tricyclic or spirocyclic.

Preferably, the term “heterocycloalkyl” includes three- to seven-membered, saturated or mono- or polyunsaturated heterocycloalkyl moieties comprising one, two, three or four heteroatoms selected from the group consisting of O, N and S. The heteroatom or heteroatoms may occupy any position in the heterocycloalkyl ring.

In one preferred variation, the term “heterocycloalkyl” includes a three- to ten-membered monocyclic heterocycloalkyl radical or a nine- to twelve-membered polycyclic heterocycloalkyl radical. In other still more preferred variants, the heterocycloalkyl moiety comprises a five-, six- or seven-membered monocyclic heterocycloalkyl moiety or a nine- to twelve-membered bicyclic heterocycloalkyl moiety.

Most preferred according to the invention are monocyclic heterocycloalkyl radicals comprising 3 to 12 carbon atoms. Most preferred are monocyclic heterocycloalkyl radicals having 5 to 7 ring atoms.

Typical heterocycloalkyl moieties include, but are not limited to: Five- or six-membered, saturated or monounsaturated heterocycloalkyl containing one or two nitrogen atoms and/or one oxygen or sulphur atom or one or two oxygen and/or sulphur atoms as ring members comprising 2-tetrahydrofuranyl, 3-tetrahydrofuranyl, 2-tetrahydrothienyl, 3-tetrahydrothienyl, 1-pyrrolidinyl, 2-pyrrolidinyl, 3-pyrrolidinyl, 3-Isoxazolidinyl, 4-Isoxazolidinyl, 5-Isoxazolidinyl, 3-Isothiazolidinyl, 4-Isothiazolidinyl, 5-Isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-Imidazolidinyl, 4-Imidazolidinyl, 2-pyrrolin-2-yl, 2-pyrrolin-3-yl, 3-pyrrolin-2-yl, 3-pyrrolin-3-yl, 1-piperidinyl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl and the like.

The heterocycloalkyl moiety or group, as defined above, may further be substituted.

The term “heteroaryl” by itself or as part of another substituent according to the present invention refers to a monovalent heteroaromatic radical obtained by removing a hydrogen atom from a single atom of a heteroaromatic ring SY-stem. Typical heteroaryl radicals, or. Heteroaryl groups include, but are not limited to, those derived from acridine, β-carboline, chroman, chromium, cinnoline, furan, imidazole, indazole, indole, indoline, indolizine, isobenzofuran, isochromium, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, thiazole, thiophene, triazole, xanthene and the like are derived.

The heteroaryl moiety can occur as a monocyclic compound having only a single ring or as a polycyclic compound having two or more rings.

In one preferred variation, the term “heteroaryl” includes a three- to ten-membered monocyclic heteroaryl radical or a nine- to twelve-membered polycyclic heteroaryl radical. In other still more preferred variants, the heteroaryl moiety comprises a five-, six- or seven-membered monocyclic heteroaryl moiety or a nine- to twelve-membered bicyclic heteroaryl moiety.

Preferably, the term “heteroaryl” includes three- to seven-membered monocyclic heteroaryl radicals comprising one, two, three or four heteroatoms selected from the group consisting of O, N and S. The heteroatom or heteroatoms may occupy any position in the heteroaryl ring.

In a preferred embodiment according to the present invention, the heteroaryl moiety or group comprises 3 to 20 ring atoms. In an even more preferred variant, the heteroaryl moiety comprises 6 to 15 ring atoms. In a most preferred embodiment, the heteroaryl group comprises 6 to 10 ring atoms. Most preferred according to the invention are monocyclic C3 to C7 heteroaryl groups.

Particularly preferred heteroaryl moieties or heteroaryl groups include, but are not limited to, those derived from furan, thiophene, pyrrole, benzothiophene, benzofuran, benzimidazole, indole, pyridine, pyrazole, quinoline, imidazole, oxazole, isoxazole, and pyrazine.

Five-membered aromatic heteroaryl radicals containing, in addition to carbon atoms, one, two or three nitrogen atoms or one or two nitrogen atoms and one sulfur or oxygen atom as ring atoms include 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-Imidazolyl, 4-Imidazolyl, and 1,3,4-triazol-2-yl.

Five-membered aromatic heteroaryl radicals containing one, two, three or four nitrogen atoms as ring atoms include 1-, 2- or 3-pyrrolyl, 1-, 3- or 4-pyrazolyl, 1-, 2- or 4-Imidazolyl, 1,2,3-[1H]-triazol-1-yl, 1,2,3-[2H]-triazol-2-yl, 1,2,3-[1H]-triazol-4-yl, 1,2,3-[1H]-triazol-5-yl, 1,2,3-[2H]-triazol-4-yl, 1,2,4-[1H]-triazol-1-yl, 1,2,4-[1H]-triazol-3-yl, 1,2,4-[1H]-triazol-5-yl, 1,2,4-[4H]-triazol-4-yl, 1,2,4-[4H]-triazol-3-yl, [1H]-tetrazol-1-yl, [1H]-tetrazol-5-yl, [2H]-tetrazol-2-yl, [2H]-tetrazol-5-yl and the like.

Five-membered aromatic heteroaryl radicals containing a heteroatom selected from oxygen or sulphur and optionally one, two or three nitrogen atoms as ring atoms include 2-furyl, 3-furyl, 2-thienyl, 3-thienyl, 3- or 4-Isoxazolyl, 3- or 4-isothiazolyl, 2-, 4- or 5-oxazolyl, 2-, 4- or 5-thiazolyl, 1,2,4-thiadiazol-3-yl, 1,2,4-thiadiazol-5-yl, 1,3,4-thiadiazol-2-yl, 1,2,4-oxadiazol-3-yl, 1,2,4-oxadiazol-5-yl and 1,3,4-oxadiazol-2-yl.

Six-membered heteroaryl radicals containing, in addition to carbon atoms, one or two or one, two or three nitrogen atoms as ring atoms, and comprising, for example. 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,2,4-triazin-3-yl; 1,2,4-triazin-5-yl, 1,2,4-triazin-6-yl and 1,3,5-triazin-2-yl.

The heteroaryl moiety or group, as defined above, may further be substituted.

The term “C-spirocycles” in the context of the present application means compounds that have at least two molecular rings with only one common atom. The simplest spiro compounds are bicyclic (having just two rings) or have a bicyclic portion as part of the larger ring system, in either case with the two rings connected through the defining single common atom. The one common atom connecting the participating rings distinguishes spiro compounds from other bicyclic structures: from isolated ring compounds like biphenyl that have no connecting atoms, from fused ring compounds like decalin having two rings linked by two adjacent atoms, and from bridged ring compounds like norbornane with two rings linked by two non-adjacent atoms. Spiro compounds may be fully carbocyclic (all carbon) or heterocyclic (having one or more non-carbon atom, such as N, O and S). The common atom that connects the two (or sometimes three) rings is called the spiro atom. Preferably, the spiro atom is a carbon atom. More preferably, the C-spirocycles compound means compounds that are fully carbocyclic (all carbon).

The term “substituted” in the context of the present invention means that one or more hydrogen atoms of the indicated radical or group is/are independently replaced by the same or a different substituent(s). Additionally, the term “substituted” specifically provides for one or more, i.e., two, three, or even more, substitutions commonly used in the art. However, it is generally known that the substituents should be selected so that they do not adversely affect the useful properties of the compound or its function.

Suitable substituents in the context of the present invention preferably include halogen, perfluoroalkyl groups, perfluoroalkoxy groups, alkyl groups, alkenyl groups, alkynyl groups, hydroxy groups, oxo groups, mercapto groups, alkylthio groups, alkoxy groups, aryl or heteroaryl groups, aryloxy groups or heteroaryloxy groups, arylalkyl or heteroarylalkyl groups, arylalkoxy or heteroarylalkoxy groups, amino groups, alkyl and dialkylamino groups, carbamoyl groups, alkylcarbonyl groups, carboxyl groups, alkoxycarbonyl groups, alkylaminocarbonyl groups, dialkylaminocarbonyl groups, arylcarbonyl groups, aryloxycarbonyl groups, alkylsulfonyl groups, arylsulfonyl groups, cycloalkyl groups, cyano groups, C1 to C6 alkylthio groups, arylthio groups, nitro groups, keto groups, acyl groups, boronate or boronyl groups, phosphate or phosphonyl groups, sulfamyl groups, sulfonyl groups, sulfinyl groups, and combinations thereof.

Substituents or substituent groups useful for substituting saturated carbon atoms in the indicated group or radical more preferably include, but are not limited to, halogen, hydroxyl, alkyl, alkenyl, alkynyl, alkoxyl, —NH₂, amino (primary, secondary, or tertiary), nitro, thiol, thioether, imine, cyano, amido, phosphonato, phosphine, carboxyl, thiocarbonyl, sulfonyl, sulfonamide, ketone, aldehyde, ester, acetyl, acetoxy, carbamoyl, oxygen (O); haloalkyl (e.g., trifluoromethyl); aminoacyl and aminoalkyl, carbocyclic cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g. pyrrolidinyl, piperidinyl, piperazinyl, morpholinyl, or thiazinyl), carbocyclic or heterocyclic, monocyclic or fused or non-fused polycyclic aryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl), —CO₂CH₃, —CONH₂, —OCH₂CONH₂; —SO₂NH₂, —OCHF₂, —CF₃, —OCF₃.

According to the invention, the substituents used to replace a particular radical or radical may in turn be further substituted, typically with one or more of the same or different radicals selected from the various groups indicated above and as defined in detail above. In the case of substituted combinations such as substituted arylalkyl, either the aryl or the alkyl group may be substituted, or both the aryl and the alkyl group may be substituted with one or more substituents.

In a preferred variant, Z in the general formula (V) is either —O—R2′ or amide′.

In a further preferred variant, R1′ in the general formula (V) is CH₂.

In a further preferred variant, R1′ in the general formula (V) is C(alkyl)₂.

In a further preferred variant, R1′ in the general formula (V) is O.

In a further preferred variant, R1′ in the general formula (V) is S.

In a further preferred variant, R1′ in the general formula (V) is SO.

In a further preferred variant, R1′ in the general formula (V) is SO₂.

In a further preferred variant, R1′ in the general formula (V) is NH.

In a still further preferred variant, R1′ in the general formula (V) is N (alkyl).

Most preferred the R1′ in the general formula (V) is either CH₂ or C (alkyl)₂.

In a preferred variant, R2′ in the general formula (V) is H.

In a further preferred variant, R2′ in the general formula (V) is methyl.

In a further preferred variant, R2′ in the general formula (V) is ethyl.

In a further preferred variant, R2′ in the general formula (V) is propyl.

In a further preferred variant, R2′ in the general formula (V) is isopropyl.

In a further preferred variant, R2′ in the general formula (V) is butyl.

In a further preferred variant, R2′ in the general formula (V) is isobutyl.

In a further preferred variant, R2′ in the general formula (V) is tert-butyl.

In a further preferred variant, R2′ in the general formula (V) is

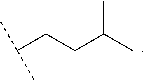

In a further preferred variant, R2′ in the general formula (V) is

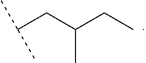

In a preferred variant, R2′ in the general formula (V) is

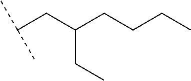

(2-ethyl-hexyl).

In a still further preferred variant, R2′ in the general formula (V) is phenyl.

Most preferred the R2′ in the general formula (V) is either H or ethyl or isopropyl or 2-ethylhexyl or phenyl.

In a preferred variant, amide′ in the general formula (V) is NH₂.

In a further preferred variant, amide′ in the general formula (V) is NH(alkyl), wherein alkyl is selected from the group consisting of methyl, ethyl, propyl, isopropyl, butyl, isobutyl, and tert-butyl.

In a further preferred variant, amide′ in the general formula (V) is N(alkyl)₂, wherein alkyl is selected from the group consisting of methyl, ethyl, propyl, isopropyl, butyl, isobutyl, and tert-butyl.

In a further preferred variant, amide′ in the general formula (V) is —N(O-alkyl)(alkyl) (Weinreb amide), wherein alkyl is selected from the group consisting of methyl, ethyl, propyl, isopropyl, butyl, isobutyl, and tert-butyl.

In a still further preferred variant, amide′ in the general formula (V) is —N(OH)(H) (hydroxamate).

Most preferred the amide′ in the general formula (V) is NH₂, N(methyl)₂ or N(ethyl)₂ or —N(O-alkyl)(alkyl) (Weinreb amide), wherein alkyl is selected from the group consisting of methyl, ethyl, propyl, isopropyl, butyl, isobutyl, and tert-butyl, or —N(OH)(H) (hydroxamate).

In a preferred variant, R3′ in the general formula (V) is H.

In a further preferred variant, R3′ in the general formula (V) is methyl.

In a further preferred variant, R3′ in the general formula (V) is ethyl.

In a further preferred variant, R3′ in the general formula (V) is —O-methyl.

In a further preferred variant, R3′ in the general formula (V) is —C(═O)—OH.

In a further preferred variant, R3′ in the general formula (V) is —C(═O)—CH₃.

In a further preferred variant, R3′ in the general formula (V) is —C(═O)—C₃H₇.

In a still further preferred variant, R3′ in the general formula (V) is —C(═O)—O-methyl.

Most preferred the R3′ in the general formula (V) is either H or methyl or —O— methyl.

In a preferred variant according to the first aspect of the present invention, the mycosporine-like amino acid compound (b) of the general formula (V) is either an acid compound or an ester compound represented by the general formula (VII)

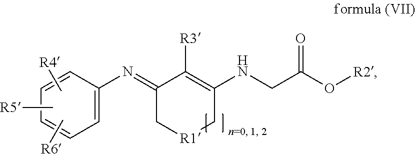

wherein R1′, R2′, R3′, R4′, R5′ and R6′ have the same meaning as defined herein for the general formula (V).

In a preferred variant according to the first aspect of the present invention, the mycosporine-like amino acid compound (b) of general the formula (V) is an acid compound represented by the general formula (VII-acid)

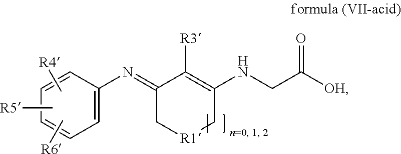

wherein R1′, R3′, R4′, R5′ and R6′ have the same meaning as defined herein for the general formula (V).

The mycosporine-like amino acid compounds according to the general formula (VII-acid) are particularly photo-stable and temperature-stable compounds and show stability in pH solution or in different emulsion types and do not degrade.

Especially, the mycosporine-like amino acid compounds as defined herein have a better pH stability in a broader pH-range compared to their respective ester compounds which hydrolize, and, thus, are not stable in alkaline solutions having a pH higher than 9.

In addition, said mycosporine-like amino acid compounds have also a better solubility in water compared to their corresponding ester compounds. Due to their better solubility in water, said mycosporine-like amino acid compounds can be incorporated into the water-phase of formulations in higher concentrations, resulting in a better photostabilizing effect and, thus, an improved sunscreen in sunscreen products, cosmetic or pharmaceutical preparations or homecare products.

In a preferred variant according to the first aspect of the present invention, the mycosporine-like amino acid compound (b) of the general formula (V) is an ester compound represented by the general formula (VII-ester)

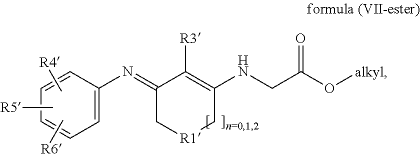

wherein R1′, R3′, R4′, R5′ and R6′ have the same meaning as defined herein for the general formula (V).

Preferably, the alkyl residue in the general formula (VII-ester) is methyl or ethyl or propyl or isopropyl or butyl or isobutyl, or tert-butyl. Most preferred the alkyl residue in the general formula (VII-ester) is methyl. Such ester compounds are particularly stable.

In an alternative preferred variant according to the first aspect of the present invention, the mycosporine-like amino acid compound (b) of the general formula (V) is an amide compound represented by the general formula (VIII)

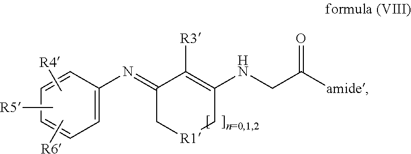

wherein R1′, amide′, R3′, R4′, R5′ and R6′ have the same meaning as defined herein for the general formula (V).

In a more preferred variant according to the first aspect of the present invention, the mycosporine-like amino acid compound (b) of the general formula (V) is an amide compound represented by the general formula (VIII-amide)

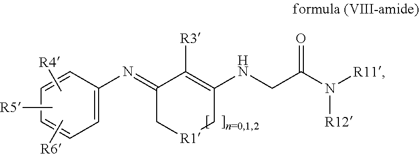

wherein R1′, R3′, R4′, R5′ and R6′ have the same meaning as defined herein for the general formula (V) and R11′ and R12′ are independently from each other selected from the group consisting of H, OH and alkyl.

In a preferred variant according to the first aspect of the present invention, in the general formula (VII-amide), R11′ and/or R12′ are H.

In a further preferred variant, the R11′ alkyl and/or R12′ alkyl in the general formula (VIII-amide) are independently from each other selected from the group consisting of methyl, ethyl, propyl, isopropyl, butyl, isobutyl, and tert-butyl.

Preferably, R11′ and/or R12′ in the general formula (VIII-amide) are each H or each methyl.

In a still more preferred variant, the mycosporine-like amino acid compound (b) of the general formula (V) is a Weinreb amide compound represented by the general formula (VIII-Weinreb amide)

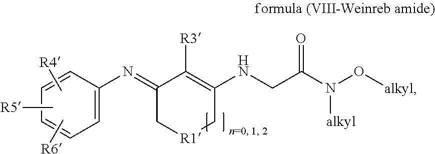

wherein R1′, R3′, R4′, R5′ and R6′ have the same meaning as defined herein for the general formula (V).

In a preferred variant, the alkyl residue in the general formula (VIII-Weinreb amide) is selected from the group consisting of methyl, ethyl, propyl, isopropyl, butyl, isobutyl and tert-butyl, more preferred the alkyl residue is methyl.

Alternatively, in a still more preferred variant, the mycosporine-like amino acid compound (b) of the general formula (V) is a hydroxamate compound represented by the general formula (VII-hydroxamate derivative)

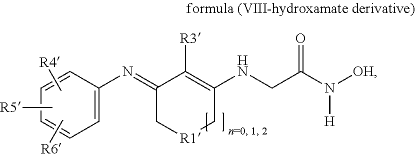

wherein R1′, R3′, R4′, R5′ and R6′ have the same meaning as defined herein for the general formula (V).

Alternatively, in a still further preferred variant, the amide compound (VIII-amide) the mycosporine-like amino acid compound (b) of the general formula (V) is an alkylated hydroxamate compound represented by the general formula (VIII-alkylated hydroxamate derivative)

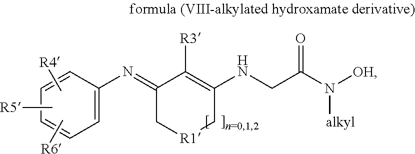

wherein R1′, R3′, R4′, R5′ and R6′ have the same meaning as defined herein for the general formula (V).

In a preferred variant, the alkyl residue in the general formula (VIII-alkylated hydroxamate derivative) is selected from the group consisting of methyl, ethyl, propyl, isopropyl, butyl, isobutyl, and tert-butyl, more preferred the alkyl is methyl.

In a preferred alternatively variant the mycosporine-like amino acid compound (b) is an alcohol compound represented by the general formula (A)

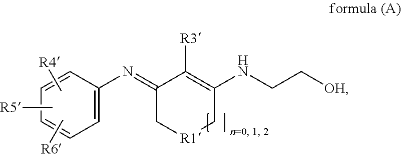

wherein R1′, R3′, R4′, R5′ and R6′ have the same meaning as defined herein for the general the formula (V).

Within the present invention, the same preferred variants as defined herein for the mycosporine-like amino acid compounds according to the general formula (V) apply analogously for the mycosporine-like amino acid compounds according to the general formulae (VII), (VIII) and their variants or general formula (A).

In a further preferred variant, R1″ in the general formula (VI) is CH₂.

In a further preferred variant, R1″ in the general formula (VI) is C(alkyl)₂.

In a further preferred variant, R1″ in the general formula (VI) is O.

In a further preferred variant, R1″ in the general formula (VI) is S.

In a further preferred variant, R1″ in the general formula (VI) is SO.

In a further preferred variant, R1″ in the general formula (VI) is SO₂.

In a further preferred variant, R1″ in the general formula (VI) is NH.

In a still further preferred variant, R1″ in the general formula (VI) is N(alkyl).

Most preferred the R1″ in the general formula (VI) is either CH₂ or C(alkyl)₂.

In a preferred variant, R2″ in the general formula (VI) is —CH₂—OH.

In a further preferred variant, R2″ in the general formula (VI) is —C(═O)—O—Y.

In a further preferred variant, R2″ in the general formula (VI) is —C(═O)-amide″.

Most preferred the R2″ in the general formula (VI) is —C(═O)—O—Y or —C(═O)-amide″.

In a further preferred variant, X in the general formula (VI) is CH₂.

In a further preferred variant, X in the general formula (VI) is C═O.

In a further preferred variant, X in the general formula (VI) is O.

In a further preferred variant, X in the general formula (VI) is S.

In a further preferred variant, X in the general formula (VI) is SO.

In a further preferred variant, X in the general formula (VI) is SO₂.

In a further preferred variant, X in the general formula (VI) is NH.

In a still further preferred variant, X in the general formula (VI) is N(alkyl), wherein alkyl is selected from the group consisting of methyl, ethyl, propyl, isopropyl, butyl, isobutyl, and tert-butyl.

Most preferred the X in the general formula (VI) is either S, SO or SO₂.

In a preferred variant, Y in the general formula (VI) is H.

In a further preferred variant, Y in the general formula (VI) is methyl.

In a further preferred variant, Y in the general formula (VI) is ethyl.

In a further preferred variant, Y in the general formula (VI) is propyl.

In a further preferred variant, Y in the general formula (VI) is isopropyl.

In a further preferred variant, Y in the general formula (VI) is butyl.

In a further preferred variant, Y in the general formula (VI) is isobutyl.

In a further preferred variant, Y in the general formula (VI) is tert-butyl.

In a further preferred variant, Y in the general formula (VI) is

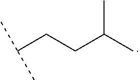

In a further preferred variant, Y in the general formula (VI) is

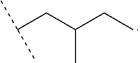

In a preferred variant, Y in the general formula (VI) is

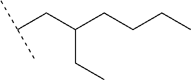

(2-ethyl-hexyl).

In a still further preferred variant, Y in the general formula (VI) is phenyl.

Most preferred the Y in the general formula (VI) is H or ethyl or isopropyl or 2-ethylhexyl or phenyl.

In a preferred variant, amide″ in the general formula (VI) is NH₂.

In a further preferred variant, amide″ in the general formula (VI) is NH(alkyl), wherein alkyl is selected from the group consisting of methyl, ethyl, propyl, isopropyl, butyl, isobutyl, and tert-butyl.

In a further preferred variant, amide″ in the general formula (VI) is N(alkyl)₂, wherein alkyl is selected from the group consisting of methyl, ethyl, propyl, isopropyl, butyl, isobutyl, and tert-butyl.

In a further preferred variant, amide″ in the general formula (VI) is —N(O-alkyl)(alkyl) (Weinreb amide), wherein alkyl is selected from the group consisting of methyl, ethyl, propyl, isopropyl, butyl, isobutyl, and tert-butyl.

In a still further preferred variant, amide″ in the general formula (V) is —N(OH)(H) (hydroxamate).

Most preferred the amide″ in the general formula (V) is NH₂, N(methyl)₂ or N(ethyl)₂ or —N(O-alkyl)(alkyl) (Weinreb amide), wherein alkyl is selected from the group consisting of methyl, ethyl, propyl, isopropyl, butyl, isobutyl, and tert-butyl, or —N(OH)(H) (hydroxamate).

In a more preferred variant according to the first aspect of the present invention, the mycosporine-like amino acid compound (b) is an alcohol compound represented by the general formula (IX-alcohol)

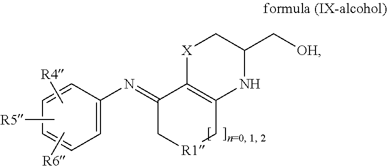

wherein R1″, X, R4″, R5″ and R6″ have the same meaning as defined herein for the general formula (VI).

Alternatively, in a more preferred variant according to the first aspect of the present invention, the mycosporine-like amino acid compound (b) of the general formula (VI) is an acid compound represented by the general formula (IX-acid)

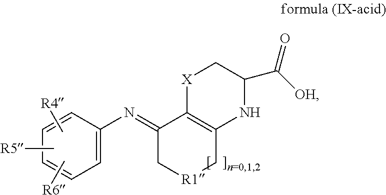

wherein R1″, X, R4″, R5″ and R6″ have the same meaning as defined herein for the general formula (VI).

Alternatively, in a more preferred variant according to the first aspect of the present invention, the mycosporine-like amino acid compound (b) of the general formula (VI) is an ester compound represented by the general formula (VII-ester)

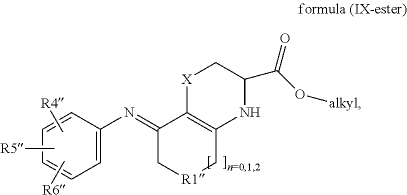

wherein R1″, X, R4″, R5″ and R6″ have the same meaning as defined herein for the general formula (VI).

Preferably, the alkyl residue in the general formula (VII-ester) is methyl or ethyl or propyl or isopropyl or butyl or isobutyl or tert-butyl, more preferred the alkyl residue is methyl.

In an alternative more preferred variant according to the first aspect of the present invention, the mycosporine-like amino acid compound (b) of the general formula (VI) is an amide compound represented by the general formula (X)

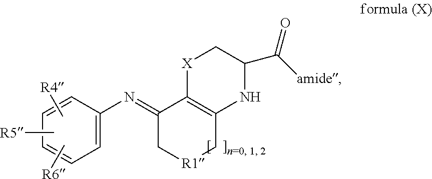

wherein R1″, amide″, X, R4″, R5″ and R6″ have the same meaning as defined herein for the general formula (VI).

In a more preferred alternatively variant according to the first aspect of the present invention, the mycosporine-like amino acid compound (b) of the general formula (VI) is an amide compound represented by the general formula (X-amide)

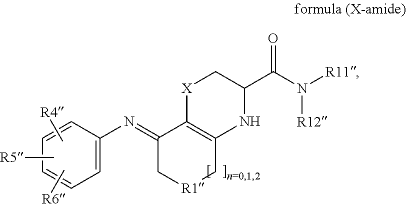

wherein R1″, X, R4″, R5″ and R6″ have the same meaning as defined herein for the general formula (VI) and R11″ and R12″ are independently from each other selected from the group consisting of H, OH and alkyl.

In a preferred variant according to the first aspect of the present invention, in the general formula (X-amide), R11″ and/or R12″ are H.

Preferably, the R11″ alkyl and/or R12″ alkyl in the general formula (X-amide) is methyl or ethyl or propyl or isopropyl, or butyl, or isobutyl or tert-butyl.

Preferably, R11″ and/or R12″ in the general formula (X-amide) are each H or each methyl.

In a still more preferred variant, the mycosporine-like amino acid compound (b) of the general formula (VI) is a Weinreb amide compound represented by the general formula (X-Weinreb amide)

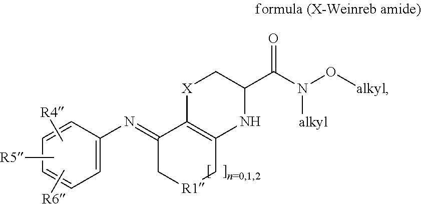

wherein R1″, X, R4″, R5″ and R6″ have the same meaning as defined herein for the general formula (VI).

In a preferred variant, the alkyl residue in the general formula (X-Weinreb amide) is selected from the group consisting of methyl, ethyl, propyl, isopropyl, butyl, isobutyl, and tert-butyl, more preferred the alkyl residue is methyl.

Alternatively, in a still more preferred variant, the mycosporine-like amino acid compound (b) of the general formula (VI) is a hydroxamate compound represented by the general formula (X-hydroxamate derivative)

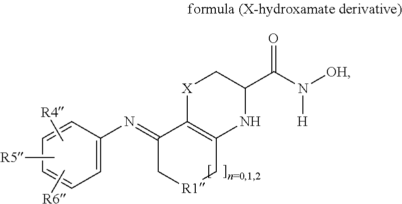

wherein R1″, X, R4″, R5″ and R6″ have the same meaning as defined herein for the general formula (VI).

Alternatively, in a still further preferred variant, the mycosporine-like amino acid compound (b) of the general formula (VI) is an alkylated hydroxamate compound represented by the general formula (VIII-alkylated hydroxamate derivative)

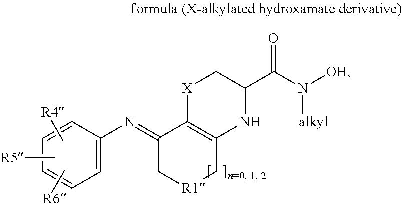

wherein R1″, X, R4″, R5″ and R6″ have the same meaning as defined herein for the general formula (VI).

In a preferred variant, the alkyl residue in the general formula (X-alkylated hydroxamate derivative) is selected from the group consisting of methyl, ethyl, propyl, isopropyl, butyl, isobutyl, and butyl, more preferred the alkyl residue is methyl.

Within the present invention, the same preferred variants as defined herein for for the mycosporine-like amino acid compounds according to the general formula (VI) apply analogously for the mycosporine-like amino acid compounds according to the general formulae (IX) or (X) and their variants.

In the following and throughout the description, the term “according to the general formulae YZ and their variants” includes any sub-formula(e) of said general formulae as disclosed herein. Accordingly, variants of the general formulae (V) and (VI) as defined herein, include formula (VII), formula (VIII), formula (IX) and formula (X) as defined herein. Variants of the general formulae (VII) and (VIII) as defined herein, include sub-formula (VII-acid), sub-formula (VII-ester), sub-formula (VIII-amide), sub-formula (VIII-Weinreb amide), sub-formula (VIII-hydroxamate derivative) and sub-formula (VIII-alkylated hydroxamate derivative) as defined herein. Variants of the general formulae (IX) and (X) as defined herein, include formula (IX-acid), formula (IX-ester), formula (X-amide), formula (X-Weinreb amide), formula (X-hydroxamate derivative) and formula (X-alkylated hydroxamate derivative) as defined herein.

In a further preferred variant, in the general formulae (VI), (IX), (X) and their variants X is selected from the group consisting of selected from the group consisting of CH₂, unsubstituted or substituted CH(alkyl), unsubstituted or substituted C(alkyl)₂, unsubstituted or substituted CH(alkenyl), unsubstituted or substituted CH(alkynyl), unsubstituted or substituted CH(alkoxy), unsubstituted or substituted CH(alkylthio), unsubstituted or substituted CH(cycloalkyl), unsubstituted or substituted CH(aryl), unsubstituted or substituted CH(heterocycloalkyl), unsubstituted or substituted CH(heteroaryl), —CR7″R8″, C═O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), unsubstituted or substituted N(heteroaryl), and unsubstituted or substituted C-spirocycles.

In a further preferred variant according to the first aspect of the present invention, the mycosporine-like amino acid compound is a compound according to general formulae (V), wherein
Z is —O—R2′ or amide′; and/or
R1′ is selected from the group consisting of CH₂, C(alkyl)₂, O, S, SO, SO₂, NH and N(alkyl); and/or
R2′ is selected from the group consisting of H, methyl, ethyl, propyl, isopropyl, butyl, isobutyl, tert-butyl,

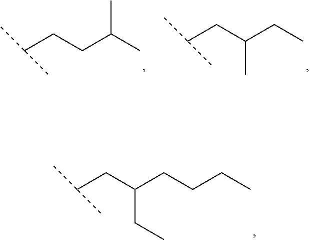

(2-ethyl-hexyl) and phenyl; or
amide′ is selected from the group consisting of NH₂, NH(alkyl), N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl.

In another preferred variant according to the first aspect of the present invention, the mycosporine-like amino acid compound is a compound according to general formulae (VII) or (VIII), wherein
R1′ is selected from the group consisting of CH₂, C(alkyl)₂, O, S, SO, SO₂, NH and N(alkyl); and/or
R2′ is selected from the group consisting of H, methyl, ethyl, propyl, isopropyl, butyl, isobutyl, tert-butyl,

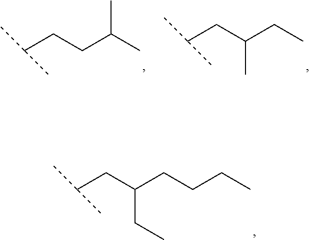

(2-ethyl-hexyl) and phenyl; or
amide′ is selected from the group consisting of NH₂, NH(alkyl), N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl.

In another preferred variant according to the first aspect of the present invention, the mycosporine-like amino acid compound is a compound according to general formula (VI), wherein
R1″ is selected from the group consisting of CH₂, C(alkyl)₂, O, S, SO, SO₂, NH and N(alkyl); and/or
R2″ is selected from the group consisting of —CH₂—OH, —C(═O)—O—Y, and —C(═O)-amide″; and/or
X is selected from the group consisting of CH₂, C═O, O, S, SO, SO₂, NH, and N(alkyl); and/or
Y is selected from the group consisting of H, methyl, ethyl, propyl, isopropyl, butyl, isobutyl, tert-butyl,

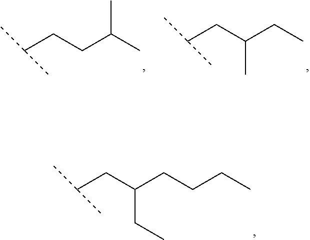

(2-ethyl-hexyl) and phenyl; and/or
amide″ is selected from the group consisting of NH₂, NH(alkyl), N(alkyl)₂, —N(O-alkyl)(alkyl) (Weinreb amide) and —N(OH)(H) (hydroxamate); and/or
the substituents R4″, R5″ and R6″ have the same meaning as defined for formula (VI);
wherein alkyl is selected from the group consisting of methyl, ethyl, propyl, isopropyl, butyl, isobutyl, and tert-butyl.

In a further preferred variant according to the first aspect of the present invention, the mycosporine-like amino acid compound is a compound according to general formulae (V), wherein
Z is —O—R2′ or amide′; and/or
R1′ is selected from the group consisting of CH₂ and C(methyl)₂; and/or
R2′ is selected from the group consisting of H, methyl, ethyl, propyl, isopropyl, butyl, isobutyl, tert-butyl,

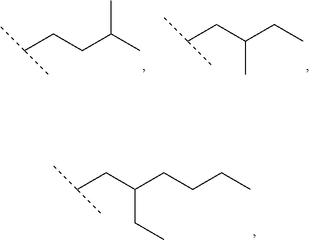

(2-ethyl-hexyl) and phenyl; or
amide′ is selected from the group consisting of NH₂, NH(alkyl), N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl.

In another preferred variant according to the first aspect of the present invention, the mycosporine-like amino acid compound is a compound according to general formulae (VII) or (VIII), wherein
R1′ is selected from the group consisting of CH₂ and C(methyl)₂; and/or
R2′ is selected from the group consisting of H, methyl, ethyl, propyl, isopropyl, butyl, isobutyl, tert-butyl,

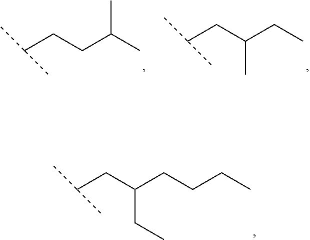

(2-ethyl-hexyl) and phenyl; or
amide′ is selected from the group consisting of NH₂, NH(alkyl), N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl.

In another preferred variant according to the first aspect of the present invention, the mycosporine-like amino acid compound is a compound according to general formula (VI), wherein
R1″ is selected from the group consisting of CH₂ and C(methyl)₂; and/or
R2″ is selected from the group consisting of —CH₂—OH, —C(═O)—O—Y, and —C(═O)-amide″; and/or
X is selected from the group consisting of CH₂, C═O, O, S, SO, SO₂, NH, and N(alkyl); and/or
Y is selected from the group consisting of H, methyl, ethyl, propyl, isopropyl, butyl, isobutyl, tert-butyl,

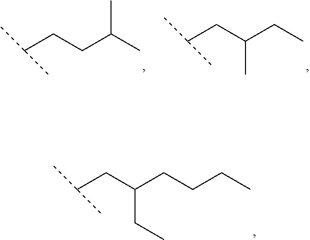

(2-ethyl-hexyl) and phenyl; and/or
amide″ is selected from the group consisting of NH₂, NH(alkyl), N(alkyl)₂, —N(O-alkyl)(alkyl) (Weinreb amide) and —N(OH)(H) (hydroxamate); and/or
the substituents R4″, R5″ and R6″ have the same meaning as defined for formula (VI);
wherein alkyl is selected from the group consisting of methyl, ethyl, propyl, isopropyl, butyl, isobutyl, and tert-butyl.

In a still more preferred variant according to the present invention, the sunscreen product, cosmetic or pharmaceutical preparation or homecare product comprises at least one mycosporine-like amino acid compound selected from the group consisting of the following compounds I-1 to I-128 according to the general formula (V) and II-1 to II-192 according to the general formula (VI): Table 1:

TABLE I

| 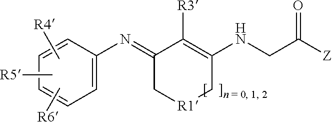 | RI′ | R′ | Z |
|---|---|---|---|
| I-1 | CH2 | H | —O—R2′ |
| I-2 | CH2 | methyl | —O—R2′ |
| I-3 | CH2 | ethyl | —O—R2′ |
| I-4 | CH2 | —O-methyl | —O—R2′ |
| I-5 | CH2 | —C(═O)—OH | —O—R2′ |
| I-6 | CH2 | —C(═O)—CH3 | —O—R2′ |
| I-7 | CH2 | —C(═O)—C3H7 | —O—R2′ |
| I-8 | CH2 | —C(═O)—O-methyl | —O—R2′ |
| I-9 | CH2 | H | amide′ |
| I-10 | CH2 | methyl | amide′ |
| I-11 | CH2 | ethyl | amide′ |
| I-12 | CH2 | —-O-methyl | amide′ |
| I-13 | CH2 | —C(═O)—OH | amide′ |
| I-14 | CH2 | —C(═O)—CH3 | amide′ |
| I-15 | CH2 | —C(═O—C3H7 | amide′ |
| I-16 | CH2 | —C(═O)—O-methyl | amide′ |
| I-17 | C(methyl)2 | H | —O—R2′ |
| I-18 | C(methyl)2 | methyl | —O—R2′ |
| I-19 | C(methyl)2 | ethyl | —O—R2′ |
| I-20 | C(methyl)2 | —O-methyl | —O—R2′ |
| I-21 | C(methyl)2 | —C(═O)—OH | —O—R2′ |
| I-22 | C(methyl)2 | —C(═O)—CH3 | —O—R2′ |
| I-23 | C(methyl)2 | —C(═O)—C3H7 | —O—R2′ |
| I-24 | C(methyl)2 | —C(═O)—O-methyl | —O—R2′ |
| I-25 | C(methyl)2 | H | amide′ |
| I-26 | C(methyl)2 | methyl | amide′ |
| I-27 | C(methyl)2 | ethyl | amide′ |
| I-28 | C(methyl)2 | —O-methyl | amide′ |
| I-29 | C(methyl)2 | —C(═O)—OH | amide′ |
| I-30 | C(methyl)2 | —C(═O)—CH3 | amide′ |
| I-31 | C(methyl)2 | —C(═O)—C3H7 | amide′ |
| I-32 | C(methyl)2 | —C(═O)—O-methyl | amide′ |
| I-33 | O | H | —O—R2′ |
| I-34 | O | methyl | —O—R2′ |
| I-35 | O | ethyl | —O—R2′ |
| I-36 | O | —O-methyl | —O—R2′ |
| I-37 | O | —C(═O)—OH | —O—R2′ |
| I-38 | O | —C(═O)—CH3 | —O—R2′ |
| I-39 | O | —C(═O)—C3H7 | —O—R2′ |
| I-40 | O | —C(═O)—O-methyl | amide′ |
| I-41 | O | H | amide′ |
| I-42 | O | methyl | amide′ |
| I-43 | O | ethyl | amide′ |
| I-44 | O | —O-methyl | amide′ |
| I-45 | O | —C(═O)—OH | amide′ |
| I-46 | O | —C(═O)—CH3 | amide′ |
| I-47 | O | —C(═O)—C3H7 | amide′ |
| I-48 | O | —C(═O)—O-methyl | amide′ |
| I-49 | S | H | —O—R2′ |
| I-50 | S | methyl | —O—R2′ |
| I-51 | S | ethyl | —O—R2′ |
| I-52 | S | —O-methyl | —O—R2′ |
| I-53 | S | —C(═O)—OH | —O—R2′ |
| I-54 | S | —C(═O)—CH3 | —O—R2′ |
| I-55 | S | —C(═O)—C3H7 | —O—R2′ |
| I-56 | S | —C(═O)—O-methyl | —O—R2′ |
| I-57 | S | H | amide′ |
| I-58 | S | methyl | amide′ |
| I-59 | S | ethyl | amide′ |
| I-60 | S | —O-methyl | amide′ |
| I-61 | S | —C(═O)—OH | amide′ |
| I-62 | S | —C(═O)—CH3 | amide′ |
| I-63 | S | —C(═O)—C3H7 | amide′ |
| I-64 | S | —C(═O)—O-methyl | amide′ |
| I-65 | SO | H | —O—R2′ |
| I-66 | SO | methyl | —O—R2′ |
| I-67 | SO | ethyl | —O—R2′ |
| I-68 | SO | —O-methyl | —O—R2′ |
| I-69 | SO | —C(═O)—OH | —O—R2′ |
| I-70 | SO | —C(═O)—CH3 | —O—R2′ |
| I-71 | SO | —C(═O)—C3H7 | —O—R2′ |
| I-72 | SO | —C(═O)—O-methyl | —O—R2′ |
| I-73 | SO | H | amide′ |
| I-74 | SO | methyl | amide′ |
| I-75 | SO | ethyl | amide′ |
| I-76 | SO | —O-methyl | amide′ |
| I-77 | SO | —C(═O)—OH | amide′ |
| I-78 | SO | —C(═O)—CH3 | amide′ |
| I-79 | SO | —C(═O)—C3H7 | amide′ |
| I-80 | SO | —C(═O)—O-methyl | amide′ |
| I-81 | SO2 | H | —O—R2′ |
| I-82 | SO2 | methyl | —O—R2′ |
| I-83 | SO2 | ethyl | —O—R2′ |
| I-84 | SO2 | —O-methyl | —O—R2′ |
| I-85 | SO2 | —C(═O)—OH | —O—R2′ |
| I-86 | SO2 | —C(═O)—CH3 | —O—R2′ |
| I-87 | SO2 | —C(═O)—C3H7 | —O—R2′ |
| I-88 | SO2 | —C(═O)—O-methyl | —O—R2′ |
| I-89 | SO2 | H | amide′ |
| I-90 | SO2 | methyl | amide′ |
| I-91 | SO2 | ethyl | amide′ |
| I-92 | SO2 | —O-methyl | amide′ |
| I-93 | SO2 | —C(═O)—OH | amide′ |
| I-94 | SO2 | —C(═O)—CH3 | amide′ |
| I-95 | SO2 | —C(═O)—C3H7 | amide′ |
| I-96 | SO2 | —C(═O)—O-methyl | amide′ |
| I-97 | NH | H | —O—R2′ |
| I-98 | NH | methyl | —O—R2′ |
| I-99 | NH | ethyl | —O—R2′ |
| I-100 | NH | —O-methyl | —O—R2′ |
| I-101 | NH | —C(═O)—OH | —O—R2′ |
| I-102 | NH | —C(═O)—CH3 | —O—R2′ |
| I-103 | NH | —C(═O)—C3H7 | —O—R2′ |
| I-104 | NH | —C(═O)—O-methyl | —O—R2′ |
| I-105 | NH | H | amide′ |
| I-106 | NH | methyl | amide′ |
| I-107 | NH | ethyl | amide′ |
| I-108 | NH | —O-methyl | amide′ |
| I-109 | NH | —C(═O)—OH | amide′ |
| I-110 | NH | —C(═O)—CH3 | amide′ |
| I-111 | NH | —C(═O)—C3H7 | amide′ |
| I-112 | NH | —C(═O)—O-methyl | amide' |
| I-113 | N(alkyl) | H | -O-R2' |
| I-114 | N(alkyl) | methyl | -O-R2' |
| I-115 | N(alkyl) | ethyl | -O-R2' |
| I-116 | N(alkyl) | —O-methyl | -O-R2' |
| I-117 | N(alkyl) | —C(═O)—OH | -O-R2' |
| I-118 | N(alkyl) | —C(═O)—CH3 | -O-R2' |
| I-119 | N(alkyl) | —C(═O)—C3H7 | -O-R2' |
| I-120 | N(alkyl) | —C(═O)—O-methyl | -O-R2' |
| I-121 | N(alkyl) | H | amide' |
| I-122 | N(alkyl) | methyl | amide' |
| I-123 | N(alkyl) | ethyl | amide' |
| I-124 | N(alkyl) | —O-methyl | amide' |
| I-125 | N(alkyl) | —C(═O)—OH | amide' |
| I-126 | N(alkyl) | —C(═O)—CH3 | amide' |
| I-127 | N(alkyl) | —C(═O)—C3H7 | amide' |
| I-128 | N(alkyl) | —C(═O)—O-methyl | amide' |
| 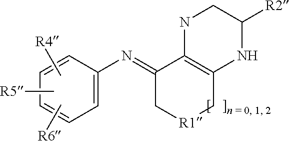 | R1″ | X | R2″ |
| II-1 | CH2 | CH2 | —CH2—OH |
| II-2 | CH2 | C═O | —CH2—OH |
| II-3 | CH2 | O | —CH2—OH |
| II-4 | CH2 | S | —CH2—OH |
| II-5 | CH2 | SO | —CH2—OH |
| II-6 | CH2 | SO2 | —CH2—OH |
| II-7 | CH2 | NH | —CH2—OH |
| II-8 | CH2 | N(alkyl) | —CH2—OH |
| II-9 | CH2 | CH2 | —C(═O)—O—Y |
| II-10 | CH2 | C═O | —C(═O)—O—Y |
| II-11 | CH2 | O | —C(═O)—O—Y |
| II-12 | CH2 | S | —C(═O)—O—Y |
| II-13 | CH2 | SO | —C(═O)—O—Y |
| II-14 | CH2 | SO2 | —C(═O)—O—Y |
| II-15 | CH2 | NH | —C(═O)—O—Y |
| II-16 | CH2 | N(alkyl) | —C(═O)—O—Y |
| II-17 | CH2 | CH2 | —C(═O)-amide″ |
| II-18 | CH2 | C═O | —C(═O)-amide″ |
| II-19 | CH2 | O | —C(═O)-amide″ |
| II-20 | CH2 | S | —C(═O)-amide″ |
| II-21 | CH2 | SO | —C(═O)-amide″ |
| II-22 | CH2 | SO2 | —C(═O)-amide″ |
| II-23 | CH2 | NH | —C(═O)-amide″ |
| II-24 | CH2 | N(alkyl) | —C(═O)-amide″ |
| II-25 | C(methyl)2 | CH2 | —CH2—OH |
| II-26 | C(methyl)2 | C═O | —CH2—OH |
| II-27 | C(methyl)2 | O | —CH2—OH |
| II-28 | C(methyl)2 | S | —CH2—OH |
| II-29 | C(methyl)2 | SO | —CH2—OH |
| II-30 | C(methyl)2 | SO2 | —CH2—OH |
| II-31 | C(methyl)2 | NH | —CH2—OH |
| II-32 | C(methyl)2 | N(alkyl) | —CH2—OH |
| II-33 | C(methyl)2 | CH2 | —C(═O)—O—Y |
| II-34 | C(methyl)2 | C═O | —C(═O)—O—Y |
| II-35 | C(methyl)2 | O | —C(═O)—O—Y |
| II-36 | C(methyl)2 | S | —C(═O)—O—Y |
| II-37 | C(methyl)2 | SO | —C(═O)—O—Y |
| II-38 | C(methyl)2 | SO2 | —C(═O)—O—Y |
| II-39 | C(methyl)2 | NH | —C(═O)—O—Y |
| II-40 | C(methyl)2 | N(alkyl) | —C(═O)—O—Y |
| II-41 | C(methyl)2 | CH2 | —C(═O)-amide″ |
| II-42 | C(methyl)2 | C═O | —C(═O)-amide″ |
| II-43 | C(methyl)2 | O | —C(═O)-amide″ |
| II-44 | C(methyl)2 | S | —C(═O)-amide″ |
| II-45 | C(methyl)2 | SO | —C(═O)-amide″ |
| II-46 | C(methyl)2 | SO2 | —C(═O)-amide″ |
| II-47 | C(methyl)2 | NH | —C(═O)-amide″ |
| II-48 | C(methyl)2 | N(alkyl) | —C(═O)-amide″ |
| II-49 | O | CH2 | —CH2—OH |
| II-50 | O | C═O | —CH2—OH |
| II-51 | O | O | —CH2—OH |
| II-52 | O | S | —CH2—OH |
| II-53 | O | SO | —CH2—OH |
| II-54 | O | SO2 | —CH2—OH |
| II-55 | O | NH | —CH2—OH |
| II-56 | O | N(alkyl) | —CH2—OH |
| II-57 | O | CH2 | —C(═O)—O—Y |
| II-58 | O | C═O | —C(═O)—O—Y |
| II-59 | O | O | —C(═O)—O—Y |
| II-60 | O | S | —C(═O)—O—Y |
| II-61 | O | SO | —C(═O)—O—Y |
| II-62 | O | SO2 | —C(═O)—O—Y |
| II-63 | O | NH | —C(═O)—O—Y |
| II-64 | O | N(alkyl) | —C(═O)—O—Y |
| II-65 | O | CH2 | —C(═O)-amide″ |
| II-66 | O | C═O | —C(═O)-amide″ |
| II-67 | O | O | —C(═O)-amide″ |
| II-68 | O | S | —C(═O)-amide″ |
| II-69 | O | SO | —C(═O)-amide″ |
| II-70 | O | SO2 | —C(═O)-amide″ |
| II-71 | O | NH | —C(═O)-amide″ |
| II-72 | O | N(alkyl) | —C(═O)-amide″ |
| II-73 | S | CH2 | —CH2—OH |
| II-74 | S | C═O | —CH2—OH |
| II-75 | S | O | —CH2—OH |
| II-76 | S | S | —CH2—OH |
| II-77 | S | SO | —CH2—OH |
| II-78 | S | SO2 | —CH2—OH |
| II-79 | S | NH | —CH2—OH |
| II-80 | S | N(alkyl) | —CH2—OH |
| II-81 | S | CH2 | —C(═O)—O—Y |
| II-82 | S | C═O | —C(═O)—O—Y |
| II-83 | S | O | —C(═O)—O—Y |
| II-84 | S | S | —C(═O)—O—Y |
| II-85 | S | SO | —C(═O)—O—Y |
| II-86 | S | SO2 | —C(═O)—O—Y |
| II-87 | S | NH | —C(═O)—O—Y |
| II-88 | S | N(alkyl) | —C(═O)—O—Y |
| II-89 | S | CH2 | —C(═O)-amide″ |
| II-90 | S | C═O | —C(═O)-amide″ |
| II-91 | S | O | —C(═O)-amide″ |
| II-92 | S | S | —C(═O)-amide″ |
| II-93 | S | SO | —C(═O)-amide″ |
| II-94 | S | SO2 | —C(═O)-amide″ |
| II-95 | S | NH | —C(═O)-amide″ |
| II-96 | S | N(alkyl) | —C(═O)-amide″ |
| II-97 | SO | CH2 | —CH2—OH |
| II-98 | SO | C═O | —CH2—OH |
| II-99 | SO | O | —CH2—OH |
| II-100 | SO | S | —CH2—OH |
| II-101 | SO | SO | —CH2—OH |
| II-102 | SO | SO2 | —CH2—OH |
| II-103 | SO | NH | —CH2—OH |
| II-104 | SO | N(alkyl) | —CH2—OH |
| II-105 | SO | CH2 | —C(═O)—O—Y |
| II-106 | SO | C═O | —C(═O)—O—Y |
| II-107 | SO | O | —C(═O)—O—Y |
| II-108 | SO | S | —C(═O)—O—Y |
| II-109 | SO | SO | —C(═O)—O—Y |
| II-110 | SC | SO2 | —C(═O)—O—Y |
| II-111 | SO | NH | —C(═O)—O—Y |
| II-112 | SO | N(alkyl) | —C(═O)—O—Y |
| II-113 | SO | CH2 | —C(═O)-amide″ |
| II-114 | SO | C═O | —C(═O)-amide″ |
| II-115 | SO | O | —C(═O)-amide″ |
| II-116 | SO | S | —C(═O)-amide″ |
| II-117 | SC | SO | —C(═O)-amide″ |
| II-118 | SO | SO2 | —C(═O)-amide″ |
| II-119 | SO | NH | —C(═O)-amide″ |
| II-120 | SO | N(alkyl) | —C(═O)-amide″ |
| II-121 | SO2 | CH2 | —CH2—OH |
| II-122 | SO2 | C═O | —CH2—OH |
| II-123 | SO2 | O | —CH2—OH |
| II-124 | SO2 | S | —CH2—OH |
| II-125 | SO2 | SO | —CH2—OH |
| II-126 | SO2 | SO2 | —CH2—OH |
| II-127 | SO2 | NH | —CH2—OH |
| II-128 | SO2 | N(alkyl) | —CH2—OH |
| II-129 | SO2 | CH2 | —C(═O)—O—Y |
| II-130 | SO2 | C═O | —C(═O)—O—Y |
| II-131 | SO2 | O | —C(═O)—O—Y |
| II-132 | SO2 | S | —C(═O)—O—Y |
| II-133 | SO2 | SO | —C(═O)—O—Y |
| II-134 | SO2 | SO2 | —C(═O)—O—Y |
| II-135 | SO2 | NH | —C(═O)—O—Y |
| II-136 | SO2 | N(alkyl) | —C(═O)—O—Y |
| II-137 | SO2 | CH2 | —C(═O)-amide″ |
| II-138 | SO2 | C═O | —C(═O)-amide″ |
| II-139 | SO2 | O | —C(═O)-amide″ |
| II-140 | SO2 | S | —C(═O)-amide″ |
| II-141 | SO2 | SO | —C(═O)-amide″ |
| II-142 | SO2 | SO2 | —C(═O)-amide″ |
| II-143 | SO2 | NH | —C(═O)-amide″ |
| II-144 | SO2 | N(alkyl) | —C(═O)-amide″ |
| II-145 | NH | CH2 | —CH2—OH |
| II-146 | NH | C═O | —CH2—OH |
| II-147 | NH | O | —CH2—OH |
| II-148 | NH | S | —CH2—OH |
| II-149 | NH | SO | —CH2—OH |
| II-150 | NH | SO2 | —CH2—OH |
| II-151 | NH | NH | —CH2—OH |
| II-152 | NH | N(alkyl) | —CH2—OH |
| II-153 | NH | CH2 | —C(═O)—O—Y |
| II-154 | NH | C═O | —C(═O)—O—Y |
| II-155 | NH | O | —C(═O)—O—Y |
| II-156 | NH | S | —C(═O)—O—Y |
| II-157 | NH | SO | —C(═O)—O—Y |
| II-158 | NH | SO2 | —C(═O)—O—Y |
| II-159 | NH | NH | —C(═O)—O—Y |
| II-160 | NH | N(alkyl) | —C(═O)—O—Y |
| II-161 | NH | CH2 | —C(═O)-amide″ |
| II-162 | NH | C═O | —C(═O)-amide″ |
| II-163 | NH | O | —C(═O)-amide″ |
| II-164 | NH | S | —C(═O)-amide″ |
| II-165 | NH | SO | —C(═O)-amide″ |
| II-166 | NH | SO2 | —C(═O)-amide″ |
| II-167 | NH | NH | —C(═O)-amide″ |
| II-168 | NH | N(alkyl) | —C(═O)-amide″ |
| II-169 | N(alkyl) | CH2 | —CH2—OH |
| II-170 | N(alkyl) | C═O | —CH2—OH |
| II-171 | N(alkyl) | O | —CH2—OH |
| II-172 | N(alkyl) | S | —CH2—OH |
| II-173 | N(alkyl) | SO | —CH2—OH |
| II-174 | N(alkyl) | SO2 | —CH2—OH |
| II-175 | N(alkyl) | NH | —CH2—OH |
| II-176 | N(alkyl) | N(alkyl) | —CH2—OH |
| II-177 | N(alkyl) | CH2 | —C(═O)—O—Y |
| II-178 | N(alkyl) | C═O | —C(═O)—O—Y |
| II-179 | N(alkyl) | O | —C(═O)—O—Y |
| II-180 | N(alkyl) | S | —C(═O)—O—Y |
| II-181 | N(alkyl) | SO | —C(═O)—O—Y |
| II-182 | N(alkyl) | SO2 | —C(═O)—O—Y |
| II-183 | N(alkyl) | NH | —C(═O)—O—Y |
| II-184 | N(alkyl) | N(alkyl) | —C(═O)—O—Y |
| II-185 | N(alkyl) | CH2 | —C(═O)-amide″ |
| II-186 | N(alkyl) | C═O | —C(═O)-amide″ |
| II-187 | N(alkyl) | O | —C(═O)-amide″ |
| II-188 | N(alkyl) | S | —C(═O)-amide″ |
| II-189 | N(alkyl) | SO | —C(═O)-amide″ |
| II-190 | N(alkyl) | SO2 | —C(═O)-amide″ |
| II-191 | N(alkyl) | NH | —C(═O)-amide″ |
| II-192 | N(alkyl) | N(alkyl) | —C(═O)-amide″ |

or a tautomer or a stereoisomer or a salt of the afore-mentioned compounds, or any mixture of the afore-mentioned compounds.

In the mycosporine-like amino acid compounds of Table 1, the phenyl ring may be unsubstituted or substituted. The substituents R4′, R5′, R6′, R4″, R5″, R6″, amide′, Y, amide″ and alkyl have the same meaning as defined for the mycosporine-like amino acid compounds according to general formulae (V) or (VI) before.

Particular preferred are those mycosporine-like amino acid compounds, wherein in the general formula (V) R1′ is CH₂ or C(methyl)₂ or wherein in the general formula (VI) R1″ is CH₂ or C(methyl)₂:

TABLE 2

| 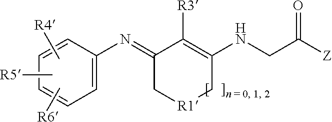 | R1′ | R3′ | Z |
|---|---|---|---|
| I-1 | CH2 | H | —O—R2′ |
| I-2 | CH2 | methyl | —O—R2′ |
| I-3 | CH2 | ethyl | —O—R2′ |
| I-4 | CH2 | —O-methyl | —O—R2′ |
| I-5 | CH2 | —C(═O)—OH | —O—R2′ |
| I-6 | CH2 | —C(═O)—CH3 | —O—R2′ |
| I-7 | CH2 | —C(═O)—C3H7 | —O—R2′ |
| I-8 | CH2 | —C(═O)—O-methyl | —O—R2′ |
| I-9 | CH2 | H | amide′ |
| I-10 | CH2 | methyl | amide′ |
| I-11 | CH2 | ethyl | amide′ |
| I-12 | CH2 | —O-methyl | amide′ |
| I-13 | CH2 | —C(═O)—OH | amide′ |
| I-14 | CH2 | —C(═O)—CH3 | amide′ |
| I-15 | CH2 | —C(═O)—C3H7 | amide′ |
| I-16 | CH2 | —C(═O)—O-methyl | amide′ |
| I-17 | C(methyl)2 | H | —O—R2′ |
| I-18 | C(methyl)2 | methyl | —O—R2′ |
| I-19 | C(methyl)2 | ethyl | —O—R2′ |
| I-20 | C(methyl)2 | —O-methyl | —O—R2′ |
| I-21 | C(methyl)2 | —C(═O)—OH | —O—R2′ |
| I-22 | C(methyl)2 | —C(═O)—CH3 | —O—R2′ |
| I-23 | C(methyl)2 | —C(═O)—C3H7 | —O—R2′ |
| I-24 | C(methyl)2 | —C(═O)—O-methyl | —O—R2′ |
| I-25 | C(methyl)2 | H | amide′ |
| I-26 | C(methyl)2 | methyl | amide′ |
| I-27 | C(methyl)2 | ethyl | amide′ |
| I-28 | C(methyl)2 | —O-methyl | amide′ |
| I-29 | C(methyl)2 | —C(═O)—OH | amide′ |
| I-30 | C(methyl)2 | —C(═O)—CH3 | amide′ |
| I-31 | C(methyl)2 | —C(═O)—C3H7 | amide′ |
| I-32 | C(methyl)2 | —C(═O)—O-methyl | amide′ |
| 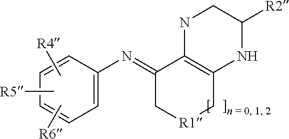 | R1″ | X | R2″ |
| II-1 | CH2 | CH2 | —CH2—OH |
| II-2 | CH2 | C═O | —CH2—OH |
| II-3 | CH2 | O | —CH2—OH |
| II-4 | CH2 | S | —CH2—OH |
| II-5 | CH2 | SO | —CH2—OH |
| II-6 | CH2 | SO2 | —CH2—OH |
| II-7 | CH2 | NH | —CH2—OH |
| II-8 | CH2 | N(alkyl) | —CH2—OH |
| II-9 | CH2 | CH2 | —C(═O)—O—Y |
| II-10 | CH2 | C═O | —C(═O)—O—Y |
| II-11 | CH2 | O | —C(═O)—O—Y |
| II-12 | CH2 | S | —C(═O)—O—Y |
| II-13 | CH2 | SO | —C(═O)—O—Y |
| II-14 | CH2 | SO2 | —C(═O)—O—Y |
| II-15 | CH2 | NH | —C(═O)—O—Y |
| II-16 | CH2 | N(alkyl) | —C(═O)—O—Y |
| II-17 | CH2 | CH2 | —C(═O)-amide″ |
| II-18 | CH2 | C═O | —C(═O)-amide″ |
| II-19 | CH2 | O | —C(═O)-amide″ |
| II-20 | CH2 | S | —C(═O)-amide″ |
| II-21 | CH2 | SO | —C(═O)-amide″ |
| II-22 | CH2 | SO2 | —C(═O)-amide″ |
| II-23 | CH2 | NH | —C(═O)-amide″ |
| II-24 | CH2 | N(alkyl) | —C(═O)-amide″ |
| II-25 | C(methyl)2 | CH2 | —CH2—OH |
| II-26 | C(methyl)2 | C═O | —CH2—OH |
| II-27 | C(methyl)2 | O | —CH2—OH |
| II-28 | C(methyl)2 | S | —CH2—OH |
| II-29 | C(methyl)2 | SO | —CH2—OH |
| II-30 | C(methyl)2 | SO2 | —CH2—OH |
| II-31 | C(methyl)2 | NH | —CH2—OH |
| II-32 | C(methyl)2 | N(alkyl) | —CH2—OH |
| II-33 | C(methyl)2 | CH2 | —C(═O)—O—Y |
| II-34 | C(methyl)2 | C═O | —C(═O)—O—Y |
| II-35 | C(methyl)2 | O | —C(═O)—O—Y |
| II-36 | C(methyl)2 | S | —C(═O)—O—Y |
| II-37 | C(methyl)2 | SO | —C(═O)—O—Y |
| II-38 | C(methyl)2 | SO2 | —C(═O)—O—Y |
| II-39 | C(methyl)2 | NH | —C(═O)—O—Y |
| II-40 | C(methyl)2 | N(alkyl) | —C(═O)—O—Y |
| II-41 | C(methyl)2 | CH2 | —C(═O)-amide″ |
| II-42 | C(methyl)2 | C═O | —C(═O)-amide″ |
| II-43 | C(methyl)2 | O | —C(═O)-amide″ |
| II-44 | C(methyl)2 | S | —C(═O)-amide″ |
| II-45 | C(methyl)2 | SO | —C(═O)-amide″ |
| II-46 | C(methyl)2 | SO2 | —C(═O)-amide″ |
| II-47 | C(methyl)2 | NH | —C(═O)-amide″ |
| II-48 | C(methyl)2 | N(alkyl) | —C(═O)-amide″ |

or a tautomer or a stereoisomer or a salt of the afore-mentioned compounds, or any mixture of the afore-mentioned compounds.

In the mycosporine-like amino acid compounds of Table 2, the phenyl ring may be unsubstituted or substituted. The substituents R4′, R5′, R6′, R4″, R5″, R6″, amide′, Y, amide″ and alkyl have the same meaning as defined for the mycosporine-like amino acid compounds according to general formula (V) or (VI) before.

Preferably, in the mycosporine-like amino acid compounds according to the general formula (V) and formula (VI) specified in Table 1 and Table 2
R2′ is selected from the group consisting of H, methyl, ethyl, propyl, isopropyl, butyl, isobutyl, tert-butyl,

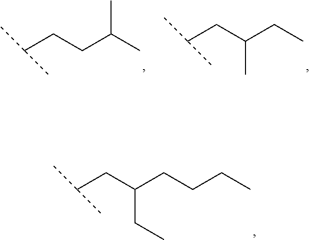

(2-ethylhexyl) and phenyl;
amide′ is selected from the group consisting of NH₂, NH(alkyl), N(alkyl)₂, —N(O-alkyl)(alkyl) (Weinreb amide) and —N(OH)(H) (hydroxamate);
Y is selected from the group consisting of H, methyl, ethyl, propyl, isopropyl, butyl, isobutyl, tert-butyl,

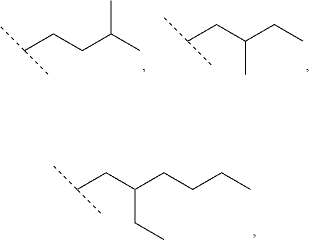

(2-ethylhexyl) and phenyl;
amide″ is selected from the group consisting of NH₂, NH(alkyl), N(alkyl)₂, —N(O-alkyl)(alkyl) (Weinreb amide) and —N(OH)(H) (hydroxamate); and
alkyl is selected from the group consisting of methyl, ethyl, propyl, isopropyl, butyl, isobutyl, and tert-butyl.

In a most preferred variant according to the first aspect of the present invention, the mycosporine-like amino acid compound is a compound according to general formula (V) and its variants or formula (VI) and its variants, wherein R1′ or R1″ are CH₂. Such mycosporine-like amino acid compounds have a particular good water solubility.

In a still further preferred variant according to the first aspect of the present invention, the mycosporine-like amino acid compound is a compound according to general formulae (VI), (IX), (X) and their variants, wherein X is selected from the group consisting of S, SO, and SO₂.

Such mycosporine-like amino acid compounds alter the UV absorption profile of the sunscreen product or cosmetic or pharmaceutical preparation and broaden the UV absorption spectrum of the formulation across the UV radiation spectrum.

In a still more preferred variant according to the first aspect of the present invention, the mycosporine-like amino acid compound is a compound according to general formulae (VI), (IX, (X) and their variants, wherein X is S. However, the S atom in said compounds is prone to oxidation.

In a most preferred variant according to the first aspect of the present invention, the mycosporine-like amino acid compound is a compound according to general formulae (VI), (IX), (X) and their variants, wherein X is either SO or SO₂. Such mycosporine-like amino acid compounds are particular stable, due to their oxidation state.

In a still further preferred variant according to the present invention, in the general formulae (V) to (X) and their variants n is 1.

In a still more preferred variant according to the first aspect of the present invention, the mycosporine-like amino acid compound is a compound according to general formula (V), wherein
Z is —O—R2′ or amide′; and/or
R1′ is either CH₂ or C(methyl)₂; and/or
R2′ is either H or ethyl or isopropyl or 2-ethylhexyl or phenyl; or
amide′ is either NH₂ or N(methyl)₂ or N(ethyl)₂ or —N(O-alkyl)(alkyl) (Weinreb amide) or —N(OH)(H) (hydroxamate); and/or
R3′ is either H or —O-methyl or —C(═O)—OH or —C(═O)—CH₃ or —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl.

In a more preferred variant according to the first aspect of the present invention, the mycosporine-like amino acid compound is a compound according to general formulae (VII) or (VIII), wherein
R1′ is either CH₂ or C(methyl)₂; and/or
R2′ is either H or ethyl or isopropyl or 2-ethylhexyl or phenyl; or
amide′ is either NH₂ or N(methyl)₂ or N(ethyl)₂ or —N(O-alkyl)(alkyl) (Weinreb amide) or —N(OH)(H) (hydroxamate); and/or
R3′ is either H or —O-methyl or —C(═O)—OH or —C(═O)—CH₃ or —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl.

In a more preferred variant according to the first aspect of the present invention, the mycosporine-like amino acid compound is a compound according to general formula (VI), wherein
R1″ is either CH₂ or C(methyl)₂; and/or
R2″ is either —C(═O)—O—Y, or —C(═O)-amide″; and/or
X is S, SO or SO₂; and/or
Y is either H or ethyl or isopropyl or 2-ethylhexyl or phenyl; or
amide″ is either NH₂ or N(methyl)₂ or N(ethyl)₂ or —N(O-alkyl)(alkyl) (Weinreb amide) or —N(OH)(H) (hydroxamate); and/or
the substituents R4″, R5″ and R6″ have the same meaning as defined for formula (VI);
wherein alkyl is selected from the group consisting of methyl, ethyl, propyl, isopropyl, butyl, isobutyl, and butyl.

The phenyl ring in the mycosporine-like amino acid compounds according to the general formulae (V) to (X) and their variants can be unsubstituted or substituted.

In a preferred variant, the phenyl ring in the mycosporine-like amino acid compounds according to the general formulae (V) to (X) and their variants is unsubstituted, i.e. the substituents R4′, R5′ and R6′ on the phenyl ring in the general formula (V) are H; or the substituents R4″, R5″ and R6″ on the phenyl ring in the general formula (VI) are H.

In a further preferred variant, the phenyl ring in the mycosporine-like amino acid compounds according to the general formulae (V) to (X) and their variants is substituted.

Most preferred, the phenyl ring in the mycosporine-like amino acid compounds according to the general formula (V), (VII), (VIII) and their variants is unsubstituted, i.e. the substituents R4′, R5′ and R6′ are each H. Alternatively, most preferred, the phenyl ring in the mycosporine-like amino acid compounds according to the general formula (VI), (IX), (X) and their variants is unsubstituted, i.e. the substituents R4″, R5″ and R6″ are each H. Said compounds have an improved water solubility.

Preferably, the phenyl ring in the mycosporine-like amino acid compounds according to the general formulae (V) to (X) and their variants is monosubstituted, disubstituted or trisubstituted.

In a still more preferred variant according to the first aspect of the present invention, the mycosporine-like amino acid is a compound according to general formulae (V), (VII), (VIII) and their variants, wherein the substituents R4′, R5′ and R6′ which may be identical or different, are independently from each other selected from the group consisting of H, OH, alkyl, preferably methyl, ethyl, propyl, iso-propyl, butyl, isobutyl or tert.-butyl, alkoxy, preferably, O-methyl, O-ethyl, or O-butyl, —S(═O)₂OH, and sulfonyl.

Still more preferred, the substituents R4′, R5′ and R6′ in the general formulae (V), (VII), (VIII) and their variants, which may be identical or different, are independently from each other selected from the group consisting of H, OH, methyl, O-methyl, —S(═O)₂OH, and sulfonyl.

Alternatively, the mycosporine-like amino acid compound is a compound according to general formulae (VI), (IX), (X) and their variants, wherein the substituents R4″, R5″ and R6″ which may be identical or different, are independently from each other selected from the group consisting of H, OH, alkyl, preferably methyl, ethyl, propyl, iso-propyl, butyl, isobutyl, tert-butyl, alkoxy, preferably O-methyl, O-ethyl, or O-butyl, —S(═O)₂OH, and sulfonyl.

Still more preferred, the substituents R4″, R5″ and R6″ in the general formula (VI), (IX), (X) and their variants, which may be identical or different, are independently from each other selected from the group consisting of H, OH, methyl, O-methyl, —S(═O)₂OH, and sulfonyl.

In the general formulae (V) to (X) and their variants, the following substitution patterns are particularly preferred:
R4′=H and R5′=H and R6′=H; or
R4′=H and R5′=H and R6′=OH; or
R4′=H and R5′=H and R6′=methyl; or
R4′=H and R5′=H and R6′=O-methyl; or
R4′=H and R5′=H and R6′=—S(═O)₂OH; or
R4′=H and R5′=H and R6′=sulfonyl; or
R4′=H and R5′=OH and R6′=H; or
R4′=H and R5′=OH and R6′=OH; or
R4′=H and R5′=OH and R6′=methyl; or
R4′=H and R5′=OH and R6′=O-methyl; or
R4′=H and R5′=OH and R6′=—S(═O)₂OH; or
R4′=H and R5′=OH and R6′=sulfonyl; or
R4′=H and R5′=methyl and R6′=H; or
R4′=H and R5′=methyl and R6′=OH; or
R4′=H and R5′=methyl and R6′=methyl; or
R4′=H and R5′=methyl and R6′=O-methyl; or
R4′=H and R5′=methyl and R6′=—S(═O)₂OH; or
R4′=H and R5′=methyl and R6′=sulfonyl; or
R4′=H and R5′=O-methyl and R6′=H; or
R4′=H and R5′=O-methyl and R6′=OH; or
R4′=H and R5′=O-methyl and R6′=methyl; or
R4′=H and R5′=O-methyl and R6′=O-methyl; or
R4′=H and R5′=O-methyl and R6′=—S(═O)₂OH; or
R4′=H and R5′=O-methyl and R6′=sulfonyl; or
R4′=H and R5′=—S(═O)₂OH and R6′=H; or
R4′=H and R5′=—S(═O)₂OH and R6′=OH; or
R4′=H and R5′=—S(═O)₂OH and R6′=methyl; or
R4′=H and R5′=—S(═O)₂OH and R6′=O-methyl; or
R4′=H and R5′=—S(═O)₂OH and R6′=—S(═O)₂OH; or
R4′=H and R5′=—S(═O)₂OH and R6′=sulfonyl; or
R4′=H and R5′=sulfonyl and R6′=H; or
R4′=H and R5′=sulfonyl and R6′=OH; or
R4′=H and R5′=sulfonyl and R6′=methyl; or
R4′=H and R5′=sulfonyl and R6′=O-methyl; or
R4′=H and R5′=sulfonyl and R6′=—S(═O)₂OH; or
R4′=H and R5′=sulfonyl and R6′=sulfonyl; or
R4′=OH and R5′=H and R6′=H; or
R4′=OH and R5′=H and R6′=OH; or
R4′=OH and R5′=H and R6′=methyl; or
R4′=OH and R5′=H and R6′=O-methyl; or
R4′=OH and R5′=H and R6′=—S(═O)₂OH; or
R4′=OH and R5′=H and R6′=sulfonyl; or
R4′=OH and R5′=OH and R6′=H; or
R4′=OH and R5′=OH and R6′=OH; or
R4′=OH and R5′=OH and R6′=methyl; or
R4′=OH and R5′=OH and R6′=O-methyl; or
R4′=OH and R5′=OH and R6′=—S(═O)₂OH; or
R4′=OH and R5′=OH and R6′=sulfonyl; or
R4′=OH and R5′=methyl and R6′=H; or
R4′=OH and R5′=methyl and R6′=OH; or
R4′=OH and R5′=methyl and R6′=methyl; or
R4′=OH and R5′=methyl and R6′=O-methyl; or
R4′=OH and R5′=methyl and R6′=—S(═O)₂OH; or
R4′=OH and R5′=methyl and R6′=sulfonyl; or
R4′=OH and R5′=O-methyl and R6′=H; or
R4′=OH and R5′=O-methyl and R6′=OH; or
R4′=OH and R5′=O-methyl and R6′=methyl; or
R4′=OH and R5′=O-methyl and R6′=O-methyl; or
R4′=OH and R5′=O-methyl and R6′=—S(═O)₂OH; or
R4′=OH and R5′=O-methyl and R6′=sulfonyl; or
R4′=OH and R5′=—S(═O)₂OH and R6′=H; or
R4′=OH and R5′=—S(═O)₂OH and R6′=OH; or
R4′=OH and R5′=—S(═O)₂OH and R6′=methyl; or
R4′=OH and R5′=—S(═O)₂OH and R6′=O-methyl; or
R4′=OH and R5′=—S(═O)₂OH and R6′=—S(═O)₂OH; or
R4′=OH and R5′=—S(═O)₂OH and R6′=sulfonyl; or
R4′=OH and R5′=sulfonyl and R6′=H; or
R4′=OH and R5′=sulfonyl and R6′=OH; or
R4′=OH and R5′=sulfonyl and R6′=methyl; or
R4′=OH and R5′=sulfonyl and R6′=O-methyl; or
R4′=OH and R5′=sulfonyl and R6′=—S(═O)₂OH; or
R4′=OH and R5′=sulfonyl and R6′=sulfonyl; or
R4′=methyl and R5′=H and R6′=H; or
R4′=methyl and R5′=H and R6′=OH; or
R4′=methyl and R5′=H and R6′=methyl; or
R4′=methyl and R5′=H and R6′=O-methyl; or
R4′=methyl and R5′=H and R6′=—S(═O)₂OH; or
R4′=methyl and R5′=H and R6′=sulfonyl; or
R4′=methyl and R5′=OH and R6′=H; or
R4′=methyl and R5′=OH and R6′=OH; or
R4′=methyl and R5′=OH and R6′=methyl; or
R4′=methyl and R5′=OH and R6′=O-methyl; or
R4′=methyl and R5′=OH and R6′=—S(═O)₂OH; or
R4′=methyl and R5′=OH and R6′=sulfonyl; or
R4′=methyl and R5′=methyl and R6′=H; or
R4′=methyl and R5′=methyl and R6′=OH; or
R4′=methyl and R5′=methyl and R6′=methyl; or
R4′=methyl and R5′=methyl and R6′=O-methyl; or
R4′=methyl and R5′=methyl and R6′=—S(═O)₂OH; or
R4′=methyl and R5′=methyl and R6′=sulfonyl; or
R4′=methyl and R5′=O-methyl and R6′=H; or
R4′=methyl and R5′=O-methyl and R6′=OH; or
R4′=methyl and R5′=O-methyl and R6′=methyl; or
R4′=methyl and R5′=O-methyl and R6′=O-methyl; or
R4′=methyl and R5′=O-methyl and R6′=—S(═O)₂OH; or
R4′=methyl and R5′=O-methyl and R6′=sulfonyl; or
R4′=methyl and R5′=—S(═O)₂OH and R6′=H; or
R4′=methyl and R5′=—S(═O)₂OH and R6′=OH; or
R4′=methyl and R5′=—S(═O)₂OH and R6′=methyl; or
R4′=methyl and R5′=—S(═O)₂OH and R6′=O-methyl; or
R4′=methyl and R5′=—S(═O)₂OH and R6′=—S(═O)₂OH; or
R4′=methyl and R5′=—S(═O)₂OH and R6′=sulfonyl; or
R4′=methyl and R5′=sulfonyl and R6′=H; or
R4′=methyl and R5′=sulfonyl and R6′=OH; or
R4′=methyl and R5′=sulfonyl and R6′=methyl; or
R4′=methyl and R5′=sulfonyl and R6′=O-methyl; or
R4′=methyl and R5′=sulfonyl and R6′=—S(═O)₂OH; or
R4′=methyl and R5′=sulfonyl and R6′=sulfonyl; or
R4′=O-methyl and R5′=H and R6′=H; or
R4′=O-methyl and R5′=H and R6′=OH; or
R4′=O-methyl and R5′=H and R6′=methyl; or
R4′=O-methyl and R5′=H and R6′=O-methyl; or
R4′=O-methyl and R5′=H and R6′=—S(═O)₂OH; or
R4′=O-methyl and R5′=H and R6′=sulfonyl; or
R4′=O-methyl and R5′=OH and R6′=H; or
R4′=O-methyl and R5′=OH and R6′=OH; or
R4′=O-methyl and R5′=OH and R6′=methyl; or
R4′=O-methyl and R5′=OH and R6′=O-methyl; or
R4′=O-methyl and R5′=OH and R6′=—S(═O)₂OH; or
R4′=O-methyl and R5′=OH and R6′=sulfonyl; or
R4′=O-methyl and R5′=methyl and R6′=H; or
R4′=O-methyl and R5′=methyl and R6′=OH; or
R4′=O-methyl and R5′=methyl and R6′=methyl; or
R4′=O-methyl and R5′=methyl and R6′=O-methyl; or
R4′=O-methyl and R5′=methyl and R6′=—S(═O)₂OH; or
R4′=O-methyl and R5′=methyl and R6′=sulfonyl; or
R4′=O-methyl and R5′=O-methyl and R6′=H; or
R4′=O-methyl and R5′=O-methyl and R6′=OH; or
R4′=O-methyl and R5′=O-methyl and R6′=methyl; or
R4′=O-methyl and R5′=O-methyl and R6′=O-methyl; or
R4′=O-methyl and R5′=O-methyl and R6′=—S(═O)₂OH; or
R4′=O-methyl and R5′=O-methyl and R6′=sulfonyl; or
R4′=O-methyl and R5′=—S(═O)₂OH and R6′=H; or
R4′=O-methyl and R5′=—S(═O)₂OH and R6′=OH; or
R4′=O-methyl and R5′=—S(═O)₂OH and R6′=methyl; or
R4′=O-methyl and R5′=—S(═O)₂OH and R6′=O-methyl; or
R4′=O-methyl and R5′=—S(═O)₂OH and R6′=—S(═O)₂OH; or
R4′=O-methyl and R5′=—S(═O)₂OH and R6′=sulfonyl; or
R4′=O-methyl and R5′=sulfonyl and R6′=H; or
R4′=O-methyl and R5′=sulfonyl and R6′=OH; or
R4′=O-methyl and R5′=sulfonyl and R6′=methyl; or
R4′=O-methyl and R5′=sulfonyl and R6′=O-methyl; or
R4′=O-methyl and R5′=sulfonyl and R6′=—S(═O)₂OH; or
R4′=O-methyl and R5′=sulfonyl and R6′=sulfonyl; or
R4′=—S(═O)₂OH and R5′=H and R6′=H; or
R4′=—S(═O)₂OH and R5′=H and R6′=OH; or
R4′=—S(═O)₂OH and R5′=H and R6′=methyl; or
R4′=—S(═O)₂OH and R5′=H and R6′=O-methyl; or
R4′=—S(═O)₂OH and R5′=H and R6′=—S(═O)₂OH; or
R4′=—S(═O)₂OH and R5′=H and R6′=sulfonyl; or
R4′=—S(═O)₂OH and R5′=OH and R6′=H; or
R4′=—S(═O)₂OH and R5′=OH and R6′=OH; or
R4′=—S(═O)₂OH and R5′=OH and R6′=methyl; or
R4′=—S(═O)₂OH and R5′=OH and R6′=O-methyl; or
R4′=—S(═O)₂OH and R5′=OH and R6′=—S(═O)₂OH; or
R4′=—S(═O)₂OH and R5′=OH and R6′=sulfonyl; or
R4′=—S(═O)₂OH and R5′=methyl and R6′=H; or
R4′=—S(═O)₂OH and R5′=methyl and R6′=OH; or
R4′=—S(═O)₂OH and R5′=methyl and R6′=methyl; or
R4′=—S(═O)₂OH and R5′=methyl and R6′=O-methyl; or
R4′=—S(═O)₂OH and R5′=methyl and R6′=—S(═O)₂OH; or
R4′=—S(═O)₂OH and R5′=methyl and R6′=sulfonyl; or
R4′=—S(═O)₂OH and R5′=O-methyl and R6′=H; or
R4′=—S(═O)₂OH and R5′=O-methyl and R6′=OH; or
R4′=—S(═O)₂OH and R5′=O-methyl and R6′=methyl; or
R4′=—S(═O)₂OH and R5′=O-methyl and R6′=O-methyl; or
R4′=—S(═O)₂OH and R5′=O-methyl and R6′=—S(═O)₂OH; or
R4′=—S(═O)₂OH and R5′=O-methyl and R6′=sulfonyl; or
R4′=—S(═O)₂OH and R5′=—S(═O)₂OH═ and R6′=H; or
R4′=—S(═O)₂OH and R5′=—S(═O)₂OH and R6′=OH; or
R4′=—S(═O)₂OH and R5′=—S(═O)₂OH and R6′=methyl; or
R4′=—S(═O)₂OH and R5′=—S(═O)₂OH and R6′=O-methyl; or
R4′=—S(═O)₂OH and R5′=sulfonyl= and R6′=H; or
R4′=—S(═O)₂OH and R5′=sulfonyl and R6′=OH; or
R4′=—S(═O)₂OH and R5′=sulfonyl and R6′=methyl; or
R4′=—S(═O)₂OH and R5′=sulfonyl and R6′=O-methyl; or
R4′=sulfonyl and R5′=H and R6′=H; or
R4′=sulfonyl and R5′=H and R6′=OH; or
R4′=sulfonyl and R5′=H and R6′=methyl; or
R4′=sulfonyl and R5′=H and R6′=O-methyl; or
R4′=sulfonyl and R5′=H and R6′=—S(═O)₂OH; or
R4′=sulfonyl and R5′=H and R6′=sulfonyl; or
R4′=sulfonyl and R5′=OH and R6′=H; or
R4′=sulfonyl and R5′=OH and R6′=OH; or
R4′=sulfonyl and R5′=OH and R6′=methyl; or
R4′=sulfonyl and R5′=OH and R6′=O-methyl; or
R4′=sulfonyl and R5′=OH and R6′=—S(═O)₂OH; or
R4′=sulfonyl and R5′=OH and R6′=sulfonyl; or
R4′=sulfonyl and R5′=methyl and R6′=H; or
R4′=sulfonyl and R5′=methyl and R6′=OH; or
R4′=sulfonyl and R5′=methyl and R6′=methyl; or
R4′=sulfonyl and R5′=methyl and R6′=O-methyl; or
R4′=sulfonyl and R5′=methyl and R6′=—S(═O)₂OH; or
R4′=sulfonyl and R5′=methyl and R6′=sulfonyl; or
R4′=sulfonyl and R5′=O-methyl and R6′=H; or
R4′=sulfonyl and R5′=O-methyl and R6′=OH; or
R4′=sulfonyl and R5′=O-methyl and R6′=methyl; or
R4′=sulfonyl and R5′=O-methyl and R6′=O-methyl; or
R4′=sulfonyl and R5′=O-methyl and R6′=—S(═O)₂OH; or
R4′=sulfonyl and R5′=O-methyl and R6′=sulfonyl; or
R4′=sulfonyl and R5′=—S(═O)₂OH and R6′=H; or
R4′=sulfonyl and R5′=—S(═O)₂OH and R6′=OH; or
R4′=sulfonyl and R5′=—S(═O)₂OH and R6′=methyl; or
R4′=sulfonyl and R5′=—S(═O)₂OH and R6′=O-methyl; or
R4′=sulfonyl and R5′=sulfonyl and R6′=H; or
R4′=sulfonyl and R5′=sulfonyl and R6′=OH; or
R4′=sulfonyl and R5′=sulfonyl and R6′=methyl; or
R4′=sulfonyl and R5′=sulfonyl and R6′=O-methyl; or
R4″=H and R5″=H and R6″=H; or
R4″=H and R5″=H and R6″=OH; or
R4″=H and R5″=H and R6″=methyl; or
R4″=H and R5″=H and R6″=O-methyl; or
R4″=H and R5″=H and R6″=—S(═O)₂OH; or
R4″=H and R5″=H and R6″=sulfonyl; or
R4″=H and R5″=OH and R6″=H; or
R4″=H and R5″=OH and R6″=OH; or
R4″=H and R5″=OH and R6″=methyl; or
R4″=H and R5″=OH and R6″=O-methyl; or
R4″=H and R5″=OH and R6″=—S(═O)₂OH; or
R4″=H and R5″=OH and R6″=sulfonyl; or
R4″=H and R5″=methyl and R6″=H; or
R4″=H and R5″=methyl and R6″=OH; or
R4″=H and R5″=methyl and R6″=methyl; or
R4″=H and R5″=methyl and R6″=O-methyl; or
R4″=H and R5″=methyl and R6″=—S(═O)₂OH; or
R4″=H and R5″=methyl and R6″=sulfonyl; or
R4″=H and R5″=O-methyl and R6″=H; or
R4″=H and R5″=O-methyl and R6″=OH; or
R4″=H and R5″=O-methyl and R6″=methyl; or
R4″=H and R5″=O-methyl and R6″=O-methyl; or
R4″=H and R5″=O-methyl and R6″=—S(═O)₂OH; or
R4″=H and R5″=O-methyl and R6″=sulfonyl; or
R4″=H and R5″=—S(═O)₂OH and R6″=H; or
R4″=H and R5″=—S(═O)₂OH and R6″=OH; or
R4″=H and R5″=—S(═O)₂OH and R6″=methyl; or
R4″=H and R5″=—S(═O)₂OH and R6″=O-methyl; or
R4″=H and R5″=—S(═O)₂OH and R6″=—S(═O)₂OH; or
R4″=H and R5″=—S(═O)₂OH and R6″=sulfonyl; or
R4″=H and R5″=sulfonyl and R6″=H; or
R4″=H and R5″=sulfonyl and R6″=OH; or
R4″=H and R5″=sulfonyl and R6″=methyl; or
R4″=H and R5″=sulfonyl and R6″=O-methyl; or
R4″=H and R5″=sulfonyl and R6″=—S(═O)₂OH; or
R4″=H and R5″=sulfonyl and R6″=sulfonyl; or
R4″=OH and R5″=H and R6″=H; or
R4″=OH and R5″=H and R6″=OH; or
R4″=OH and R5″=H and R6″=methyl; or
R4″=OH and R5″=H and R6″=O-methyl; or
R4″=OH and R5″=H and R6″=—S(═O)₂OH; or
R4″=OH and R5″=H and R6″=sulfonyl; or
R4″=OH and R5″=OH and R6″=H; or
R4″=OH and R5″=OH and R6″=OH; or
R4″=OH and R5″=OH and R6″=methyl; or
R4″=OH and R5″=OH and R6″=O-methyl; or
R4″=OH and R5″=OH and R6″=—S(═O)₂OH; or
R4″=OH and R5″=OH and R6″=sulfonyl; or
R4″=OH and R5″=methyl and R6″=H; or
R4″=OH and R5″=methyl and R6″=OH; or
R4″=OH and R5″=methyl and R6″=methyl; or
R4″=OH and R5″=methyl and R6″=O-methyl; or
R4″=OH and R5″=methyl and R6″=—S(═O)₂OH; or
R4″=OH and R5″=methyl and R6″=sulfonyl; or
R4″=OH and R5″=O-methyl and R6″=H; or
R4″=OH and R5″=O-methyl and R6″=OH; or
R4″=OH and R5″=O-methyl and R6″=methyl; or
R4″=OH and R5″=O-methyl and R6″=O-methyl; or
R4″=OH and R5″=O-methyl and R6″=—S(═O)₂OH; or
R4″=OH and R5″=O-methyl and R6″=sulfonyl; or
R4″=OH and R5″=—S(═O)₂OH and R6″=H; or
R4″=OH and R5″=—S(═O)₂OH and R6″=OH; or
R4″=OH and R5″=—S(═O)₂OH and R6″=methyl; or
R4″=OH and R5″=—S(═O)₂OH and R6″=O-methyl; or
R4″=OH and R5″=—S(═O)₂OH and R6″=—S(═O)₂OH; or
R4″=OH and R5″=—S(═O)₂OH and R6″=sulfonyl; or
R4″=OH and R5″=sulfonyl and R6″=H; or
R4″=OH and R5″=sulfonyl and R6″=OH; or
R4″=OH and R5″=sulfonyl and R6″=methyl; or
R4″=OH and R5″=sulfonyl and R6″=O-methyl; or
R4″=OH and R5″=sulfonyl and R6″=—S(═O)₂OH; or
R4″=OH and R5″=sulfonyl and R6″=sulfonyl; or
R4″=methyl and R5″=H and R6″=H; or
R4″=methyl and R5″=H and R6″=OH; or
R4″=methyl and R5″=H and R6″=methyl; or
R4″=methyl and R5″=H and R6″=O-methyl; or
R4″=methyl and R5″=H and R6″=—S(═O)₂OH; or
R4″=methyl and R5″=H and R6″=sulfonyl; or
R4″=methyl and R5″=OH and R6″=H; or
R4″=methyl and R5″=OH and R6″=OH; or
R4″=methyl and R5″=OH and R6″=methyl; or
R4″=methyl and R5″=OH and R6″=O-methyl; or
R4″=methyl and R5″=OH and R6″=—S(═O)₂OH; or
R4″=methyl and R5″=OH and R6″=sulfonyl; or
R4″=methyl and R5″=methyl and R6″=H; or
R4″=methyl and R5″=methyl and R6″=OH; or
R4″=methyl and R5″=methyl and R6″=methyl; or
R4″=methyl and R5″=methyl and R6″=O-methyl; or
R4″=methyl and R5″=methyl and R6″=—S(═O)₂OH; or
R4″=methyl and R5″=methyl and R6″=sulfonyl; or
R4″=methyl and R5″=O-methyl and R6″=H; or
R4″=methyl and R5″=O-methyl and R6″=OH; or
R4″=methyl and R5″=O-methyl and R6″=methyl; or
R4″=methyl and R5″=O-methyl and R6″=O-methyl; or
R4″=methyl and R5″=O-methyl and R6″=—S(═O)₂OH; or
R4″=methyl and R5″=O-methyl and R6″=sulfonyl; or
R4″=methyl and R5″=—S(═O)₂OH and R6″=H; or
R4″=methyl and R5″=—S(═O)₂OH and R6″=OH; or
R4″=methyl and R5″=—S(═O)₂OH and R6″=methyl; or
R4″=methyl and R5″=—S(═O)₂OH and R6″=O-methyl; or
R4″=methyl and R5″=—S(═O)₂OH and R6″=—S(═O)₂OH; or
R4″=methyl and R5″=—S(═O)₂OH and R6″=sulfonyl; or
R4″=methyl and R5″=sulfonyl and R6″=H; or
R4″=methyl and R5″=sulfonyl and R6″=OH; or
R4″=methyl and R5″=sulfonyl and R6″=methyl; or
R4″=methyl and R5″=sulfonyl and R6″=O-methyl; or
R4″=methyl and R5″=sulfonyl and R6″=—S(═O)₂OH; or
R4″=methyl and R5″=sulfonyl and R6″=sulfonyl; or
R4″=O-methyl and R5″=H and R6″=H; or
R4″=O-methyl and R5″=H and R6″=OH; or
R4″=O-methyl and R5″=H and R6″=methyl; or
R4″=O-methyl and R5″=H and R6″=O-methyl; or
R4″=O-methyl and R5″=H and R6″=—S(═O)₂OH; or
R4″=O-methyl and R5″=H and R6″=sulfonyl; or
R4″=O-methyl and R5″=OH and R6″=H; or
R4″=O-methyl and R5″=OH and R6″=OH; or
R4″=O-methyl and R5″=OH and R6″=methyl; or
R4″=O-methyl and R5″=OH and R6″=O-methyl; or
R4″=O-methyl and R5″=OH and R6″=—S(═O)₂OH; or
R4″=O-methyl and R5″=OH and R6″=sulfonyl; or
R4″=O-methyl and R5″=methyl and R6″=H; or
R4″=O-methyl and R5″=methyl and R6″=OH; or
R4″=O-methyl and R5″=methyl and R6″=methyl; or
R4″=O-methyl and R5″=methyl and R6″=O-methyl; or
R4″=O-methyl and R5″=methyl and R6″=—S(═O)₂OH; or
R4″=O-methyl and R5″=methyl and R6″=sulfonyl; or
R4″=O-methyl and R5″=O-methyl and R6″=H; or
R4″=O-methyl and R5″=O-methyl and R6″=OH; or
R4″=O-methyl and R5″=O-methyl and R6″=methyl; or
R4″=O-methyl and R5″=O-methyl and R6″=O-methyl; or
R4″=O-methyl and R5″=O-methyl and R6″=—S(═O)₂OH; or
R4″=O-methyl and R5″=O-methyl and R6″=sulfonyl; or
R4″=O-methyl and R5″=—S(═O)₂OH and R6″=H; or
R4″=O-methyl and R5″=—S(═O)₂OH and R6″=OH; or
R4″=O-methyl and R5″=—S(═O)₂OH and R6″=methyl; or
R4″=O-methyl and R5″=—S(═O)₂OH and R6″=O-methyl; or
R4″=O-methyl and R5″=—S(═O)₂OH and R6″=—S(═O)₂OH; or
R4″=O-methyl and R5″=—S(═O)₂OH and R6″=sulfonyl; or
R4″=O-methyl and R5″=sulfonyl and R6″=H; or
R4″=O-methyl and R5″=sulfonyl and R6″=OH; or
R4″=O-methyl and R5″=sulfonyl and R6″=methyl; or
R4″=O-methyl and R5″=sulfonyl and R6″=O-methyl; or
R4″=O-methyl and R5″=sulfonyl and R6″=—S(═O)₂OH; or
R4″=O-methyl and R5″=sulfonyl and R6″=sulfonyl; or
R4″=—S(═O)₂OH and R5″=H and R6″=H; or
R4″=—S(═O)₂OH and R5″=H and R6″=OH; or
R4″=—S(═O)₂OH and R5″=H and R6″=methyl; or
R4″=—S(═O)₂OH and R5″=H and R6″=O-methyl; or
R4″=—S(═O)₂OH and R5″=H and R6″=—S(═O)₂OH; or
R4″=—S(═O)₂OH and R5″=H and R6″=sulfonyl; or
R4″=—S(═O)₂OH and R5″=OH and R6″=H; or
R4″=—S(═O)₂OH and R5″=OH and R6″=OH; or
R4″=—S(═O)₂OH and R5″=OH and R6″=methyl; or
R4″=—S(═O)₂OH and R5″=OH and R6″=O-methyl; or
R4″=—S(═O)₂OH and R5″=OH and R6″=—S(═O)₂OH; or
R4″=—S(═O)₂OH and R5″=OH and R6″=sulfonyl; or
R4″=—S(═O)₂OH and R5″=methyl and R6″=H; or
R4″=—S(═O)₂OH and R5″=methyl and R6″=OH; or
R4″=—S(═O)₂OH and R5″=methyl and R6″=methyl; or
R4″=—S(═O)₂OH and R5″=methyl and R6″=O-methyl; or
R4″=—S(═O)₂OH and R5″=methyl and R6″=—S(═O)₂OH; or
R4″=—S(═O)₂OH and R5″=methyl and R6″=sulfonyl; or
R4″=—S(═O)₂OH and R5″=O-methyl and R6″=H; or
R4″=—S(═O)₂OH and R5″=O-methyl and R6″=OH; or
R4″=—S(═O)₂OH and R5″=O-methyl and R6″=methyl; or
R4″=—S(═O)₂OH and R5″=O-methyl and R6″=O-methyl; or
R4″=—S(═O)₂OH and R5″=O-methyl and R6″=—S(═O)₂OH; or
R4″=—S(═O)₂OH and R5″=O-methyl and R6″=sulfonyl; or
R4″=—S(═O)₂OH and R5″=—S(═O)₂OH═ and R6″=H; or
R4″=—S(═O)₂OH and R5″=—S(═O)₂OH and R6″=OH; or
R4″=—S(═O)₂OH and R5″=—S(═O)₂OH and R6″=methyl; or
R4″=—S(═O)₂OH and R5″=—S(═O)₂OH and R6″=O-methyl; or
R4″=—S(═O)₂OH and R5″=sulfonyl= and R6″=H; or
R4″=—S(═O)₂OH and R5″=sulfonyl and R6″=OH; or
R4″=—S(═O)₂OH and R5″=sulfonyl and R6″=methyl; or
R4″=—S(═O)₂OH and R5″=sulfonyl and R6″=O-methyl; or
R4″=sulfonyl and R5″=H and R6″=H; or
R4″=sulfonyl and R5″=H and R6″=OH; or
R4″=sulfonyl and R5″=H and R6″=methyl; or
R4″=sulfonyl and R5″=H and R6″=O-methyl; or
R4″=sulfonyl and R5″=H and R6″=—S(═O)₂OH; or
R4″=sulfonyl and R5″=H and R6″=sulfonyl; or
R4″=sulfonyl and R5″=OH and R6″=H; or
R4″=sulfonyl and R5″=OH and R6″=OH; or
R4″=sulfonyl and R5″=OH and R6″=methyl; or
R4″=sulfonyl and R5″=OH and R6″=O-methyl; or
R4″=sulfonyl and R5″=OH and R6″=—S(═O)₂OH; or
R4″=sulfonyl and R5″=OH and R6″=sulfonyl; or
R4″=sulfonyl and R5″=methyl and R6″=H; or
R4″=sulfonyl and R5″=methyl and R6″=OH; or
R4″=sulfonyl and R5″=methyl and R6″=methyl; or
R4″=sulfonyl and R5″=methyl and R6″=O-methyl; or
R4″=sulfonyl and R5″=methyl and R6″=—S(═O)₂OH; or
R4″=sulfonyl and R5″=methyl and R6″=sulfonyl; or
R4″=sulfonyl and R5″=O-methyl and R6″=H; or
R4″=sulfonyl and R5″=O-methyl and R6″=OH; or
R4″=sulfonyl and R5″=O-methyl and R6″=methyl; or
R4″=sulfonyl and R5″=O-methyl and R6″=O-methyl; or
R4″=sulfonyl and R5″=O-methyl and R6″=—S(═O)₂OH; or
R4″=sulfonyl and R5″=O-methyl and R6″=sulfonyl; or
R4″=sulfonyl and R5″=—S(═O)₂OH and R6″=H; or
R4″=sulfonyl and R5″=—S(═O)₂OH and R6″=OH; or
R4″=sulfonyl and R5″=—S(═O)₂OH and R6″=methyl; or
R4″=sulfonyl and R5″=—S(═O)₂OH and R6″=O-methyl; or
R4″=sulfonyl and R5″=sulfonyl and R6″=H; or
R4″=sulfonyl and R5″=sulfonyl and R6″=OH; or
R4″=sulfonyl and R5″=sulfonyl and R6″=methyl; or
R4″=sulfonyl and R5″=sulfonyl and R6″=O-methyl.

In the general formulae (V), (VI), (VII), (VIII), (IX), formula (X) and their variants, the substituted phenyl ring bonded to the imino functionality is preferably selected from the group consisting of

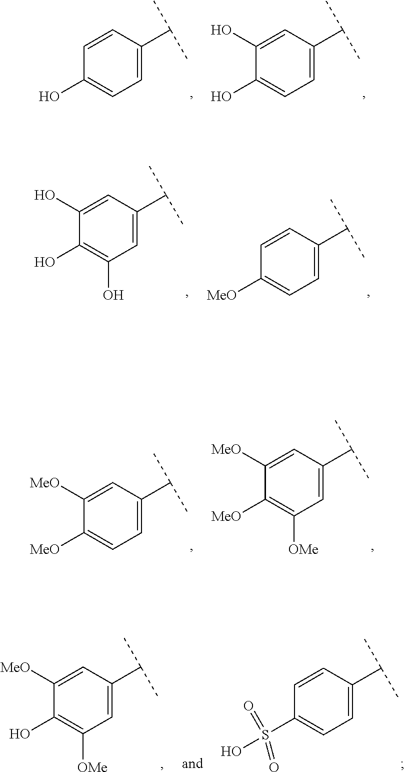

wherein the dotted line designates the binding site to the cyclohexene imine ring.

In a still more preferred variant according to the first aspect of the present invention, in the general formulae (V), (VII), (VIII) and their variants, the substituents R4′, R5′ and R6′, which may be identical or different, are independently from each other selected from the group consisting of H, OH, alkyl, preferably methyl, ethyl, propyl, iso-propyl, butyl, isobutyl or tert.-butyl, alkoxy, preferably, O-methyl, O-ethyl or O-butyl, —S(═O)₂OH, and sulfonyl; with the proviso, that if the substituted phenyl ring is monosubstituted, the substituent is either in the ortho or meta-position to the imino-functionality of the general formulae (V), (VII) and (VIII), but not in the para position to the imino-functionality.

Alternatively, in a still more preferred variant according to the first aspect of the present invention, in the general formulae (VI), (IX), (X) and their variants, the substituents R4″, R5″ and R6″, which may be identical or different, are independently from each other selected from the group consisting of H, OH, alkyl, preferably methyl, ethyl, propyl, iso-propyl, butyl, isobutyl or tert.-butyl, alkoxy, preferably, O-methyl, O-ethyl or O-butyl, —S(═O)₂OH, and sulfonyl; with the proviso, that if the substituted phenyl ring is monosubstituted, the substituent is either in the ortho or meta-position to the imino-functionality of the general formulae (VI), (IX) and (X), but not in the para position to the imino-functionality.

Still more preferred according to the first aspect of the present invention, the sunscreen product, cosmetic or pharmaceutical preparation or homecare product comprises at least one mycosporine-like amino acid compound selected from the group consisting of:

TABLE 3

| Compound | Structure | INCI/chemical name |
|---|---|---|
| MAA-1 | 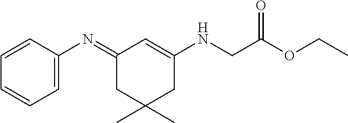 [SMILES: CCOC(=O)CNC1=C/C(=N/c2ccccc2)CC(C)(C)C1] | ethyl 2-[[5,5-dimethyl-3-phen- ylimino-cyclohexen-1-yl]amino] acetate |
| MAA-2 | 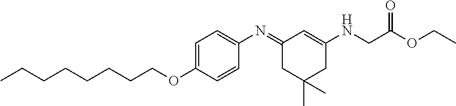 [SMILES: CCCCCCCCOc1ccc(/N=C2/C=C(NCC(=O)OCC)CC(C)(C)C2)cc1] | ethyl 2-((5,5-dimethyl-3-((4- octyoxy)phenyl)imino)cyclo- hex-1-en-1-yl)-amino)acetate |
| MAA-3 | 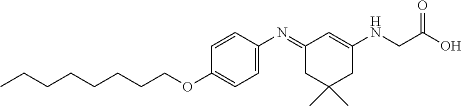 [SMILES: CCCCCCCCOc1ccc(/N=C2/C=C(NCC(=O)O)CC(C)(C)C2)cc1] | 5,5-dimethyl-3-((4-(octyloxy)- phenyl)imino)cyclohex-1-en-1- yl-glycine |
| MAA-4 | 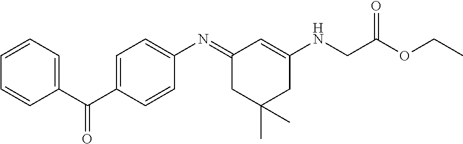 [SMILES: CCOC(=O)CNC1=C/C(=N/c2ccc(C(=O)c3ccccc3)cc2)CC(C)(C)C1] | ethyl 2-((3-((4-benzoylphenyl)- imino)-5,5-dimethylcyclohex-1- en-1-yl)-amino)acetate |
| MAA-5 | 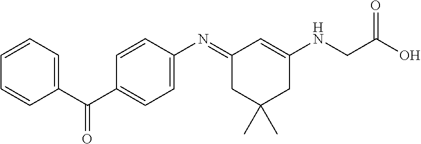 [SMILES: CC1(C)CC(NCC(=O)O)=C/C(=N/c2ccc(C(=O)c3ccccc3)cc2)C1] | 3-((4-benzoylphenyl)imino)- 5,5-dimethylcyclohex-1-en-1- yl-glycine |
| MAA-6 | 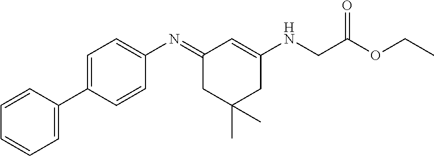 [SMILES: CCOC(=O)CNC1=C/C(=N/c2ccc(-c3ccccc3)cc2)CC(C)(C)C1] | ethyl 2-((3-([1,1′-biphenyl]-4- ylimino)-5,5-dimethylcyclo- hex-1-en-1.yl)amino)acetate |
| MAA-7 | 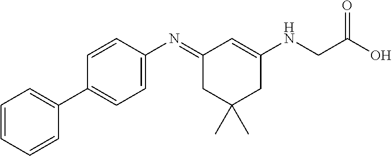 [SMILES: CC1(C)CC(NCC(=O)O)=C/C(=N/c2ccc(-c3ccccc3)cc2)C1] | 3-([1,1′-biphenyl]-4-ylimino)- 5,5-dimethylcyclohex-1-en-1- yl-glycine |
| MAA-8 | 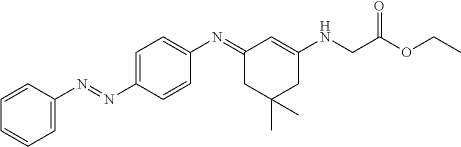 [SMILES: CCOC(=O)CNC1=C/C(=N/c2ccc(/N=N/c3ccccc3)cc2)CC(C)(C)C1] | ethyl 2-((5,5-dimethyl-3-((4- (phenyldiazenyl)phenyl)inino- (cyclohex-1-en-1-yl)amino)- acetate |
| MAA-9 | 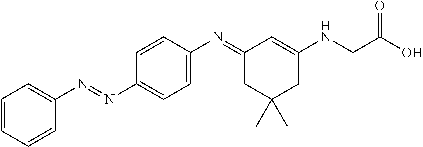 [SMILES: CC1(C)CC(NCC(=O)O)=C/C(=N/c2ccc(/N=N/c3ccccc3)cc2)C1] | 5,5-dimethyl-3-((4-((E)-phen- yldiazenyl)phenyl)imino)cyclo- hex-1-en-1-yl-glycine |
| MAA-11 | 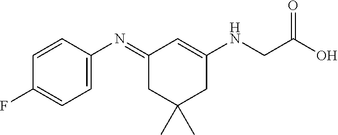 [SMILES: CC1(C)CC(NCC(=O)O)=C/C(=N/c2ccc(F)cc2)C1] | 3-((4-fluorophenyl)imino)-5,5- dimethylcyclohex-1-en-1-yl- glycine |
| MAA-12 | 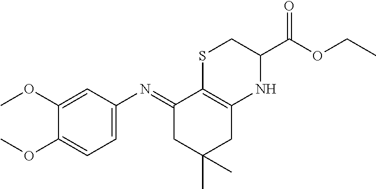 [SMILES: CCOC(=O)C1CSC2=C(CC(C)(C)C/C2=N\c2ccc(OC)c(OC)c2)N1] | ethyl 8-((3,4-dimethoyxphen- yl)imino)-6,6-dimethyl- 3,4,5,6,7,8-hexahydro-2-H- benzol[b][1,4]thiazine-3-car- boxylate |
| MAA-13 | 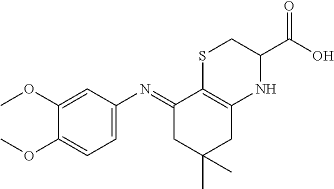 [SMILES: COc1ccc(/N=C2\CC(C)(C)CC3=C2SCC(C(=O)O)N3)cc1OC] | 8-((3,4-dimethoyxphenyl)- imino)-6,6-dimethyl- 3,4,5,6,7,8-hexahydro-2-H- benzol[b][1,4]thiazine-3-car- boxylic acid |
| MAA-14 | 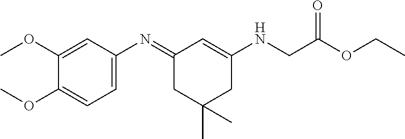 [SMILES: CCOC(=O)CNC1=C/C(=N/c2ccc(OC)c(OC)c2)CC(C)(C)C1] | ethyl 2-((3-((3,4-dimethoxy- phenyl)imino)-5,5-dimethyl- cyclohex-1-en-1-yl)amino)- acetate |
| MAA-16 | 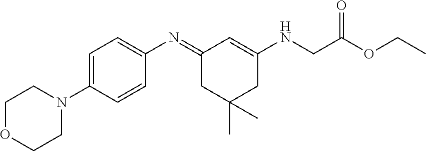 [SMILES: CCOC(=O)CNC1=C/C(=N/c2ccc(N3CCOCC3)cc2)CC(C)(C)C1] | ethyl 2-((5,5-dimethyl-3-((4- morpholinophenyl)imino)- cyclohex-1-en-1-yl)amino)- acetate |
| MAA-17 | 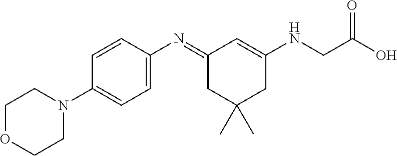 [SMILES: CC1(C)CC(NCC(=O)O)=C/C(=N/c2ccc(N3CCOCC3)cc2)C1] | 5,5-dimethyl-3-((4-morpholino- phenyl)imino)cyclohex-1-en-1- yl-glycine |
| MAA-20 | 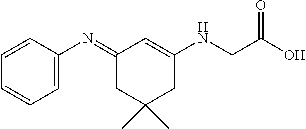 [SMILES: CC1(C)CC(NCC(=O)O)=C/C(=N/c2ccccc2)C1] | 2-[(5,5-dimethyl-3-phenyl- imino-cyclohexen-1-yl)- amino]acetic acid |
| MAA-21 | 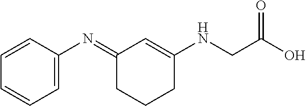 [SMILES: O=C(O)CNC1=C/C(=N/c2ccccc2)CCC1] | 2-[(3-phenyliminocyclohexen- 1-yl)-amino]acetic acid |
| MAA-22 | 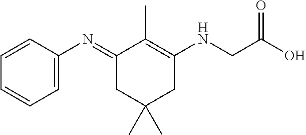 [SMILES: CC1=C(NCC(=O)O)CC(C)(C)C/C1=N\c1ccccc1] | 2,5,5-trimethyl-3-(phenyl- imino)cyclohex-1-en-1-yl- glycine |
| MAA-23 | 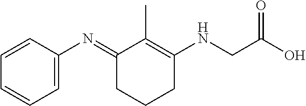 [SMILES: CC1=C(NCC(=O)O)CCC/C1=N\c1ccccc1] | 2-[(2-methyl-3-phenylimino- cyclohexen-1-yl)amino]acetic acid |
| MAA-24 | 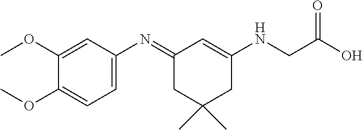 [SMILES: COc1ccc(/N=C2/C=C(NCC(=O)O)CC(C)(C)C2)cc1OC] | 3-((3,4-dimethoxyphenyl)- imino)-5,5-dimethylcyclohex- 1-en-1-yl-glycine |
| MAA-25 | 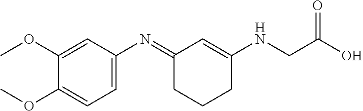 [SMILES: COc1ccc(/N=C2/C=C(NCC(=O)O)CCC2)cc1OC] | 3-((3,4-dimethoxyphenyl)- imino)cyclohex-1-en-1-yl- glycine |
| MAA-26 | 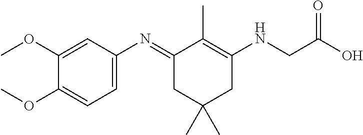 [SMILES: COc1ccc(/N=C2\CC(C)(C)CC(NCC(=O)O)=C2C)cc1OC] | 3-((3,4-dimethoxyphenyl)- imino)-2,5,5-trimethylcyclo- hex-1-en-1-yl-glycine |
| MAA-27 | 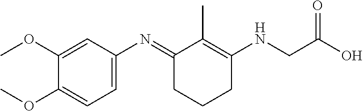 [SMILES: COc1ccc(/N=C2\CCCC(NCC(=O)O)=C2C)cc1OC] | 2-[(3-(3,4-dimethoxyphenyl)- imino-2-methyl-cyclohexen-1- yl)amino]acetic acid |
| MAA-28 | 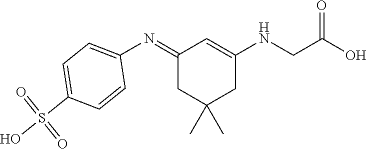 [SMILES: CC1(C)CC(NCC(=O)O)=C/C(=N/c2ccc(S(=O)(=O)O)cc2)C1] | 2-[(5,5-dimethyl-3-(4-sulfo- phenyl)imino-cyclohexen-1- yl)amino]acetic acid |
| MAA-29 | 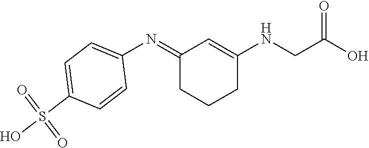 [SMILES: O=C(O)CNC1=C/C(=N/c2ccc(S(=O)(=O)O)cc2)CCC1] | 2-[(3-(4-sulfophenyl)imino- cyclohexen-1-yl)amino]acetic acid |
| MAA-30 | 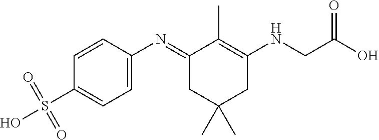 [SMILES: CC1=C(NCC(=O)O)CC(C)(C)C/C1=N\c1ccc(S(=O)(=O)O)cc1] | 2,5,5-trimethyl-3-((4-sulfo- phenyl)imino)cyclohex-1-en- 1-yl-glycine |
| MAA-31 | 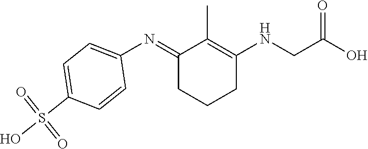 [SMILES: CC1=C(NCC(=O)O)CCC/C1=N\c1ccc(S(=O)(=O)O)cc1] | 2-methyl-3-((4-sulfophenyl)- imino)cyclohex-1-en-1-yl- glycine |
| MAA-32 | 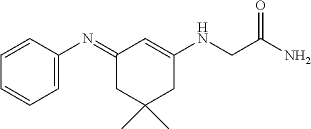 [SMILES: CC1(C)CC(NCC(N)=O)=C/C(=N/c2ccccc2)C1] | 2-[(5,5-dimethyl-3-phenyl- imino-cyclohexen-1-yl)- amino]acetamide |
| MAA-33 | 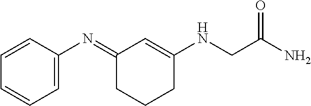 [SMILES: NC(=O)CNC1=C/C(=N/c2ccccc2)CCC1] | 2-[(3-phenyliminocyclohexen- 1-yl)amino]acetamide |
| MAA-34 | 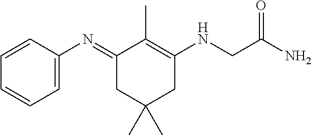 [SMILES: CC1=C(NCC(N)=O)CC(C)(C)C/C1=N\c1ccccc1] | 2-((2,5,5-trimethyl-3-(phenyl- imino)cyclohex-1-en-1-yl)- amino)acetamide |
| MAA-35 | 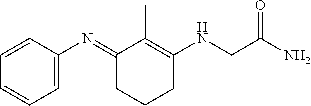 [SMILES: CC1=C(NCC(N)=O)CCC/C1=N\c1ccccc1] | 2-((2-methyl-3-(phenylimino)- cyclohex-1-en-1-yl)amino)- acetamide |
| MAA-36 | 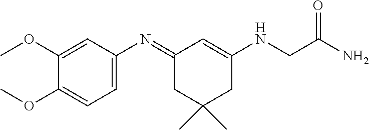 [SMILES: COc1ccc(/N=C2/C=C(NCC(N)=O)CC(C)(C)C2)cc1OC] | 2-((3-((3,4-dimethoxyphenyl)- imino)-5,5-dimethylcyclohex- 1-en-1-yl)amino)acetamide |
| MAA-37 | 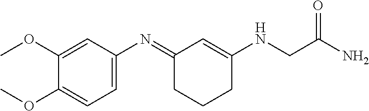 [SMILES: COc1ccc(/N=C2/C=C(NCC(N)=O)CCC2)cc1OC] | 2-[(3-(3,4-dimethoxyphenyl)- iminocyclohexen-1-yl)amino] acetamide |
| MAA-38 | 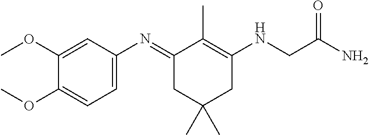 [SMILES: COc1ccc(/N=C2\CC(C)(C)CC(NCC(N)=O)=C2C)cc1OC] | 2-((3-((3,4-dimethoxyphenyl)- imino)-2,5,5-trimethylcyclo- hex-1-en-1-yl)amino)acetamide |
| MAA-39 | 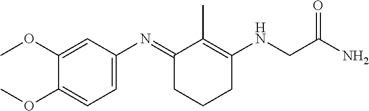 [SMILES: COc1ccc(/N=C2\CCCC(NCC(N)=O)=C2C)cc1OC] | 2-((3-((3,4-dimethoxyphenyl)- imino)-2-methylcyclohex-1- en-1-yl)amino)acetamide |

or a tautomer or a stereoisomer or a salt thereof, or any mixture of the afore-mentioned compounds.

Among the above specified mycosporine-like amino acid compounds of Table 3 the compounds MAA-1 and MAA-13 are particularly preferred, since they have a pronounced photo-stabilizing effect. Most preferred are the acid compounds MAA-20, MAA-21, MAA-22, MAA-23, MAA-24, MAA-24, MAA-26, MAA-27, MAA-28, MAA-29, MAA-30, MAA-31, and the amide compounds MAA-32, MAA-33, MAA-34, MAA-35, MAA-36, MAA-37, MAA-38, and MAA-39. Said compounds having an acid functionality or an amide functionality are photo-stable and temperature-stable and show stability in pH solution or in oil-in-water emulsions and does not degrade compared to the respective ester analogues. Particularly preferred are the acid compounds MAA-20, MAA-21, MAA-22, MAA-23, MAA-24, MAA-24, MAA-26, MAA-27, MAA-28, MAA-29, MAA-30 and MAA-31 due to their stability and their improved solubility in water.

Especially, the mycosporine-like amino acid compounds MAA-20, MAA-21, MAA-22, MAA-23, MAA-24, MAA-24, MAA-26, MAA-27, MAA-28, MAA-29, MAA-30 and MAA-31 have a better stability in a broader pH-range compared to their respective ester compounds which hydrolyze, and, thus, are not stable in alkaline solutions/formulations having a pH higher than 9.

Due to their better solubility in water, said mycosporine-like amino acid compounds can be incorporated into the water-phase of formulations in higher concentrations, resulting in a better photostabilizing effect and, thus, an improved sunscreen in sunscreen products, cosmetic or pharmaceutical preparations or homecare products, compared to their respective ester compounds.

Still more preferred according to the first aspect of the present invention, the sunscreen product, cosmetic or pharmaceutical preparation or homecare product comprises at least one mycosporine-like amino acid compound selected from the group consisting of:

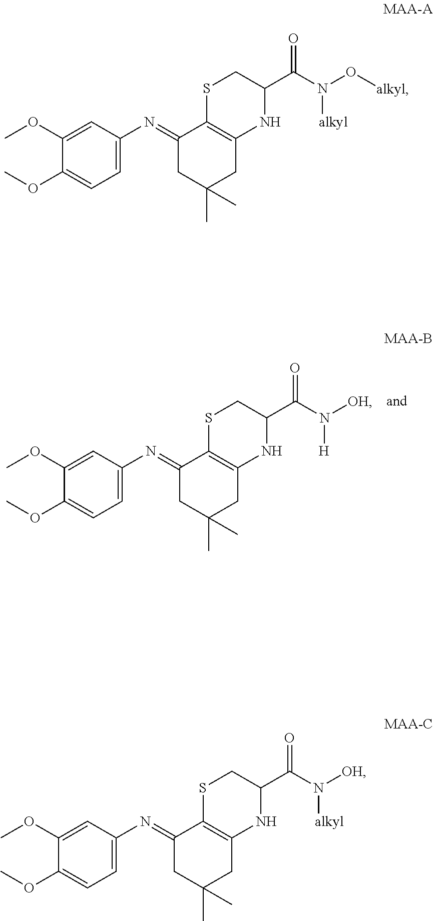

or a tautomer or a stereoisomer or a salt thereof, or any mixture of the afore-mentioned compounds, wherein alkyl is preferably either methyl or ethyl or propyl or isopropyl or butyl or isobutyl or tert-butyl, most preferred methyl.

The mycosporine-like amino acid compounds according to the present invention and defined herein are used either as single substance or in a mixture with one, two or more different mycosporine-like amino acid compounds as defined herein.

Particular preferred are mixtures of two mycosporine-like amino acid compounds according to the present invention and defined herein. Such mixtures show a particular enhanced photostabilizing effect, as it is demonstrated by the following examples.

The sunscreen product, cosmetic or pharmaceutical preparation or homecare product according to the first aspect of the present invention is further characterized in that it does not contain one or more of the following mycosporine-like amino acid compounds:

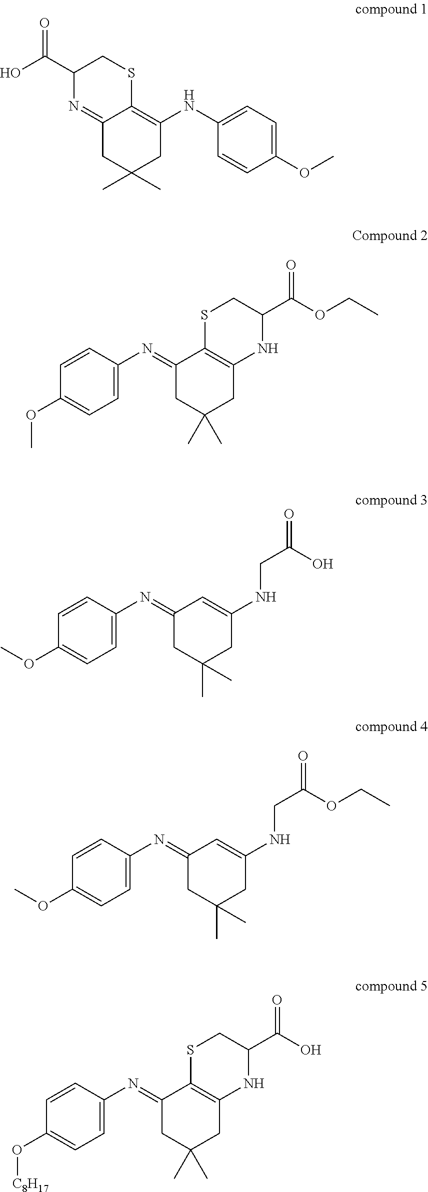

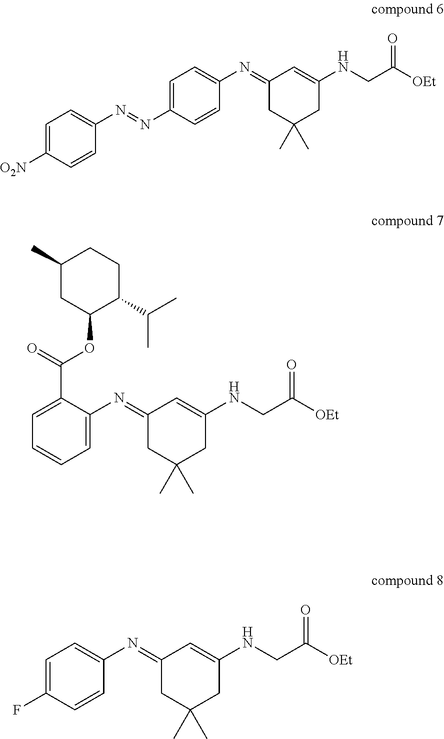 [SMILES: CCOC(=O)CNC1=C/C(=N/c2ccc(/N=N/c3ccc([N+](=O)[O-])cc3)cc2)CC(C)(C)C1 ; CCOC(=O)CNC1=C/C(=N/c2ccccc2C(=O)O[C@H]2C[C@@H](C)CC[C@@H]2C(C)C)CC(C)(C)C1 ; CCOC(=O)CNC1=C/C(=N/c2ccc(F)cc2)CC(C)(C)C1]

Various mycosporine-like amino acid compounds (b) as described herein contain one or more chiral centers, and, thus, can exist as racemic mixtures of enantiomers, mixtures of diastereomers or enantiomerically or optically pure compounds. It should also be noted the compounds of the invention include E and Z isomers, or a mixture thereof, and cis and trans isomers or a mixture thereof.

Accordingly, the chemical structures of the mycosporine-like amino acid compounds according to the general formulae (V) to (X) and their variants and compounds explicitly disclosed herein include all possible enantiomers and diastereomers or stereoisomers thereof.

In certain embodiments, the mycosporine-like amino acid compounds (b) of the invention are isolated as either the E or Z isomer. In other embodiments, the compounds of the invention are a mixture of the E and Z isomers.

Alternatively, stereomerically pure compounds are used in the sunscreen product or cosmetic or pharmaceutical preparation according to the present invention. As used herein and unless otherwise indicated, the term “stereomerically pure” means that one stereoisomer of a compound is substantially free of other stereoisomers of that compound or one geometric isomer (e.g., about a double bond) is substantially free of the other geometric isomer. For example, a stereomerically pure compound of the invention having one chiral center, will be substantially free of the opposite enantiomer of the compound. A stereomerically pure compound of the invention having two chiral centers, or a composition thereof, will be substantially free of other diastereomers of the compound. A stereomerically pure compound of the invention having a double bond capable of E/Z isomerism, or a composition thereof, will be substantially free of one of the E/Z isomers.

A typical stereomerically pure compound comprises greater than about 80% by weight of one stereoisomer or E/Z isomer of the compound and less than about 20% by weight of other stereoisomers or E/Z isomer of the compound, more preferably greater than about 90% by weight of one stereoisomer or E/Z isomer of the compound and less than about 10% by weight of the other stereoisomers or E/Z isomer of the compound, even more preferably greater than about 95% by weight of one stereoisomer or E/Z isomer of the compound and less than about 5% by weight of the other stereoisomers or E/Z isomer of the compound, and most preferably greater than about 97% by weight of one stereoisomer or E/Z isomer of the compound and less than about 3% by weight of the other stereoisomers or E/Z isomer of the compound.

As used herein and unless otherwise indicated, the term “stereomerically enriched” means a compound of the invention, or a composition thereof, that comprises greater than about 60% by weight of one stereoisomer or E/Z isomer of a compound of the invention, preferably greater than about 70% by weight, more preferably greater than about 80% by weight of one stereoisomer or E/Z isomer of a compound of the invention.

Thus, it should be noted that unless stated otherwise, the term mycosporine-like amino acid compound according to the general formulae (I) to (X) and their variants as well as individual mycosporine-like amino acid compounds specified herein, are to be interpreted as encompassing racemic mixtures of enantiomers, mixtures of diastereomers or enantiomerically or optically pure compounds.

Due to their amino cyclohexene imine structure and their capability of electron delocalization, the mycosporine-like amino acid compounds according to general formulae (V) to (X) and their variants are in equilibrium with their tautomer structures, in which the hydrogen of the amino group changes its places with the double bond of the cyclohexene ring.

Tautomerism is defined as each of two or more isomers of a compound which exist together in equilibrium and are readily interchanged by migration of an atom or group within the molecule. Tautomeric pure or enriched systems will readily interchange into the equilibrium state over time.

The tautomers of general formula (V) are represented by general formulae (V-tau):

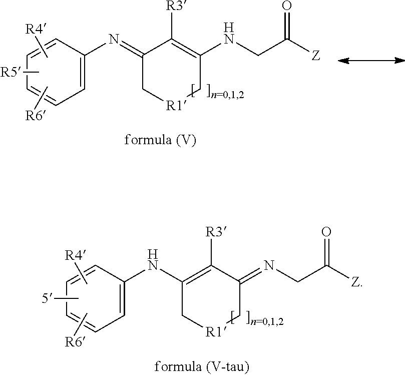

The tautomers of general formula (VI) are represented by general formula (VI-tau):

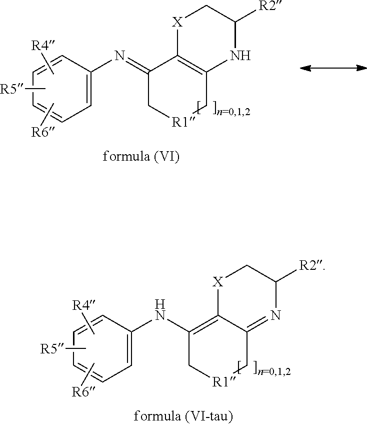

The above principles of tautomerism and redeposition explained on the basis of the mycosporine-like amino acid compounds according to the general formula (V) and (VI) are likewise applicable with respect to any of the mycosporine-like amino acid compounds defined by the general formulae (VII), (VIII), (IX), (X) and their variants or with respect to any individual mycosporine-like amino acid compounds specified herein.

Thus, in the following and throughout the description, the reference to a mycosporine-like amino acid compound represented by any general formulae (V) to (X) and their variants or to an individual mycosporine-like amino acid compound specified herein encompasses likewise its tautomer isomer(s).

The mycosporine-like amino acid compounds according to the general formulae (V) to (X) and their variants as well as individual mycosporine-like amino acid compounds specified herein are used according to the first aspect of the present invention either in neutral, i.e. uncharged form, or in the form of their salts, such as an acid addition salt, with inorganic or organic acids.

The term “salt” in the context of the present invention refers to a salt of a compound that possesses the desired effect or pharmacological activity of the parent compound. Such salts include:
(1) acid addition salts formed with inorganic acids, or formed with organic acids, preferably monovalent or polyvalent carboxylic acids; or
(2) salts formed when an acidic proton present in the starting compound is replaced by a metal ion, e.g., an alkali metal ion, an alkaline earth ion, or an aluminium ion; or coordinated with an organic base.

Among the salts, acid addition salts are again particularly preferred, since the mycosporine-like amino acid compounds according to the general formulae (V) to (X) and their variants or the mycosporine-like amino acid compounds specified herein comprise a protonable N atom.

The inorganic acids that form acid addition salts with the mycosporine-like amino acid compounds used according to the present invention are preferably selected from the group consisting of hydrochloric acid, hydrobromic acid, sulfuric acid, nitric acid, phosphoric acid and the like. Particularly preferred is the hydrochloride salt or the sulfate salt or the phosphate salt.

Even more preferred are acid addition salts with organic mono- or polycarboxylic acids. Further preferred are acid addition salts with organic mono- or polycarboxylic acids, wherein the carboxylic acid is selected from saturated or mono- or polyunsaturated C1 to C30 monocarboxylic acids, saturated or mono- or polyunsaturated C3 to 10 di- or tricarboxylic acids. The carboxylic acid may be mono- or poly-substituted with hydroxy groups, preferably α-hydroxycarboxylic acids in which the hydroxy group is located on the carbon atom adjacent to the carboxy group. Many representatives occur naturally as so-called fruit acids. Preferred α-hydroxycarboxylic acids are malic acid, citric acid, 2-hydroxy-4-methylmercaptobutyric acid, glycolic acid, isocitric acid, mandelic acid, lactic acid, tartronic acid, or tartaric acid.

The organic acids that form acid addition salts with the mycosporine-like amino acid compounds used according to the present invention are preferably selected from the group consisting of amino acids, acetic acid, trifluoroacetic acid, propionic acid, hexanoic acid, cyclopentane propionic acid, glycolic acid, pyruvic acid, lactic acid, malonic acid, oxalic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tert. butylacetic acid, laurylsulfuric acid, gluconic acid, glutamic acid, hydroxynaphthoic acid, salicylic acid, stearic acid, muconic acid, 4-hydroxybutanoic acid, and the like.

Suitable anionic counterions are in particular chloride, bromide, hydrogensulfate, sulfate, dihydrogenphosphate, hydrogenphosphate, phosphate, nitrate, bicarbonate, carbonate, hexafluorosilicate, hexafluorophosphate, benzoate, and the anions of C1- to C4-alkanoic acids, preferably formate, acetate, trifluoroacetate, propionate and butyrate, furthermore lactate, gluconate, and the anions of poly acids such as succinate, oxalate, maleate, fumarate, malate, tartrate and citrate, furthermore sulfonate anions such as besylate (benzenesulfonate), tosylate (p-toluenesulfonate), napsylate (naphthalene-2-sulfonate), mesylate (methanesulfonate), esylate (ethanesulfonate), and ethanedisulfonate. They can be formed by reacting compounds according to the invention that have a basic functionality with an acid of the corresponding anion. Preferred salts of the compounds of formulae (I) to (III) as well as of the individual mycosporine-like amino acid compounds specified herein are chloride salts.

The metal ions for salt formation that replace an acidic proton present in the starting compound are selected from the group consisting of alkali metal ions, preferably Na+, K+, or Li+, alkaline earth metal ions, preferably Ca++ or Mg++, aluminium+++, or NH4+.

The coordinating organic base for salt formation is selected from the group consisting of ethanolamine, diethanolamine, triethanolamine, N-methylglucamine, triethylamine, and the like.

In the following description and claims, and unless stated otherwise, the term mycosporine-like amino acid compound includes both the neutral, uncharged form of the compound/molecule and equally the salt form of the compound.

The salt form of the mycosporine-like amino acid compounds leads to a lower logPow and, thus, making the compounds more hydrophilic, which results in a better water solubility. In particular, the cationic salts of the mycosporine-like amino acid compounds support emulsification processes in emulsions, due to their surface reducing activity, i.e. co-emulsifying, properties. In addition, the cationic salt form of the mycosporine-like compounds shows excellent substantivity behaviour on skin and hair and other non-biological surfaces: most conditioning actives are cationic; the conditioning effect leads to a softer skin feel, which makes the end products more accepted by the consumer. In addition, the mycosporine-like amino acid compounds in the cationic salt form exhibit antimicrobial activity.

In a particular advantageously variant, the sunscreen product, cosmetic or pharmaceutical preparation or homecare product according to the first aspect of the present invention comprises one of the following combinations of components (a) and (b):
Ethylhexyl-methoxycinnamate plus at least one mycosporine-like amino acid compound according to any one of formula (V) as defined herein; or
Butyl-methoxydibenzoylmethane plus at least one mycosporine-like amino acid compound according to any one of formula (V) as defined herein; or
Isoamyl-p-Methoxycinnamate plus at least one mycosporine-like amino acid compound according to any one of formula (V); or
Diethylamino-hydroxybenzoyl-hexyl-benzoate plus at least one mycosporine-like amino acid compound according to any one of formula (V) as defined herein; or
Ethylhexyl-methoxycinnamate plus at least one mycosporine-like amino acid compound according to any one of formula (VI) as defined herein; or
Butyl-methoxydibenzoylmethane plus at least one mycosporine-like amino acid compound according to any one of formula (VI) as defined herein; or
Isoamyl-p-Methoxycinnamate plus at least one mycosporine-like amino acid compound according to any one of formula (VI); or
Diethylamino-hydroxybenzoyl-hexyl-benzoate plus at least one mycosporine-like amino acid compound according to any one of formula (VI) as defined herein; or
Ethylhexyl-methoxycinnamate plus at least one mycosporine-like amino acid compound according to any one of formula (VII) as defined herein; or
Butyl-methoxydibenzoylmethane plus at least one mycosporine-like amino acid compound according to any one of formula (VII) as defined herein; or
Isoamyl-p-Methoxycinnamate plus at least one mycosporine-like amino acid compound according to any one of formula (VII); or
Diethylamino-hydroxybenzoyl-hexyl-benzoate plus at least one mycosporine-like amino acid compound according to any one of formula (VII) as defined herein; or
Ethylhexyl-methoxycinnamate plus at least one mycosporine-like amino acid compound according to any one of formula (VIII) as defined herein; or
Butyl-methoxydibenzoylmethane plus at least one mycosporine-like amino acid compound according to any one of formula (VIII) as defined herein; or
Isoamyl-p-Methoxycinnamate plus at least one mycosporine-like amino acid compound according to any one of formula (VIII); or
Diethylamino-hydroxybenzoyl-hexyl-benzoate plus at least one mycosporine-like amino acid compound according to any one of formula (VIII) as defined herein.

More preferred combinations of component (a) and (b) in the sunscreen product or cosmetic or pharmaceutical preparation according to the first aspect of the present invention are:
Ethylhexyl-methoxycinnamate plus compound MAA-1 as depicted in Table 3; or
Butyl-methoxydibenzoylmethane plus compound MAA-1 as depicted in Table 3; or
Isoamyl-p-Methoxycinnamate plus compound MAA-1 as depicted in Table 3; or
Diethylamino-hydroxybenzoyl-hexyl-benzoate plus compound MAA-1 as depicted in Table 3; or
Ethylhexyl-methoxycinnamate plus compound MAA-2 as depicted in Table 3; or
Butyl-methoxydibenzoylmethane plus compound MAA-2 as depicted in Table 3; or
Isoamyl-p-Methoxycinnamate plus compound MAA-2 as depicted in Table 3; or
Diethylamino-hydroxybenzoyl-hexyl-benzoate plus compound MAA-2 as depicted in Table 3; or
Ethylhexyl-methoxycinnamate plus compound MAA-3 as depicted in Table 3; or
Butyl-methoxydibenzoylmethane plus compound MAA-3 as depicted in Table 3; or
Isoamyl-p-Methoxycinnamate plus compound MAA-3 as depicted in Table 3; or
Diethylamino-hydroxybenzoyl-hexyl-benzoate plus compound MAA-3 as depicted in Table 3; or
Ethylhexyl-methoxycinnamate plus compound MAA-4 as depicted in Table 3; or
Butyl-methoxydibenzoylmethane plus compound MAA-4 as depicted in Table 3; or
Isoamyl-p-Methoxycinnamate plus compound MAA-4 as depicted in Table 3; or
Diethylamino-hydroxybenzoyl-hexyl-benzoate plus compound MAA-4 as depicted in Table 3; or
Ethylhexyl-methoxycinnamate plus compound MAA-5 as depicted in Table 3; or
Butyl-methoxydibenzoylmethane plus compound MAA-5 as depicted in Table 3; or
Isoamyl-p-Methoxycinnamate plus compound MAA-5 as depicted in Table 3; or
Diethylamino-hydroxybenzoyl-hexyl-benzoate plus compound MAA-5 as depicted in Table 3; or
Ethylhexyl-methoxycinnamate plus compound MAA-6 as depicted in Table 3; or
Butyl-methoxydibenzoylmethane plus compound MAA-6 as depicted in Table 3; or
Isoamyl-p-Methoxycinnamate plus compound MAA-6 as depicted in Table 3; or
Diethylamino-hydroxybenzoyl-hexyl-benzoate plus compound MAA-6 as depicted in Table 3; or
Ethylhexyl-methoxycinnamate plus compound MAA-7 as depicted in Table 3; or
Butyl-methoxydibenzoylmethane plus compound MAA-7 as depicted in Table 3; or
Isoamyl-p-Methoxycinnamate plus compound MAA-7 as depicted in Table 3; or
Diethylamino-hydroxybenzoyl-hexyl-benzoate plus compound MAA-7 as depicted in Table 3; or
Ethylhexyl-methoxycinnamate plus compound MAA-8 as depicted in Table 3; or
Butyl-methoxydibenzoylmethane plus compound MAA-8 as depicted in Table 3; or
Isoamyl-p-Methoxycinnamate plus compound MAA-8 as depicted in Table 3; or
Diethylamino-hydroxybenzoyl-hexyl-benzoate plus compound MAA-8 as depicted in Table 3; or
Ethylhexyl-methoxycinnamate plus compound MAA-9 as depicted in Table 3; or
Butyl-methoxydibenzoylmethane plus compound MAA-9 as depicted in Table 3; or
Isoamyl-p-Methoxycinnamate plus compound MAA-9 as depicted in Table 3; or
Diethylamino-hydroxybenzoyl-hexyl-benzoate plus compound MAA-9 as depicted in Table 3; or
Ethylhexyl-methoxycinnamate plus compound MAA-11 as depicted in Table 3; or
Butyl-methoxydibenzoylmethane plus compound MAA-11 as depicted in Table 3; or
Isoamyl-p-Methoxycinnamate plus compound MAA-11 as depicted in Table 3; or
Diethylamino-hydroxybenzoyl-hexyl-benzoate plus compound MAA-11 as depicted in Table 3; or
Ethylhexyl-methoxycinnamate plus compound MAA-12 as depicted in Table 3; or
Butyl-methoxydibenzoylmethane plus compound MAA-12 as depicted in Table 3; or
Isoamyl-p-Methoxycinnamate plus compound MAA-12 as depicted in Table 3; or
Diethylamino-hydroxybenzoyl-hexyl-benzoate plus compound MAA-12 as depicted in Table 3; or
Ethylhexyl-methoxycinnamate plus compound MAA-13 as depicted in Table 3; or
Butyl-methoxydibenzoylmethane plus compound MAA-13 as depicted in Table 3; or
Isoamyl-p-Methoxycinnamate plus compound MAA-13 as depicted in Table 3; or
Diethylamino-hydroxybenzoyl-hexyl-benzoate plus compound MAA-13 as depicted in Table 3; or
Ethylhexyl-methoxycinnamate plus compound MAA-14 as depicted in Table 3; or
Butyl-methoxydibenzoylmethane plus compound MAA-14 as depicted in Table 3; or
Isoamyl-p-Methoxycinnamate plus compound MAA-14 as depicted in Table 3; or
Diethylamino-hydroxybenzoyl-hexyl-benzoate plus compound MAA-14 as depicted in Table 3; or
Ethylhexyl-methoxycinnamate plus compound MAA-16 as depicted in Table 3; or
Butyl-methoxydibenzoylmethane plus compound MAA-16 as depicted in Table 3; or
Isoamyl-p-Methoxycinnamate plus compound MAA-16 as depicted in Table 3; or
Diethylamino-hydroxybenzoyl-hexyl-benzoate plus compound MAA-16 as depicted in Table 3; or
Ethylhexyl-methoxycinnamate plus compound MAA-17 as depicted in Table 3; or
Butyl-methoxydibenzoylmethane plus compound MAA-17 as depicted in Table 3; or
Isoamyl-p-Methoxycinnamate plus compound MAA-17 as depicted in Table 3; or
Diethylamino-hydroxybenzoyl-hexyl-benzoate plus compound MAA-17 as depicted in Table 3; or
Ethylhexyl-methoxycinnamate plus compound MAA-20 as depicted in Table 3; or
Butyl-methoxydibenzoylmethane plus compound MAA-20 as depicted in Table 3; or
Isoamyl-p-Methoxycinnamate plus compound MAA-20 as depicted in Table 3; or
Diethylamino-hydroxybenzoyl-hexyl-benzoate plus compound MAA-20 as depicted in Table 3; or
Ethylhexyl-methoxycinnamate plus compound MAA-21 as depicted in Table 3; or
Butyl-methoxydibenzoylmethane plus compound MAA-21 as depicted in Table 3; or
Isoamyl-p-Methoxycinnamate plus compound MAA-21 as depicted in Table 3; or
Diethylamino-hydroxybenzoyl-hexyl-benzoate plus compound MAA-21 as depicted in Table 3; or
Ethylhexyl-methoxycinnamate plus compound MAA-22 as depicted in Table 3; or
Butyl-methoxydibenzoylmethane plus compound MAA-22 as depicted in Table 3; or
Isoamyl-p-Methoxycinnamate plus compound MAA-22 as depicted in Table 3; or
Diethylamino-hydroxybenzoyl-hexyl-benzoate plus compound MAA-22 as depicted in Table 3; or
Ethylhexyl-methoxycinnamate plus compound MAA-23 as depicted in Table 3; or
Butyl-methoxydibenzoylmethane plus compound MAA-23 as depicted in Table 3; or
Isoamyl-p-Methoxycinnamate plus compound MAA-23 as depicted in Table 3; or
Diethylamino-hydroxybenzoyl-hexyl-benzoate plus compound MAA-23 as depicted in Table 3; or
Ethylhexyl-methoxycinnamate plus compound MAA-24 as depicted in Table 3; or
Butyl-methoxydibenzoylmethane plus compound MAA-24 as depicted in Table 3; or
Isoamyl-p-Methoxycinnamate plus compound MAA-24 as depicted in Table 3; or
Diethylamino-hydroxybenzoyl-hexyl-benzoate plus compound MAA-24 as depicted in Table 3; or
Ethylhexyl-methoxycinnamate plus compound MAA-25 as depicted in Table 3; or
Butyl-methoxydibenzoylmethane plus compound MAA-25 as depicted in Table 3; or
Isoamyl-p-Methoxycinnamate plus compound MAA-25 as depicted in Table 3; or
Diethylamino-hydroxybenzoyl-hexyl-benzoate plus compound MAA-25 as depicted in Table 3; or
Ethylhexyl-methoxycinnamate plus compound MAA-26 as depicted in Table 3; or
Butyl-methoxydibenzoylmethane plus compound MAA-26 as depicted in Table 3; or
Isoamyl-p-Methoxycinnamate plus compound MAA-26 as depicted in Table 3; or
Diethylamino-hydroxybenzoyl-hexyl-benzoate plus compound MAA-26 as depicted in Table 3; or
Ethylhexyl-methoxycinnamate plus compound MAA-27 as depicted in Table 3; or
Butyl-methoxydibenzoylmethane plus compound MAA-27 as depicted in Table 3; or
Isoamyl-p-Methoxycinnamate plus compound MAA-27 as depicted in Table 3; or
Diethylamino-hydroxybenzoyl-hexyl-benzoate plus compound MAA-27 as depicted in Table 3; or
Ethylhexyl-methoxycinnamate plus compound MAA-28 as depicted in Table 3; or
Butyl-methoxydibenzoylmethane plus compound MAA-28 as depicted in Table 3; or
Isoamyl-p-Methoxycinnamate plus compound MAA-28 as depicted in Table 3; or
Diethylamino-hydroxybenzoyl-hexyl-benzoate plus compound MAA-28 as depicted in Table 3; or
Ethylhexyl-methoxycinnamate plus compound MAA-29 as depicted in Table 3; or
Butyl-methoxydibenzoylmethane plus compound MAA-29 as depicted in Table 3; or
Isoamyl-p-Methoxycinnamate plus compound MAA-29 as depicted in Table 3; or
Diethylamino-hydroxybenzoyl-hexyl-benzoate plus compound MAA-29 as depicted in Table 3; or
Ethylhexyl-methoxycinnamate plus compound MAA-30 as depicted in Table 3; or
Butyl-methoxydibenzoylmethane plus compound MAA-30 as depicted in Table 3; or
Isoamyl-p-Methoxycinnamate plus compound MAA-30 as depicted in Table 3; or
Diethylamino-hydroxybenzoyl-hexyl-benzoate plus compound MAA-30 as depicted in Table 3; or
Ethylhexyl-methoxycinnamate plus compound MAA-31 as depicted in Table 3; or
Butyl-methoxydibenzoylmethane plus compound MAA-31 as depicted in Table 3; or
Isoamyl-p-Methoxycinnamate plus compound MAA-31 as depicted in Table 3; or
Diethylamino-hydroxybenzoyl-hexyl-benzoate plus compound MAA-31 as depicted in Table 3; or
Ethylhexyl-methoxycinnamate plus compound MAA-32 as depicted in Table 3; or
Butyl-methoxydibenzoylmethane plus compound MAA-32 as depicted in Table 3; or
Isoamyl-p-Methoxycinnamate plus compound MAA-32 as depicted in Table 3; or
Diethylamino-hydroxybenzoyl-hexyl-benzoate plus compound MAA-32 as depicted in Table 3; or
Ethylhexyl-methoxycinnamate plus compound MAA-33 as depicted in Table 3; or
Butyl-methoxydibenzoylmethane plus compound MAA-33 as depicted in Table 3; or
Isoamyl-p-Methoxycinnamate plus compound MAA-33 as depicted in Table 3; or
Diethylamino-hydroxybenzoyl-hexyl-benzoate plus compound MAA-33 as depicted in Table 3; or
Ethylhexyl-methoxycinnamate plus compound MAA-34 as depicted in Table 3; or
Butyl-methoxydibenzoylmethane plus compound MAA-34 as depicted in Table 3; or
Isoamyl-p-Methoxycinnamate plus compound MAA-34 as depicted in Table 3; or
Diethylamino-hydroxybenzoyl-hexyl-benzoate plus compound MAA-34 as depicted in Table 3; or
Ethylhexyl-methoxycinnamate plus compound MAA-35 as depicted in Table 3; or
Butyl-methoxydibenzoylmethane plus compound MAA-35 as depicted in Table 3; or
Isoamyl-p-Methoxycinnamate plus compound MAA-35 as depicted in Table 3; or
Diethylamino-hydroxybenzoyl-hexyl-benzoate plus compound MAA-35 as depicted in Table 3; or
Ethylhexyl-methoxycinnamate plus compound MAA-36 as depicted in Table 3; or
Butyl-methoxydibenzoylmethane plus compound MAA-36 as depicted in Table 3; or
Isoamyl-p-Methoxycinnamate plus compound MAA-36 as depicted in Table 3; or
Diethylamino-hydroxybenzoyl-hexyl-benzoate plus compound MAA-36 as depicted in Table 3; or
Ethylhexyl-methoxycinnamate plus compound MAA-37 as depicted in Table 3; or
Butyl-methoxydibenzoylmethane plus compound MAA-37 as depicted in Table 3; or
Isoamyl-p-Methoxycinnamate plus compound MAA-37 as depicted in Table 3; or
Diethylamino-hydroxybenzoyl-hexyl-benzoate plus compound MAA-37 as depicted in Table 3; or
Ethylhexyl-methoxycinnamate plus compound MAA-38 as depicted in Table 3; or
Butyl-methoxydibenzoylmethane plus compound MAA-38 as depicted in Table 3; or
Isoamyl-p-Methoxycinnamate plus compound MAA-38 as depicted in Table 3; or
Diethylamino-hydroxybenzoyl-hexyl-benzoate plus compound MAA-38 as depicted in Table 3; or
Ethylhexyl-methoxycinnamate plus compound MAA-39 as depicted in Table 3; or
Butyl-methoxydibenzoylmethane plus compound MAA-39 as depicted in Table 3; or
Isoamyl-p-Methoxycinnamate plus compound MAA-39 as depicted in Table 3; and
Diethylamino-hydroxybenzoyl-hexyl-benzoate plus compound MAA-39 as depicted in Table 3.

Most preferred combined are the photo-unstable UV-filter Ethylhexyl-methoxycinnamate, Butyl-methoxydibenzoylmethane, Isoamyl-p-Methoxycinnamate or Diethylamino-hydroxybenzoyl-hexyl-benzoate with one of the MAA-compounds MAA-20 to MAA-39.

In the above specified combinations, the photo-unstable UV-filter is particularly stabilized against photodegradation by the mycosporine-like amino acid compound. In some instances, the combination of the photo-unstable UV-filters and the mycosporine-like amino acid compound have a synergistic stabilizing effect. The photostabilization effect is particularly pronounced by mycosporine-like amino acid compounds having an acid functionality, compared to the respective ester analogues.

In the preparation of the sunscreen products, cosmetic or pharmaceutical preparations or homecare products according to the present invention, the compounds defined herein may be used in combination with other UV-absorbing agent(s), which is/are different from the at least one photo-unstable UV-filter (a), in order to further optimize the SPF, i.e. to obtain a high SPF in a range of 6 to 100, preferably in a range of 6 to 70, and to cover a broad UVA and UVB range.

Therefore, the photo-unstable UV-filter(s) (a) in the sunscreen product, cosmetic or pharmaceutical preparation or homecare product as defined herein, is/are advantageously combined with at least one further primary sun protection UV-filters, which are different to the photo-unstable UV-filter (a) and/or with at least one further secondary sun protection ingredients or secondary sun protection UV-filters. The combination of effective sun protection UV-filters of different categories such as UVA-filter, UVB-filter, broadband filter, inorganic pigments provides reliable protection against the different UV rays in the wavelength range of 290 to 400 nm.

Primary sun protection UV-filters in the context of the invention are, for example, organic substances (light filters) which are liquid or crystalline at room temperature and which are capable of absorbing ultraviolet radiation and of releasing the energy absorbed in the form of longer-wave radiation, for example heat.

The sunscreen product or cosmetic or pharmaceutical preparation according to the invention advantageously contains at least one further UVA filter and/or at least one further UVB filter and/or a broadband filter and/or at least one inorganic pigment, preferably at least one UVA filter and at least one UVB filter, for their use in stopping UV radiation.

For example, the UV-filter may be one or more organic UV-filters and/or one or more inorganic UV-filters. Non-limiting examples of UV-filters include:
(i) sparingly soluble UV-filters (not appreciably soluble in either water or oil) such as Methylene Bis-benzotriazolyl Tetramethylbutylphenol, Tris-Biphenyl Triazine, Methanone, 1,1′-(1,4-piperazinediyl)bis[1-[2-[4-(diethylamino)-2-hydroxybenzoyl-]phenyl];
(ii) oil soluble organic UV-filters (at least partially soluble in oil or organic solvent), such as Bis-Ethylhexyloxyphenol Methoxyphenyl Triazine, Butyl Methoxydibenzoylmethane (BMBM), Oxybenzone, Sulisobenzone, Diethylhexyl Butamido Triazone (DBT), Drometrizole Trisiloxane, Ethylhexyl Methoxycinnamate (EHMC), Ethylhexyl Salicylate (EHS), Ethylhexyl Triazone (EHT), Homosalate, Isoamyl p-Methoxycinnamate, 4-Methylbenzylidene Camphor, Octocrylene (OCR), Polysilicone-15, and Diethylamino Hydroxy Benzoyl Hexyl Benzoate (DHHB);
(iii) inorganic UV-filters such as titanium oxide and zinc oxide, iron oxide, zirconium oxide and cerium oxide; and
(iv) water-soluble UV-filters such as Phenylbenzimidazole Sulfonic Acid (PBSA), Sulisobenzone-sodium salt, Benzydilene Camphor Sulfonic Acid, Camphor Benzalkonium Methosulfate, Cinoxate, Disodium Phenyl Dibenzylmidazole Tetrasulfonate, Terephthalylidene Dicamphor Sulfonic Acid, PABA, and PEG-25 PAB; and any mixture thereof.

In some instances, the UV-filter is one or more of a para-aminobenzoic acid derivative, a salicylic derivative, a cinnamic derivative, a benzophenone or an aminobenzophenone, an anthranillic derivative, a b,b-diphenylacrylate derivative, a benzylidenecamphor derivative, a phenylbenzimidazole derivative, a benzotriazole derivative, a triazine derivative, a bisresorcinyl triazine, an imidazoline derivative, a benzalmalonate derivative, a 4,4-diarylbutadiene derivative, a benzoxazole derivative, a merocyanine, malonitrile or a malonate diphenyl butadiene derivative, a chalcone, and any mixture thereof.

The inorganic UV-filter used as further primary UV-filter is selected from the group of pigments consisting of titanium dioxide (TiO₂) (amorphous or crystallized in rutile and/or anatase form), zinc oxide (ZnO), iron oxide (Fe₂O₃), zirconium oxide (ZrO₂), silicon dioxide (SiO₂), manganese oxide (e.g. MnO), aluminium oxide (Al₂O₃), cerium oxide (Ce₂O₃), barium carbonate (BaCO₃), calcium carbonate (CaCO₃), and mixtures thereof.

Preferably, the inorganic UV-filter is titanium dioxide, zinc oxide, and mixtures thereof, more preferably the at least one inorganic UV-filter is titanium oxide and/or zinc oxide, and most preferably, the at least one inorganic UV-filter is zinc oxide. ZnO has a broad UVA/UVB absorption curve, while TiO2 provides better UVB protection.

According to another variant, the inorganic UV-filter is in form of particles having a weight medium particle size d₅₀ from 1 nm to 1000 nm, preferably from 3 nm to 800 nm, more preferably from 5 nm to 600 nm, and most preferably from 10 nm to 400 nm.

The inorganic UV-filters also encompass nano pigments (mean size of the primary particles: generally from 1 nm to 100 nm, preferably from 3 nm to 90 nm, more preferred from 5 nm to 80 nm and most preferred from 10 to 70 nm) of untreated or treated metal oxides such as, for example, nano pigments of titanium dioxide (TiO₂) (amorphous or crystallized in rutile and/or anatase form), zinc oxide (ZnO), iron oxide (Fe₂O₃), zirconium oxide (ZrO₂), silicon dioxide (SiO₂), manganese oxide (e.g. MnO), aluminium oxide (Al₂O₃), cerium oxide (Ce₂O₃), barium carbonate (BaCO₃), calcium carbonate (CaCO₃), or mixtures thereof.

The treated nano pigments and non-nano pigments are pigments that have undergone one or more surface treatments of chemical, electronic, mechanochemical and/or mechanical nature with compounds, such as amino acids, beeswax, fatty acids, fatty acid esters, fatty alcohols, anionic surfactants, lecithins, sodium, potassium, zinc, iron or aluminium salts of fatty acids, metal (titanium or aluminium) alkoxides, polyethylene, silicones, proteins (collagen or elastin), alkanolamines, silicon oxides, metal oxides, sodium hexametaphosphate, alumina or glycerol, hydrated silica, stearic acid, jojoba esters, or glutamic acid derivates,

The treated nano pigments and non-nano pigments may more particularly be titanium oxides treated with silica and alumina, alumina and aluminium stearate, alumina and aluminium laurate, aluminium hydroxide, iron oxides and iron stearate, silica, alumina and silicone, sodium hexametaphosphate, octyltrimethoxysilane, alumina and stearic acid, alumina and glycerol, or alumina and silicone or polyhydroxy stearic acid, hydrated silica, jojoba esters, steaoryl glutamic acid, glutamic acid and derivates, trimethoxycaprylylsilane, glycerin, dimethicone, hydrogen dimethicone, simethicone or combinations thereof. Other titanium oxide nano pigments treated with a silicone are preferably TiO2 treated with octyltrimethylsilane, preferably for which the mean size of the elementary particles is from 25 to 40 nm; TiO2 treated with a polydimethylsiloxane, preferably for which the mean size of the elementary particles is 21 nm; or TiO2 treated with a polydimethylhydrogenosiloxane, preferably for which the mean size of the elementary particles is 25 nm.

The coated zinc oxide nano pigments and zinc oxide non-nano pigments are for example ZnO coated with polymethylhydrogenosiloxane; ZnO dispersions in cyclopolymethylsiloxane/oxyethylenated polydimethylsiloxane, containing 30% or 80% of nano or non-nano zinc oxides coated with silica and polymethylhydrogenosiloxane; ZnO coated with perfluoroalkyl phosphate and copolymer based on perfluoroalkylethyl as a dispersion in cyclopentasiloxane; ZnO coated with dimethoxydiphenylsilanetriethoxycaprylylsilane cross-polymer; ZnO coated with glutamic acid; ZnO coated with octyltriethoxy silane; ZnO coated with dimethicone; ZnO coated with silicone-grafted acrylic polymer, dispersed in cyclodimethylsiloxane; ZnO coated with triethoxycaprylylsilane; ZnO coated with polyhydroxystearic acid; alumina-treated ZnO dispersed in an ethylhexyl methoxycinnamate/PVP-hexadecene/methicone copolymer mixture; ZnO coated with silica and polymethylsilsesquioxane; ZnO dispersed in hydroxystearic acid polycondensate; or ZnO coated with a mixture of steaoryl glutamic acid, jojoba esters, polyhydroxystearic acid, isopropyl titanium triisostearate, hydrogenated olive oil stearyl esters, cetearyl nonanoate, cera alba, dimethicone, dimethoxydiphenylsilanetriethoxycaprylylsilane cross-polymer, octyltriethoxysilane, glutamic acid and sodium stearoyl lactylate.

Also preferred are particulate UV-filters or inorganic pigments, which can optionally be hydrophobed, such as the oxides of iron (Fe₂O₃), zirconium oxide (ZrO₂), silicon dioxide (SiO₂), manganese oxide (e.g. MnO), aluminium oxide (Al₂O₃), cerium oxide (e.g. Ce₂O₃), barium carbonate (BaCO₃), calcium carbonate (CaCO₃), or mixtures thereof.

Preferably, the one, two, three or more additional primary organic and/or inorganic UV-filters, which are different from the photo-unstable UV-filter (a) are selected from the group consisting of Camphor Benzalkonium Methosulfate, Homosalate, Benzophenone-3, Phenylbenzimidazole Sulfonic Acid, Terephthalylidene Dicamphor Sulfonic Acid, Benzylidene Camphor Sulfonic Acid, Octocrylene, Polyacrylamidomethyl Benzylidene Camphor, PEG-25 PABA, Ethylhexyl Triazone, Drometrizole Trisiloxane, Diethylhexyl Butamido Triazone, 4-Methylbenzylidene Camphor, Ethylhexyl Salicylate, Ethylhexyl Dimethyl PABA, Benzophenone-4, Benzophenone-5, Methylene Bis-Benzotriazolyl Tetramethylbutylphenol (nano), Disodium Phenyl Dibenzimidazole Tetrasulfonate, Bis-Ethylhexyloxyphenol Methoxyphenyl Triazine, Polysilicone-15, Titanium Dioxide, Titanium Dioxide (nano), Tris-biphenyl triazine (nano), Zinc Oxide, Zinc Oxide (nano), Phenylene Bis-Diphenyltriazine, Methoxypropylamino Cyclohexenylidene Ethoxyethylcyanoacetate, Bis-(Diethylaminohydroxybenzoyl Benzoyl) Piperazine, TEA-Salicylate, Benzophenone-8, PABA, Ethylhexyl Dimethyl PABA, Menthyl Anthranilate, and any mixture thereof.

More preferred, the one, two, three or more additional primary organic and/or inorganic UV-filter(s) is/are selected from the group consisting of Camphor Benzalkonium Methosulfate, Benzophenone-3, Terephthalylidene Dicamphor Sulfonic Acid, Benzylidene Camphor Sulfonic Acid, Polyacrylamidomethyl Benzylidene Camphor, PEG-25 PABA, Drometrizole Trisiloxane, Ethylhexyl Dimethyl PABA, Benzophenone-4, Benzophenone-5, Methylene Bis-Benzotriazolyl Tetramethylbutylphenol, Polysilicone-15, Titanium Dioxide (nano), Tris-biphenyl triazine (nano), Phenylene Bis-Diphenyltriazine, Methoxypropylamino Cyclohexenylidene Ethoxyethylcyanoacetate, Bis-(Diethylaminohydroxybenzoyl Benzoyl) Piperazine, Benzophenone-8, Ethylhexyl Dimethyl PABA, Homosalate, Phenylbenzimidazole Sulfonic Acid, Butyl Methoxydibenzoylmethane, Octocrylene, Ethylhexyl Methoxycinnamate, Isoamyl p-Methoxycinnamate, Ethylhexyl Triazone, Diethylhexyl Butamido Triazone, 4-Methylbenzylidene Camphor, Ethylhexyl Salicylate, Disodium Phenyl Dibenzimidazole Tetrasulfonate, Bis-Ethylhexyloxyphenol Methoxyphenyl Triazine, Titanium Dioxide, Diethylamino Hydroxy benzoyl Hexyl Benzoate, Zinc Oxide, Zinc Oxide (nano), Menthyl Anthranilate, and any mixture thereof.

Most preferred, the one, two, three or more additional primary organic and/or inorganic UV-filter(s) is/are selected from the group consisting of Homosalate, Phenylbenzimidazole Sulfonic Acid, Butyl Methoxydibenzoylmethane, Octocrylene, Ethylhexyl Methoxycinnamate, Isoamyl p-Methoxycinnamate, Ethylhexyl Triazone, Diethylhexyl Butamido Triazone, 4-Methylbenzylidene Camphor, Ethylhexyl Salicylate, Disodium Phenyl Dibenzimidazole Tetrasulfonate, Bis-Ethylhexyloxyphenol Methoxyphenyl Triazine, Titanium Dioxide, Diethylamino Hydroxy benzoyl Hexyl Benzoate, Zinc Oxide, Zinc Oxide (nano), and any mixture thereof.

In a preferred variant the sunscreen product, cosmetic or pharmaceutical preparation or homecare product according to the present invention comprises a combination with one or more broadband filters which are selected from the group consisting of 2-ethylhexyl-2-cyano-3,3-diphenyl acrylate, ethyl-2-cyano-3,3′-diphenyl acrylate, dihydroxy-4-methoxybenzophenone, 2,4-dihydroxybenzophenone, tetrahydroxybenzophenone, 2,2′-dihydroxy-4,4′-dimethoxybenzophenone, 2-hydroxy-4-n-octoxybenzophenone, 2-hydroxy-4-methoxy-4′-methylbenzophenone, sodium hydroxymethoxybenzophenone sulfonate, disodium-2,2′-dihydroxy-4,4′-dimethoxy-5,5′-disulfobenzophenone, phenol, 2-(2H-benzotriazol-2-yl)-4-methyl-6-(2-methyl-3(1,3,3,3-tetramethyl-1-(trimethylsilyl)oxy)disiloxyanyl) propyl), 2,2′-methylene-bis-(6-(2H-benzotriazol-2-yl)-4-1,1,3,3-tetramethylbutyl)phenol) (Methylene-Bis-Benzotriazolyl Tetramethylbutylphenol), 2,4-bis-[4-(2-ethylhexyloxy)-2-hydroxyphenyl]-1,3,5-triazine, 2,4-bis-[{(4-(2-ethylhexyloxy)-2-hydroxy}phenyl]-6-(4-methoxyphenyl)-1,3,5-triazine (INCI: Aniso Triazin), 2,4-bis-[{(4-(3-sulfonato)-2-hydroxypropyloxy)-2-hydroxy}phenyl]-6-(4-methoxyphenyl)-1,3,5-triazine sodium salt, 2,4-bis-[{(3-(2-propyloxy)-2-hydroxypropyloxy)-2-hydroxy}phenyl]-6-(4-methoxy-phenyl)-1,3,5-triazine, 2,4-bis-[{4-(2-ethylhexyloxy)-2-hydroxy}phenyl]-6-[4-(2-methoxyethylcarbonyl) phenylamino]-1,3,5-triazine, 2,4-bis-[{4-(3-(2-propyloxy)-2-hydroxypropyloxy)-2-hydroxy}phenyl]-6-[4-(2-ethylcarboxyl) phenylamino]-1,3,5-triazine, 2,4-bis-[{4-(2-ethylhexyloxy)-2-hydroxy}phenyl]-6-(1-methylpyrrol-2-yl)-1,3,5-triazine, 2,4-bis-[{4-tris-(trimethylsiloxysilylpropyloxy)-2-hydroxy}phenyl]-6-(4-methoxyphenyl)-1,3,5-triazine, 2,4-bis-[{4-(2″-methylpropenyloxy)-2-hydroxy}phenyl]-6-(4-methoxyphenyl)-1,3,5-triazine, 2,4-bis-[{4-(1′,1′,1′,3′,5′,5′,5′-heptamethylsiloxy-2″-methylpropyloxy)-2-hydroxy}phenyl]-6-(4-methoxyphenyl)-1,3,5-triazine, dioctylbutylamidotriazone (INCI: Dioctylbutamidotriazone), 2,4-bis-[5-1(dimethylpropyl)benzoxazol-2-yl-(4-phenyl)-imino]-6-(2-ethylhexyl)-imino-1,3,5-triazine, 4,4′,4″-(1,3,5-triazine-2,4,6-triyltriimino)tris-benzoic acid-tris(2-ethyl-hexylester) (also: 2,4,6-tris[anilino(p-carbo-2′-ethyl-1′-hexyloxy)]-1,3,5-triazine (INCI: Octyl Triazone), 2,4,6-tribiphenyl-4-yl-1,3,5-triazine, 2-Ethylhexyl 4-methoxycinnamate (Ethylhexyl Methoxycinnamate), Benzoic acid, 2-hydroxy-, 3,3,5-trimethylcyclohexyl ester (Homosalate), 2-Ethylhexyl salicylate (Ethylhexyl Salicylate), 2-Ethylhexyl 4-(dimethylamino)benzoate (Ethylhexyl Dimethyl PABA), N,N,N-Trimethyl-4-(2-oxoborn-3-ylidenemethyl) anilinium methyl sulfate (Camphor Benzalkonium Methosulfate), Dimethicodiethylbenzalmalonate (Polysilicone-15), and any mixtures thereof.

The additional primary organic and/or inorganic UV-filters, which are different from the photo-unstable UV-filter (a) are generally present in the compositions according to the invention in proportions ranging from 0.01 to 80.0% by weight relative to the total weight of the composition, preferably ranging from 0.1 to 75.0% by weight, more preferably ranging from 0.5 to 70% by weight, and most preferably ranging from 1.0 to 60.0% by weight, relative to the total weight of the ready-to-use formulation.

In a further preferred variant, the sunscreen product, cosmetic or pharmaceutical preparation or homecare product according to the invention contains the primary UV-filters, i.e. in particular organic UV-filters and/or inorganic pigments (UV-filtering pigments), in a total amount so that the product or preparation according to the invention has a sun protection factor SPF of 6 to 100, preferably 6 to 70. Such compositions according to the invention are particularly suitable for protecting the skin, hair and nails.

Besides the group of primary organic and inorganic UV-filters mentioned above, secondary sun protection ingredients of the antioxidant type may also be advantageously used in the sunscreen product, cosmetic or pharmaceutical preparation or homecare product according to the present invention in order to further optimize UV protection.

Secondary sun protection ingredients of the antioxidant type interrupt the photochemical reaction chain which is initiated when UV rays penetrate into the skin.

Typical examples of secondary sun protection ingredients are amino acids (for example arginine, lysine, glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotenoids, carotenes (for example alpha-carotene, beta-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, alpha-linoleyl, cholesteryl and glyceryl esters thereof) and their salts, dilaurylthiodipropionate, distearyl-thiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages, also (metal) chelators (for example alpha-hydroxyfatty acids, palmitic acid, phytic acid, lactoferrine), alpha-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, superoxide dismutase, sinapic acid, sinapoyl malate, sinapate ester derivatives, selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

The group of secondary sun protection ingredients also encompasses plant-based extract(s). Said plant-based extracts have antioxidative and in general photoprotective properties and interrupt the photochemical reaction chain which is initiated when UV rays penetrate into the skin, thus, are effective in preventing skin aging. The plant extracts with UV protection properties are selected from the group consisting of Lignin/Cellulose, Lignin, Lignin Hydrolyzed, Propolis and green propolis, Galanga Extract, macro and micro algae (Porphyra, red algae (Porphyra Umbilicalis), Palmaria palmata, Saccharina latissimi, Corallina pilulifera, Eckloina cava, Sargassum sagamianum, Porphyra rosengurttii, Sargassum siliquastrum, Thalassiosira weissflogii, Green Microalgae (e.g. Interfilum and Klebsormidium), Seaweed (e.g. Padina australis, Euchema, cottonii), Pongamia Glabra Seed Oil, Karanja oil (Millettia pinnata), Rhatania Extrakt (Krameria lappacea), Lichens, Soybean, Caper (Capparis spinosa), Bamboo, Green Coffee oil, Buriti Oil (Mauritia flexuosa), Black Sea Cucumber (Holothuria atra), Cyanobacteria, Fernblock (Polypodium leucotomos), Green Tee, Black Tea (Camellia sinensis), Aloe vera, Walnut, Borage oil, Evening primrose oil, Avocado oil, Tea tree oil, Red Clover (Trifolium pratense L.), Soybean (Glycine max L.), Caper (Capparis spinosa), Almonds, Flame of the forest (Spathodea campanulata), Milk thistle (Silybum marianum L.), Cashew nut shell, Grape, Red Orange, Pomegranate (Punica granatu), Bilberry (Vaccinium myrtillus L.), Bog blueberry (Vaccinium uliginosum L.), Strawberry (Fragaria ananassa), Foti (Polygonum multiflorum Thunb), Turmeric (Curcuma longa L.), Ginseng (Panax ginseng), English ivy (Hedera helix), Broccoli (Brassica oleracea var italica), Tamarind (Tamarindus indica), Opuntia ficus-indica, Coffee, Basil (Ocimum basilicum, Linn.), pomegranate seed oil (Punica granatum), wheat germ oil (Triticum vulgare), blackcurrant seed oil (Ribes nigrum), sesameoil (Sesamum indicum), carrot root (Daucuscarota sativa), raspberry seed oil (Rubus idaeus), traditional medicinal plants from Sri Lanka (Aporosa lindleyana (Euphorbiaceae), Atalantia ceylanica (Rutaceae), Hibiscus furcatus (Malvaceae), Olaxzeylanica (Olacaceae), Ophiorrhiza mungos (Rubiaceae), Argyreia populifolia (Convolvulaceae), Ipomoea mauritiana (Convolvulaceae), Lasia spinosa (Araceae), Leucas zeylanica (Lamiaceae), Plectranthus zeylanicus (Lamiaceae) and Brazil (D. gardneriana seeds, L. microphylla leafs, Amburana cearensis, Aspidosperma cuspa, Aspidosperma pyrifolium, Croton sonderianus, Curatella americana, Dimophandra gardneriana, Lippia microphylla, Luehea paniculata, Sida galheirensis and any mixture thereof.

Preferably, the plant extracts with UV protection properties are selected from the group consisting of Lignin/Cellulose, Lignin, Lignin Hydrolyzed, Galanga Extract, Porphyra, red algae (Porphyra Umbilicalis), Green Microalgae (e.g. Interfilum and Klebsormidium), Seaweed (e.g. Padina australis, Euchema, cottonii), Pongamia Glabra Seed Oil, Karanja oil (Millettia pinnata), Rhatania Extrakt (Krameria lappacea), Soybean, Bamboo, Buriti Oil (Mauritia flexuosa), Cyanobacteria, Fernblock (Polypodium leucotomos), Green Tee, Black Tea (Camellia sinensis), Evening primrose oil, Cashew nut shell, Pomegranate (Punica granatu), and any mixture thereof.

Advantageously, the amount of secondary sun protection substances in the sunscreen product, cosmetic or pharmaceutical preparation or homecare product according to the present invention is advantageously from 0.005 to 5.0% by weight, preferably 0.01 to 4.0% by weight, and most preferred 0.05 to 3.0% by weight, based on the total weight of the ready-to-use formulation.

The UV-filters are incorporated either in the aqueous or in the lipophilic part of the sunscreen product, cosmetic or pharmaceutical preparation or homecare product, depending on whether they are water or oil (fat) soluble/miscible UV-filters or may even be added to the final product by standard methods known to a person skilled in the art.

The photo-unstable UV-filter (a) according to the first aspect of the present invention is present in the sunscreen product or cosmetic or pharmaceutical preparation in an amount of 0.01 to 35.0% by weight, based on the total weight of the final formulation. In a preferred variant, the sunscreen product or cosmetic or pharmaceutical preparation comprises the photo-unstable UV-filter (a) in an amount of 0.1 to 30.0% by weight, based on the total weight of the final formulation. In a more preferred variant, the photo-unstable UV-filter (a) is advantageously used in the sunscreen product or cosmetic or pharmaceutical preparation in an amount of at 0.5 to 25.0% by weight, based on the total weight of the final formulation. In a most preferred variant, the photo-unstable UV-filter is advantageously used in the sunscreen product or cosmetic or pharmaceutical preparation in an amount of 1.0 to 20.0% by weight, based on the total weight of the final formulation.

For a mixture of photo-unstable UV-filters, the above amounts relate to the total content of the photo-unstable UV-filters in the mixture, i.e. the amount is the sum of the content of all photo-unstable UV-filters in the mixture.

The photo-unstable UV-filter (a) and additional UV-filter components, such as primary organic and/or inorganic UV-filters and secondary sun protection ingredients, defined herein, i.e. the total amount of all UV-filters, are present in the sunscreen product, cosmetic or pharmaceutical preparation or homecare product according to the present invention, in an amount of 0.1 to 85.0% by weight, based on the total weight of the final formulation. In a preferred variant, the sunscreen product, cosmetic or pharmaceutical preparation or homecare product comprises the photo-unstable UV-filter (b) and further UV-filter components as defined herein in an amount of 0.3 to 80.0% by weight, based on the total weight of the final formulation. In a more preferred variant, the photo-unstable UV-filter (b) and further UV-filter components as defined herein is/are advantageously used in the sunscreen product, cosmetic or pharmaceutical preparation or homecare product in an amount of 0.5 to 75.0% by weight, based on the total weight of the final formulation. Thus, the above amounts relate to the sum of the content of all UV-filters present in the sunscreen, cosmetic or pharmaceutical preparation or homecare product.

The at least one mycosporine-like amino acid compound (b) according to the first aspect of the present invention is present in the sunscreen product, cosmetic or pharmaceutical preparation or homecare product in an amount of 0.001 to 15.0% by weight, based on the total weight of the final formulation. In a preferred variant, the sunscreen product, cosmetic or pharmaceutical preparation or homecare product comprises the at least one mycosporine-like amino acid compound (b) in an amount of 0.01 to 10.0% by weight, based on the total weight of the final formulation. In a more preferred variant, the at least one mycosporine-like amino acid compound (b) is advantageously used in the sunscreen product, cosmetic or pharmaceutical preparation or homecare product in an amount of at 0.05 to 7.5% by weight, based on the total weight of the final formulation. In a most preferred variant, the at least one mycosporine-like amino acid compound is advantageously used in the sunscreen product, cosmetic or pharmaceutical preparation or homecare product in an amount of at 0.1 to 5.0% by weight, based on the total weight of the final formulation.

For a mixture of mycosporine-like amino acid compounds (b), the above amounts relate to the total content of the mycosporine-like amino acid compounds in the mixture, i.e. the amount is the sum of the content of all mycosporine-like amino acid compounds (b) in the mixture.

The sunscreen product, cosmetic or pharmaceutical preparation or homecare product according to the present invention provide broad spectrum photo-protection to skin und therefore provide protection from both UVA and UVB radiation. Sun protection factor (SPF) is a measure of UVB protection. Typically, the compositions of the present invention have an SPF of at least 6. The SPF however, can vary as needed. The type and amount of UVB filters can be varied to obtain a desired level of SPF. Preferably, the SPF of the sunscreen product or cosmetic or pharmaceutical preparation according to the present invention is at least 6, 10, 15, 20, 25, 30, 35, 40, 45, 50, 55, 60, 65, 70, 75, 80 or even more.

Within the context of the present invention, it is also possible and in some cases advantageous to combine the sunscreen product, cosmetic or pharmaceutical preparation or homecare product according to the first aspect of the present invention with other customary active substances, adjuvants or additives, customarily used for sunscreen products, cosmetic or pharmaceutical compositions or homecare products, as further described below, in order to obtain a ready-for-use preparation or formulation.

The other active agents and/or adjuvants and/or additives or auxiliaries are for example abrasives, anti-acne agents, agents against ageing of the skin, anti-cellulitis agents, anti-dandruff agents, anti-inflammatory agents, irritation-preventing agents, irritation-inhibiting agents, antioxidants, alkanediols, astringents, odour absorbers, perspiration-inhibiting agents, antiseptic agents, anti-statics, antimicrobial agents, binders, buffers, carrier materials, oil components, chelating agents, cell stimulants, cleansing agents, depilatory agents, surface-active substances, deodorizing agents, antiperspirants, softeners, emulsifiers, enzymes, enzyme inhibitors, essential oils, fibres, film-forming agents, water resistance improving agents fixatives, foam-forming agents, foam stabilizers, substances for preventing foaming, foam boosters, gelling agents, gel-forming agents, hair care agents, hair-setting agents, hair-straightening agents, moisture-donating agents, moisturizing substances, moisture-retaining substances, bleaching agents, strengthening agents, stain-removing agents, optically brightening agents, impregnating agents, dirt-repellent agents, dyes, friction-reducing agents, lubricants, moisturizing creams, ointments, opacifying agents, plasticizing agents, covering agents, polish, preservatives, gloss agents, green and synthetic polymers, powders, proteins, re-oiling agents, abrading agents, silicones, skin-soothing agents, skin-cleansing agents, skin care agents, skin-healing agents, skin-lightening agents, skin-protecting agents, skin-softening agents, hair promotion agents, cooling agents, skin-cooling agents, warming agents, skin-warming agents, stabilizers, surfactants, antimicrobial agents, detergents/surfactants, fabric conditioning agents, suspending agents, skin-tanning agents, actives modulating skin or hair pigmentation, matrix-metalloproteinase inhibitors, skin moisturizing agents, glycosaminoglycan stimulators, TRPV1 antagonists, desquamating agents, anti-cellulite agents or fat enhancing agents, hair growth activators or inhibitors, thickeners, rheology additives, vitamins, oils, waxes, pearlizing waxes, fats, phospholipids, saturated fatty acids, mono- or polyunsaturated fatty acids, α-hydroxy acids, polyhydroxy fatty acids, liquefiers, dyestuffs, colour-protecting agents, pigments, anti-corrosives, fragrances or perfume oils, aromas, flavouring substances, odoriferous substances, polyols, electrolytes, organic solvents, and mixtures of two or more of the aforementioned substances, as further described below.

Of the above cosmetically or pharmaceutically excipients and/or active substances and/or additives and/or auxiliaries carriers, oil components, rheology additives, hydrotropes, solubilizing agents, powders, film formers, water resistance improving agents, skin moisturizing and/or moisture-retaining substances, lenitive substances, physiological cooling agents, alkanediols, antioxidants, agents against ageing of the skin, matrix-metalloproteinase inhibitors (MMPI), anti-inflammatory agents, TRPV1 antagonists, emulsifiers, antimicrobial agents, preservatives, antibacterial or antimycotic active substances, chelating agents, surfactants, fragrances/aroma or perfume oils, and any mixture of two or more of the afore-mentioned substances are particularly preferred in the preparation of sunscreen products, cosmetic and pharmaceutical preparations or homecare products.

Carriers: The sunscreen products, cosmetic or pharmaceutical, in particular dermatological, preparations or homecare products as defined herein, in the form of ointments, pastes, creams and gels, etc., are preferably based on a carrier. Most common acceptable carrier is water. Acceptable carriers other than water include glycerin, C1-4 alcohols, organic solvents, fatty alcohols, fatty ethers, fatty esters, polyols, glycols, vegetable oils, mineral oils, liposomes, laminar lipid materials, water, or a mixture thereof. Non-limiting examples of organic solvents include mono alcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds. The total amount of carrier in the compositions can vary but is typically 40 to 90% by weight, based on the total weight of the composition.

The compositions of the present invention may include at least one water-soluble or organic solvent. Non-limiting examples of organic solvents include monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, 1,2-pentanediol, 1,2-hexanediol, 1,2-heptanediol, 1,2-octanediol, 1,2-decanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and any mixture thereof. The total amount water-soluble or organic solvent(s) in the composition according to the present invention may vary but is typically 0.1 to 50% by weight, based on the total weight of the composition.

Solubilizing agents: The compositions of the present invention may include at least one solubilizing agent. Solubilizing agents are compounds that help solubilize the UV-filter(s) and/or other components in the compositions. A particularly useful but non limiting example of a solubilizing agent is a hydrotrope. Hydrotropes (or hydrotropic agents) are a diverse class of typically water-soluble compounds that may be characterized by an amphiphilic molecular structure and an ability to dramatically increase the solubility of poorly soluble organic molecules in water. Non-limiting examples of hydrotopes include sodium 1,3-benzenedisulfonate, sodium benzoate, sodium 4-pyridinecarboxylate, sodium salicylate, sodium benzene sulfonate, caffeine, sodium p-toluene sulfonate, sodium butyl monoglycolsulfate, 4-aminobenzoic acid HCl, sodium cumene sulfonate, N,N-diethylnicotinamide, N-picolylnicotinamide, N-allylnicotinamide, 2-methacryloyloxyethyl phosphorylcholine, resorcinol, butylurea, pyrogallol, N-picolylacetamide 3.5, procaine HCl, proline HCl, nicotinamide, pyridine, 3-picolylamine, sodium ibuprofen, sodium xylenesulfonate, ethyl carbamate, pyridoxal hydrochloride, sodium benzoate, 2-pyrrolidone, ethylurea, N,N-dimethylacetamide, N-methylacetamide, and isoniazid and any mixture thereof. In some cases, particularly useful hydrotropes include nicotinamide (niacinamide), caffeine, sodium PCA, sodium salicylate, urea, and dhydroxyethyl urea, in particular, nicotinamide (niacinamide) and/or caffeine. A combination of two or more, three or more, or four or more hydrotopes may also be used in the compositions according to the present invention. The total amount of solubilizing agent(s) in the compositions of the present invention may vary but are typically in an amount of 0.01 to 20% by weight, based on the total weight of the composition.

Oil components: The sunscreen product or cosmetic or pharmaceutical preparation may include at least one oil phase or at least one oil component, waxes, starch, tragacanth, cellulose derivatives, polyethylene glycols, silicones, bentonites, silica, talc and zinc oxide or any mixture thereof. Powders include carriers such as lactose, talc, silica, aluminium hydroxide, calcium silicate and polyamide powder or any mixture thereof. Solutions and emulsions include carriers such as solvents such as water, ethanol, isopropanol, ethyl carbonate, ethyl acetate, benzyl alcohol, propylene glycol, etc. or any mixture thereof. However, preparations solely based on water are also possible. Oils, oil-in-water and water-in-oil emulsions, etc. are preferred.

The oil phase or the oil component in the sunscreen product, cosmetic or pharmaceutical preparation or homecare product according to the present invention which may be suitable are for example plant oils, hydrocarbons, fatty alcohols, fatty acid esters, or mixtures of two or more of the aforesaid oil components.

The oil phase or oil component in the cosmetic or pharmaceutical preparation is preferably a plant oil and even more preferably a liquid plant oil. It can also advantageously be a mixture of two or more plant oils components, especially liquid plant oil mixtures.

Plant oils or vegetable oils are oils extracted from seeds, or less often, from other parts of fruits. Like animal fats, plant oils are mixtures of triglycerides. Soybean oil, rapeseed oil and cocoa butter are examples of plant oils from seeds. Olive oil, palm oil and rice bran oil are examples of oils from other parts of fruits. In common usage, plant oil or vegetable oil may refer exclusively to vegetable fats which are liquid at room temperature or at 35 to 37° C. skin temperature. Vegetable oils are usually edible.

The term “plant oils” also includes unsaturated plant oils. Unsaturated oils or vegetable oils can be transformed through partial or complete “hydrogenation” into oils of higher melting point. The hydrogenation process involves “sparging” the oil at high temperature and pressure with hydrogen in the presence of a catalyst, typically a powdered nickel compound. As each carbon-carbon double-bond is chemically reduced to a single bond, two hydrogen atoms each form single bonds with the two carbon atoms. The elimination of double bonds by adding hydrogen atoms is called saturation; as the degree of saturation increases, the oil progresses toward being fully hydrogenated. An oil may be hydrogenated to increase resistance to rancidity (oxidation) or to change its physical characteristics. As the degree of saturation increases, the oil's viscosity and melting point increase.

In a preferred variant, the plant oil is selected from the group consisting of Persea Gratissima (Avocado Oil), Abies Alba Seed Oil, Acacia Victoriae Seed Oil, Actinidia Chinensis (Kiwi) Seed Oil, Amaranthus Hypochondriacus Seed Oil, Arachis Hypogaea (Peanut) Oil, Astrocaryum Murumuru Seed Butter, Astrocaryum Tucuma Seed Butter, Astrocaryum Tucuma Seed Oil, Astrocaryum Vulgare Fruit Oil, Astrocaryum Vulgare Kernel Oil, Avena Sativa (Oat) Kernel Oil, Brassica Alba Seed Oil, Brassica Campestris (Rapeseed) Seed Oil, Butyrospermum Parkii (Shea) Butter, Butyrospermum Parkii (Shea) Oil, Calendula Officinalis Seed Oil, Calophyllum Inophyllum Seed Oil, Calophyllum Tacamahaca Seed Oil, Camellia Oleifera Seed Oil, Camellia Reticulata Seed Oil, Camellia Sinensis Seed Oil, Cannabis Sativa Seed Oil, Cannabis Sativa Seed/Stem Oil, Canola Oil, Carthamus Tinctorius (Safflower) Seed Oil, Chlorella Vulgaris Oil, Citrullus Lanatus (Watermelon) Seed Oil, Citrus Aurantifolia (Lime) Seed Oil, Citrus Aurantium Dulcis (Orange) Seed Oil, Citrus Grandis (Grapefruit) Seed Oil, Cocos Nucifera (Coconut) Oil, Cocos Nucifera (Coconut) Seed Butter, Coffea Arabica (Coffee) Seed Oil, Chlorella Oil (biotech), Corylus Americana (Hazelnut) Seed Oil, Corylus Avellana (Hazelnut) Seed Oil, Cucumis Melo (Melon) Seed Oil, Cucumis Sativus (Cucumber) Seed Oil, Cucurbita Pepo (Pumpkin) Seed Oil, Elaeis Guineensis (Palm) Oil, Elaeis (Palm) Fruit Oil, Glycine Soja (Soybean) Oil, GosSY-pium Herbaceum (Cotton) Seed Oil, GosSY-pium Hirsutum (Cotton) Seed Oil, Helianthus Annuus (Sunflower) Seed Oil, Macadamia Integrifolia Seed Oil, Macadamia Ternifolia Seed Oil, Mangifera Indica (Mango) Seed Butter, Mangifera Indica (Mango) Seed Oil, Melissa Officinalis Seed Oil, Microalgae Oil, Moringa Oleifera Seed Oil, Moringa Peregrina Seed Oil, Oenothera Biennis (Evening Primrose) Oil, Olea Europaea (Olive) Fruit Oil, Olus Oil, Orbignya Oleifera Seed Oil, Orbignya Speciosa Kernel Oil, Oryza Sativa (Rice) Bran/Germ Oil, Oryza Sativa (Rice) Bran Oil, Oryza Sativa (Rice) Germ Oil, Oryza Sativa (Rice) Lipids, Oryza Sativa (Rice) Seed Oil, Papaver Somniferum Seed Oil, Passiflora Edulis Seed Oil, Persea Gratissima (Avocado) Butter, Persea Gratissima (Avocado) Oil, Prunus Amygdalus Dulcis (Sweet Almond) Oil, Prunus Armeniaca (Apricot) Kernel Oil, Prunus Persica (Peach) Kernel Oil, Punica Granatum Seed Oil, Pyrus Malus (Apple) Seed Oil, Ricinoleic/Caproic/Caprylic/Capric Triglyceride(s), Ricinus Communis (Castor) Seed Oil, Rosa Canina Fruit Oil, Rosa Moschata Seed Oil, Rubus Idaeus (Raspberry) Seed Oil, Sesamum Indicum (Sesame) Seed Butter, Soybean Glycerides, Theobroma Cacao (Cocoa) Seed Butter, Theobroma Grandiflorum Seed Butter, Triticum Vulgare (Wheat) Bran Lipids, Triticum Vulgare (Wheat) Germ Oil, Vitis Vinifera (Grape) Seed Oil, Zea Mays (Corn) Germ Oil, and Zea Mays (Corn) Oil.

The above specified plant oils are the most common natural lipid components used as basic substances for the manufacture of cosmetics or pharmaceutical formulations.

Hydrocarbons (mineral oils) are in general organic compounds consisting entirely of hydrogen and carbon. As defined by IUPAC nomenclature or organic chemistry, the classifications for hydrocarbons are:
1. Saturated hydrocarbons are the simplest of the hydrocarbon species. They are composed entirely of single bonds and are saturated with hydrogen. The formula for acyclic saturated hydrocarbons (i.e., alkanes) is CₙH₂ₙ₊₂. The most general form of saturated hydrocarbons is CₙH₂ₙ₊₂₍₁₋ᵣ₎, where r is the number of rings. Those with exactly one ring are the cycloalkanes. Saturated hydrocarbons are the basis of petroleum fuels and are found as either linear or branched species.
2. Unsaturated hydrocarbons have one or more double or triple bonds between carbon atoms. Those with double bond are called alkenes. Those with one double bond have the formula CₙH₂ₙ (assuming non-cyclic structures). Those containing triple bonds are called alkynes. Those with one triple bond have the formula CₙH₂ₙ₋₂.
3. Aromatic hydrocarbons, also known as arenes, are hydrocarbons that have at least one aromatic ring.

Hydrocarbons can be inter alia liquids (e.g. hexane and benzene), waxes or low melting solids (e.g. paraffin wax and naphthalene). The term ‘aliphatic’ refers to non-aromatic hydrocarbons. Saturated aliphatic hydrocarbons are sometimes referred to as “paraffins”. Mineral oils and waxes are mixtures of predominantly saturated hydrocarbons consisting of straight-chain, branched and ring structures with carbon chain lengths greater than C14. Mineral oils and waxes are chemical substances prepared from naturally occurring crude petroleum oil. They mainly consist of mineral oil saturated hydrocarbons (MOSH) and mineral oil aromatic hydrocarbons (MOAH). Hydrocarbons have been used for many decades in skin and lip care cosmetic products due to their excellent skin tolerance as well as their high protecting and cleansing performance and broad viscosity options. In contrast to vegetable oils, mineral oils are non-allergenic since they are highly stable and not susceptible to oxidation or rancidity.

A fatty alcohol (or long-chain alcohol) is usually a high-molecular-weight, straight-chain primary alcohol, but can also range from as few as 4 to 6 carbons to as many as 22 to 26, derived from natural fats and oils. The precise chain length varies with the source. Some commercially important fatty alcohols are lauryl, stearyl and oleyl alcohols. They are colourless oily liquids (for smaller carbon numbers) or waxy solids, although impure samples may appear yellow. Fatty alcohols usually have an even number of carbon atoms and a single alcohol group (—OH) attached to the terminal carbon. Some are unsaturated and some are branched. Most fatty alcohols in nature are found as waxes which are esters with fatty acids and fatty alcohols. The traditional sources of fatty alcohols have largely been various vegetable oils and these remain a large-scale feedstock. The alcohols are obtained from the triglycerides (fatty acid triesters), which form the bulk of the oil. The process involves the transesterification of the triglycerides to give methyl esters which are then hydrogenated to give the fatty alcohols. Fatty alcohols are also prepared from petrochemical sources. In the Ziegler process, ethylene is oligomerized using triethylaluminium followed by air oxidation. Alternatively, ethylene can be oligomerized to give mixtures of alkenes, which are subjected to hydroformylation, this process affording odd-numbered aldehyde, which is subsequently hydrogenated. Fatty alcohols are mainly used in the production of detergents and surfactants. They are components also of cosmetic solvents. They find use as co-emulsifiers, emollients and thickeners in cosmetics.

In a preferred variant, the fatty alcohol is selected from the group consisting of phenyl propanol, dimethyl phenylbutanol, hexyldecanol, octyldodecanol, octyldecanol, tridecylalcohol, isostearyl alcohol, phenylisohexanol, phenylpropanol, trimethylbenzenepropanol, isoamylalcohol, isostearyl alcohol, and isotridecyl alcohol. In a more preferred variant, the fatty alcohol is selected from the group consisting of hexyldecanol, octyldodecanol, phenylpropanol, isoamylalcohol, and mixtures of two or more of the aforesaid fatty alcohols. The fatty alcohol can be used either as a single component or in a mixture with one or more further different fatty alcohol(s) as specified above.

A fatty acid ester is a type of ester that results from the combination of a fatty acid with an alcohol. When the alcohol component is glycerol, the fatty acid esters produced can be monoglycerides, diglycerides or triglycerides. Fatty acid esters have a conditioning effect of softening the skin to create a smoothing sensation. They are also added to cosmetics to dissolve high-polarity active ingredients and UV absorbers. Esters of straight-chain fatty acids and lower alcohols are effective for dissolving slightly soluble ingredients for oils with a light touch during application. Isostearic acids and other liquid oils with branched fatty acids and unsaturated fatty acids are commonly used as emollients. Higher fatty acid esters and esters of higher alcohols with relatively high melting points are added to skin creams to adjust the application touch.

Oil bodies and emollients: The sunscreen product, cosmetic or pharmaceutical preparation or homecare product according to the present invention advantageously includes one or more oil bodies or emollients. An emollient is an oleaginous or oily substance which helps to smooth and soften the skin, and may also reduce its roughness, cracking or irritation. Especially, in sunscreen products emollients keep the crystalline and oil-soluble organic and inorganic UV-filters solubilized and prevent them from recrystallisation. In addition, emollients function as wetting agents for inorganic UV-filters for a homogeneous dispersion in the formulations. Suitable oil bodies are, for example, Guerbet alcohols based on fatty alcohols having 6 to 18, preferably 8 to 10, carbon atoms, esters of linear C₆-C₂₂-fatty acids with linear or branched C₆-C₂₂-fatty alcohols or esters of branched C₆-C₁₃-carboxylic acids with linear or branched C₆-C₂₂-fatty alcohols, such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆-C₂₂-fatty acids with branched alcohols, in particular 2-ethylhexanol, esters of C₁₈-C₃₈-alkylhydroxy carboxylic acids with linear or branched C₆-C₂₂-fatty alcohols, in particular Dioctyl Malate, esters of linear and/or branched fatty acids with polyhydric alcohols (such as, for example, propylene glycol, dimerdiol or trimertriol) and/or Guerbet alcohols, triglycerides based on C₆-C₁₀-fatty acids, liquid mono-/di-/triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C₆-C₂₂-fatty alcohol carbonates, such as, for example, Dicaprylyl Carbonate (Cetiol® CC), Guerbet carbonates, based on fatty alcohols having 6 to 18, preferably 8 to 10, carbon atoms, esters of benzoic acid with linear and/or branched C₆-C₂₂-alcohols (e.g. Finsolv® TN), linear or branched, symmetrical or a symmetrical dialkyl ethers having 6 to 22 carbon atoms per alkyl group, such as, for example, dicaprylyl ether (Cetiol® OE), ring-opening products of epoxidized fatty acid esters with polyols, silicone oils (cyclomethicones, silicone methicone grades, etc.) and/or aliphatic or naphthenic hydrocarbons, such as, for example, squalane, squalene or dialkylcyclohexanes and any mixture thereof.

Powders: The compositions as defined herein may optionally include powders. The optional powders provide formulas that are smoother and softer on the skin. Representative powders include talc, mica, magnesium carbonate, calcium carbonate, magnesium silicate, aluminium magnesium silicate, silica, titanium dioxide, zinc oxide, red iron oxide, yellow iron oxide, black iron oxide, polyethylene powder, methacrylate powder, polystyrene powder, silk powder, Preferred solid powder materials, which may be a component of the composition according to the invention are hydrocolloids, such as starches, degraded starches, chemically or physically modified starches, dextrins, (powdery) maltodextrins (preferably with a dextrose equivalent value of 5 to 25, preferably of 10-20), lactose, silicon dioxide, glucose, modified celluloses, gum arabic, ghatti gum, traganth, karaya, carrageenan, pullulan, curdlan, xanthan gum, gellan gum, guar flour, carob bean flour, alginates, agar, pectin, inulin, and mixtures of two or more of these solids, in particular maltodextrins (preferably with a dextrose equivalent value of 15-20), lactose, silicon dioxide and/or glucose.

Rheology modifiers: The sunscreen product, cosmetic or pharmaceutical preparation or homecare products according to the present invention advantageously includes one or more thickening agents and/or rheology modifier. The rheology modifier is present in an amount that prevents significant dripping or pooling of the composition after application to the skin or to surfaces. Preferably, the rheology modifier is a carbomer. In some embodiments, the rheology modifier is selected from stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

Additional example of rheology modifiers include thickener or gelling agents, including substances which can increase the viscosity of a composition. Thickening agents include those that can increase the viscosity of a composition without substantially modifying the efficacy of the active ingredient within the formulation. Thickening agents can also increase the stability of the formulations of the present application. In certain aspects of the present application, thickening agents include hydrogenated polyisobutene or trihydroxy stearin, or a mixture of both. Additional non-limiting examples of additional thickening agents that can be used in the context of the present application include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see CTFA International Cosmetic Ingredient Dictionary, Fourth Edition, 1991, pp. 12 and 80). Examples of commercially available carboxylic acid polymers include carbomers, which are homopolymers of acrylic acid crosslinked with allyl ethers of sucrose or pentaerythritol (e.g., Carbopol™ 900 series from B. F. Goodrich). Non-limiting examples of crosslinked polyacrylate polymers include cationic and non-ionic polymers.

Non-limiting examples of polyacrylamide polymers (including non-ionic polyacrylamide polymers including substituted branched or unbranched polymers) include polyacrylamide, isoparaffin and Laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropyl cellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer are hydroxyalkylated (preferably hydroxy ethylated or hydroxypropylated) to form a hydroxyalkylated cellulose which is then further modified with a C10-C30 straight chain or branched chain alkyl group through an ether linkage. Typically, these polymers are ethers of C10-C30 straight or branched chain alcohols with hydroxyalkylcelluloses. Other useful polysaccharides include scleroglucans comprising a linear chain of (1-3) linked glucose units with a (1-6) linked glucose every three unit.

Non-limiting examples of gums that can be used with the present compositions include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof. In one embodiment, the thickening agent is Chondrus crispus (carrageenan) extract.

Film formers: The sunscreen product, cosmetic or pharmaceutical preparation or homecare product according to the present invention may advantageously include one or more film formers or film forming agent. A film forming agent is a hydrophobic material that imparts water resistance and film forming characteristics to the sunscreen products, cosmetic or pharmaceutical preparations or homecare products. Standard film formers are preferably chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds. Preferred film formers for improving water resistance of the composition are selected from the group consisting of Polyamide-8 (such as -Oleocraft LP PA (MV)), Polyamide-3 (such as Oleocraft MP 32 PA (MV)), Trimethylpentanediol/Adipic Acid/Glycerin Crosspolymer (such as WetFilm), Octyldodecyl Citrate Crosspolymer (such as CosmoSurf CE 100), Polyglyceryl-3 Methyl Glucose Distearate (such as TegoCare 450), C₂₈₋₅₂ Olefin/Undecylenic Acid Copolymer (such as Performa V6112), Hydrolyzed Jojoba esters, Jojoba Esters, Aqua (such as Floraester Jojoba K100), Cyclopentasiloxane Acrylates/Dimethicone copolymer (such as KP545), Acrylates/Beheneth-25 Methacylate Copolymer (such as Volarest), Styrene/Acrylates Copolymer (such as Dermacryl E Polymer), Polyurethane (such as Baycusan C1001), Polyurethane-2 and Polymethyl Methacrylate (such as Hybridur 875 Polymer), Acrylates/Octylacrylamide Copolymer (such as Dermacryl 2.0 Polymer), Beeswax (Cera Alba) (and) Sodium Stearoyl Lactylate (such as SymEffect Sun), Pullulan, Xanthan Gum, Hydrogenated Palm Oil, Saccharum Officinarum Extract (SymEffect™ UV), Microcrystalline cellulose (and) Sphingomonas ferment extract (and) Cellulose gum (such as PemuPur™ START Polymer), Acacia Senegal Gum and Xanthan Gum (such as SOLAGUM™ AX), Microcrystalline Cellulose (and) Cellulose Gum (such as Natpure Cellgum Plus), Sodium Stearoyl Glutamate (such as AMISOFT® HS-11P), Hydrogenated Polycyclopentadiene (and) Caprylic/Capric Triglyceride (such as Koboguard®5400 CCT), Glyceryl Hydrogenated Rosinate (and) Caprylic/Capric Triglyceride (and) Tocopherol (such as KOBOGUARD® NATURAL 2063-CCT), Galactoarabinan (such as LaraCare® A 200), C26-28 Alkyl Dimethicone (such as Wacker BELSIL® CDM 3526 VP), Caprylyl Methicone (such as Silsoft 034), Stearyl Dimethicone (and) Octadecene (such as Dowsil 2503 Cosmetic Wax), VP/Hexadecene Copolymer (such as Antaron™ V 216), Triacontanyl PVP (such as Antaron™ WP-660), Capryloyl Glycerin/Sebacic Acid Copolymer (such as LexFilm™ Sun Natural MB), Poly C₁₀₋₃₀ Alkyl Acrylate (such as Tego SP 13 Sun Up), Microcrystalline Cellulose (such as Sunspheres™ BIO SPF Booster), and any mixture thereof. The aforesaid film forming agents are used in the sunscreen, cosmetic or pharmaceutical preparation or homecare product either as a single component or preferably in a mixture with two or more further of said film forming agents as specified above. The film forming agents are used in amounts effective to allow the final formulation to remain on the skin or surface after exposure to circulating water for at least 40 minutes, preferably 80 minutes. The film forming agent is present in the sunscreen product, cosmetic or pharmaceutical preparation or homecare product in an amount of 0.01 to 10.0% by weight, preferably 0.05 to 8.0% by weight, based on the total weight of the final formulation.

Water resistance improving agents: The sunscreen product, cosmetic or pharmaceutical preparation or homecare product according to the present invention may advantageously include one or more agents for improving water resistance. An agent for improving water resistance is a hydrophobic material that imparts film forming and water resistance characteristics to an emulsion. Typical suitable water resistance improving agents include copolymers derived from polymerization of octadecene-1 and maleic anhydride. A preferred water resistance improving agent is a polyanhydride resin. Another preferred water resistance improving agent is a copolymer of vinyl pyrrolidone and eicosene monomers. The water resistance improving agent(s) is/are used in amounts effective to allow the final formulation to remain on the skin or surface after exposure to circulating water for at least 40 minutes, preferably 80 minutes. One or more water resistance improving agents can optionally be included in the final formulation in an amount ranging from 0.01 to 10.0% by weight, preferably 0.05 to 8.0% by weight, more preferably 0.1 to 5.0% by weight, based on the total weight of the final formulation.

Silicones: In order to impart a silky, spreadable, and luxurious texture and to make skin look and feel smoother, and additionally to improve processability (antifoaming) the sunscreen product, cosmetic or pharmaceutical preparation or homecare product according to the present invention preferably includes one or more silicones or silicone derivatives. The total amount of silicone compound(s) in the compositions according to the present invention can vary but is typically 0.1 to 35.0% by weight, based on the total weight of the composition.

Dry-feel modifiers: The sunscreen product or cosmetic or pharmaceutical preparation according to the present invention is preferably combined with dry-feel modifiers. A dry-feel modifier is an agent which, when incorporated in an emulsion, imparts a “dry feel” to the skin when the emulsion dries. Dry-feel modifiers may also reduce sunscreen migration on the skin. Dry feel modifiers can include starches, talc, kaolin, chalk, zinc oxide, Hydroxyapatite (such as Tinomax CC), silicone fluids, inorganic salts such as barium sulfate and sodium chloride, C6 to C12 alcohols such as octanol; sulfonated oils; surface treated silica, precipitated silica, fumed silica such as Aerosil® available from the Degussa Inc. of New York, N.Y. U.S.A. or mixtures thereof; dimethicone, a mixture of mixture of methylated linear siloxane polymers, available as DC200 fluid, tradename of Dow Corning, Midland, Mich. U.S.A. One or more dry-feel modifiers can optionally be included in the sunscreen in amounts ranging from 0.01 to 20% by weight, more preferably from 0.1 to 10.0% by weight.

Lenitive substances: The sunscreen product or cosmetic or pharmaceutical preparation according to the present invention can also contain advantageously one or more lenitive substances, wherein any lenitive substances can be used which are suitable or customary in cosmetic or pharmaceutical applications such as alpha-bisabolol, azulene, guaiazulene, 18-beta-glycyrrhetinic acid, allantoin, Aloe vera juice or gel, extracts of Hamamelis virginiana (witch hazel), Echinacea species, Centella asiatica, chamomile, Arnica montana, Glycyrrhiza species, algae, seaweed and Calendula officinalis, and vegetable oils such as sweet almond oil, baobab oil, olive oil and panthenol, Laureth-9, Trideceth-9 and 4-t-butylcyclohexanol.

Physiological cooling agents: The sunscreen product or cosmetic or pharmaceutical preparation according to the present invention can be particularly advantageously combined with one or more physiological cooling agent(s). The use of cooling agents can alleviate itching. Preferred individual cooling agents for use within the framework of the present invention are listed below. The person skilled in the art can add many other cooling agents to this list; the cooling agents listed can also be used in combination with one another: which are preferably selected here from the following list: menthol and menthol derivatives (for example L-menthol, D-menthol, racemic menthol, isomenthol, neoisomenthol, neomenthol) menthylethers (for example (I-menthoxy)-1,2-propanediol, (1-menthoxy)-2-methyl-1,2-propanediol, 1-menthyl-methylether), menthone glyceryl acetal, menthone glyceryl ketal or mixtures of both, menthylesters (for example menthylformiate, menthylacetate, menthylisobutyrate, menthyhydroxyisobutyrat, menthyllactates, L-menthyl-L-lactate, L-menthyl-D-lactate, menthyl-(2-methoxy)acetate, menthyl-(2-methoxyethoxy)acetate, menthylpyroglutamate), menthylcarbonates (for example menthylpropyleneglycolcarbonate, menthylethyleneglycolcarbonate, menthylglycerolcarbonate or mixtures thereof), the semi-esters of menthols with a dicarboxylic acid or derivatives thereof (for example mono-menthylsuccinate, mono-menthylglutarate, mono-menthylmalonate, O-menthyl succinic acid ester-N,N-(dimethyl)amide, O-menthyl succinic acid ester amide), menthanecarboxylic acid amides (in this case preferably menthanecarboxylic acid-N-ethylamide [WS3] or N^{α}-(menthanecarbonyl)glycinethylester [WS5], menthanecarboxylic acid-N-(4-cyanophenyl)amide or menthanecarboxylic acid-N-(4-cyanomethylphenyl)amide, menthanecarboxylic acid-N-(alkoxyalkyl)amides), menthone and menthone derivatives (for example L-menthone glycerol ketal), 2,3-dimethyl-2-(2-propyl)-butyric acid derivatives (for example 2,3-dimethyl-2-(2-propyl)-butyric acid-N-methylamide [WS23]), isopulegol or its esters (I—(−)-isopulegol, I—(−)-isopulegolacetate), menthane derivatives (for example p-menthane-3,8-diol), cubebol or synthetic or natural mixtures, containing cubebol, pyrrolidone derivatives of cycloalkyldione derivatives (for example 3-methyl-2(1-pyrrolidinyl)-2-cyclopentene-1-one) or tetrahydropyrimidine-2-one (for example iciline or related compounds, as described in WO 2004/026840), further carboxamides (for example N-(2-(pyridin-2-yl)ethyl)-3-p-menthanecarboxamide or related compounds), (1R,2S,5R)—N-(4-Methoxyphenyl)-5-methyl-2-(1-isopropyl)cyclohexane-carboxamide [WS12], oxamates and [(1R,2S,5R)-2-isopropyl-5-methyl-cyclohexyl]2-(ethylamino)-2-oxo-acetate (X Cool). Cooling agents which are preferred due to their particular synergistic effect are I-menthol, d-menthol, racemic menthol, menthone glycerol acetal (trade name: Frescolat® MGA), menthyl lactate (preferably I-menthyl lactate, in particular I-menthyl I-lactate (trade name: Frescolat® ML)), substituted menthyl-3-carboxamides (such as menthyl-3-carboxylic acid N-ethyl amide), 2-isopropyl-N-2,3-trimethyl butanamide, substituted cyclohexane carboxamides, 3-menthoxypropane-1,2-diol, 2-hydroxyethyl menthyl carbonate, 2-hydroxypropyl menthyl carbonate and isopulegol. Particularly preferred cooling agents are I-menthol, racemic menthol, menthone glycerol acetal (trade name: Frescolat® MGA), menthyl lactate (preferably I-menthyl lactate, in particular I-menthyl I-lactate (trade name: Frescolat® ML)), 3-menthoxypropane-1,2-diol, 2-hydroxyethyl menthyl carbonate and 2-hydroxypropyl menthyl carbonate. Very particularly preferred cooling agents are I-menthol, menthone glycerol acetal (trade name: Frescolat® MGA) and menthyl lactate (preferably I-menthyl lactate, in particular I-menthyl I-lactate (trade name: Frescolat® ML).

Alkanediols: In a further preferred variant, the sunscreen product, cosmetic or pharmaceutical preparation or homecare product according to the invention comprises one or more linear alkanediol(s) having a carbon chain of 5 to 12 carbon atoms. Preferably, said one or more linear alkanediol(s) is/are selected from the group consisting of 1,2-alkanediols, 2,3-alkanediols, 3,4-alkanediols and 1,3-alkanediols, more preferably of 2,3-alkanediols and 1,3-alkanediols. Most preferably, said linear alkanediol(s) is/are selected from the group consisting of 1,2-pentanediol, 1,2-hexanediol, 1,2-heptanediol, 1,2-octanediol, 1,2-nonanediol, 1,2-decanediol, 1,2-undecanediol, 1,2-dodecanediol, 2,3-pentanediol, 2,3-hexanediol, 2,3-heptanediol, 2,3-octanediol, 2,3-nonanediol, 2,3-decanediol, 2,3-undecanediol and 2,3-dodecanediol.

Surprisingly, it was found that the photostability in general, in particular the photostability of an UV-filter, is improved with the addition of the above alkanediols.

Initial observations of the inventors surprisingly indicated that compositions according to the invention comprising one or more linear alkanediol(s) having a carbon chain of 5 to 12 carbon atoms, preferably as defined herein, display particularly advantageous emulsifying properties. In particular, it was surprisingly found that the one or more of said linear alkanediol(s) (if not emulsions themselves) support(s) the formation of water or oil droplets with a particularly small average size in emulsions or (if emulsions themselves) contain water or oil droplets with a particularly small average size.

Additionally, it was found that the presence of an alkanediol, in particular a 1,2-alkanediol or 2,3-alkanediol, improves the solubility of oil components, such as one or more oil body/bodies or wax(es) in the sunscreen product, cosmetic or pharmaceutical preparation or homecare product according to the present invention. Preferably the oil body/bodies or wax(es) are selected from the group consisting of natural fats and oils, fatty alcohols and alcohols, glyceryl esters and derivatives thereof, esters, ethers, siloxanes and silanes, waxes, and naphthenic hydrocarbons, preferably selected from the group consisting of squalene, dialkylcyclohexanes, tetradecane, isohexadecane, dodecane, docosane, paraffin, and mineral oils, and further described below.

A combination of one or more of said alkanediol(s) with 4-hydroxyacetophenon, typically in amounts from 0.1 to 2% by weight, more preferably from 0.1 to 1% by weight, improves the solubility of the oil components even in a synergistic manner.

Preferably, the composition comprises from 0.01 to 10% by weight, more preferably from 0.1 to 5% by weight, still more preferably from 0.3 to 3% by weight, most preferably from 0.5 to 1% by weight, of said linear alkanediol(s) having a carbon chain of 5 to 12 carbon atoms and defined herein, based on the total weight of the composition.

Antioxidants: The sunscreen product, cosmetic or pharmaceutical preparation or homecare product according to the present invention comprises advantageously one or more antioxidants. Suitable antioxidants encompass amino acids (preferably glycine, histidine, tyrosine, tryptophan) and derivatives thereof, imidazoles (preferably urocanic acid) and derivatives thereof, peptides, preferably D,L-carnosine, D-carnosine, L-carnosine and derivatives thereof (preferably anserine), carnitine, creatine, matrikine peptides (preferably lysyl-threonyl-threonyl-lysyl-serine) and palmitoylated pentapeptides, carotenoids, carotenes (preferably alpha-carotene, beta-carotene, lycopene) and derivatives thereof, lipoic acid and derivatives thereof (preferably dihydrolipoic acid), aurothioglucose, propyl thiouracil and other thiols (preferably thioredoxine, glutathione, cysteine, cystine, cystamine and glycosyl, N-acetyl, methyl, ethyl, propyl, amyl, butyl and lauryl, palmitoyl, oleyl, gamma-linoleyl, cholesteryl, glyceryl and oligoglyceryl esters thereof) and salts thereof, dilauryl thiodipropionate, distearyl thiodipropionate, thiodipropionic acid and derivatives thereof (preferably esters, ethers, peptides, lipids, nucleotides, nucleosides and salts) and sulfoximine compounds (preferably buthionine sulfoximines, homocysteine sulfoximine, buthionine sulfones, penta-, hexa-, heptathionine sulfoximine) in very small tolerated doses (e.g. pmol to μmol/kg), also (metal) chelators (preferably alpha-hydroxy fatty acids, palmitic acid, phytic acid, lactoferrin, alpha-hydroxy acids (preferably citric acid, lactic acid, malic acid), humic acid, bile acid, bile extracts, tannins, bilirubin, biliverdin, EDTA, EGTA and derivatives thereof), unsaturated fatty acids and derivatives thereof (preferably gamma-linolenic acid, linoleic acid, oleic acid), folic acid and derivatives thereof, ubiquinone and derivatives thereof, ubiquinol and derivatives thereof, vitamin C and derivatives (preferably ascorbyl palmitate, Mg ascorbyl phosphate, ascorbyl acetate, ascorbyl glucoside), tocopherols and derivatives (preferably vitamin E acetate), vitamin A and derivatives (vitamin A palmitate) and coniferyl benzoate of benzoic resin, rutinic acid and derivatives thereof, flavonoids and glycosylated precursors thereof, in particular quercetin and derivatives thereof, preferably alpha-glucosyl rutin, rosmarinic acid, carnosol, carnosolic acid, resveratrol, caffeic acid and derivatives thereof, sinapic acid and derivatives thereof, ferulic acid and derivatives thereof, curcuminoids, chlorogenic acid and derivatives thereof, retinoids, preferably retinyl palmitate, retinol or tretinoin, ursolic acid, levulinic acid, butyl hydroxytoluene, butyl hydroxyanisole, nordihydroguaiac acid, nordihydroguaiaretic acid, trihydroxybutyrophenone, uric acid and derivatives thereof, mannose and derivatives thereof, zinc and derivatives thereof (preferably ZnO, ZnSO₄), selenium and derivatives thereof (preferably selenium methionine), superoxide dismutase, stilbenes and derivatives thereof (preferably stilbene oxide, trans-stilbene oxide) and the derivatives (salts, esters, ethers, sugars, nucleotides, nucleosides, peptides and lipids) of these cited active ingredients which are suitable according to the invention or extracts or fractions of plants having an antioxidant effect, preferably green tea, rooibos, honeybush, grape, rosemary, sage, melissa, thyme, lavender, olive, oats, cocoa, ginkgo, ginseng, liquorice, honeysuckle, sophora, pueraria, pinus, citrus, Phyllanthus emblica or St. John's wort, grape seeds, wheat germ, Phyllanthus emblica, coenzymes, preferably coenzyme Q10, plastoquinone and menaquinone. Preferred antioxidants are selected from the group consisting of vitamin A and derivatives, vitamin C and derivatives, tocopherol and derivatives, preferably tocopheryl acetate, and ubiquinone.

Emulsifiers: In addition, the sunscreen product, cosmetic or pharmaceutical preparation or homecare product according to the present invention can also advantageously contain one or more emulsifiers. Emulsifiers include amphoteric, anionic, cationic or nonionic emulsifiers, used alone or as a mixture. The emulsifiers are chosen in an appropriate manner according to the emulsion to be obtained. Preferred examples include:
products of the addition of 2 to 30 mol ethylene oxide and/or 0 to 5 mol propylene oxide onto linear C₈₋₂₂ fatty alcohols, onto C₁₂₋₂₂ fatty acids and onto alkyl phenols containing 8 to 15 carbon atoms in the alkyl group;
C_{12/18} fatty acid monoesters and diesters of addition products of 1 to 30 mol ethylene oxide onto glycerol;
glycerol mono- and diesters and sorbitan mono- and diesters of saturated and unsaturated fatty acids containing 6 to 22 carbon atoms and ethylene oxide addition products thereof;
addition products of 15 to 60 mol ethylene oxide onto castor oil and/or hydrogenated castor oil;
polyol esters and, in particular, polyglycerol esters such as, for example, polyglycerol polyricinoleate, polyglycerol poly-12-hydroxystearate or polyglycerol dimerate isostearate. Mixtures of compounds from several of these classes are also suitable;
addition products of 2 to 15 mol ethylene oxide onto castor oil and/or hydrogenated castor oil;
partial esters based on linear, branched, unsaturated or saturated C_{6/22} fatty acids, ricinoleic acid and 12-hydroxystearic acid and glycerol, polyglycerol, pentaerythritol, dipentaerythritol, sugar alcohols (for example sorbitol), alkyl glucosides (for example methyl glucoside, butyl glucoside, lauryl glucoside) and polyglucosides (for example cellulose);
mono-, di and trialkyl phosphates and mono-, di- and/or tri-PEG-alkyl phosphates and salts thereof;
wool wax alcohols;
polysiloxane/polyalkyl polyether copolymers and corresponding derivatives;
mixed esters of pentaerythritol, fatty acids, citric acid and fatty alcohol and/or mixed esters of C₆₋₂₂ fatty acids, methyl glucose and polyols, preferably glycerol or polyglycerol,
polyalkylene glycols and
glycerol carbonate.

The addition products of ethylene oxide and/or propylene oxide onto fatty alcohols, fatty acids, alkylphenols, glycerol mono- and diesters and sorbitan mono- and diesters of fatty acids or onto castor oil are known commercially available products. They are homologue mixtures of which the average degree of alkoxylation corresponds to the ratio between the quantities of ethylene oxide and/or propylene oxide and substrate with which the addition reaction is carried out. C_{12/18} fatty acid monoesters and diesters of addition products of ethylene oxide onto glycerol are known as lipid layer enhancers for cosmetic formulations. The preferred emulsifiers are described in more detail as follows:

Partial glycerides: Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide onto the partial glycerides mentioned are also suitable.

Sorbitan esters: Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable.

Polyglycerol esters: Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan@PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylol propane or pentaerythritol with lauric acid, cocofatty acid, tallow fatty acid, palmitic acid, stearic acid, oleic acid, behenic acid and the like optionally reacted with 1 to 30 mol ethylene oxide.

Anionic emulsifiers: Typical anionic emulsifiers are aliphatic C12 to C 22 fatty acids, such as palmitic acid, stearic acid or behenic acid for example, and C12 to C22 dicarboxylic acids, such as azelaic acid or sebacic acid for example, potassium cetyl phosphate, and hydrogenated palm glycerides (Emulsiphos/Emulsiphos F).

Amphoteric emulsifiers: Other suitable emulsifiers are amphoteric or zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C12/18 acyl sarcosine.

Anti-ageing actives: A composition according to the present invention is preferably combined with one or more anti-ageing actives. In the context of the invention, anti-ageing or biogenic agents are, for example antioxidants, matrix-metalloproteinase inhibitors (MMPI), skin moisturizing agents, glycosaminglycan stimulators, anti-inflammatory agents, TRPV1 antagonists and plant extracts.

Matrix-Metalloproteinase inhibitors (MMPI): The compositions according to the present invention are preferably combined with one or more matrix-metalloproteinase inhibitors, especially those inhibiting matrix-metalloproteinases enzymatically cleaving collagen, selected from the group consisting of ursolic acid, retinyl palmitate, propyl gallate, precocenes, 6-hydroxy-7-methoxy-2,2-dimethyl-1 (2H)-benzopyran, 3,4-dihydro-6-hydroxy-7-methoxy-2,2-dimethyl-1(2H)-benzopyran, benzamidine hydrochloride, the cysteine proteinase inhibitors N-ethylmalemide and epsilon-amino-n-caproic acid of the serinprotease inhibitors: phenylmethylsufonylfluoride, collhibin (company Pentapharm; INCI: hydrolysed rice protein), oenotherol (company Soliance; INCI: propylene glycol, aqua, Oenothera biennis root extract, ellagic acid and ellagitannins, for example from pomegranate), phosphoramidone hinokitiol, EDTA, MGDA (Trisodiumdicarboxymethyl alaninante), galardin, EquiStat (company Collaborative Group; apple fruit extract, soya seed extract, ursolic acid, soya isoflavones and soya proteins), sage extracts, MDI (company Atrium; INCI: glycosaminoglycans), fermiskin (company Silab/Mawi; INCI: water and lentinus edodes extract), actimp 1.9.3 (company Expanscience/Rahn; INCI: hydrolysed lupine protein), lipobelle soyaglycone (company Mibelle; INCI: alcohol, polysorbate 80, lecithin and soy isoflavones), extracts from green and black tea and further plant extracts, proteins or glycoproteins from soya, hydrolysed proteins from rice, pea or lupine, plant extracts which inhibit MMPs, preferably extracts from shitake mushrooms, extracts from the leaves of the Rosaceae family, sub-family Rosoideae, quite particularly extracts of blackberry leaf, as e.g. SymMatrix (company Symrise, INCI: Maltodextrin, Rubus Fruticosus (Blackberry) Leaf Extract). Preferred actives of are selected from the group consisting of retinyl palmitate, ursolic acid, extracts from the leaves of the Rosaceae family, sub-family Rosoideae, genistein and daidzein.

Skin moisturizing and/or moisture-retaining substances: The sunscreen product or cosmetic or pharmaceutical preparation according to the present invention comprises advantageously one or more skin-moisturizing and/or moisture-retaining substances. Preferred skin moisturizing and/or moisture-retaining substances are selected from the group consisting of alkane diols or alkane triols comprising 3 to 12 carbon atoms, preferably C₃-C₁₀-alkane diols and C₃-C₁₀-alkane triols. More preferably the skin moisturizing agents are selected from the group consisting of glycerol, 1,2-propylene glycol, 1,2-butylene glycol, 1,3-butylene glycol, 1,2-pentanediol, 1,2-hexanediol, 1,2-heptanediol, 1,2-octanediol, and 1,2-decanediol. Further skin moisturizing and/or moisture-retaining substances include sodium lactate, urea, urea derivatives, alcohols, glycerol, diols such as propylene glycol, hexylene glycol, collagen, elastin or hyaluronic acid, diacyl adipates, petrolatum, urocanic acid, lecithin, panthenol, phytantriol, lycopene, (pseudo-)ceramides, glycosphingolipids, cholesterol, phytosterols, chitosan, chondroitin sulfate, lanolin, lanolin esters, amino acids, alpha-hydroxy acids (such as citric acid, lactic acid, malic acid) and their derivatives, mono-, di- and oligosaccharides such as glucose, galactose, fructose, mannose, fructose and lactose, polysugars such as R-glucans, in particular 1,3-1,4-β-glucan from oats, alpha-hydroxy fatty acids, triterpene acids such as betulinic acid or ursolic acid, and algae extracts.

Glycosaminoglycan stimulators: Preferred compositions according to the present invention comprise one or more substances stimulating the synthesis of glycosaminoglycans which are selected from the group consisting of hyaluronic acid and derivatives or salts, Subliskin (Sederma, INCI: Sinorhizobium Meliloti Ferment Filtrate, Cetyl Hydroxyethylcellulose, Lecithin), Hyalufix (BASF, INCI: Water, Butylene Glycol, Alpinia galanga leaf extract, Xanthan Gum, Caprylic/Capric Triglyceride), Stimulhyal (Soliance, INCI: Calcium ketogluconate), Syn-Glycan (DSM, INCI: Tetradecyl Aminobutyroylvalylaminobutyric Urea Trifluoroacetate, Glycerin, Magnesium chloride), Kalpariane (Biotech Marine), DC Upregulex (Distinctive Cosmetic Ingredients, INCI: Water, Butylene Glycol, Phospholipids, Hydrolyzed Sericin), glucosamine, N-acetyl glucosamine, retinoids, preferably retinol and vitamin A, Arctium lappa fruit extract, Eriobotrya japonica extract, Genkwanin, N-Methyl-L-serine, (−)-alpha-bisabolol or synthetic alpha-bisabolol such as e.g. Dragosantol and Dragosantol 100 from Symrise, oat glucan, Echinacea purpurea extract and soy protein hydrolysate. Preferred actives are selected from the group consisting of hyaluronic acid and derivatives or salts, retinol and derivatives, (−)-alpha-bisabolol or synthetic alpha-bisabolol such as e.g. Dragosantol and Dragosantol 100 from Symrise, oat glucan, Echinacea purpurea extract, Sinorhizobium Meliloti Ferment Filtrate, Calcium ketogluconate, Alpinia galanga leaf extract and tetradecyl aminobutyroylvalylaminobutyric urea trifluoroacetate.

Anti-inflammatory agents: The compositions according to the present invention are preferably combined with anti-inflammatory and/or redness and/or itch ameliorating ingredients, in particular steroidal substances of the corticosteroid type selected from the group consisting of hydrocortisone, dexamethasone, dexamethasone phosphate, methyl prednisolone or cortisone, are advantageously used as anti-inflammatory active ingredients or active ingredients to relieve reddening and itching, the list of which can be extended by the addition of other steroidal anti-inflammatories. Non-steroidal anti-inflammatories can also be used. More particularly:
(i) steroidal anti-inflammatory substances of the corticosteroid type, in particular hydrocortisone, hydrocortisone derivatives such as hydrocortisone 17-butyrate, dexamethasone, dexamethasone phosphate, methylprednisolone or cortisone,
(ii) non-steroidal anti-inflammatory substances, in particular oxicams such as piroxicam or tenoxicam, salicylates such as aspirin, disalcid, solprin or fendosal, acetic acid derivatives such as diclofenac, fenclofenac, indomethacin, sulindac, tolmetin or clindanac, fenamates such as mefenamic, meclofenamic, flufenamic or niflumic, propionic acid derivatives such as ibuprofen, naproxen or benoxaprofen, pyrazoles such as phenylbutazone, oxyphenylbutazone, febrazone or azapropazone,
(iii) natural or naturally occurring anti-inflammatory substances or substances that alleviate reddening and/or itching, in particular extracts or fractions from camomile, Aloe vera, Commiphora species, Rubia species, willow, willow-herb, oats, calendula, arnica, St John's wort, honeysuckle, rosemary, Passiflora incarnata, witch hazel, ginger or Echinacea, or single active compounds thereof,
(iv) histamine receptor antagonists, serine protease inhibitors (e.g. of Soy extracts), TRPV1 antagonists (e.g. 4-t-Butylcyclohexanol), NK1 antagonists (e.g. Aprepitant, Hydroxyphenyl Propamidobenzoic Acid), cannabinoid receptor agonists (e.g. Palmitoyl Ethanolamine) and TRPV3 antagonists.

Examples which can be cited here are oxicams such as piroxicam or tenoxicam; salicylates such as aspirin, disalcid, solprin or fendosal; acetic acid derivatives such as diclofenac, fenclofenac, indomethacin, sulindac, tolmetin or clindanac; fenamates such as mefenamic, meclofenamic, flufenamic or niflumic; propionic acid derivatives such as ibuprofen, naproxen, benoxaprofen or pyrazoles such as phenylbutazone, oxyphenylbutazone, febrazone or azapropazone. Anthranilic acid derivatives are preferred anti-itch ingredients in a composition according to the present invention.

Also useful are natural or naturally occurring anti-inflammatory mixtures of substances or mixtures of substances that alleviate reddening and/or itching, in particular extracts or fractions from camomile, Aloe vera, Commiphora species, Rubia species, willow, willow-herb, oats, calendula, arnica, St John's wort, honeysuckle, rosemary, Passiflora incarnata, witch hazel, ginger or Echinacea; preferably selected from the group consisting of extracts or fractions from camomile, Aloe vera, oats, calendula, arnica, honeysuckle, rosemary, witch hazel, ginger or Echinacea, and/or pure substances, preferably alpha-bisabolol, apigenin, apigenin-7-glucoside, gingerols, shogaols, gingerdiols, dehydrogingerdiones, paradols, natural or naturally occurring avenanthramides, preferably tranilast, avenanthramide A, avenanthramide B, avenanthramide C, non-natural or non-naturally occurring avenanthramides, preferably dihydroavenanthramide D, dihydroavenanthramide E, avenanthramide D, avenanthramide E, avenanthramide F, boswellic acid, phytosterols, glycyrrhizin, glabridin and licochalcone A; preferably selected from the group consisting of alpha-bisabolol, natural avenanthramides, non-natural avenanthramides, preferably dihydroavenanthramide D (as described in WO 2004 047833 A1), boswellic acid, phytosterols, glycyrrhizin, and licochalcone A, and/or allantoin, panthenol, lanolin, (pseudo-)ceramides [preferably Ceramide 2, hydroxypropyl bispalmitamide MEA, cetyloxypropyl glyceryl methoxypropyl myristamide, N-(1-hexadecanoyl)-4-hydroxy-L-proline (1-hexadecyl) ester, hydroxyethyl palmityl oxyhydroxypropyl palmitamide], glycosphingolipids, phytosterols, chitosan, mannose, lactose and ß-glucans, in particular 1,3->1,4-ß-glucan from oats.

TRPV1 antagonists: Preferred compositions according to the present invention comprise one or more TRPV1 antagonists. Suitable compounds which reduce the hypersensitivity of skin nerves based on their action as TRPV1 antagonists, encompass e.g., trans-4-tert-butyl cyclohexanol, or indirect modulators of TRPV1 by an activation of the μ-receptor, e.g., acetyl tetrapeptide-15, are preferred.

Antimicrobial agents: The sunscreen product, cosmetic or pharmaceutical preparation or homecare product according to the present invention can advantageously combined with an antimicrobial agent which act primarily against microorganisms, in particular bacteria, yeast and/or fungi and includes one or more antimicrobial agents such as Caprylhydroxamic Acid, o-Cymen-5-ol, Isopropylparaben, Capryloyl Glycine, Phenylpropanol, Tropolone, PCA Ethyl Cocoyl Arginate, 2-Methyl 5-Cyclohexylpentanol, Phenoxyethanol, Disodium EDTA, Methylparaben and its salts, Sodium Benzoate, Benzyl Alcohol, Potassium Sorbate, Benzyl Salicylate, Propylparaben, Sodium Methylparaben, Methylchloroisothiazolinone, Methylisothiazolinone, Ethylhexylglycerin, Butylparaben, Ethylparaben, Sodium Propylparaben, DMDM Hydantoin, Dehydroacetic Acid, Salicylic Acid, Chlorphenesin, Isobutylparaben, Sodium Ethylparaben, Diazolidinyl Urea, Diazolidinyl Urea, Farnesol, Bisabolol, Glyceryl Caprylate, Sodium Phytate or Phytic Acid, Sodium Levulinate or Levulinic Acid and esters and/or ketals thereof, Chlorhexidine, Glyceryl Laurate, Anisic Acid and its salts, Chlorhexidine Digluconate, TEA-salicylate, Phenethyl Alcohol, Glyceryl Caprate, Sorbitan Caprylate, Tetrasodium Glutamate Diacetate, Trisodium Ethylenediamine Disuccinate, Hydroxyethoxyphenyl Butanone, Hydroxyethoxyphenyl Butanol, Itaconic Acid, Octopirox, Propanediol Caprylate, Climbazole, Undecylenoyl Glycine, Thymol, 4-Hydroxyacetophenone, Lactic Acid, Sodium Lactate, Trisodium Dicarboxymethyl Alaninate, Frambinon, Xylityl caprylate, Benzoic acid 3-hyroxypropylester, Anisic acid 3-hydroxypropylester, 3-Hydroxypropyl 2-hydroxybenzoate,1,3-propanediol, 1,2-pentanediol, 1,2-hexanediol, 1,2-heptanediol, 1,2-octanediol, 1,2-nonanediol, 1,2-decanediol, 1,2-undecanediol, 1,2-dodecanediol, 2,3-pentanediol, 2,3-hexanediol, 2,3-heptanediol, 2,3-octanediol, 2,3-nonanediol, 2,3-decanediol, 2,3-undecanediol, 2,3-dodecanediol, and any mixture thereof.

Preferably, the sunscreen product, cosmetic or pharmaceutical preparation or homecare product according to the present invention comprises one or more antimicrobial agents selected from the group consisting of 1,2-pentanediol, 1,2-hexanediol, 1,2-heptanediol, 1,2-octanediol, 1,2-nonanediol, 1,2-decanediol, 1,2-undecanediol, 1,2-dodecanediol, 2,3-pentanediol, 2,3-hexanediol, 2,3-heptanediol, 2,3-octanediol, 2,3-nonanediol, 2,3-decanediol, 2,3-undecanediol, 2,3-dodecanediol, 2-benzylheptanol, 2-hydroxyacetophenone, 2-methyl 5-cyclohexylpentanol, 3-hydroxypropyl 2-hydroxybenzoate, 4-hydroxyacetophenone, and any mixture thereof. More preferably, the sunscreen product, cosmetic or pharmaceutical preparation or homecare product according to the present invention comprises one or more antimicrobial agent selected from the group consisting of 1,2-pentanediol, 1,2-hexanediol, 1,2-heptanediol, 1,2-octanediol, 1,2-nonanediol, 1,2-decanediol, 1,2-undecanediol, 1,2-dodecanediol, 1,2-tridecanediol, 4-hydroxyacetophenone, and any mixture thereof.

In order to further enhance antimicrobial efficacy, the sunscreen product, cosmetic or pharmaceutical preparation or homecare product as defined herein, is advantageously combined with at least one further antimicrobial agent which is different from the antimicrobial agents specified above. The combination with a further antimicrobial agent provides reliable protection against microbial degradation and deterioration of the preparation, in particular during storage. Additionally, the further different antimicrobial agent provides reliable protection against other microorganisms as described above, for example Corynebacterium, Anaerococcus, Finegoldia, Moraxella, Porphyromonas, Fusobacterium, Malassezia, Peptoniphilus, Streptococcus, Lactobacillus, Gardnerella, Fannyhessea, Epidermophyton, Trichophyton, Cutibacterium. Hence, the combination with a further different antimicrobial agent allows antimicrobial protection against different groups of microorganisms, and, thus, a broader spectrum of microorganism. The further different antimicrobial agent in the context of the present invention is preferably selected from the group consisting of 2-benzylheptanol, alkyl (C 12-22) trimethyl ammonium bromide and chloride, ascorbic acid and salts thereof, benzalkonium bromide, benzalkonium chloride, benzalkonium saccharinate, benzethonium chloride, Benzoic Acid, camphor, cetylpyridinium chloride, chlorhexidine diacetate, chlorhexidine dihydrochloride, chlorocresol, chloroxylenol, Cyclohexylglycerin, Dimethyl phenylbutanol, Dimethyl phenylpropanol, ethanol, ethyl lauroyl arginate HCl, Ethyl Lauroyl Arginate Laurate, eucalyptol, gluconic acid and salts thereof, glycerin, hexamidine, hexamidine diisethionate, Hexylglycerin, lodopropynyl Butylcarbamate, jasmol, lauryl alcohol, Levulinic Acid and esters and/or ketals thereof, mannitol, menthol, methyl salicylate, Octenidine HCl, polyarginine, Polyglyceryl-10 Laurate, polyglyceryl-2 caprate, Polyglyceryl-3 caprylate, polylysine, Salvia Officinalis (Sage) Oil, silver chloride, silver citrate, sodium caproyl lactylate, Sodium Caproyl/Lauroyl Lactylate, sodium lauroyl lactylate, Sorbic Acid, sorbitol, Tetraselmis extract, triclocarban, triclosan, Triethyl citrate, Xylityl Sesquicaprylate, zinc citrate, zinc pyrithione, Zinc ricinoleate, and any mixture thereof.

The aforesaid antimicrobial agents are used in the sunscreen product, cosmetic or pharmaceutical preparation or homecare product either as a single component or preferably in a mixture with two or more further of said antimicrobial agents as specified above.

The antimicrobial agent is present in the sunscreen product, cosmetic or pharmaceutical preparation or homecare product in an amount of 0.01 to 25.0% by weight, preferably 0.02 to 15.0% by weight, most preferred in an amount of 0.05 to 6.0% by weight, based on the total weight of the final formulation.

Advantageously, the at least one antimicrobial alkanediol is present in the sunscreen product, cosmetic or pharmaceutical preparation or homecare product in an amount of 0.1 to 5% by weight, based on the total weight of the final formulation and/or the 4-hydroxyacetophenone in an amount of 0.01 to 2% by weight, based on the total weight of the final formulation.

Preservatives: For preservative purposes, the sunscreen product, cosmetic or pharmaceutical preparation or homecare product according to the present invention preferably includes one or more preservatives which are suitable or customary in sunscreen products, cosmetic or pharmaceutical preparations or homecare products. Suitable and advantageously preservatives are, for example, benzoic acid, sodium benzoate, ammonium benzoate, butyl benzoate, calcium benzoate, ethyl benzoate, isobutyl benzoate, isopropyl benzoate, magnesium benzoate, mea-benzoate, methyl benzoate, phenyl benzoate, potassium benzoate, propyl benzoate, propionic acid, ammonium propionate, calcium propionate, magnesium propionate, potassium propionate, sodium propionate, salicylic acid, calcium salicylate, magnesium salicylate, measalicylate, sodium salicylate, potassium salicylate, teasalicylate, sorbic acid, calcium sorbate, sodium sorbate, potassium sorbate, o-phenylphenol, sodium sulfite, ammonium bisulfite, ammonium sulfite, potassium sulfite, potassium hydrogen sulfite, sodium bisulfite, sodium metabisulfite, potassium metabisulfite, chlorobutanol, 4-hydroxybenzoic acid, methylparaben, potassium ethylparaben, potassium paraben. sodium methylparaben, sodium ethylparaben, ethylparaben, sodium paraben, potassium methylparaben, calcium paraben, butylparaben, propylparaben, sodium propoylparaben, sodium butylparaben, potassium butylparaben, potassium propylparaben, dehydroacetic acid, sodium dehydroacetate, formic acid, sodium formate, dibromohexamidine isethionate, thimerosal, phenyl mercuric acetate, phenyl mercuric benzoate, undecylenic acid, potassium undecylenate, sodium undecylenate, calcium undecylenate, mea-undecylenate, tea-undecylenate, hexetidine, 5-bromo-5-nitro-1,3-dioxane, 2-bromo-2-nitropropane-1,3-diol, dichlorobenzyl alcohol, triclocarban, p-chloro-m-cresol, triclosan, chloroxylenol, imidazolidinyl urea, polyaminopropyl; biguanide, phenoxyethanol, methenamine, climbazole, DMDM hydantoin, benzyl alcohol, 1-hydroxy-4-methyl-6-(2,4,4-trimethylpentyl)-2 pyridon, piroctone olamine, bromochlorophene, o-cymen-5-ol, methylchloroisothiazolinone and methylisothiazolinone, chlorhexidine, chlorhexidine diacetate, chlorhexidine digluconate, chlorhexidine dihydrochloride, phenoxyisopropanol, behentrimonium chloride(1), cetrimonium bromide, cetrimonium chloride(2), laurtrimoniumbromide, laurtrimonium chloride, steartrimonium bromide, steartrimonium chloride (2), dimethyl oxazolidine, diazolidinyl urea, hexamidine, hexamidine diisethionate, hexamidine diparaben, hexamidine paraben, glutaral, 7-ethylbicyclooxazolidine, chlorphenesin, sodium hydroxymethylglycinate, silver chloride, benzethonium chloride, benzalkonium chloride, benzalkonium bromide, benzalkonium saccharinate, benzylhemiformal, iodopropynyl butylcarbamate, methylisothiazolinone, citric acid, silver citrate, hydroxyethoxy-phenyl butanone (HEPB) and any mixture thereof.

Drugs: The sunscreen product or cosmetic or pharmaceutical preparation according to the present invention are preferably combined with one or more drugs. In certain such embodiments, the pharmaceutically active agent is selected from anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including eflornithine and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, etc.

Especially useful are natural or naturally occurring anti-inflammatory mixtures of substances, in particular extracts or fractions from camomile, Aloe vera, Commiphora species, Rubia species, willow, willow-herb, oats, calendula, arnica, St John's wort, honeysuckle, rosemary, Passiflora incarnata, witch hazel, ginger or Echinacea; preferably selected from the group consisting of extracts or fractions from camomile, Aloe vera, oats, calendula, arnica, honeysuckle, rosemary, witch hazel, ginger or Echinacea, and/or pure substances, preferably alpha-bisabolol, apigenin, apigenin-7-glucoside, gingerols, shogaols, gingerdiols, dehydrogingerdiones, paradols, natural or naturally occuring avenanthramides, preferably tranilast, avenanthramide A, avenanthramide B, avenanthramide C, non-natural or non-naturally occuring avenanthramides, preferably dihydroavenanthramide D, dihydroavenanthramide E, avenanthramide D, avenan-thramide E, avenanthramide F, boswellic acid, phytosterols, glycyrrhizin, glabridin and licochalcone A; preferably selected from the group consisting of alpha-bisabolol, natural avenanthramides, non-natural avenanthramides, preferably dihydroavenanthramide D, boswellic acid, phytosterols, glycyrrhizin, and licochalcone A, and/or allantoin, panthenol, lanolin, (pseudo-)ceramides, preferably Ceramide 2, hydroxypropyl bispalmitamide MEA, cetyloxypropyl glyceryl methoxypropyl myristamide, N-(1-hexadecanoyl)-4-hydroxy-L-proline (1-hexadecyl) ester, hydroxyethyl palmityl oxyhydroxypropyl palmitamide], glycosphingolipids, phytosterols, chitosan, mannose, lactose and ß-glucans, in particular 1,3->1,4-ß-glucan from oats.

In addition to the above-described substances, further ingredients commonly used in sunscreen products, cosmetic or pharmaceutical preparations or homecare products, which are suitable or customary in the compositions of the present invention, can be used.

The sunscreen product or cosmetic or pharmaceutical, in particular dermatological, preparation according to the first aspect of the present invention is intended for topical applications. The term “topical” is understood to mean external applications on a mammal's skin or mucosa, which are in particular for the protection, treatment, care and cleansing of the skin, scalp, eyelashes, eyebrows, nails, mucous membranes and hair. The mammal is preferably a human.

For topical application, the cosmetic or pharmaceutical preparation is either a rinse off or a leave on preparation.

The sunscreen products according to the present invention may be in the form of an aqueous solution, dispersions, a hydro alcoholic vehicle, a stick, an ointment, a gel, an aerosol (foams, sprays, propellant pump) and the like.

The sunscreen product, cosmetic or pharmaceutical, in particular dermatological, preparation or homecare product according to the first aspect of the present invention can be present in different forms, e.g. in the form of a dispersion, in the form of a water free formulation, or in the form of an aqueous, aqueous/alcoholic, in particular aqueous/ethanolic, aqueous/glycolic or alcoholic/glycolic, in particular ethanolic/glycolic, based solution.

In a preferred variant, the sunscreen product, cosmetic or pharmaceutical preparation or homecare product according to the first aspect of the invention is a dispersion. The term “dispersion” in the context of the present invention means, that the sunscreen product, cosmetic or pharmaceutical preparation or homecare product is a disperse two-phase system consisting of colloidal particles (disperse phase) and a medium in which they are suspended (disperse medium). Both phases are not miscible with each other, only with an emulsifier. Such dispersions, for example emulsions, comprise the at least one oil component (without UV-filters) preferably in an amount of ≥1% by weight, more preferably in an amount of 3% by weight.

Preferably, the sunscreen product, cosmetic or pharmaceutical preparation or homecare product according to the first aspect of the present invention takes various forms such as an emulsion, in particular a O/W emulsion, a W/O emulsion, a multiple emulsion, a hydrodispersion gel, a balm, a multiple emulsion of the water-in-oil type (W/O/WO) or of the oil-in-water type (O/W/O), PIT emulsion, Pickering emulsion, a micro-emulsion, a liquid, a lotion, a suspension, a milk, an ointment, a paste, a gel, a cream based, an oil based or a liposome-based formulation or a an aerosol such as foams and sprays, including all types of silicon based emulsions.

In a most preferred variant, the sunscreen product, cosmetic or pharmaceutical preparation or homecare product according to the present invention is in the form of an emulsion as defined herein, advantageously in the form of an oil-in-water (O/W) emulsion comprising an oily phase dispersed in an aqueous phase in the presence of an O/W or polymeric emulsifier.

If the sunscreen product, cosmetic or pharmaceutical preparation or homecare product according to the present invention is a dispersion, preferably an emulsion, the oil component (without UV-filters) is present in the cosmetic or pharmaceutical preparation in an amount of 0.01 to 50.0% by weight, based on the total weight of the composition. In a preferred variant, the cosmetic or pharmaceutical preparation comprises the oil component in an amount of 0.1 to 45.0% by weight, based on the total weight of the composition. In a particular preferred variant, the oil component is advantageously used in the cosmetic or pharmaceutical preparation in an amount of at 1.0 to 40% by weight, based on the total weight of the composition.

In a further variant, the sunscreen product, cosmetic or pharmaceutical, preferably dermatological, preparation or homecare product according to the first of the invention is a water free formulation, i.e. an oil formulation.

If the sunscreen product, cosmetic or pharmaceutical preparation or homecare product according to the present invention is a water free formulation, i.e. an oil formulation, the oil component is present in the cosmetic or pharmaceutical preparation in an amount of ≥60% by weight, preferably in an amount of ≥75% by weight, more preferably in an amount of ≥90% by weight, based on the total weight of the composition.

Such water free formulations include e.g. oils, skin butters, powders, lip stick, antiperspirant/deo sticks, and decorative cosmetics.

In addition, further water free formulations are likewise formulations on the basis of ethanol/diols/triols/glycols such as sprays or gels.

Alternatively, the sunscreen product, cosmetic or pharmaceutical preparation or homecare product as disclosed herein is an aqueous or an aqueous/alcoholic, preferably aqueous/ethanolic, or an aqueous/glycolic, or an alcoholic/glycolic, preferably ethanolic/glycolic, based solution. Typically, this could be glycerin in water or alcohol compositions. Said solutions are homogeneous one phase system of water/alcohol/glycol and additional components. The aqueous/alcoholic or aqueous/glycolic or alcoholic/glycolic based solution comprises an aliphatic alcohol or a glycol in an amount of 0.1 to 70.0% by weight, preferably in an amount of 0.5 to 60.0% by weight, more preferably in an amount of 1.0 to 50.0% by weight, based on the total weight of the solution. The aliphatic alcohol is preferably selected from the group consisting of ethanol, isopropanol, n-propanol. The glycol is preferably selected from the group consisting of glycerin, propylene glycol, 1,3-Propanediol, 1,2-Propanediol, 1,2-C5 to C10-alkanediols, butylene glycol or dipropylene glycol.

Preferably, the overall water content in the final aqueous based solutions can be ≥30% by weight, more preferably ≥40% by weight, most preferably ≥50% by weight.

Such aqueous or aqueous/alcoholic or aqueous/glycolic or alcoholic/glycolic based solutions include for example deo/antiperspirant preparations, after shave, cleansing preparations, or anti-acne preparations.

In a preferred variant, the inventive composition is an impregnation solution in the form of an emulsion spray or ethanol/oil spray for wet wipes.

The above formulations or compositions are prepared according to usual and known methods.

The products and preparations according to the present invention are for cosmetic and/or non-therapeutic use for personal care, skin protection, skin care, sun care, scalp protection, scalp care, hair care, nail care, in particular for the prevention and treatment of skin conditions, intolerant or sensitive skin, skin irritation, skin reddening, wheals, pruritis (itching), skin aging, wrinkle formation, loss of skin volume, loss of skin elasticity, piment spots, pigment abnormalities, skin dryness, flaking, greasiness, hypopigmentation and/or hyperpigmentation of the skin; or a preparation for animal care.

Examples of personal care products are preferably anti-ageing preparations, skin care emulsions, body oils, body lotions, cleansing lotions, face or body balms, after shave balms, after sun balms, deo emulsions, cationic emulsions, body gels, treatment creams, skin protection ointments, moisturizing gels, face and/or body moisturizers, light protective preparations (sunscreens), micellar water, hair sprays, color protection hair care products, skin lightning products, anti-dark-spot reparation, etc.

The cosmetic preparations include also make-ups, eye-care preparation, eye shadows, mascara, eyeliner, lip care preparation such as lip stick, lip gloss, mail care preparations, such as nail varnish, nail varnish removers,

Alternatively, the preparation according to the present invention is a preparation for medical use.

The pharmaceutical, in particular dermatological, preparation according to the present invention is preferably a preparation for the prevention and treatment of a condition of the skin, mucosa, hair or nails.

In order to be used, the sunscreen product or cosmetic or pharmaceutical, in particular dermatological composition according to the first aspect of the present invention is applied to the skin, hair, scalp and/or nails in an adequate amount in such manner as is customary with sunscreen products or cosmetics and dermatological products.

In addition, the products and preparations according to the present invention are homecare products. The home care products generally include laundry detergents (powder, liquid and tablet), fabric conditioners, dishwashing detergents (liquid and tablet), hard floor and surface cleaners, glass cleaners, carpet cleaners, oven cleaners, air fresheners, disinfectants, stain removers, car wash products. These products are usually manufactured in the form of a liquid, powder, spray, granules and others.

Surprisingly, it was found that the mycosporine-like amino acid compounds or their salts have the common property of being effective in stabilizing photo-unstable UV-filters, i.e. act as stabilizer when combined with a photo-unstable UV-filter, making them more effective for longer periods of time and to provide a constant protection during prolonged exposure to the sun. The compositions according to the present invention exhibits greater UV absorbance than a composition comprising the at least photo-unstable UV-filter material without the addition of at least one mycosporine-like amino acid compound.

With the admixture of a mycosporine-like amino acid compound to a sunscreen product or cosmetic or pharmaceutical preparation and stabilization effect against a photo-unstable UV-filter, the SPF of the sunscreen product, the cosmetic or pharmaceutical preparation or homecare product remains almost constant even during exposure to the sun, compared to products or preparations without the addition of the specified mycosporine-like amino acid compounds. The higher the SPF the more protective the sunscreen is.

Furthermore, the sunscreen product, cosmetic or pharmaceutical preparation or homecare product has an increase in pre-irradiation SPF as compared to a composition comprising the at least one photo-unstable UV-filter without any one of the mycosporine-like amino acid compound.

In addition, by stabilizing the UV-filter, its degradation upon exposure to UV-radiation can be reduced and the formation of phototoxic or photoallergic degradation products with a risk of adverse health effects when coming into direct contact with the skin can be reduced.

The above-described combinations of a photo-unstable UV-filter with any of a mycosporine-like amino acid compound defined herein result in distinguished stabilized sunscreen products or cosmetic or pharmaceutical products where degradation of the photo-unstable UV-filter is considerably reduced, as it is demonstrated by the following examples. Hence, a highly photostable sunscreen product is obtained with the addition of a mycosporine-like amino acid.

In addition, due to the smaller degradation of the UV-filter, the ready-to-use products according to the present invention have a higher SPF, and, consequently an improved UV protection as compared to those ready-to-use products comprising the photostable UV-filter without any of the mycosporine-like amino acid stabilizing compound.

Conversely, due to the smaller degradation of the UV-filter, less amount of UV-filter has to be used in the ready-to-use products in order to obtain the same SPF, compared to those ready-to-use products comprising the photo-unstable UV-filter without any of the mycosporine-like amino acid stabilizing compound.

Advantageously, the addition of a mycosporine-like amino acid compound to a sunscreen product or cosmetic or pharmaceutical preparation comprising said photo-unstable UV-filter also leads to an increase in absorbance, as it is demonstrated by the following examples. The higher the amplitude of the absorption curve the greater the absorbance and the more protection provided at that wavelength. This effect results in a higher SPF of the ready-to-use product, and consequently to an improved UV protection as compared to those ready-to-use products comprising the photo-unstable UV-filter without any of the mycosporine-like amino acid stabilizing compound.

Furthermore, the addition of a mycosporine-like amino acid compound to a sunscreen product or cosmetic or pharmaceutical preparation comprising said photo-unstable UV-filter alters, i.e. broaden, the absorption spectrum across the ultraviolet radiation spectrum. In other words: the greater the breadth of the absorbance curve, the broader the spectrum of sun protection provided.

The absorbance loss is particularly lower for a mixture of MAA-1 and Ethylhexyl Methoxycinnamate: The loss of Ethylhexyl Methoxycinnamate is reduced from 37.88% to 18.82%, i.e. by approximately 50%, in combination with MAA-1 (see FIG. 8). Especially, said mixture has an increase in pre-irradiation SPF: mixture: absorbance: 1.6 relative to MAA-1 alone: 0.9 and Ethylhexyl Methoxycinnamate alone: 0.8.

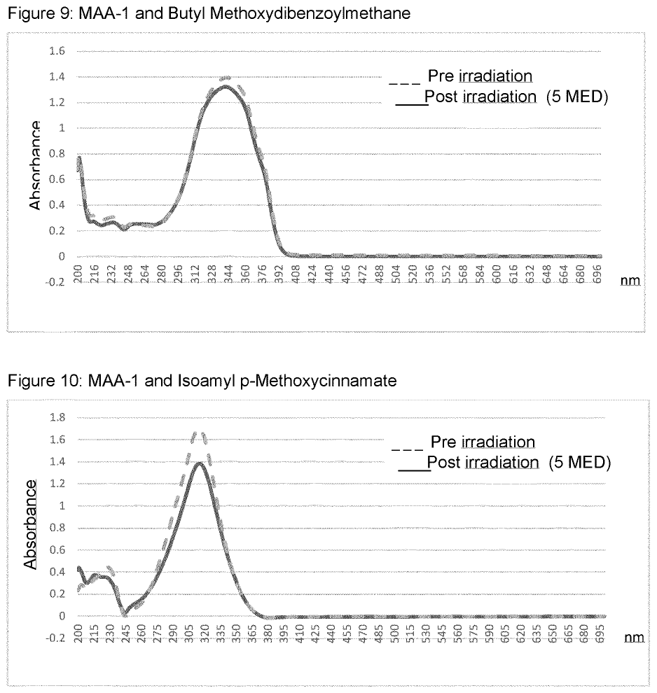

The same effect could be observed for a mixture of MAA-1 and Butyl Methoxydibenzoylmethane it is possible to reduce the loss of Butylmethoxydibenzoylmethane from 7.05% to 5,27%, i.e. by approximately 25% in combination with MAA-1 (see FIG. 9). Said mixture also has an increase in pre-irradiation SPF: mixture: absorbance: 1.4 relative to MAA-1 alone: 0.9 and Butyl Methoxydibenzoylmethane alone: 0.8.

In combination with MAA-1 it is possible to reduce the loss of Isoamyl p-Methoxycinnamate from 30.50% to 18.60% by approximately 39% (see FIG. 10). Also said mixture has an increase in pre-irradiation SPF: mixture: absorbance: 1.7 relative to MAA-1 alone: 0.9 and Isoamyl p-Methoxycinnamate alone: 1.0.

With the admixture of MAA-13 it is possible to reduce the loss of Isoamyl p-Methoxycinnamate from 30.50% to 20.45% by approximately 32%.

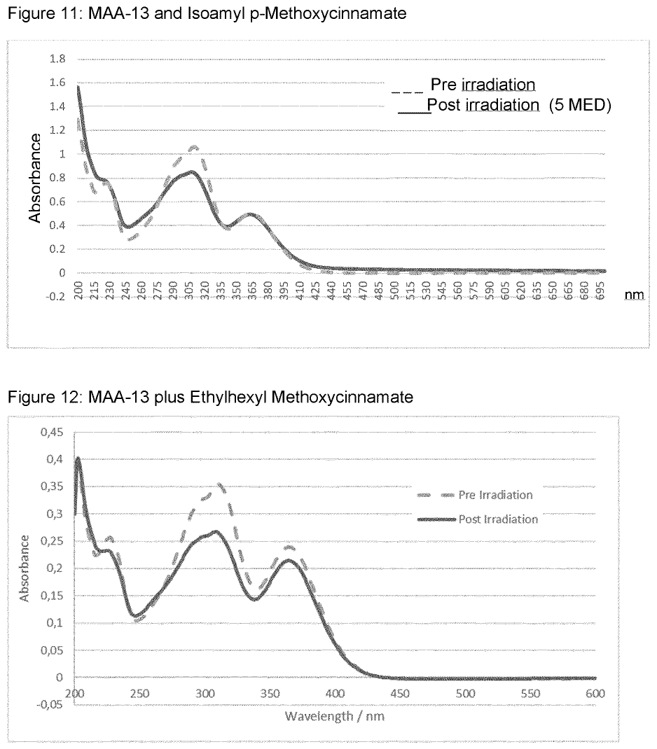

In addition, the critical wavelength is shifted from 324 nm for isoamyl p-Methoxycinnamate alone to 371 nm for the above mixture, which indicates that the protection of the sunscreen composition is not limited to the wavelengths of UV-B, i.e. wavelengths from 280-320 nm, but extends to 371 nm in such a way that 90% of the total area under the protective curve in the UV region are reached at 371 nm. Accordingly, the combination results in a broader spectrum of protection across the ultraviolet radiation spectrum showing both UVB and UVA activity relative to Isoamyl p-Methoxycinnamate alone (see FIG. 11).

In combination with MAA-13 it is possible to reduce the loss Ethylhexyl Methoxycinnamate from 37,88.50% to 24.69% by approximately 35%. The combination shows both UVB and UVA activity relative to Ethylhexyl Methoxycinnamate alone.

Additionally, the critical wavelength is shifted from 324 nm for Ethylhexyl Methoxycinnamate alone to 372 nm for the above mixture, which indicates that the protection of the sunscreen composition is not limited to the wavelengths of UV-B, i.e. wavelengths from 280-320 nm, but extends to 372 nm in such a way that 90% of the total area under the protective curve in the UV region are reached at 372 nm. Accordingly, the combination results in a broader spectrum of protection across the ultraviolet radiation spectrum showing both UVB and UVA activity relative to Ethylhexyl Methoxycinnamate alone (see FIG. 12).

In combination with MAA-20 it is possible to reduce the loss of Isoamyl p-Methoxycinnamate from 30.50% to 14.41% by approximately 52%. The combination shows both UVB and UVA activity relative to Isoamyl p-Methoxycinnamate alone.

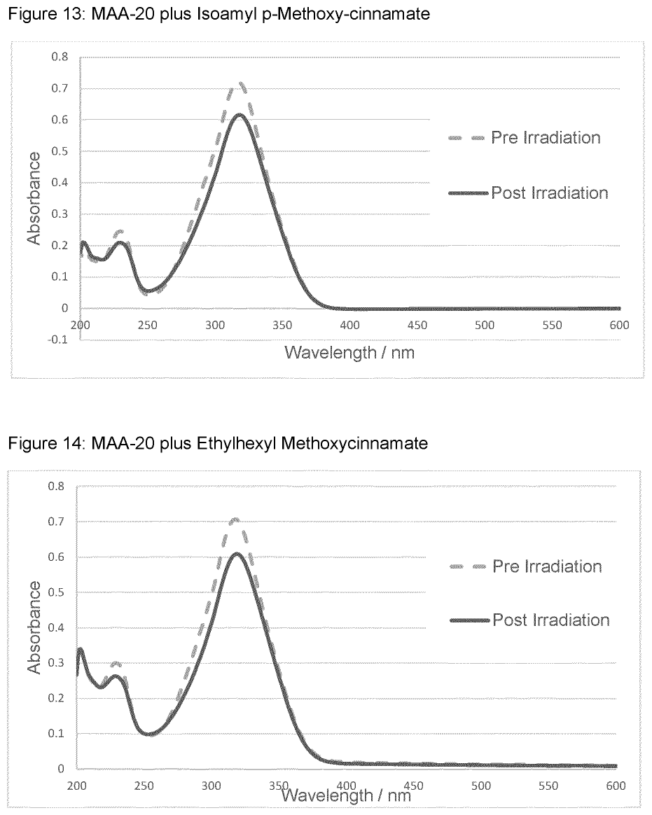

Additionally, the critical wavelength is shifted from 324 nm for Isoamyl p-Methoxycinnamate alone to 344 nm for the above mixture, which indicates that the protection of the sunscreen composition is not limited to the wavelengths of UV-B, i.e. wavelengths from 280-320 nm, but extends to 344 nm in such a way that 90% of the total area under the protective curve in the UV region are reached at 344 nm. Accordingly, the combination results in a broader spectrum of protection across the ultraviolet radiation spectrum showing both UVB and UVA activity relative to Isoamyl p-Methoxycinnamate alone (see FIG. 13).

In combination with MAA-20 it is possible to reduce the loss Ethylhexyl Methoxycinnamate from 37.88% to 13.80% by approximately 64%. The combination shows both UVB and UVA activity relative to Ethylhexyl Methoxycinnamate alone.

Additionally, the critical wavelength is shifted from 324 nm for Ethylhexyl Methoxycinnamate alone to 346 nm for the above mixture, which indicates that the protection of the sunscreen composition is not limited to the wavelengths of UV-B, i.e. wavelengths from 280-320 nm, but extends to 346 nm in such a way that 90% of the total area under the protective curve in the UV region are reached at 346 nm. Accordingly, the combination results in a broader spectrum of protection across the ultraviolet radiation spectrum showing both UVB and UVA activity relative to Ethylhexyl Methoxycinnamate alone (see FIG. 14).

In combination with MAA-28 it is possible to reduce the loss of Isoamyl p-Methoxycinnamate from 30.50% to 19.80% by approximately 35%. The combination shows both UVB and UVA activity relative to Isoamyl p-Methoxycinnamate alone.

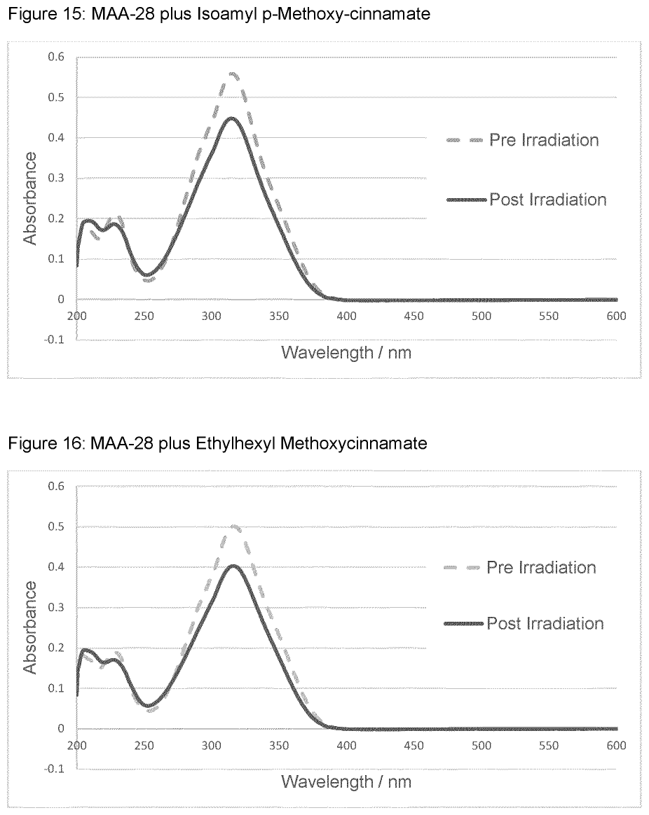

Additionally, the critical wavelength is shifted from 324 nm for Isoamyl p-Methoxycinnamate alone to 347 nm for the above mixture, which indicates that the protection of the sunscreen composition is not limited to the wavelengths of UV-B, i.e. wavelengths from 280-320 nm, but extends to 347 nm in such a way that 90% of the total area under the protective curve in the UV region are reached at 347 nm. Accordingly, the combination results in a broader spectrum of protection across the ultraviolet radiation spectrum showing both UVB and UVA activity relative to Isoamyl p-Methoxycinnamate alone (see FIG. 15).

In combination with MAA-28 it is possible to reduce the loss of Ethylhexyl Methoxycinnamate from 37.88% to 19.54% by approximately 48%. The combination shows both UVB and UVA activity relative to Ethylhexyl Methoxycinnamate alone.

Additionally, the critical wavelength is shifted from 324 nm for Ethylhexyl Methoxycinnamate alone to 349 nm for the above mixture, which indicates that the protection of the sunscreen composition is not limited to the wavelengths of UV-B, i.e. wavelengths from 280-320 nm, but extends to 349 nm in such a way that 90% of the total area under the protective curve in the UV region are reached at 349 nm. Accordingly, the combination results in a broader spectrum of protection across the ultraviolet radiation spectrum showing both UVB and UVA activity relative to Ethylhexyl Methoxycinnamate alone (see FIG. 16).

From the above data it is obvious that the water-soluble MAA compounds according to the present invention show a distinguished stabilization of the oil soluble photo-unstable UV-filters. Thus, with the water-soluble MAA compounds according to the present invention it is possible to stabilize oil-soluble unstable UV-filters with water soluble MAA stabilizers via the water phase in an emulsion formulation. Since then, a stabilization of oil-soluble unstable UV-filters was only possible with oil-soluble stabilizers in the oil phase of an emulsion formulation.

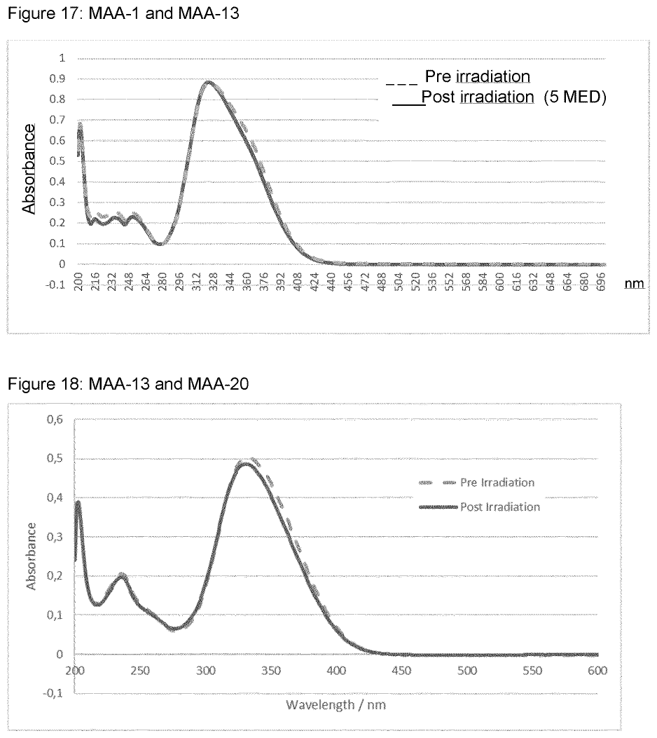

A mixture of MAA-1 and MAA-13 shows no absorbance loss at 325 nm. However, the absorption spectrum of the mixture alters in comparison to the absorption spectrum of MAA-1 and MAA-13 alone. The combination covers the complete UVA and UVB spectrum from 284 nm to 396 nm and the gap between the spectrum of MAA-1 alone and MAA-13 alone is closed (see FIG. 17).

A mixture of MAA-13 and MAA-20 shows no absorbance loss at 325 nm. However, the absorption spectrum of the mixture alters in comparison to the absorption spectrum of MAA-20 and MAA-13 alone. The combination covers the complete UVA and UVB spectrum from 284 nm to 396 nm and the gap between the spectrum of MAA-13 alone and MAA-20 alone is closed (see FIG. 18).

Thus, with a mixture of two MAA compounds according to the invention, an improved photostability can be obtained, compared to the photostability of the respective single MAA compounds.

Hence, the present invention provides for a sunscreen product or cosmetic or pharmaceutical preparation wherein the reduction in degradation after irradiation at 5 MED determined as described in the application is reduced by at least 10%, compared to a composition comprising the at least one photo-unstable UV-filter without the mycosporine-like amino acid compound. More beneficial, in the sunscreen products, cosmetic or pharmaceutical preparations or homecare products according to the present invention, the reduction in degradation of the photo-unstable UV-filter is at least 15%, more preferred by at least 20% and most preferred by at least 25%.

In particular, this photostability stabilization effect is observed without the use of further photostabilizing agents.

Without the addition of a mycosporine-like amino acid compound, the photo-unstable UV-filter undergoes UV-induced photochemical interaction or reaction that result in the loss of UV absorbance of said UV-filter and severely degrade the UV protective capacity of the sunscreen product, cosmetic or pharmaceutical preparation or homecare product.

By stabilizing the UV-filter, its degradation upon exposure to UV-radiation can be reduced and the formation of phototoxic or photoallergic degradation products with a risk of adverse health effects when coming into direct contact with the skin can be reduced.

With the stabilization of a photo-unstable UV-filter, the initial UV-absorbing capacity in a sunscreen product, cosmetic or pharmaceutical or homecare product is almost retained. With the present invention it is therefore possible to offer sunscreen products, cosmetic or pharmaceutical products or homecare product having an almost consistently good SPF.

On the other hand, the stabilization of the photo-unstable UV-filter by a mycosporine-like amino acid compound as defined herein, allows to reduce the amount of the UV-filter, without affecting the envisaged SPF.

Due to this beneficial effect as described before, the present invention relates in a further aspect to the use of a mycosporine-like amino acid compound (b) as defined herein or a mixture thereof for stabilizing the photostability of a photo-unstable UV-filter.

By the admixture of a mycosporine-like amino acid compound (b) as defined herein or a mixture thereof the degradation loss of the photo-unstable UV-filter after irradiation at 5 MED, determined as described in the present application, can be reduced by at least 10%. Preferably, the degradation loss of the photo-unstable UV-filter can be reduced by at least 15%, more preferably by at least 20% and most preferably by at least 25%.

Advantageously, more than 50%, 55%, 60%, 65%, 70%, 75%, 80%, 85% or 90% or even more of the initial UV-absorbing capacity is retained for the photo-unstable UV-absorbing compound with the addition of a mycosporine-like amino acid, in comparison to products or preparations without the addition of at least one mycosporine-like amino acid compound.

In a further aspect, the present invention relates to a method of stabilizing a photo-unstable UV-filter against the degradative effects of UV irradiation or of enhancing the UV-absorbing properties of a UV-absorbing compound. In the method the photo-unstable UV-filter is admixed with an effective amount of at least one mycosporine-like amino acid as defined herein or a mixture thereof.

The present invention shall now be described in detail with reference to the following examples, which are merely illustrative of the present invention, such that the content of the present invention is not limited by or to the following examples.

### EXAMPLES

### Example 1—Photostability Test

UV-filters are usually tested for photostability to ensure that no degradation of the UV-filter itself or in combination with other substances takes place.

The UV-filter stabilizing properties of mycosporine-like amino acid compounds were explored by comparing the behaviour of UV-filter alone relative to UV-filters in combination with a certain mycosporine-like amino acid compound.

In the present case single photo-unstable UV-filters as well as formulations of photo-unstable filters with certain mycosporine-like amino acid compounds were subjected to photostability testing, described in detail below, to determine the retention of absorption of UVA and UVB radiation (full spectrum analysis) after exposing the sample to 5 MED (minimal erythema) dose (1 MED=250 J/m²).

The minimum erythema dose (MED) (also erythema threshold dose) is a measure of the tolerance of human skin to solar radiation, especially to the short-wave ultraviolet portion of the spectrum around about 300 nm wavelength. Dermatologists determine MED by irradiating a test area, usually on the untanned back with gradually (so-called “light staircase”) increased doses of a powerful UV lamp. 1 MED corresponds to the lowest irradiation dose that caused a sharply defined erythema (redness) of the skin when read after 24 hours.

Sample preparation: The substance or the formulation to be tested was dissolved in a suitable solvent. For this purpose, a certain amount of the substance or formulation was transferred by means of an analytical balance/pipette into a graduated flask and filled with the selected solvent. The filling up to the calibration mark of the graduated flask was carried out only after complete dissolution of the substance and temperature control at 20° C.

Preparation of solution E 1%/cm: 100 mg of the substance or formulation to be tested were weighed into a graduated flask and filled up to 100 ml with the corresponding solvent. A clear solution was obtained. If necessary, an ultrasonic bath can be used to improve solubility. For further dilution the sample was adjusted to a temperature of 20° C. 1 ml of the dissolved solution was added into a second graduated 100 ml flask, filled up to 100 ml at a temperature of 20° C. The final concentration of the thus obtained solution was 0.001%. (E1%/cm). This solution was used for absorbance measurement and irradiation. Said solution was transferred to a 10 mm quartz cuvette with closure.

Irradiation: Irradiation of the sample was performed for 32 min with 700 W/m² emitted by an Atlas SUN Test CPS. This corresponds to an amount of energy of 5 MED (minimal erythema) dose (=1,250 J/m²). At the same time, the temperature of the devise was adjusted to max 30° C. in order to avoid strong heating of the sample.

Measurement: The absorbance measurement of the sample in the quartz cuvette with closure (10 mm) was carried out in a wavelength range of 200 nm to 700 nm with a Lambda 25 UV spectrometer. A blank absorbance test was carried out with the corresponding solvent for preparing the samples as described above. The absorbance measurement of the samples was carried out before (pre-irradiation) and after irradiation emitted by a Atlas SUN Test CPS (see above).

The UV absorbance curves obtained demonstrates the amplitude and breadth of protection provided from 200 nm to 700 nm across the UV spectrum for each sample both before (pre-irradiation) and after irradiation as described above. The amplitude of the absorbance curve reflects the degree of protection. The higher the amplitude of the curve, the greater the absorbance and the more protection provided at that wavelength. Within the UVB portion of the spectrum (290 nm to 320 nm) this amplitude correlates with the SPF. The greater the breadth of the curve, the more protection provided against longer wave UV ration. In other words, the greater the breadth of the curve, the broader the spectrum of sun protection provided.

The Tables show the changes in UV absorbance performance of either the individual components or the various combinations.

Samples with an absorbance loss<20% after irradiation can be classified as photostable.

### Tested Samples:

### a) E1%/cm Solution of Pure Substances:

1) MAA-1 (mycosporine-like amino acid compound)
2) MAA-13 (mycosporine-like amino acid compound)
3) MAA-20 (mycosporine-like amino acid compound)
4) MAA-28 (mycosporine-like amino acid compound)
5) Ethylhexyl Methoxycinnamate (UV-filter)
6) Butyl Methoxydibenzoylmethane (UV-filter)
7) Isoamyl p-Methoxycinnamate (UV-filter)

### b) E1%/cm Solution of the Following Mixtures:

1) MAA-1 plus Ethylhexyl Methoxycinnamate
2) MAA-1 plus Butyl Methoxydibenzoylmethane
3) MAA-1 plus Isoamyl p-Methoxycinnamate

### c) E1%/cm Solution of the Following Mixture:

1) MAA-13 plus Isoamyl p-Methoxycinnamate
2) MAA-13 plus Ethylhexyl Methoxycinnamate

### d) E1%/cm Solution of the Following Mixture:

1) MAA-20 plus Isoamyl p-Methoxycinnamate
2) MAA-20 plus Ethylhexyl Methoxycinnamate

### e) E1%/cm Solution of the Following Mixture:

1) MAA-28 plus Isoamyl p-Methoxycinnamate
2) MAA-28 plus Ethylhexyl Methoxycinnamate

### f) E1%/cm Solution of the Following Mixture:

1) MAA-13 plus MAA-1
2) MAA-13 plus MAA 20

### Results

### 1) Photostability E1%/cm Measurement of MAA-1 (Mycosporine-Like Amino Acid Compound):

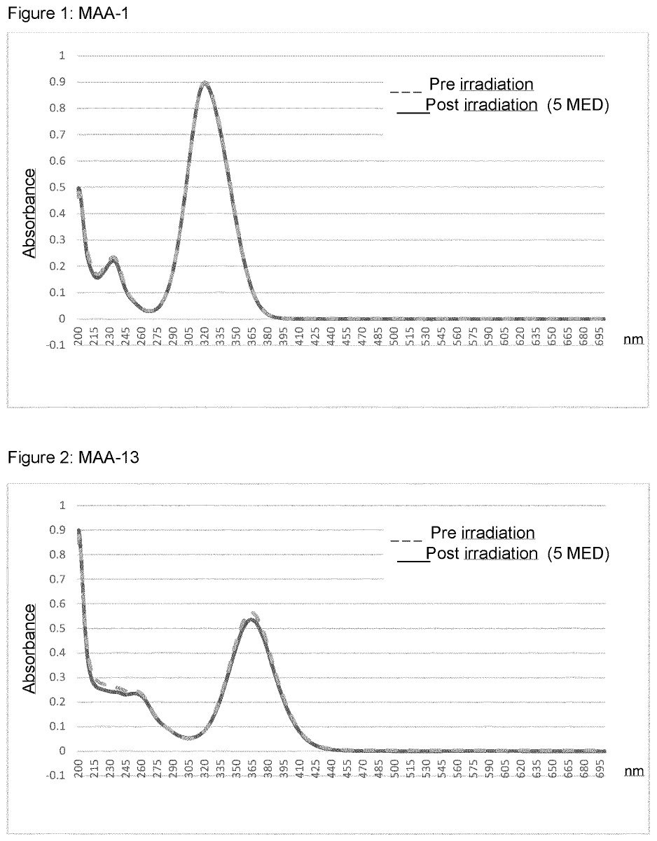

Results: Absorbance loss 0.53% at 321 nm. Very photostable molecule (see FIG. 1).

### 2) Photostability E1%/cm Measurement of MAA-13 (Mycosporine-Like Amino Acid Compound):

Results: Absorbance loss 5.13% at 364 nm. Very photostable molecule (see FIG. 2).

### 3) Photostability E1%/cm Measurement of MAA-20 (Mycosporine-Like Amino Acid Compound):

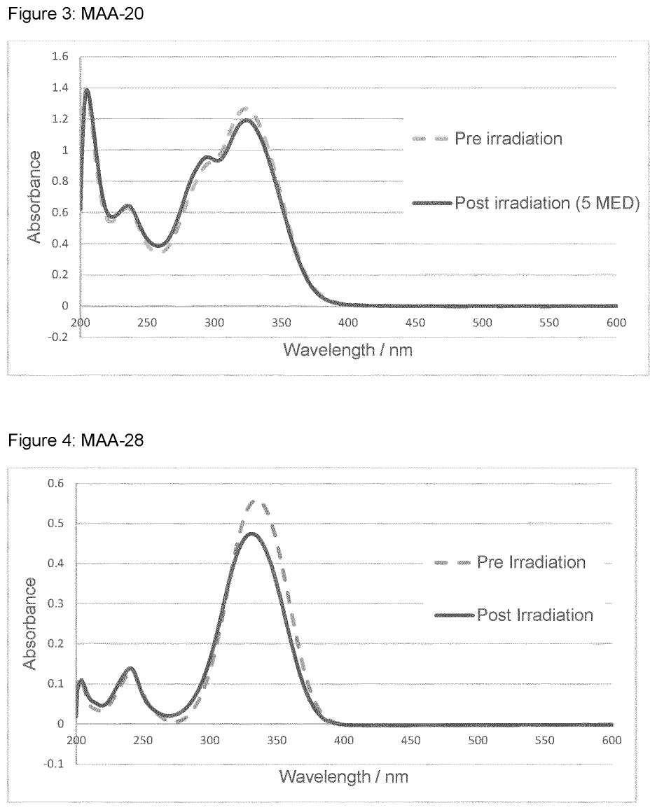

Results: Absorbance loss 5.95% at 324 nm. Very photostable molecule (see FIG. 3).

### 4) Photostability E1%/cm Measurement of MAA-28 (Mycosporine-Like Amino Acid Compound):

Results: Absorbance loss 15.03% at 335 nm. A photostable molecule (see FIG. 4).

### 5) Photostability E1%/cm Measurement of Ethylhexyl Methoxycinnamate (UV-Filter):

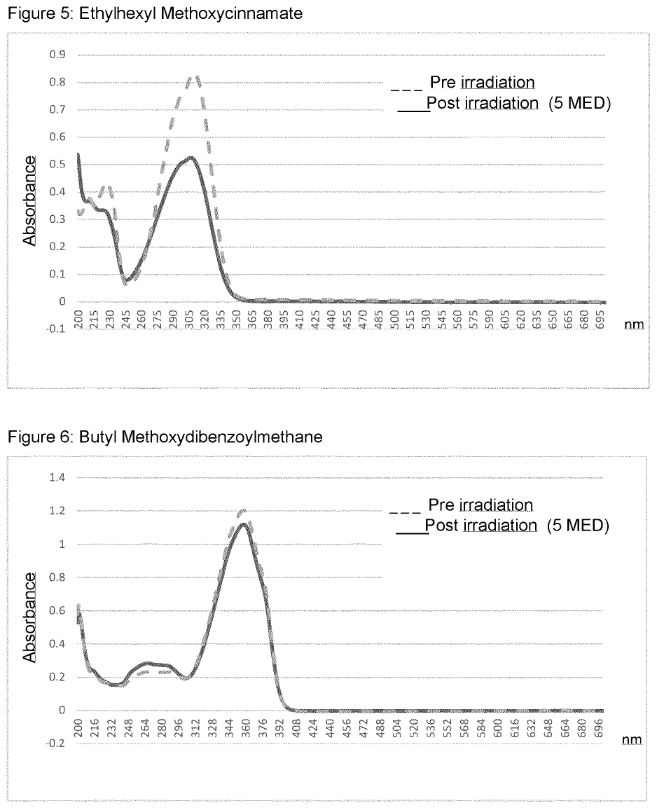

Results: Absorbance loss 37.88% at 310 nm. Not photostable (see FIG. 5).

### 6) Photostability E1%/cm Measurement of Butyl Methoxydibenzoylmethane (UV-Filter):

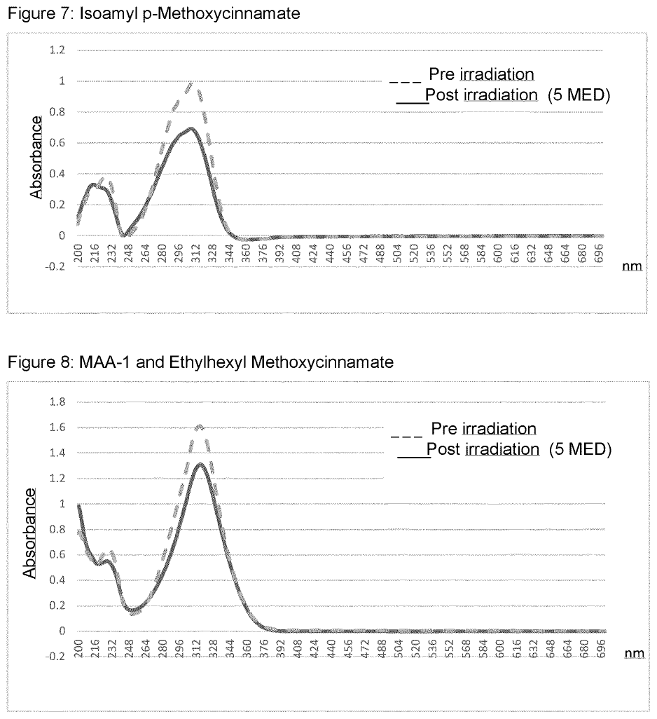

Results: Absorbance loss 7.05% at 358 nm. Butyl Methoxydibenzoylmethane is photostable in this kind of medium (see FIG. 6).
   7) Photostability E1%/cm Measurement of Isoamyl p-Methoxycinnamate (UV-Filter):
Results: Absorbance loss 30.5% at 310 nm. Not photostable (see FIG. 7).

### 8) Photostability E1%/cm Measurement of a Mixture of MAA-1 and Ethylhexyl Methoxycinnamate:

Results: Absorbance loss 18.82% at 316 nm. The mixture seems photostable.
The loss of Ethylhexy Methoxycinnamate is reduced from 37.88% to 18.82%, i.e. by approximately 50% in combination with MAA-1 (see FIG. 8):

| | MAA-1 and | |
|---|---|---|
| | Ethylhexyl | Ethylhexyl |
| Product | Methoxycinnamate | Methoxycinnamate |
| E max (nm) | 316 | 310 |
| Lost (%) after irradiation with | 18.82 | 37.88 |
| 5 MED (1,250 J/m²) | | |
| Results | photostable | Placebo |

### 9) Photostability E1%/cm Measurement of a Mixture of MAA-1 and Butyl Methoxydibenzoylmethane:

Results: Absorbance loss 5.27% at 342 nm. The mixture is photostable.
It is possible to reduce the loss of Butylmethoxydibenzoylmethane from 7.05% to 5.27%, i.e. by approximately 25% in combination with MAA-1 (see FIG. 9).

| | MAA-1 and Butyl | Butyl |
|---|---|---|
| | Methoxydibenzoyl- | Methoxydibenzoyl- |
| Product | methane | methane |
| E max (nm) | 342 | 358 |
| Lost (%) after irradiation with | 5.27 | 7.05 |
| 5 MED (1,250 J/m²) | | |
| Results | photostable | Placebo |

10) Photostability E1%/cm Measurement of a Mixture of MAA-1 and Isoamyl p-Methoxycinnamate:
Results: Absorbance loss 18.6% at 315 nm. The mixture is photostable.
In combination with MAA-1 it is possible to reduce the loss of Isoamyl p-Methoxycinnamate from 30.50% to 18.60% by approximately 39% (see FIG. 10).

| | MAA-1 plus | |
|---|---|---|
| | Isoamyl p- | Isoamyl p- |
| Product | Methoxycinnamate | Methoxycinnamate |
| E max (nm) | 315 | 310 |
| Lost (%) after irradiation with | 18.60 | 30.50 |
| 5 MED (1,250 J/m²) | | |
| Results | photostable | Placebo |

11) Photostability E1%/cm Measurement of a Mixture of MAA-13 and Isoamyl p-Methoxycinnamate:
Results: Absorbance loss 20.45% at 310 nm.
In combination with MAA-13 it is possible to reduce the loss of Isoamyl p-Methoxycinnamate from 30.50% to 20.45% by approximately 32%.
The combination shows both UVB and UVA activity relative to Isoamyl p-Methoxycinnamate alone (see FIG. 11).
Additionally, the critical wavelength is shifted from 324 nm for isoamyl p-Methoxycinnamate alone to 371 nm for the above mixture, which indicates that the protection of the sunscreen composition is not limited to the wavelengths of UV-B, i.e. wavelengths from 280-320 nm, but extends to 371 nm in such a way that 90% of the total area under the protective curve in the UV region are reached at 371 nm.

| | MAA-13 plus | |
|---|---|---|
| | Isoamyl p- | Isoamyl p- |
| Product | Methoxycinnamate | Methoxycinnamate |
| E max (nm) | 310 | 310 |
| Lost (%) after irradiation with | 20.45 | 30.50 |
| 5 MED (1,250 J/m²) | | |
| Critical Wavelength (nm) | 371 | 324 |
| Results | photostable | Placebo |

### 12) Photostability E1%/cm Measurement of a Mixture of MAA-13 and Ethylhexyl Methoxycinnamate:

Results: Absorbance loss 20.45% at 310 nm.
In combination with MAA-13 it is possible to reduce the loss Ethylhexyl Methoxycinnamate from 37.88 to 24.69% by approximately 35%.
The combination shows both UVB and UVA activity relative to Ethylhexyl Methoxycinnamate alone (see FIG. 12).
Additionally, the critical wavelength is shifted from 324 nm for Ethylhexyl Methoxycinnamate alone to 372 nm for the above mixture, which indicates that the protection of the sunscreen composition is not limited to the wavelengths of UV-B, i.e. wavelengths from 280-320 nm, but extends to 372 nm in such a way that 90% of the total area under the protective curve in the UV region are reached at 372 nm.

| | MAA-13 plus | |
|---|---|---|
| | Ethylhexyl | Ethylhexyl |
| Product | Methoxycinnamate | Methoxycinnamate |
| E max (nm) | 310 | 310 |
| Lost (%) after irradiation | 24.69 | 37.88 |
| with 5 | | |
| MED (1,250 J/m²) | | |
| Critical Wavelength (nm) | 372 | 324 |
| Results | Improved | Placebo |
| | photostability | |

13) Photostability E1%/cm Measurement of a Mixture of MAA-20 and Isoamyl p-Methoxycinnamate:
Results: Absorbance loss 16.41% at 310 nm.
In combination with MAA-20 it is possible to reduce the loss of Isoamyl p-Methoxycinnamate from 30.50% to 14.41% by approximately 52%.
The combination shows both UVB and UVA activity relative to Isoamyl p-Methoxycinnamate alone (see FIG. 13).
Additionally, the critical wavelength is shifted from 324 nm for Isoamyl p-Methoxycinnamate alone to 344 nm for the above mixture, which indicates that the protection of the sunscreen composition is not limited to the wavelengths of UV-B, i.e. wavelengths from 280-320 nm, but extends to 344 nm in such a way that 90% of the total area under the protective curve in the UV region are reached at 344 nm.

| | MAA-20 plus | |
|---|---|---|
| | Isoamyl p- | Isoamyl |
| Product | Methoxycinnamate | p-Methoxycinnamate |
| E max (nm) | 318 | 310 |
| Lost (%) after irradiation | 14.41 | 30.50 |
| with 5 | | |
| MED (1,250 J/m²) | | |
| Critical Wavelength (nm) | 344 | 324 |
| Results | photostable | Placebo |

### 14) Photostability E1%/cm Measurement of a Mixture of MAA-20 and Ethylhexyl Methoxycinnamate:

Results: Absorbance loss 13.80% at 318 nm.
In combination with MAA-20 it is possible to reduce the loss Ethylhexyl Methoxycinnamate from 37.88% to 13.80% by approximately 64%.
The combination shows both UVB and UVA activity relative to Ethylhexyl Methoxycinnamate alone (see FIG. 14).
Additionally, the critical wavelength is shifted from 324 nm for Ethylhexyl Methoxycinnamate alone to 346 nm for the above mixture, which indicates that the protection of the sunscreen composition is not limited to the wavelengths of UV-B, i.e. wavelengths from 280-320 nm, but extends to 346 nm in such a way that 90% of the total area under the protective curve in the UV region are reached at 346 nm.

| | MAA-20 plus | |
|---|---|---|
| | Ethylhexyl | Ethylhexyl |
| Product | Methoxycinnamate | Methoxycinnamate |
| E max (nm) | 318 | 310 |
| Lost (%) after irradiation | 13.80 | 37.88 |
| with 5 | | |
| MED (1,250 J/m²) | | |
| Critical Wavelength (nm) | 346 | 324 |
| Results | photostable | Placebo |

15) Photostability E1%/cm Measurement of a Mixture of MAA-28 and Isoamyl p-Methoxycinnamate:
Results: Absorbance loss 16.41% at 310 nm.
In combination with MAA-28 it is possible to reduce the loss of Isoamyl p-Methoxycinnamate from 30.50% to 19.80% by approximately 35%.
The combination shows both UVB and UVA activity relative to Isoamyl p-Methoxycinnamate alone (see FIG. 15).
Additionally, the critical wavelength is shifted from 324 nm for Isoamyl p-Methoxycinnamate alone to 347 nm for the above mixture, which indicates that the protection of the sunscreen composition is not limited to the wavelengths of UV-B, i.e. wavelengths from 280-320 nm, but extends to 347 nm in such a way that 90% of the total area under the protective curve in the UV region are reached at 347 nm.

| | MAA-28 plus | |
|---|---|---|
| | Isoamyl p- | |
| | Methoxy- | Isoamyl p- |
| Product | cinnamate | Methoxycinnamate |
| E max (nm) | 315 | 310 |
| Lost (%) after irradiation | 19.80 | 30.50 |
| with 5 | | |
| MED (1,250 J/m²) | | |
| Critical Wavelength (nm) | 347 | 324 |
| Results | photostable | Placebo |

### 16) Photostability E1%/cm Measurement of a Mixture of MAA-28 and Ethylhexyl Methoxycinnamate:

Results: Absorbance loss 13.80% at 318 nm.
In combination with MAA-28 it is possible to reduce the loss of Ethylhexyl Methoxycinnamate from 37.88% to 19.54% by approximately 48%.
The combination shows both UVB and UVA activity relative to Ethylhexyl Methoxycinnamate alone (see FIG. 16).
Additionally, the critical wavelength is shifted from 324 nm for Ethylhexyl Methoxycinnamate alone to 349 nm for the above mixture, which indicates that the protection of the sunscreen composition is not limited to the wavelengths of UV-B, i.e. wavelengths from 280-320 nm, but extends to 349 nm in such a way that 90% of the total area under the protective curve in the UV region are reached at 349 nm.

| | MAA-20 plus | |
|---|---|---|
| | Ethylhexyl | Ethylhexyl |
| Product | Methoxycinnamate | Methoxycinnamate |
| E max (nm) | 317 | 310 |
| Lost (%) after irradiation | 19.54 | 37.88 |
| with 5 | | |
| MED (1,250 J/m²) | | |
| Critical Wavelength (nm) | 349 | 324 |
| Results | photostable | Placebo |

### 17) Photostability E1%/cm Measurement of a Mixture of MAA-1 and MAA-13:

No absorbance loss at 325 nm. The absorption spectrum has been changed. The combination covers the complete UVA and UVB spectrum from 284 nm to 396 nm (see FIG. 17).

### 18) Photostability E1%/cm Measurement of a Mixture of MAA-20 and MAA-13:

No absorbance loss at 325 nm. The absorption spectrum has been changed. The combination covers the complete UVA and UVB spectrum from 284 nm to 396 nm (see FIG. 18).

### Formulation Examples

In the following formulation examples the following five perfume oils PFO1, PFO2, PFO3, PFO4 or PFO5 were each used as fragrance.

TABLE F1
Composition of perfume oil 1 (PO1; amounts in ‰ by weight)

| Ingredients | Amount |
|---|---|
| Aldehyde C14 | 2.0 |
| Allyl amyl glycolate 10% dpg | 5.0 |
| Anisic aldehyde pure | 5.0 |
| Apple oliffac type | 10.0 |
| Benzylacetate | 50.0 |
| Bergamot identoil ® colourless | 15.0 |
| Canthoxal | 5.0 |
| Cetalox 10% ipm | 3.0 |
| Citronellol 950 | 40.0 |
| Damascenone total 1% dpg | 5.0 |
| Damascone alpha 10% dpg | 5.0 |
| Damascone delta 10% dpg | 2.0 |
| Dimethyl benzyl carbinyl butyrate | 2.0 |
| Dipropylene glycol | 178.0 |
| Ebanol | 2.0 |
| Ethyl decadienoate trans cis-2.4 10% ipm | 2.0 |
| Florosa | 5.0 |
| Frambinon ® 10% dpg | 7.0 |
| Galaxolide 50% in ipm | 100.0 |
| Galbex type base | 1.0 |
| Geranyl acetate pure | 2.0 |
| Hedione | 30.0 |
| Heliotropin | 10.0 |
| Hexenyl acetate cis-3 10% dpg | 1.0 |
| Hexenyl salicylate cis-3 | 5.0 |
| Hexyl cinnamic aldehyde alpha | 70.0 |
| Hexyl salicylate | 50.0 |
| Hydroxy citronellal | 10.0 |
| Iso e super | 15.0 |
| Isoraldeine 70 | 20.0 |
| Leafovert ® | 1.0 |
| Lilial | 60.0 |
| Linalool | 60.0 |
| Linalyl acetate | 20.0 |
| Lyral | 7.0 |
| Manzanate | 2.0 |
| Phenoxanol | 7.0 |
| Phenylethyl alcohol | 120.0 |
| Sandal mysore core | 2.0 |
| Sandranol ® | 7.0 |
| Styralyl acetate | 3.0 |
| Tagetes rco 10% tec | 2.0 |
| Terpineol pure | 20.0 |
| Tetrahydrogeraniol 10% dpg | 5.0 |
| Tonalide | 7.0 |
| Vertocitral 10% dpg | 5.0 |
| Vertofix | 15.0 |
| Total | 1000.0 |

TABLE F2
Composition of perfume oil 2 (PO2; amounts in ‰ by weight)

| Ingredients | Amount |
|---|---|
| Acetophenone. 10% in DPG | 10.0 |
| n-Undecanal | 5.0 |
| Aldehyde C14 | 15.0 |
| Allylamyl glycolate, 10% in DPG | 20.0 |
| Amyl salicylate | 25.0 |
| Benzyl acetate | 60.0 |
| Citronellol | 80.0 |
| d-Limonene | 50.0 |
| Decenol trans-9 | 15.0 |
| Dihydromyrcenol | 50.0 |
| Dimethylbenzylcarbinyl acetate | 30.0 |
| Diphenyloxide | 5.0 |
| Eucalyptol | 10.0 |
| Geraniol | 40.0 |
| Nerol | 20.0 |
| Geranium oil | 15.0 |
| Hexenol cis-3. 10% in DPG | 5.0 |
| Hexenyl salicylate cis-3 | 20.0 |
| Indole. 10% in DPG | 10.0 |
| Alpha-ionone | 15.0 |
| Beta-ionone | 5.0 |
| Lilial ^{®} (2-methyl-3-(4-tert-butyl-phenyl)propanal) | 60.0 |
| Linalool | 40.0 |
| Methylphenyl acetate | 10.0 |
| Phenylethyl alcohol | 275.0 |
| Styrolyl acetate | 20.0 |
| Terpineol | 30.0 |
| Tetrahydrolinalool | 50.0 |
| Cinnamyl alcohol | 10.0 |
| Total: | 1000.0 |

TABLE F3
Composition of perfume oil 3 (PO3; amounts in ‰ by weight)

| Ingredients | Amount |
|---|---|
| Benzyl acetate | 60.0 |
| Citronellyl acetate | 60.0 |
| Cyclamenaldehyde (2-methyl-3-(4- | 20.0 |
| isopropylphenyl)propanal | |
| Dipropylene glycol (DPG) | 60.0 |
| Ethyllinalool | 40.0 |
| Florol (2-isobutyl-4-methyltetrahydro-2H-pyran- | 30.0 |
| 4-ol) | |
| Globanone ^{®} [(E/Z)-8-cyclohexadecen-1-one] | 180.0 |
| Hedione ^{®} (methyldihydrojasmonate) | 140.0 |
| Hexenyl salicylate. cis-3 | 10.0 |
| Vertocitral (2.4-dimethyl-3- | 5.0 |
| cyclohexenecarboxaldehyde) | |
| Hydratropaldehyde. 10% in DPG | 5.0 |
| Isodamascone (1-(2.4.4-trimethyl-2-cyclohexen- | 5.0 |
| 1-yl)-2-buten-1-one. 10% in DPG | |
| Isomuscone (cyclohexadecanone) | 40.0 |
| Jacinthaflor (2-methyl-4-phenyl-1.3-dioxolane) | 10.0 |
| Cis-jasmone. 10% in DPG | 20.0 |
| Linalool | 50.0 |
| Linalyl acetate | 30.0 |
| Methyl benzoate. 10% in DPG | 25.0 |
| para-Methyl cresol. 10% in DPG | 10.0 |
| Nerol | 20.0 |
| Phenylpropylaldehyde | 5.0 |
| 2-Phenylethyl alcohol | 82.0 |
| Tetrahydrogeraniol | 13.0 |
| 2.2-Dimethyl-3-cyclohexyl-1-propanol | 80.0 |
| Total: | 1000.0 |

TABLE F4
Composition of perfume oil 4 (PO4; amounts in ‰ by weight)

| Ingredients | Amount |
|---|---|
| Ambrettolide (macro) | 10.0 |
| Ambroxide 10% in ipm | 10.0 |
| Benzyl acetate | 20.0 |
| Benzyl salicylate | 15.0 |
| Bergamot oil. Bergapten-free | 60.0 |
| Calone ^{®} 1951 10% in dpg | 15.0 |
| Coumarin | 5.0 |
| Cyclogalbanate ^{®} 10% in dpg | 10.0 |
| Alpha-damascone 1% in dpg | 20.0 |
| Dihydromyrcenol | 10.0 |
| Ethyl linalool | 75.0 |
| Ethyl linalylacetate | 50.0 |
| Ethyl maltol 1% in dep | 10.0 |
| Ethylene brassylate (macro) | 80.0 |
| Florosa | 40.0 |
| Geranylacetate | 10.0 |
| Hedione ^{®} hc/30 | 35.0 |
| Hedione ^{®} | 210.0 |
| Helional ^{®} | 15.0 |
| Helvetolide ^{®} (alicyc) | 30.0 |
| Hexenylsalicylate cis-3 | 20.0 |
| Iso e super ^{®} | 40.0 |
| Leafovert ^{®} 10% in dep | 10.0 |
| Lilial ^{®} | 80.0 |
| Lyral ^{®} | 20.0 |
| Mandarin oil | 10.0 |
| Styralyl acetate | 5.0 |
| Symrose ^{®} | 15.0 |
| Vanillin 10% in dep | 20.0 |
| Dipropylene glycol (dpg) | 50.0 |
| Total | 1000.0 |

TABLE F5
Composition of perfume oil 5 (PO5; amounts in ‰ by weight)

| Ingredients | Amount |
|---|---|
| Amarocite | 10.0 |
| Ambrocenide ^{®} 10% in dpg | 5.0 |
| Ambroxide | 15.0 |
| Aurelione ^{®} (7/8-cyclohexadecenone) (macro) | 70.0 |
| Bergamot oil. Bergapten-free | 90.0 |
| Calone ^{®} 1951 10% in dpg | 20.0 |
| Caraway oil | 10.0 |
| Citral | 20.0 |
| Coumarin | 10.0 |
| Alpha-damascone 1% in dpg | 15.0 |
| Dihydromyrcenol | 70.0 |
| Estragon oil | 10.0 |
| Ethyl linalool | 100.0 |
| Ethyl linalylacetate | 90.0 |
| Eugenol | 10.0 |
| Evernyl ^{®} | 5.0 |
| Fructate ^{®} | 5.0 |
| Geranium oil | 5.0 |
| Hedione ^{®} hc/30 | 100.0 |
| Helional ^{®} | 10.0 |
| Indole 10% in dpg | 5.0 |
| Iso e super ^{®} | 100.0 |
| Kephalis ^{®} | 5.0 |
| Lavender oil | 40.0 |
| Citrus oil | 80.0 |
| Lilial ^{®} | 30.0 |
| Mandarin oil | 20.0 |
| Muscenone (macro) | 5.0 |
| Sandranol ^{®} | 10.0 |
| Vanillin 10% in dpg | 5.0 |
| Dipropylene glycol | 30.0 |
| Total | 1000.0 |

The above perfume oils PO1, PO2, PO3, PO4 or PO5 were incorporated into the formulations presented below.

Sunscreen products, cosmetic and pharmaceutical preparations and homecare products (formulations); amounts are indicated as % by weight for all formulations:
Sunscreen Oil Spray with Ethanol, expected SPF 30, UVA/UVB balanced
Urban Sun Spray expected SPF 50+
Sunscreen Spray (O/W), expected SPF 30 (all in one pot/cold manufacturing process)
UV protection water spray SPF 50
Sunscreen Spray, *SPF 50+, Very Water Resistant**
Sunscreen Lotion (O/W), expected SPF 50* (cold/cold manufacturing process)
Beach Time Lotion SPF 30
Anti-Sweat Sunscreen Lotion
Sun protection milk (w/o)
Face Protecting Watery Mousse expected SPF 30*
Full protection drops expected SPF 50
Glow Brightener, expected SPF 50plus*
Sunscreen Fluid for acne-prone Skin, expected SPF 30
No More Shine Fluid expected SPF50
Light Sunscreen Fluid expected SPF 50
Refreshing Gel Sunscreen, with expected SPF 30, UVA/UVB balanced
Sun protection balm, SPF 40
Anti-Pollution Hair Care (SPF 15)
Perfect Hand with expected SPF 15, UVA/UVB balanced
Tinted Sunscreen cream against hyperpigmentation expected SPF 50*
Bb Cream Dark Tone SPF-20 (expected)
Daily Well Aging, with SPF 30, UVA/UVB balanced
Anti-Wrinkle Day Emulsion expected SPF 15
Daily Tattoo Care with expected SPF 10, UVA/UVB balanced
Sensitive Day Care Cream expected SPF 15
Cream with expected SPF 15, UVA/UVB
All-purpose cleaner
APC alkaline
Detergent liquid light duty
Dishwash liquid, manual
Fabric softener
Fabric softener concentrate, encapsulated
Hand soap, liquid
Scent lotion, encapsulated
Cleaner, liquid, citric acid

TABLE F6
Sunscreen Oil Spray with Ethanol, expected SPF 30, UVA/UVB
balanced

| Raw material | INCI | Amount |
|---|---|---|
| Neo Heliopan ^{®} 357 | Butyl methoxydibenzoylmethane | 5.0 |
| Neo Heliopan ^{®} BMT | Bis-ethylhexyloxyphenol | 3.5 |
| | methoxyphenyl triazine | |
| Neo Heliopan ^{®} OS | Ethylhexyl salicylate | 5.0 |
| Neo Heliopan ^{®} EHT | Ethylhexyl triazone | 5.0 |
| SymMollient ^{®} PDCC | Propanediol dicaprylate/caprate | 10.0 |
| Cetiol ^{®} Sensoft | Propylheptyl caprylate | 6.0 |
| Cetiol ^{®} AB | C12-15 alkyl benzoate | Ad |
| | | 100 |
| Isopropyl Palmitate | Isopropyl palmitate | 20.7 |
| Sym Decanox ™ HA | Caprylic/capric triglyceride | 1.0 |
| | Hydroxymethoxyphenyl decanone | |
| Ethanol 96.5% | Alcohol, Aqua | 25.0 |
| Hydrolite ^{®} 5 green | Pentylene glycol | 3.0 |
| MAA-1 | | 0.1 |
| Hydrolite ^{®} 7 green | 1,2-heptanediol | 2.0 |
| Perfume oil PO1; PO2; PO3; | Perfume oil PO1; PO2; | 0.3 |
| PO4; or PO5 | PO3; PO4; or PO5 | |

TABLE F7
Urban Sun Spray, o/w emulsion expected SPF 50+

| Ingredients | INCI | Amount |
|---|---|---|
| Aqua/Water | Aqua | Ad |
| | | 100 |
| MAA-1 | | 1.0 |
| Vivapur ^{®} CS Tex sun | Microcrystalline Cellulose, Cellulose Gum | 1.0 |
| Vivapur ^{®} CS 032 XV | Microcrystalline Cellulose, Xanthan Gum | 1.0 |
| Sodium Hydroxide solution | Aqua, Sodium Hydroxide | 0.5 |
| 10% aq. | | |
| SymOcide ^{®} PH | Phenoxyethanol, Hydroxyacetophenone, | 1.4 |
| | Caprylyl Glycol, Aqua | |
| Emulsiphos ^{®} F | Potassium cetyl phosphate, | 1.5 |
| | Hydrogenatedpalm glycerides | |
| Dragoxat ^{®} 89 | Ethylhexyl Isononanoate | 2.0 |
| SymMollient ^{®} PDCC | Propanediol Dicaprylate/Caprate | 7.0 |
| SymSol ^{®} PF-3 | Aqua, Pentylene Glycol, Sodium Lauryl | 2.0 |
| | Sulfoacetate, Sodium Oleoyl Sarcosinate, | |
| | Sodium Chlrodie, Sodium Oleate | |
| Prisorine ^{®} 3505 | Isostearic acid | 1.5 |
| Neo Heliopan ^{®} BMT | Bis-Ethylhexyloxyphenol Methoxypenyl | 5.0 |
| | Triazine | |
| Neo Heliopan ^{®} 357 | Butyl Methoxydibenzoylmethane | 4.5 |
| Neo Heliopan ^{®} 303 | Octocrylene | 10.0 |
| Neo Heliopan ^{®} HMS | Homosalate | 10.0 |
| Neo Heliopan ^{®} OS | Ethylhexyl Salicylate | 5.0 |
| Dragosine ^{®} | Carnosine | 0.2 |
| SymUrban ^{®} | Benzylidene Dimethoxydimetylindanone | 0.2 |
| Hydrolite ^{®} 5 green | Pentylene glycol | 0.5 |
| SymVital ^{®} AR 3040 (974839) | Zingiber officinale (Ginger) Root extract | 0.1 |
| SymDecanox HA (972276) | Caprylic/Capric Triglyceride, | 1.0 |
| | Hydroxymethoxyphenyl decanone | |
| Floraesters ^{®} K-100 Jojoba | Hydrolyzed jojoba esters, Jojoba esters, Aqua | 0.5 |
| EDTA ^{®} BD | Disodium EDTA | 0.1 |
| Keltrol ^{®} CG-SFT | Xanthan Gum | 0.3 |
| Perfume oil PO1; PO2; PO3; | Perfume oil PO1; PO2; PO3; PO4; or PO5 | 0.5 |
| PO4; or PO5 | | |
| Farmal MS6135 | Calcium Starch Octenylsuccinate | 2.0 |
| Tapioca Pure | Tapioca starch | 1.5 |

TABLE F8
Sunscreen Spray (O/W), expected SPF 30* (cold manufacturing process)

| Ingredients | INCI | Amount |
|---|---|---|
| Dracorin ^{®} GOC | Glyceryl Oleate Citrate Caprylic/ | 2.0 |
| | Capric Triglyceride | |
| Dragosine ^{®} | Carnosine | 0.2 |
| Neo Heliopan ^{®} Flat | Homosalate, Octocrylene, Bis- Ethylhexyloxyphenol Methoxyphenyl | 25.0 |
| | Triazine, Butyl | |
| | Methoxydibenzoylmethane Ethylhexyl | |
| | Salicylate | |
| SymMollient ^{®} PDCC | Propanediol Dicaprylate/Caprate | 7.0 |
| Aristoflex ^{®} Silk | Sodium Polyacryloyldiemthyl taurate | 0.6 |
| Hydrolite ^{®} 5 green | Pentylene glycol | 3.0 |
| Aqua/Water | Aqua | Ad |
| | | 100 |
| MAA-13 | | 1.0 |
| Sodium Hydroxide solution 10% aq. | Aqua, Sodium Hydroxide | 0.50 |
| SymOcide ^{®} PH | Phenoxyethanol, | 1.45 |
| | Hydroxyacetophenone, Caprylyl | |
| | Glycol, Aqua | |
| SymMollient ^{®} PDCC | Propanediol Dicaprylate/ Caprate | 3.0 |
| Glycerin 99.5 % | Glycerin | 3.0 |
| Dragoxat ^{®} 89 | Ethylhexyl Isononanoate | 2.0 |
| PCL-Liquid ^{®} 100 | Cetearyl Ethylhexanoate | 2.0 |
| EDTA ^{®} BD | Disodium EDTA | 0.1 |
| Farmal MS | Calcium Starch Octenylsuccinate | 2.0 |
| Keltrol ^{®} CG-BT | Xanthan Gum | 0.4 |
| Perfume oil PO1; PO2; PO3; PO4; or | Perfume oil PO1; PO2; PO3; PO4; or | 0.3 |
| PO5 | PO5 | |

TABLE F9
UV protection water spray SPF 50

| Ingredients | INCI | Amount |
|---|---|---|
| Isolan GPS | Isododecane | 10.0 |
| Dermofeel sensolv MB | Diethylhexyl Carboate | 5.0 |
| Tegosoft ^{®} DC MB | Phenoxyethyl Caprylate | 5.0 |
| Neo Heliopan ^{®} 357 | Butyl Methoxydibenzoylmethane | 5.0 |
| Neo Heliopan ^{®} HMS | Homosalate | 5.0 |
| Neo Heliopan ^{®} 303 | Octocrylene | 5.0 |
| Neo Heliopan ^{®} OS | Ethylhexyl Salicylate | 5.0 |
| Neo Heliopan ^{®} BMT | Bis-Ethylhexyloxyphenol Methoxypenyl Triazine | 4.0 |
| Neo Heliopan ^{®} EHT | Ethylhexyl Triazone | 3.0 |
| Dermofeel ^{®} Toco 70 non GMO | Tocopherol; Helianthus Annuus | 0.5 |
| | (Sunflower) Seed oil | |
| Tego ^{®} SP 13 Sun Up | Poly C10-30 Alkyl Acrylate | 0.4 |
| SymEffect ^{®} Sun | Cera Alba. Sodium Stearoyl Lactylate | 1.5 |
| SymRepair ^{®} 100 | Hexyldecanol. Bisabolol. | 1.0 |
| | Cethylhydroxyproline Palmitamide. | |
| | Stearic acid. Brassica campestris | |
| | (Rapeseed) sterols | |
| SymDecanox ^{®} HA | Caprylic/Capric Triglyceride, | 1.0 |
| | Hydroxymethoxyphenyl decanone | |
| PCL Liquid ^{®} 100 | Cetearyl Ethlyhexanoate | 2.0 |
| Hydrolite ^{®} 5 green | Pentylene glycol | 0.5 |
| Perfume oil PO1; PO2; PO3; | Perfume oil PO1; PO2; PO3; PO4; or | 0.5 |
| PO4; or PO5 | PO5 | |
| Dermosoft ^{®} OMP | Methylpropandiol; Capryly Glycol; | 3.0 |
| | Phenylpropanol | |
| Water | Water (Aqua) | Ad |
| | | 100 |
| Neo Heliopan ^{®} Hydro | Phenylbenzimidazole Sulfonic Acid | 2.0 |
| MAA-1 | | 1.00 |
| MAA-13 | | 1.00 |
| Glycerin | Glycerin, Aqua | 0.5 |
| Sodium hydroxide 10 % sol. | Aqua, Sodium hydroxicde | 2.5 |
| EDTA ^{®} BD | Disosium EDTA | 0.1 |
| Dragosine ^{®} | Carnosine | 0.1 |
| Triethanolamine | Triethanolamine | 0.1 |

TABLE F10
Sunscreen Spray, *SPF 50+, Very Water Resistant**

| Ingredients | INCI | Amount |
|---|---|---|
| Emulsiphos ^{®} F | Potassium Cetyl Phosphate Hydrogenated Palm Glycerides | 2.0 |
| SymMollient ^{®} PDCC | Propanediol Dicaprylate/Caprate | 5.0 |
| SymMollient ^{®} S green | Cetearyl Nonanoate | 2.5 |
| Neo Heliopan ^{®} 357 | Butyl Methoxydibenzoylmethane | 5.0 |
| Neo Heliopan ^{®} HMS | Homosalate | 8.0 |
| Neo Heliopan ^{®} 303 | Octocrylene | 8.0 |
| Neo Heliopan ^{®} OS | Ethylhexyl Salicylate | 4.0 |
| Neo Heliopan ^{®} BMT | Bis-Ethylhexyloxyphenol Methoxypenyl | 4.0 |
| | Triazine | |
| Tocopherol Alpha DL | Tocopherol | 0.2 |
| Edeta ^{®} BD | EDTA | 0.1 |
| Sym Effect ^{®} Sun | Cera Alba, Sodium Stearoyl Lactylate | 3.0 |
| Keltrol ^{®} CG-SFT | Xanthan Gum | 0.2 |
| Water/Aqua | Aqua | Ad |
| | | 100 |
| Neo Heliopan ^{®} Hydro | Phenylbenzimidazole Sulfonic Acid | 1.5 |
| MAA-4 | | 2 |
| Biotive ^{®} L-Arginine | Arginine | 1.5 |
| Dragosine ^{®} | Carnosine | 0.1 |
| SymSave ^{®} H | Hydroxyacetophenone | 0.5 |
| Hydrolite ^{®} 5 green | Pentylene glycol | 1.8 |
| SymLite ^{®} G8 | Glyceryl caprylate | 0.6 |
| Cranberry Oil CC | Vaccinium Macrocappon seed oil | 1.0 |
| Tapioca Pure | Tapioca starch | 2.0 |
| VITACEL ^{®} CS 20 FC | Cellulose | 2.0 |
| Perfume oil PO1; PO2; PO3; | Perfume oil PO1; PO2; PO3; PO4; or | 0.3 |
| PO4; or PO5 | PO5 | |

TABLE F11
Sunscreen Lotion (O/W), expected SPF 50*
(cold/cold manufacturing process)

| Ingredients | INCI | Amount |
|---|---|---|
| Dracorin ® GOC | Glyceryl Oleate Citrate Caprylic/Capric | 2.0 |
| | Triglyceride | |
| KP-545 | Cyclopentasiloxane | 1.0 |
| | Acrylates/Dimethicone copolymer | |
| Neo Heliopan ® Flat | Homosalate, Octocrylene, Bis- | 30.0 |
| | Ethylhexyloxyphenol Methoxyphenyl | |
| | Triazine, Butyl | |
| | Methoxydibenzoylmethane Ethylhexyl | |
| | Salicylate | |
| SymMollient ® PDCC | Propanediol Dicaprylate/Caprate | 7.0 |
| Aristoflex ® Silk | Sodium Polyacryloyldiemthyl taurate | 0.6 |
| Aqua/Water | Aqua | Ad 100 |
| MAA-1 | | 3.00 |
| Sodium Hydroxide | Aqua, Sodium Hydroxide | 1.25 |
| solution 10% aq. | | |
| SymOcide ® PH | Phenoxyethanol, Hydroxyacetophenone, | 1.5 |
| | Caprylyl Glycol, Aqua | |
| Hydrolite ® 5 green | Pentylene glycol | 1.8 |
| Glycerin 99.5% | Glycerin | 2.0 |
| VITACEL ® CS 20 FC | Cellulose | 1.0 |
| Perfume oil PO1; PO2; | Perfume oil PO1; PO2; PO3; PO4; or | 0.3 |
| PO3; PO4; or PO5 | PO5 | |

TABLE F12
Beach Time Lotion SPF 30

| Ingredients | INCI | Amount |
|---|---|---|
| Isolan GPS | Polyglyceryl-4 Diisostearate/ | 3.5 |
| | Polyhydroxystearate/Sebacate | |
| Dermofeel ® sensolv MB | Isoamyl Laurate | 4.5 |
| TEGOSOFT ® DC MB | Decyl Cocoate | 4.0 |
| Neo Heliopan ® BMT | Bis-Ethylhexyloxyphenol | 3.0 |
| | Methoxypenyl Triazine | |
| Uvinul ® A plus | Diethylamino Hydroxybenzoyl | 3.0 |
| | Hexyl Benzoate | |
| Neo Heliopan ® OS | Ethylhexyl Salicylate | 4.0 |
| Neo Heliopan ® EHT | Ethylhexyl Triazone | 3.0 |
| Neo Heliopan ® HMS | Homosalate | 5.0 |
| Verstatil TBO | Triethylcitrate; Caprylyl Glycol; | 1.0 |
| | benzoic Acid | |
| Zinc Stearate | Zinc Stearate | 0.5 |
| Water | Aqua | Ad 100 |
| MAA-8 | | 1.00 |
| Sodium Hydroxide | Aqua, Sodium Hydroxide | 0.50 |
| solution 10% aq. | | |
| Glycerin | Glycerin | 3.0 |
| Zinc Sulfate Heptahydrate | Zinc Sulfate Heptahydrate | 1.2 |

TABLE F13
Anti-Sweat Sunscreen Lotion

| Raw material | INCI | Amount |
|---|---|---|
| Aqua/Water | Aqua | Ad 100 |
| MAA-12 | | 2.0 |
| Sodium Hydroxide solution 10% | Aqua, Sodium Hydroxide | 1.00 |
| aq. | | |
| Hydrolite ® 7 Green | 1,2-Heptanediol | 0.5 |
| Citric Acid 30% Sol. | Aqua, Citric acid | 0.6 |
| Aristoflex ® Velvet | Polyacrylate crosspolymer 11 | 0.6 |
| Glycerin 99.5% | Glycerin, Aqua | 2.0 |
| SymSol ® PF-3 (358712) | Aqua, Pentylene glycol, Sodium lauryl | 1.0 |
| | sulfoacetate, Sodium oleoyl sarcosinate, | |
| | Sodium chloride, Sodium oleate | |
| SymSave ® H | Hydroxyacetophenone | 0.3 |
| Neo Heliopan ® 357 | Butyl methoxydibenzoylmethane | 3.0 |
| Neo Heliopan ® 303 | Octocrylene | 10.0 |
| Neo Heliopan ® OS | Ethylhexyl salicylate | 5.0 |
| Neo Heliopan ® HMS | Homosalate | 10.0 |
| Neo Heliopan ® E1000 | Isoamyl p-methoxycinnamate | 2.0 |
| Neo Heliopan ® BMT | Bis-ethylhexyloxyphenol methoxyphenyl | 3.0 |
| | triazine | |
| Floraesters ® K-100 Jojoba | Hydrolyzed jojoba esters | 2.0 |
| | Jojoba esters | |
| | Aqua | |
| Dragoxat ® 89 | Ethylhexyl isononanoate | 6.0 |
| SymMollient ® S | Cetearyl nonanoate | 1.0 |
| EDTA ® BD | Disodium edta | 0.1 |
| Sym Decanox ™ HA | Caprylic/capric triglyceride | 1.0 |
| | Hydroxymethoxyphenyl decanone | |
| Frescolat ® Plus | Menthol | 0.3 |
| | Menthyl lactate | |
| SymDeo ® Plus | Lauryl alcohol, Phenoxyethanol, 2- | 1.0 |
| | benzylheptanol, Decylene glycol | |
| Hydroviton ® PLUS 2290 | Aqua, Pentylene glycol, Glycerin, | 4.0 |
| | Fructose, Urea, Citric acid, Sodium | |
| | hydroxide, Maltose, Sodium pca, Sodium | |
| | chloride, Sodium lactate, Trehalose, | |
| | Allantoin, Sodium hyaluronate, Glucose | |
| Sensocel ® 10 | Cellulose | 2.0 |

TABLE F14
Sun protection milk (w/o)

| Ingredients | INCI | Amount |
|---|---|---|
| Dehymuls ® PGPH | Polyglyceryl-2 | 3.0 |
| | dipolyhydroxystearate | |
| Beeswax 8100 | Beeswax | 1.0 |
| Monomuls ® 90-0-18 | Glyceryl oleate | 1.0 |
| Zinc stearate | Zinc stearate | 1.0 |
| Cetiol ® SN | Cetearyl isononanoate | 5.0 |
| Cetiol ® OE | Dicaprylyl ether | 5.0 |
| Tegosoft ® TN | C12-15 alkyl benzoate | 4.0 |
| Vitamin E | Tocopherol | 0.5 |
| Neo Heliopan ® OS | Ethylhexyl salicylate | 5.0 |
| Neo Heliopan ® AV | Ethylhexyl methoxycinnamate | 7.5 |
| Neo Heliopan ® EHT | Ethylhexyl triazone | 1.5 |
| Water, distilled | Water (Aqua) | ad 100 |
| MAA-4 | | 2.5 |
| Trilon ® BD | Disodium EDTA | 0.1 |
| Glycerol | Glycerol | 5.0 |
| Neo Heliopan ® AP 10% | Disodium phenyl dibenzimidazole | 15.0 |
| solution. neutralized with | tetrasulfonate | |
| NaOH | | |
| Sodium Hydroxide | Aqua, Sodium Hydroxide | 1.5 |
| solution 10% aq. | | |
| Perfume oil PO1; PO2; | Perfume oil PO1; PO2; PO3; | 0.3 |
| PO3; PO4; or PO5 | PO4; or PO5 | |
| Alpha Bisabolol | Bisabolol | 0.1 |
| SymOcide ® PT | Phenoxyethanol. Tropolone | 0.3 |
| Hydrolite ® 7 Green | 1,2-Heptanediol | 0.8 |

TABLE F15
Protecting Watery Mousse expected SPF 30*

| Ingredients | INCI | Amount |
|---|---|---|
| Aqua/Water | Aqua | Ad 100 |
| MAA-1 | | 3.00 |
| Neo Heliopan ® Hydro | Phenylbenzimidazole sulfonic acid | 2.0 |
| Neo Heliopan ® AP | Disodium phenyl dibenzimidazole | 1.0 |
| | tetrasulfonate | |
| Biotive ® L-Arginine | Arginine | 1.8 |
| Sodium Hydroxide | Aqua, Sodium Hydroxide | 1.5 |
| solution 10% aq. | | |
| SymSol ® PF-3 | Aqua, Pentylene glycol, Sodium | 2.5 |
| | lauryl, sulfoacetate, Sodium oleoyl | |
| | sarcosinate, Sodium chloride, Sodium | |
| | oleate | |
| Dragosine® | Carnosine | 0.2 |
| Aqua Keep 10SH-NFC | Sodium acrylates crosspolymer-2 | 2.5 |
| SymSave ® H | Hydroxyacetophenone | 0.5 |
| Phenoxyethanol | Phenoxyethanol | 0.5 |
| Hydrolite ® 5 Green | Pentylene glycol | 1.5 |
| Neo Heliopan ® 357 | Butyl methoxydibenzoylmethane | 3.0 |
| Neo Heliopan ® OS | Ethylhexyl salicylate | 5.0 |
| Neo Heliopan ® BMT | Bis-ethylhexyloxyphenol | 3.0 |
| | methoxyphenyl triazine | |
| Uvasorb ® HEB | Diethylhexyl butamido triazone | 3.0 |
| SymMollient ® S | Cetearyl nonanoate | 3.0 |
| EDTA ® BD | Disodium edta | 0.1 |
| Isoadipate | Diisopropyl adipate | 4.0 |
| Cetiol ® AB | C12-15 alkyl benzoate | 7.0 |
| Caprylic/Capric | Caprylic/capric triglyceride | 6.0 |
| Triglyceride | | |
| Frescolat ® ML | Menthyl Lactate | 2.0 |
| SymGlucan ® | Aqua, Glycerin, 1,2-Hexanediol, | 2.0 |
| | Caprylyl glycol, Beta-glucan | |
| Hydrolite ®-6 Green | 1,2-Hexanediol | 0.5 |

TABLE F16
Full protection emulsion, expected SPF 50

| Ingredients | INCI | Amount |
|---|---|---|
| Dracorin ® GOC | Glyceryl oleate citrate, Caprylic/capric | 2.5 |
| | triglyceride | |
| Neo Heliopan ® 303 | Octocrylene | 8.0 |
| Neo Heliopan ® 357 | Butyl methoxydibenzoylmethane | 4.5 |
| Neo Heliopan ® HMS | Homosalate | 10.0 |
| Neo Heliopan ® OS | Ethylhexyl salicylate | 5.0 |
| Neo Heliopan ® BMT | Bis-ethylhexyloxyphenol | 5.0 |
| | methoxyphenyl triazine | |
| SymMollient ® PDCC | Propanediol dicaprylate/caprate | 5.0 |
| Velvesil ® DM | Dimethicone, Cetearyl dimethicone | 3.0 |
| | crosspolymer | |
| Dow Corning ® 9041 | Dimethicone, Dimethicone | 2.0 |
| Silicone Elastomer Blend | crosspolymer | |
| EDTA ® BD | Disodium edta | 0.1 |
| Keltrol ® CG-SFT | Xanthan gum | 0.2 |
| Pemulen ™ TR-2 | Acrylates/c10-30 alkyl acrylate | 0.1 |
| Polymeric Emulsifier | crosspolymer | |
| Aqua/Water | Aqua | Ad 100 |
| MAA-13 | | 3.0 |
| Glycerin 99.5% | Glycerin, Aqua | 3.0 |
| Sodium Hydroxide | Aqua, Sodium Hydroxide | 1.3 |
| solution 10% aq. | | |
| Dragosine ® | Carnosine | 0.2 |
| SymSave ® H | Hydroxyacetophenone | 0.5 |
| Hydrolite ® 5 green | Pentylene glycol | 0.8 |
| Hydrolite ® CG | Caprylyl glycol | 0.3 |
| Hydrolite ® 8 Green | Caprylyl Glycol | 0.8 |
| Sym Vital ® AR | Zingiber officinale (ginger) root extract | 0.2 |
| Tapioca Pure | Tapioca starch | 2.0 |
| Perfume oil PO1; PO2; | Perfume oil PO1; PO2; PO3; | 0.5 |
| PO3; PO4; or PO5 | PO4; or PO5 | |

TABLE F17
Whitening emulsion, expected SPF 50plus*

| Ingredients | INCI | Amount |
|---|---|---|
| Emulsiphos ® F | Potassium cetyl phosphate | 2.0 |
| | Hydrogenated palm glycerides | |
| Lanette ® 16 | Cetyl alcohol | 0.5 |
| Neo Heliopan ® 357 | Butyl methoxydibenzoylmethane | 4.9 |
| Neo Heliopan ® HMS | Homosalate | 10.0 |
| Neo Heliopan ® 303 | Octocrylene | 10.0 |
| Neo Heliopan ® OS | Ethylhexyl salicylate | 5.0 |
| Neo Heliopan ® BMT | Bis-ethylhexyloxyphenol | 4.0 |
| | methoxyphenyl triazine | |
| SymBright ™ 2036 | Sclareolide | 0.2 |
| SymWhite ® PLUS | Caprylic/capric triglyceride, Pentylene | 1.0 |
| | glycol | |
| | Phenylethyl resorcinol, Bisabolol, Butyl | |
| | methoxydibenzoylmethane | |
| Isoadipate | Diisopropyl adipate | 5.0 |
| KP-545 | Cyclopentasiloxane | 1.0 |
| | Acrylates/dimethicone copolymer | |
| Velvesil ® DM | Dimethicone | 2.0 |
| | Cetearyl dimethicone crosspolymer | |
| Prisorine ® 3505 | Isostearic acid | 1.0 |
| EDTA ® BD | Disodium edta | 0.1 |
| Hydrolite ® 5 green | Pentylene glycol | 3.0 |
| Biotive ® Resveratrol | Resveratrol | 0.1 |
| Keltrol ® CG-BT | Xanthan gum | 0.5 |
| Pemulen ® EZ-4U | Acrylates/c10-30 alkyl acrylate | 0.1 |
| | crosspolymer | |
| Aqua/Water | Aqua | Ad 100 |
| MAA-20 | | 1.0 |
| Biotive ® L-Arginine | Arginine | 0.6 |
| SymSave ® H | Hydroxyacetophenone | 0.5 |
| VITACEL ® CS 20 FC | Cellulose | 2.0 |
| SymOcide ® C | O-cymen-5-ol | 0.1 |
| Propylene Glycol | Propylene glycol | 2.0 |
| Cosmetic Color | Mica, Titanium dioxide | 3.0 |
| Pearlescent Silver AA | | |
| Perfume oil PO1; PO2; | Perfume oil PO1; PO2; PO3; PO4; or | 0.5 |
| PO3; PO4; or PO5 | PO5 | |

TABLE F18
Sunscreen Fluid for acne-prone Skin, expected SPF 30

| Ingredients | INCI | Amount |
|---|---|---|
| Dracorin ® GOC | Glyceryl oleate citrate | 2.5 |
| | Caprylic/capric triglyceride | |
| Neo Heliopan ® 357 | Butyl methoxydibenzoylmethane | 4.2 |
| Neo Heliopan ® OS | Ethylhexyl salicylate | 5.0 |
| Neo Heliopan ® BMT | Bis-ethylhexyloxyphenol | 3.5 |
| | methoxyphenyl triazine | |
| Uvasorb ® HEB | Diethylhexyl butamido triazone | 7.0 |
| SymMollient ® S | Cetearyl nonanoate | 1.0 |
| Caprylic/Capric | Caprylic/capric triglyceride | 8.0 |
| Triglyceride | | |
| EDTA ® BD | Disodium edta | 0.1 |
| SymDecanox ™ HA | Caprylic/capric triglyceride | 1.0 |
| | Hydroxymethoxyphenyl decanone | |
| Isoadipate | Diisopropyl adipate | 4.0 |
| SymClariol ® | Decylene glycol | 0.3 |
| Cetiol ® AB | C12-15 alkyl benzoate | 13.5 |
| Keltrol ® CG-SFT | Xanthan gum | 0.2 |
| Keltrol ® CG-BT | Xanthan gum | 0.1 |
| Food Color Brown | Titanium dioxides (ci 77891), Iron | 3.0 |
| E172 + E171 Powder | oxides (ci 77492), Iron oxides (ci | |
| | 77491), Iron oxides (ci. 77499) | |
| Eusolex ® T-AVO | Titanium dioxide, Silica | 2.0 |
| Aqua/Water | Aqua | Ad 100 |
| MAA-20 | | 1.0 |
| Glycerin 99.5% | Glycerin, Aqua | 3.0 |
| Lanette ® E | Sodium cetearyl sulfate | 0.8 |
| Neo Heliopan ® Hydro | Phenylbenzimidazole sulfonic acid | 2.0 |
| Biotive ® L-Arginine | Arginine | 1.0 |
| Sodium Hydroxide 10% | Aqua | 1.0 |
| solution | Sodium hydroxide | |
| SymSave ® H | Hydroxyacetophenone | 0.5 |
| Hydrolite ® 5 | Pentylene glycol | 1.5 |
| Phenoxyethanol | Phenoxyethanol | 0.5 |
| SymGlucan ® | Aqua, Glycerin, 1,2-hexanediol, | 2.0 |
| | Caprylyl glycol, Beta-glucan | |
| Sensocel ® 10 | Cellulose | 2.0 |

TABLE F19
No More Shine Fluid expected SPF50

| Ingredients | INCI | Amount |
|---|---|---|
| Dracorin ® GOC | Glyceryl oleate citrate | 2.0 |
| | Caprylic/capric triglyceride | |
| Neo Heliopan ® 357 | Butyl methoxydibenzoylmethane | 4.5 |
| Neo Heliopan ® OS | Ethylhexyl salicylate | 5.0 |
| Neo Heliopan ® HMS | Homosalate | 10.0 |
| Neo Heliopan ® 303 | Octocrylene | 8.0 |
| Neo Heliopan ® BMT | Bis-ethylhexyloxyphenol methoxyphenyl | 3.0 |
| | triazine | |
| SymMollient ® PDCC | Propanediol dicaprylate/caprate | 5.0 |
| KP-545 L | Dimethicone | 1.0 |
| | Acrylates/dimethicone copolymer | |
| EDTA ® BD | Disodium edta | 0.1 |
| Velvesil ® DM | Dimethicone | 2.5 |
| | Cetearyl dimethicone crosspolymer | |
| Silsoft ™ 034 | Caprylyl methicone | 1.0 |
| Keltrol ® CG-BT | Xanthan gum | 0.3 |
| Aristoflex ® Silk | Sodium polyacryloyldimethyl taurate | 0.5 |
| Aqua/Water | Aqua | Ad 100 |
| MAA-12 | | 2.5 |
| Neo Heliopan ® Hydro | Phenylbenzimidazole sulfonic acid | 1.0 |
| Biotive ® L-Arginine | Arginine | 1.1 |
| Lanette ® E | Sodium cetearyl sulfate | 0.8 |
| Dragosine ® | Carnosine | 0.2 |
| SymSave ® H | Hydroxyacetophenone | 0.5 |
| Hydrolite ® 5 green | Pentylene glycol | 1.0 |
| Hydrolite ® CG | Caprylyl glycol | 0.3 |
| Glycerin 99.5% | Glycerin, Aqua | 3.0 |
| Food color brown e172 + e171 | Titanium dioxides (ci 77891), Iron oxides | 2.5 |
| powder | (ci 77492), Iron oxides (ci 77491), Iron | |
| | oxides (ci. 77499) | |
| Cosmetic color titanium dioxide | Titanium dioxide | 4.0 |
| ci77891 | | |
| SymDecanox ™ HA | Caprylic/capric triglyceride | 0.5 |
| | Hydroxymethoxyphenyl decanone | |
| Sensocel ® 10 | Cellulose | 1.5 |
| Phytoconcentrole ® Carrot | Helianthus annuus seed oil, Beta- | 0.3 |
| | carotene | |
| | Daucus carota sativa root extract | |
| Perfume oil PO1; PO2; PO3; | Perfume oil PO1; PO2; PO3; PO4; or | 0.3 |
| PO4; or PO5 | PO5 | |
| Citric Acid 10% Sol. | Aqua, Citric acid | 0.2 |

TABLE F20
Light Sunscreen Fluid expected SPF 50

| Ingredients | INCI | Amount |
|---|---|---|
| Aqua/Water | Aqua | Ad 100 |
| MAA-20 | | 1.00 |
| MAA-13 | | 1.00 |
| Sodium Hydroxide | Aqua, Sodium Hydroxide | 1.00 |
| solution 10% aq. | | |
| Glycerin 99.5% | Glycerin, Aqua | 3.0 |
| Hydrolite ® 5 green | Pentylene glycol | 0.8 |
| SymSave ® H | Hydroxyacetophenone | 0.5 |
| Hydrolite ® CG | Caprylyl glycol | 0.3 |
| Neo Heliopan ® Hydro | Phenylbenzimidazole sulfonic acid | 2.0 |
| Biotive ® L-Arginine | Arginine | 1.4 |
| Emulsiphos ® F | Potassium cetyl phosphate | 1.5 |
| | Hydrogenated palm glycerides | |
| Neo Heliopan ® 303 | Octocrylene | 10.0 |
| Neo Heliopan ® 357 | Butyl methoxydibenzoylmethane | 5.0 |
| Neo Heliopan ® HMS | Homosalate | 10.0 |
| Neo Heliopan ® BMT | Bis-ethylhexyloxyphenol | 4.0 |
| | methoxyphenyl triazine | |
| Neo Heliopan ® OS | Ethylhexyl salicylate | 5.0 |
| Neo Heliopan ® E1000 | Isoamyl p-methoxycinnamate | 2.0 |
| Dragoxat ® 89 | Ethylhexyl isononanoate | 3.0 |
| Tocopheryl Acetate | Tocopheryl acetate | 0.5 |
| Keltrol ® CG-BT | Xanthan gum | 0.1 |
| Keltrol ® CG-SFT | Xanthan gum | 0.3 |
| VITACEL ® CS 20 FC | Cellulose | 2.0 |
| Tapioca Pure | Tapioca starch | 2.0 |
| Ethanol 96% | Alcohol denat. | 5.0 |
| Silsoft ™ 034 | Caprylyl methicone | 3.0 |
| Perfume oil PO1; PO2; | Perfume oil PO1; PO2; PO3; PO4; or | 0.3 |
| PO3; PO4; or PO5 | PO5 | |

TABLE F21
Refreshing Gel Sunscreen, with expected SPF 30, UVA/UVB balanced

| Raw material | INCI | Amount |
|---|---|---|
| Aqua/Water | Aqua | Ad 100 |
| MAA-13 | | 1.5 |
| Neo Heliopan ® Hydro | Phenylbenzimidazole sulfonic acid | 2.0 |
| Sodium Hydroxide solution | Aqua, Sodium Hydroxide | 0.9 |
| 10% aq. | | |
| Hydrolite ® 5 green | Pentylene glycol | 2.0 |
| Biotive ® L-Arginine | Arginine | 1.0 |
| Sodium Hydroxide 10% sol. | Aqua, Sodium hydroxide | 0.2 |
| Dragosine ® | Carnosine | 0.2 |
| SymGuard ® CD | Phenylpropanol, O-cymen-5-ol, | 0.5 |
| | Decylene glycol | |
| SymDeo ® B125 | 2-methyl 5-cyclohexylpentanol | 0.5 |
| SymSave ® H | Hydroxyacetophenone | 0.5 |
| SymSol ® PF-3 | Aqua, Pentylene glycol, Sodium lauryl | 1.5 |
| | sulfoacetate, Sodium oleoyl sarcosinate, | |
| | Sodium chlorid, Sodium oleate | |
| Neo Heliopan ® 357 | Butyl methoxydibenzoylmethane | 3.0 |
| Neo Heliopan ® OS | Ethylhexyl salicylate | 5.0 |
| Neo Heliopan ® BMT | Bis-ethylhexyloxyphenol methoxyphenyl | 1.5 |
| | triazine | |
| Neo Heliopan ® HMS | Homosalate | 2.5 |
| Neo Heliopan ® 303 | Octocrylene | 10.0 |
| EDTA ® BD | Disodium edta | 0.1 |
| SymMollient ® PDCC | Propanediol dicaprylate/caprate | 2.0 |
| Floraesters ® K-100 ® Jojoba | Hydrolyzed jojoba esters, Jojoba esters, | 1.0 |
| | Aqua | |
| Frescolat ® X-Cool | Menthyl ethylamido oxalate | 0.8 |
| Aristoflex ® AVC | Ammonium acryloyoldimethyltaurate/vp | 1.4 |
| | copolymer | |
| Keltrol ® CG-T | Xanthan gum | 0.1 |
| Sensocel ® 10 | Cellulose | 3.0 |
| Perfume oil PO1; PO2; PO3; | Perfume oil PO1; PO2; PO3; PO4; or | 0.4 |
| PO4; or PO5 | PO5 | |
| Phytoconcentrole ® Carrot | Helianthus annuus seed oil, Beta- | 0.3 |
| | carotene, | |
| | Daucus carota sativa root extract | |

TABLE F22
Sun protection balm, SPF 40

| Raw material | INCI | Amount |
|---|---|---|
| Aqua/Water | Aqua | Ad 100 |
| MAA-6 | | 2.0 |
| Neo Heliopan ® Hydro | Phenylbenzimidazole sulfonic acid | 1.0 |
| Sodium Hydroxide | Aqua, Sodium Hydroxide | 1.4 |
| solution 10% aq. | | |
| Hydrolite ®-5 | Pentylene glycol | 2.0 |
| Biotive ® L-Arginine | Arginine | 0.6 |
| Hydrolite ® CG | Caprylyl glycol | 0.25 |
| EDTA ® BD | Disodium EDTA | 0.1 |
| SymSave ® H | Hydroxyacetophenone | 0.3 |
| Hyaluronic Acid | Hyaluronic Acid | 0.1 |
| Dragosine ® | Carnosine | 0.2 |
| Aristoflex ® Silk | Sodium Polyacrydimethyl Taurate | 1.1 |
| Neo Heliopan ® 357 | Butyl methoxydibenzoylmethane | 5.0 |
| Neo Heliopan ® 303 | Octocrylene | 8.0 |
| Neo Heliopan ® OS | Ethylhexyl salicylate | 5.0 |
| Neo Heliopan ® HMS | Homosalate | 3.0 |
| Neo Heliopan ® BMT | Bis-ethylhexyloxyphenol | 3.0 |
| | methoxyphenyl triazine | |
| Dragoxat ® 89 | Ethylhexyl isononanoate | 2.0 |
| SymMollient ® PDCC | Cetearyl nonanoate | 3.0 |
| Vitacel ® CS 20 FC | Cellulose | 2.0 |

TABLE F23
Anti-Pollution Hair Care (SPF 15)

| Raw material | INCI | Amount |
|---|---|---|
| Caprylic/Capric | Caprylic/capric triglyceride | 18.0 |
| Triglyceride | Glycine soja oil, Gossypium herbaceum | 2.0 |
| SymOleo ® Vita7 | seed oil, Mangifera indica seed butter, | |
| | Olea europaea fruit oil, Persea | |
| | gratissima oil, Prunus amygdalus dulcis | |
| | (sweet almond) oil, Theobroma cacao | |
| | seed butter | |
| SymMollient ® PDCC | Propanediol dicaprylate/caprate | 5.0 |
| Neo Heliopan ® BMT | Bis-ethylhexyloxyphenol | 2.0 |
| | methoxyphenyl triazine | |
| Neo Heliopan ® 303 | Octocrylene | 5.0 |
| Neo Heliopan ® OS | Ethylhexyl salicylate | 5.0 |
| Cetiol ® CC | Dicaprylyl carbonate | 5.0 |
| Sweet Almond Oil | Prunus amygdalus dulcis (sweet | 15.0 |
| | almond) oil | |
| Cotton Seed Oil | Gossypium herbaceum seed oil | 15.0 |
| Grape Seed Oil | Vitis vinifera seed oil | 12.0 |
| (Refined) | | |
| Xiameter ® PMX-345 | Cyclopentasiloxane, Cyclohexasiloxane | 10.0 |
| SymHair ® Shape & | Cetearyl nonanoate, Triticum vulgare | 1.0 |
| Color | germ oil, Caprylic/capric triglyceride, | |
| | Linoleic acid, Triticum vulgare bran | |
| | extract, Triticum vulgare germ extract | |
| | Camellia oleifera seed oil | |
| Sym Decanox ™ HA | Caprylic/capric triglyceride, | 2.0 |
| | Hydroxymethoxyphenyl decanone | |
| MAA-13 | | 0.05 |
| Hydrolite ® 5 | Pentylene Glycole | 2.5 |
| Perfume oil PO1; PO2; | Perfume oil PO1; PO2; PO3; PO4; or | 2.4 |
| PO3; PO4; or PO5 | PO5 | |

TABLE F24
Perfect Hand emulsion with expected SPF 15, UVA/UVB balanced

| Raw material | INCI | Amount |
|---|---|---|
| Emulsiphos ® F | Potassium cetyl phosphate | 1.5 |
| | Hydrogenated palm glycerides | |
| Lanette ® O | Cetearyl alcohol | 1.0 |
| SymWhite ® 377 | Phenylethyl Resorcinol | 0.4 |
| Tegosoft ® MM | Myristyl myristate | 0.5 |
| Neo Heliopan ® BMT | Bis-ethylhexyloxyphenol methoxyphenyl | 2.0 |
| | triazine | |
| Neo Heliopan ® OS | Ethylhexyl salicylate | 5.0 |
| Edeta ® BD | Disodium EDTA | 0.1 |
| Dragoxat ® 89 | Ethylhexyl isononanoate | 2.0 |
| Isoadipate | Diisopropyl adipate | 6.0 |
| PCL-Liquid ® 100 | Cetearyl ethylhexanoate | 3.0 |
| Corapan ® TQ | Diethylhexyl 2,6-naphthalate | 2.0 |
| Cetiol ® SB 45 | Butyrospermum parkii butter | 1.5 |
| SymBright ™ 2036 | Sclareolide | 0.2 |
| SymDecanox ™ HA | Caprylic/capric triglyceride, | 1.0 |
| | Hydroxymethoxyphenyl decanone | |
| SymCare ® O | Hexyldecanol, Pentylene glycol, 4-t- | 2.0 |
| | butylcyclohexanol, Bisabolol, | |
| | Cetylhydroxyproline palmitamide, | |
| | Hydroxyphenyl propamidobenzoic acid, | |
| | Stearic acid, Brassica campestris | |
| | (rapeseed) sterols, Zingiber officinale | |
| | (ginger) root extract | |
| SymDetox ® | Methylcyclohexyl Succinate | 0.5 |
| | Dimethylamide | |
| Genuvisco ® Carrageenan | Chondrus crispus powder | 0.4 |
| CG-131 | | |
| Keltrol ® CG-SFT | Xanthan gum | 0.2 |
| Aqua/Water | Aqua | Ad 100 |
| MAA-1 | | 2.00 |
| MAA-13 | | 1.00 |
| Neo Heliopan ® Hydro | Phenylbenzimidazole sulfonic acid | 2.5 |
| Biotive ® L-Arginine | Arginine | 1.0 |
| Sodium Hydroxide 10% | Aqua | 2.6 |
| solution | Sodium hydroxide | |
| Glycerin | Glycerin | 2.0 |
| SymSave ® H | Hydroxyacetophenone | 0.5 |
| SymDiol ® 68 | 1,2-hexanediol, Caprylyl glycol | 0.5 |
| RonaFlair ® Flawless | Silica, Ci 77892, Ci 77491 | 1.5 |
| Extrapone ® Moroccan Rose | Aqua, Glycerin, Peg-40 hydrogenated | 1.0 |
| GW | castor oil, Rosa damascena flower | |
| | extract | |
| Tego ® Feel C10 | Cellulose | 1.5 |
| Perfume oil PO1; PO2; PO3; | Perfume oil PO1; PO2; PO3; PO4; or | 0.5 |
| PO4; or PO5 | PO5 | |

TABLE F25
Tinted Sunscreen cream against hyperpigmentation

| Raw material | INCI | Amount |
|---|---|---|
| Emulsiphos ® F | Potassium cetyl phosphate | 2.0 |
| | Hydrogenated palm glycerides | |
| Neo Heliopan ® 357 | Butyl methoxydibenzoylmethane | 4.2 |
| Neo Heliopan ® OS | Ethylhexyl salicylate | 5.0 |
| Neo Heliopan ® BMT | Bis-ethylhexyloxyphenol methoxyphenyl | 3.5 |
| | triazine | |
| Uvasorb ® HEB | Diethylhexyl butamido triazone | 7.0 |
| SymMollient ® S | Cetearyl nonanoate | 1.0 |
| Isoadipate | Diisopropyl adipate | 4.0 |
| SymBright ™ 2036 | Sclareolide | 0.2 |
| SymSitive ® 1609 | Pentylene glycol, 4-t-butylcyclohexanol | 1.0 |
| Caprylic/Capric Triglyceride | Caprylic/capric triglyceride | 8.0 |
| Sym Decanox ™ HA | Caprylic/capric triglyceride | 1.0 |
| | Hydroxymethoxyphenyl decanone | |
| Cetiol ® AB | C12-15 alkyl benzoate | 13.5 |
| EDTA ® BD | Disodium edta | 0.1 |
| Keltrol ® CG-BT | Xanthan gum | 0.3 |
| Genuvisco ® Carrageenan | Chondrus crispus powder | 0.2 |
| CG-131 | | |
| Food Color Brown | Titanium dioxides (ci 77891), Iron oxides | 2.0 |
| E172 + E171 | (ci 77492), Iron oxides (ci 77491), Iron | |
| Powder | oxides (ci. 77499) | |
| Eusolex ® T-AVO | Titanium dioxide, Silica | 2.0 |
| Aqua/Water | Aqua | 31.4 |
| MAA-1 | | 1.0 |
| Neo Heliopan ® Hydro | Phenylbenzimidazole sulfonic acid | 2.0 |
| Biotive ® L-Arginine | Arginine | 1.0 |
| Sodium Hydroxide 10% solution | Aqua, Sodium hydroxide | 1.2 |
| Dragosine ® | Carnosine | 0.2 |
| Glycerin 99.5 % | Glycerin, Aqua | 3.0 |
| SymSave ® H | Hydroxyacetophenone | 0.5 |
| Hydrolite ® 5 green | Pentylene glycol | 1.5 |
| Phenoxyethanol | Phenoxyethanol | 0.5 |
| Lanette ® E | Sodium cetearyl sulfate | 0.8 |
| Perfume oil PO1; PO2; PO3; | Perfume oil PO1; PO2; PO3; PO4; or | 0.5 |
| PO4; or PO5 | PO5 | |
| SymOat ® PLUS | Aqua, Glycerin, Maltodextrin, 1,2- | 2.0 |
| | hexanediol, | |
| | Beta-glucan, Caprylyl glycol | |
| Sensocel ® 10 | Cellulose | 1.0 |

Table F26: BB Cream Dark Tone SPF-20 (expected)

| Raw material | INCI | Amount |
|---|---|---|
| Emulsiphos^{®} F | Potassium cetyl phosphate | 2.5 |
| | Hydrogenated palm glycerides | |
| Eumulgin^{®} SG | Sodium stearoyl glutamate | 0.5 |
| PCL-Liquid^{®} 100 | Cetearyl ethylhexanoate | 2.0 |
| Lanette^{®} O | Cetearyl alcohol | 1.0 |
| Dragoxat^{®} 89 | Ethylhexyl isononanoate | 1.0 |
| Cutina^{®} PES | Pentaerythrityl distearate | 1.0 |
| SymMollient^{®} S | Cetearyl nonanoate | 1.0 |
| Xiameter^{®} PMX-0245 | Cyclopentasiloxane | 2.0 |
| Cyclopentasiloxane | | |
| Xiameter^{®} PMX-200 | Dimethicone | 1.0 |
| Silicone Fluid 50 cs | | |
| Neo Heliopan^{®} 357 | Butyl methoxydibenzoylmethane | 3.0 |
| Neo Heliopan^{®} 303 | Octocrylene | 7.0 |
| Neo Heliopan^{®} HMS | Homosalate | 7.0 |
| Neo Heliopan^{®} BMT | Bis-ethylhexyloxyphenol | 1.0 |
| | methoxyphenyl triazine | |
| Neo Heliopan^{®} OS | Ethylhexyl salicylate | 5.0 |
| Sym Decanox ™ HA | Caprylic/capric triglyceride | 2.0 |
| | Hydroxymethoxyphenyl decanone | |
| Talcum | Talc | 2.0 |
| Food Color Titanium | Ci 77891 | 0.8 |
| Dioxide Powder E171 | | |
| Keltrol^{®} CG-SFT | Xanthan gum | 0.4 |
| Carbopol^{®} Ultrez 20 | Acrylates/c10-30 alkyl acrylate | 0.3 |
| Polymer | crosspolymer | |
| EDTA^{®} BD | Disodium EDTA | 0.2 |
| Aqua/Water | Aqua | Ad 100 |
| MAA-20 | | 0.5 |
| MAA-13 | | 2.0 |
| Neo Heliopan^{®} Hydro | Phenylbenzimidazole sulfonic acid | 1.0 |
| Biotive® L-Arginine | Arginine | 1.0 |
| Sodium Hydroxide 10% | Aqua | 1.5 |
| solution | Sodium hydroxide | |
| COSMETIC COLOR | Ci 77891, Ci 77492, Ci 77491, | 1.5 |
| BROWN POWDER | Ci 77499 | |
| Food Color Iron Oxide | Ci 77492 | 0.3 |
| Yellow Glycerin | Glycerin | 3.0 |
| Hydrolite^{®} 5 Green | Pentylene glycol | 1.5 |
| Hydrolite^{®} CG | Caprylyl glycol | 0.2 |
| SymSave^{®} H | Hydroxyacetophenone | 0.5 |
| Hydroviton^{®} PLUS 2290 | Aqua, Pentylene glycol, Glycerin, | 2.0 |
| | Fructose, Urea, Citric acid, Sodium | |
| | hydroxide, Maltose, Sodium pca, | |
| | Sodium chloride, Sodium lactate, | |
| | Trehalose, Allantoin, Sodium | |
| | hyaluronate, Glucose | |
| Perfume oil PO1; PO2; PO3; | Perfume oil PO1; PO2; PO3; PO4; | 0.5 |
| PO4; or PO5 | or PO5 | |

Table F27: Daily Well Aging, with SPF 30, UVA/UVB balanced, w/o Octocrylene

| Raw material | INCI | Amount |
|---|---|---|
| Lanette^{®} O | Cetearyl alcohol | 1.5 |
| Emulsiphos^{®} F | Potassium cetyl phosphate | 2.5 |
| | Hydrogenated palm glycerides | |
| SymUrban^{®} | Benzylidene | 0.3 |
| | dimethoxydimethylindanone | |
| Neo Heliopan^{®} 357 | Butyl | 4.0 |
| | methoxydibenzoylmethane | |
| Neo Heliopan^{®} OS | Ethylhexyl salicylate | 5.0 |
| Neo Heliopan^{®} BMT | Bis-ethylhexyloxyphenol | 3.0 |
| | methoxyphenyl triazine | |
| Uvasorb^{®} HEB | Diethylhexyl butamido triazone | 2.0 |
| SymMollient^{®} S | Cetearyl nonanoate | 1.0 |
| Cetiol^{®} Sensoft | Propylheptyl caprylate | 4.0 |
| Tegosoft^{®} TN | C12-15 alkyl benzoate | 11.0 |
| SymDecanox ™ HA | Caprylic/capric triglyceride | 1.0 |
| | Hydroxymethoxyphenyl | |
| | decanone | |
| Isoadipate | Diisopropyl adipate | 7.5 |
| EDTA^{®} BD | Disodium edta | 0.1 |
| Carbopol^{®} ETD 2050 Polymer | Carbomer | 0.3 |
| Keltrol^{®} CG-T | Xanthan gum | 0.2 |
| Aqua/Water | Aqua | Ad 100 |
| MAA-13 | | 3.0 |
| Hydrolite^{®} 5 Green | Pentylene glycol | 2.0 |
| Glycerin 99.5 % | Glycerin, Aqua | 1.0 |
| Lanette^{®} E | Sodium cetearyl sulfate | 0.8 |
| Neo Heliopan^{®} Hydro | Phenylbenzimidazole | 3.0 |
| | sulfonic acid | |
| Biotive^{®} L-Arginine | Arginine | 1.5 |
| Propylene Glycol | Propylene glycol | 3.0 |
| Sodium Hydroxide 10% | Aqua, Sodium hydroxide | 3.1 |
| solution | | |
| SymSave^{®} H | Hydroxyacetophenone | 0.6 |
| Phenoxyethanol | Phenoxyethanol | 0.5 |
| Dragosine^{®} | Carnosine | 0.2 |
| SymVital^{®} AR | Zingiber officinale (ginger) | 0.2 |
| | root extract | |
| Frescolat^{®} ML Cryst | Menthyl lactate | 0.5 |
| Perfume oil PO1; PO2; PO3; | Perfume oil PO1; PO2; PO3; | 1.0 |
| PO4; or PO5 | PO4; or PO5 | |
| Tapioca Pure | Tapioca starch | 2.0 |

Table F28: Anti-Wrinkle Day Emulsion expected SPF 15

| Raw material | INCI | Amount |
|---|---|---|
| Emulsiphos® F | Potassium cetyl phosphate, | 2.5 |
| | Hydrogenated palm glycerides | |
| Neo Heliopan^{®} BMT | Bis-ethylhexyloxyphenol | 2.0 |
| | methoxyphenyl triazine | |
| Neo Heliopan^{®} 303 | Octocrylene | 2.0 |
| Neo Heliopan^{®} OS | Ethylhexyl salicylate | 5.0 |
| Isoadipate | Diisopropyl adipate | 2.0 |
| SymMollient^{®} S | Cetearyl nonanoate | 2.0 |
| Caprylic/Capric | Caprylic/capric triglyceride | 3.0 |
| Triglyceride | | |
| Dragoxat^{®} 89 | Ethylhexyl isononanoate | 1.0 |
| PCL-Liquid^{®} 100 | Cetearyl ethylhexanoate | 3.0 |
| SymUrban^{®} | Benzylidene | 0.3 |
| | dimethoxydimethylindanone | |
| Sym Decanox™ HA | Caprylic/capric triglyceride | 2.0 |
| | Hydroxymethoxyphenyl | |
| | decanone | |
| Edeta^{®} BD | Disodium edta | 0.1 |
| Keltrol^{®} CG-SFT | Xanthan gum | 0.2 |
| Cosmedia^{®} SP | Sodium Polyacrylate | 0.4 |
| Aqua/Water | Aqua | Ad 100 |
| Hydrolite^{®} 5 Green | Pentylene glycol | 3.0 |
| Glycerin | Glycerin | 3.0 |
| MAA-28 | | 0.7 |
| Neo Heliopan^{®} Hydro | Phenylbenzimidazole | 2.0 |
| | sulfonic acid | |
| Neo Heliopan^{®} AP | Disodium Phenyl | 1.0 |
| | Dibenzimidazole Tetrasulfonate | |
| Biotive^{®} L-Arginine | Arginine | 1.0 |
| Sodium Hydroxide 10% | Aqua, Sodium hydroxide | 1.5 |
| solution | | |
| Dragosine^{®} | Carnosine | 0.2 |
| SymSave^{®} H | Hydroxyacetophenone | 0.5 |
| SymDiol^{®} 68 | 1,2-hexanediol, Caprylyl glycol | 0.3 |
| Phenoxyethanol | Phenoxyethanol | 0.2 |
| Hydroviton^{®} PLUS 2290 | Aqua, Pentylene glycol, Glycerin, | 3.0 |
| | Fructose, Urea, Citric acid, Sodium | |
| | hydroxide, Maltose, Sodium pca | |
| | Sodium chloride, Sodium lactate, | |
| | Trehalose, Allantoin, Sodium | |
| | hyaluronate, Glucose | |
| SymLift^{®} | Aqua, Trehalose, Glycerin, | 5.0 |
| | Pentylene glycol, Beta-glucan, | |
| | Hordeum vulgare seed | |
| | extract, Sodium hyaluronate, 1,2- | |
| | hexanediol, Caprylyl glycol | |
| Macadamia Oil (refined) | Macadamia integrifolia seed oil | 0.2 |
| SymVital^{®} AR | Zingiber officinale (ginger) | 0.2 |
| | root extract | |
| Actipone^{®} Laminaria | Glycerin, Aqua, | 1.0 |
| | Laminaria saccharina | |
| Saccharina GW | extract | |
| Ethanol 96 % | Alcohol denat. | 3.0 |
| Sensocel^{®} 10 | Cellulose | 1.0 |
| Sipernat^{®} 11 PC | Hydrated silica | 1.0 |
| Perfume oil PO1; PO2; | Perfume oil PO1; PO2; PO3; | 0.4 |
| PO3; PO4; or PO5 | PO4; or PO5 | |

Table F29: Daily Tattoo Care with expected SPF 10, UVA/UVB balanced

| Raw material | INCI | Amount |
|---|---|---|
| Aqua/Water | Aqua | Ad 100 |
| Hydrolite^{®} 5 Green | Pentylene glycol | 2,0 |
| Neo Heliopan^{®} Hydro | Phenylbenzimidazole sulfonic acid | 2.5 |
| Biotive^{®} L-Arginine | Arginine | 1.0 |
| Sodium Hydroxide 10% | Aqua, Sodium hydroxide | 3.5 |
| solution | | |
| MAA-1 | | 3.00 |
| SymSave^{®} H | Hydroxyacetophenone | 0.5 |
| SymDiol^{®} 68 | 1,2-hexanediol, Caprylyl glycol | 0.5 |
| Edeta^{®} BD | Disodium EDTA | 0.1 |
| Emulsiphos^{®} F | Potassium cetyl phosphate | 2.5 |
| | Hydrogenated palm glycerides | |
| Cetiol^{®} SB 45 | Butyrospermum parkii butter | 2.0 |
| Lanette^{®} 16 | Cetyl alcohol | 2.0 |
| Lanette^{®} O | Cetearyl alcohol | 3.5 |
| SymMollient^{®} S | Cetearyl nonanoate | 1.0 |
| Caprylic/Capric | Caprylic/capric triglyceride | 6.0 |
| Triglyceride | | |
| Corapan^{®} TQ | Diethylhexyl 2,6-naphthalate | 1.0 |
| Dragoxat^{®} 89 | Ethylhexyl isononanoate | 2.0 |
| Isoadipate | Diisopropyl adipate | 3.0 |
| Neo Heliopan^{®} BMT | Bis-ethylhexyloxyphenol | 2.0 |
| | methoxyphenyl triazine | |
| Neo Heliopan^{®} OS | Ethylhexyl salicylate | 5.0 |
| SymCare^{®} | Hexyldecanol, Pentylene glycol, | 4.0 |
| | 4-t-butylcyclohexanol, Bisabolol, | |
| | Cetylhydroxyproline palmitamide, | |
| | Hydroxyphenyl | |
| | propamidobenzoic acid, | |
| | Stearic acid, Brassica campestris | |
| | (rapeseed) sterols, | |
| | Zingiber officinale | |
| | (ginger) root extract | |
| SymDecanox™ HA | Caprylic/capric triglyceride | 1.0 |
| | Hydroxymethoxyphenyl decanone | |
| Cetiol^{®} Ultimate | Tridecane, Undecane | 4.0 |
| Carbopol^{®} Ultrez 21 | Acrylates/c10-30 alkyl acrylate | 0.4 |
| Polymer | crosspolymer | |
| Keltrol^{®} CG-T | Xanthan gum | 0.2 |
| SymGlucan^{®} | Aqua, Glycerin, | 2.0 |
| | 1,2-hexanediol, Caprylyl | |
| | glycol, Beta-glucan | |
| Perfume oil PO1; PO2; | Perfume oil PO1; PO2; | 0.5 |
| PO3; PO4; or PO5 | PO3; PO4; or PO5 | |
| Tapioca Pure | Tapioca starch | 2.0 |

Table F30: Sensitive Day Care Cream expected SPF 15

| Raw material | INCI | Amount |
|---|---|---|
| Lanette^{®} O | Cetearyl alcohol | 4.0 |
| Emulsiphos^{®} F | Potassium cetyl phosphate, | 2.0 |
| | Hydrogenated palm glycerides | |
| Cetiol^{®} AB | C12-15 alkyl benzoate | 1.0 |
| Cetiol^{®} OE | Dicaprylyl ether | 1.0 |
| SymMollient® S | Cetearyl nonanoate | 1.0 |
| Neo Heliopan® BMT | Bis-ethylhexyloxyphenol | 2.0 |
| | methoxyphenyl triazine | |
| Neo Heliopan^{®} 303 | Octocrylene | 3.0 |
| Grape Seed Oil (Refined) | Vitis vinifera seed oil | 1.0 |
| Corapan^{®} TQ | Diethylhexyl 2,6-naphthalate | 3.0 |
| Eumulgin^{®} SG | Sodium stearoyl glutamate | 1.0 |
| SymCare^{®} O | Hexyldecanol, Pentylene glycol, | 3.0 |
| | 4-t-butylcyclohexanol, Bisabolol, | |
| | Cetylhydroxyproline palmitamide, | |
| | Hydroxyphenyl | |
| | propamidobenzoic acid, | |
| | Stearic acid, Brassica campestris | |
| | (rapeseed) sterols, | |
| | Zingiber officinale | |
| | (ginger) root extract | |
| Softisan^{®} 100 | Hydrogenated coco-glycerides | 1.0 |
| Eutanol^{®} G | Octyldodecanol | 2.0 |
| Caprylic/Capric | Caprylic/capric triglyceride | 5.0 |
| Triglyceride | | |
| Carbopol^{®} Ultrez | Carbomer | 0.3 |
| 10 Polymer | | |
| Aqua/Water | Aqua | Ad 100 |
| Hydrolite^{®} 5 Green | Pentylene glycol | 1.0 |
| Hydrolite^{®} 7 Green | 1,2-heptanediol | 0.5 |
| MAA-8 | | 1.0 |
| MAA-1 | | 1.0 |
| Neo Heliopan^{®} Hydro | Phenylbenzimidazole sulfonic acid | 3.0 |
| Biotive^{®} L-Arginine | Arginine | 1.0 |
| Sodium Hydroxide | Aqua, Sodium hydroxide | 2.8 |
| 10% solution | | |
| Edeta^{®} BD | Disodium EDTA | 0.1 |
| Glycerin | Glycerin | 5.0 |
| SymSave^{®} H | Hydroxyacetophenone | 0.5 |
| Phenoxyethanol | Phenoxyethanol | 0.5 |
| Hydroviton^{®} | Aqua, Pentylene glycol, Glycerin, | 3.0 |
| PLUS 2290 | Fructose, Urea, Citric acid, Sodium | |
| | hydroxide, Maltose, Sodium pca, | |
| | Sodium chloride, Sodium lactate, | |
| | Trehalose, Allantoin, Sodium | |
| | hyaluronate, Glucose | |
| D-Panthenol 75 W | Panthenol, Aqua | 2.0 |
| Tapioca Pure | Tapioca starch | 4.0 |
| Actipone® Licorice | Aqua, Butylene glycol, | 2.0 |
| | Glycyrrhiza glabra root extract | |
| Sodium Hydroxide | Aqua, Sodium hydroxide | 1.5 |
| 10% solution | | |

Table F31: Cream with expected SPF 15, UVA/UVB

| Raw material | INCI | Amount |
|---|---|---|
| Aqua/Water | Aqua | Ad 100 |
| MAA-13 | | 1.0 |
| MAA-1 | | 1.5 |
| SymSol^{®} PF-3 | Aqua, Pentylene glycol, | 2.0 |
| | Sodium lauryl sulfoacetate, | |
| | Sodium oleoyl sarcosinate, | |
| | Sodium chloride, Sodium oleate | |
| Neo Heliopan^{®} Hydro | Phenylbenzimidazole sulfonic acid | 2.5 |
| Biotive^{®} L-Arginine | Arginine | 1.0 |
| Sodium Hydroxide 10% | Aqua, Sodium hydroxide | 2.1 |
| solution | | |
| Aqua Keep 10SH-NFC | Sodium acrylates crosspolymer-2 | 2.0 |
| SymSave^{®} H | Hydroxyacetophenone | 0.5 |
| Dragosine^{®} | Carnosine | 0.1 |
| Hydrolite^{®} 5 Green | Pentylene glycol | 2.5 |
| Neo Heliopan^{®} OS | Ethylhexyl salicylate | 3.0 |
| Neo Heliopan^{®} 303 | Octocrylene | 3.0 |
| Neo Heliopan^{®} BMT | Bis-ethylhexyloxyphenol | 1.5 |
| | methoxyphenyl triazine | |
| Neo Heliopan^{®} HMS | Homosalate | 3.0 |
| SymMollient^{®} S | Cetearyl nonanoate | 1.0 |
| Isoadipate | Diisopropyl adipate | 2.5 |
| SymRelief^{®} 100 | Bisabolol | 0.2 |
| | Zingiber officinale root extract | |
| Sym Decanox ™ HA | Caprylic/capric triglyceride | 1.5 |
| | Hydroxymethoxyphenyl decanone | |
| SymDetox^{®} | 3,3,5-trimethylcyclohexyl | 0.5 |
| | succinate dimethylamide | |
| SymUrban^{®} | Benzylidene | 0.5 |
| | dimethoxydimethylindanone | |
| EDETA^{®} BD | Disodium edta | 0.1 |
| Frescolat^{®} X-Cool | Menthyl ethylamido oxalate | 0.5 |
| SymBright™ 2036 | Sclareolide | 0.1 |
| SymWhite^{®} PLUS | Caprylic/capric triglyceride, | 1.3 |
| | Pentylene glycol | |
| | Phenylethyl resorcinol, Bisabolol, | |
| | Butyl methoxydibenzoylmethane | |
| Keltrol^{®} CG-BT | Xanthan gum | 0.2 |
| FOOD COLOR BROWN | Titanium dioxides (ci 77891), Iron | 2.0 |
| E172+E171 POWDER | oxides (ci 77492), Iron oxides (ci | |
| | 77491), Iron oxides (ci. 77499) | |
| Perfume oil PO1; PO2; | Perfume oil PO1; PO2; PO3; | 0.5 |
| PO3; PO4; or PO5 | PO4; or PO5 | |
| SymGlucan^{®} | Aqua, Glycerin, 1,2-hexanediol, | 5.0 |
| | Caprylyl glycol, Beta-glucan | |

Table F32: All-purpose cleaner

| No | Ingredients | Amount |
|---|---|---|
| 1 | Fettalkoholethoxylat | 4 |
| 2 | Sulfonic acid, C 13-17-sec-Alkyl-, Sodium salt | 6.2 |
| 3 | 2-octyl-2H-isothiazol-3-one | 0.1 |
| 4 | Fragrance | 3.5 |
| 5 | MAA-13 | 1.0 |
| 6 | 1,2-heptanediol | 0.9 |
| 7 | Aqua | add 100 |

Colorless liquid, pH 7

Table F33: APC alkaline

| No | Ingredients | Amount |
|---|---|---|
| 1 | Coco-Glycoside | 1 |
| 2 | Benzalkonium Chloride | 0.2 |
| 3 | Sodiumbicarbonat | 0.1 |
| 4 | Tri-sodiumcitrate-dihydrate | 0.1 |
| 5 | Methylglycinediacetic acid | 0.1 |
| 6 | 1,2-Benzisothiazol-3(2H)-on | 0.1 |
| 7 | Fragrance | 0.1 |
| 8 | 1,2-heptanediol | 0.5 |
| 9 | MAA-2 | 0.5 |
| 10 | Hydroxyacetophenone | 0.3 |
| 11 | Aqua | add 100 |

Colorless liquid, pH 9

Table F34: Detergent liquid light duty

| No | Ingredients | Amount |
|---|---|---|
| 1 | Cocos fatty acid | 0.85 |
| 2 | potassium hydroxide | 0.45 |
| 3 | 2-octyl-2H-isothiazol-3-one | 0.2 |
| 4 | Sulfonic acid, C 13-17-sec-Alkyl-, Sodium salt | 20 |
| 5 | Sodium Laureth Sulfate | 3 |
| 6 | Trideceth-9 | 5 |
| 7 | Sodium Chloride | 1.3 |
| 8 | MAA-7 | 0.1 |
| 9 | 1,2-heptanediol | 0.9 |
| 10 | Fragrance | 0.5 |
| 11 | Aqua | add 100 |
| 12 | Hydroxyacetophenone | 0.2 |

Liquid, pH 7.3

Table F35: Dishwash liquid manual

| No | Ingredients | Amount |
|---|---|---|
| 1 | Sodium Laureth Sulfate | add 100 |
| 2 | Ethanol | 2.9 |
| 3 | lauramine oxide | 7.7 |
| 4 | propylene glycol | 1.9 |
| 5 | Phenoxyethanol | 0.1 |
| 6 | Benzisothiazolinone, Methylisothiazolinone, | 0.1 |
| | Laurylamine Dipropylenediamine | |
| 7 | 1,2-heptanediol | 0.5 |
| 8 | sodium chloride | 3.85 |
| 9 | Fragrance | 0.3 |
| 10 | Benzyl Alcohol | 0.2 |
| 11 | MAA-1 | 0.5 |
| 12 | MAA-13 | 0.5 |

Colorless liquid, pH 8.5

Table F36: Fabric softener

| No | Ingredients | Amount |
|---|---|---|
| 1 | Di-(Talg Carboxyethyl) Hydroxyethyl | 5.5 |
| | methylammonium- methosulfat | |
| 2 | Simethicone | 0.3 |
| 3 | 2-octyl-2H-isothiazol-3-one | 0.1 |
| 4 | 1,2-heptanediol | 0.5 |
| 5 | Fragrance | 0.2 |
| 6 | Aqua | add 100 |
| 7 | Phenoxyethanol | 0.2 |
| 8 | MAA-20 | 1.5 |

Liquid, pH 3

Table F37: Fabric softener concentrate, encapsulated

| No | Ingredients | Amount |
|---|---|---|
| 1 | Di-(Talg Carboxyethyl) Hydroxyethyl | 16.6 |
| | methylammonium-methosulfat | |
| 2 | 2-octyl-2H-isothiazol-3-one | 0.1 |
| 3 | Simethicone | 0.3 |
| 4 | magnesium chloride | 0.8 |
| 5 | encapsulated perfume oil | 0.3 |
| 6 | crosslinked cationic polymer | 0.15 |
| 7 | MAA-28 | 1.0 |
| 8 | 1,2-heptanediol | 0.5 |
| 9 | Fragrance | 0.6 |
| 10 | Aqua | add 100 |
| 11 | Glyceryl Caprylate | 0.1 |

White liquid, pH 2.5

Table F38: Hand soap, liquid

| No | Ingredients | Amount |
|---|---|---|
| 1 | Sodium Laureth Sulfate | 18 |
| 2 | Cocoamidopropylbetaine | 6 |
| 3 | Cocamide DEA | 3 |
| 4 | citric acid | 0.5 |
| 5 | sodium chloride | 1.9 |
| 6 | Glycerol | 2 |
| 7 | Fragrance | 0.2 |
| 8 | MAA-13 | 0.25 |
| 9 | Aqua | add 100 |
| 10 | Propanediol Caprylate | 0.3 |

Slightly colored liquid, pH 6

Table F39: Scent Lotion with capsules

| No | Ingredients | Amount |
|---|---|---|
| 1 | microcrystalline cellulose/cellulose gum | 1 |
| 2 | PEG-40 hydrogenated Castor oil/ Trideceth-9 | 0.5 |
| 3 | encapsulated perfume oil | 1 |
| 4 | 2-octyl-2H-isothiazol-3-one | 0.1 |
| 5 | Fragrance | 5 |
| 6 | 1,2-heptanediol | 1 |
| 7 | Aqua | add 100 |
| 8 | MAA-20 | 0.4 |

White emulsion, PH neutral

Table F40: Cleaner, liquid, citric acid

| No | Ingredients | Amount |
|---|---|---|
| 1 | Sulfonic acid, C 13-17-sec-Alkyl-, Sodium salt | 1 |
| 2 | Xanthan gum | 0-3 |
| 3 | citric acid | 5 |
| 4 | 1,2 heptanediol | 0.25 |
| 5 | Fragrance | 0.3 |
| 6 | Aqua | add 100 |
| 7 | Anisic Acid | 0.1 |
| 8 | MAA-13 | 0.5 |

Liquid, pH 2

1. A sunscreen product, cosmetic or pharmaceutical preparation or homecare product, comprising
(a) at least one photo-unstable UV-filter; and
(b) at least one mycosporine-like amino acid compound,
represented either by the general formula (V) 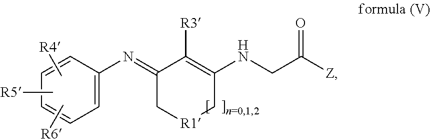
wherein
Z is selected from the group consisting of O—R2′ or amide′;
R1′ is selected from the group consisting of CH₂, unsubstituted or substituted CH(alkyl), unsubstituted or substituted C(alkyl)₂, unsubstituted or substituted CH(cycloalkyl), unsubstituted or substituted CH(aryl), unsubstituted or substituted CH(heterocycloalkyl), unsubstituted or substituted CH(heteroaryl), CR7′R8′, C═O, halogen, O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), and unsubstituted or substituted N(heteroaryl), and unsubstituted or substituted C-spirocycles;
R2′ is selected from the group consisting of H, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted cycloalkyl, unsubstituted or substituted aryl, unsubstituted or substituted heterocycloalkyl, and unsubstituted or substituted heteroaryl;
amide′ is selected from the group consisting of NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), an unsubstituted or substituted NR9′R10′, unsubstituted or substituted —N(O-alkyl)(alkl) (Weinreb amide), —N(OH)(H) (hydroxamate), and unsubstituted or substituted —N(OH)(alkyl) (alkylated hydroxamate);
R3′ is selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9′R10′, and unsubstituted or substituted C-spirocycles;
the substituents R4′, R5′ and R6′ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, —S(═O)₂OH, sulfonyl, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9′R10′, and unsubstituted or substituted C-spirocycles;
R7′ and R8′ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl) and unsubstituted or substituted NR9′R10′;
R9′ und R10′ which may be identical or different, are independently from each other selected from the group consisting of H, OH, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, unsubstituted or substituted cycloalkyl, unsubstituted or substituted aryl, unsubstituted or substituted heterocycloalkyl, and unsubstituted or substituted heteroaryl;
Y is selected from the group consisting of H and unsubstituted or substituted alkyl; and
R is unsubstituted or substituted alkyl;
or a tautomer or a stereoisomer or a salt of the afore-mentioned compounds;
or represented by the general formula (VI) 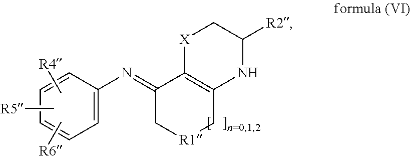
wherein
R1″ is selected from the group consisting of CH₂, unsubstituted or substituted CH(alkyl), unsubstituted or substituted C(alkyl)₂, unsubstituted or substituted CH(cycloalkyl), unsubstituted or substituted CH(aryl), unsubstituted or substituted CH(heterocycloalkyl), unsubstituted or substituted CH(heteroaryl), —CR7″R8″, C═O, halogen, O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), unsubstituted or substituted N(heteroaryl), and unsubstituted or substituted C-spirocycles;
R2″ is selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —C(═O)-amide″, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9″R10″, and unsubstituted or substituted C-spirocycles;
X is selected from the group consisting of CH₂, unsubstituted or substituted CH(alkyl), unsubstituted or substituted CH(alkenyl), unsubstituted or substituted CH(alkynyl), unsubstituted or substituted CH(alkoxy), unsubstituted or substituted CH(alkylthio), unsubstituted or substituted CH(cycloalkyl), unsubstituted or substituted CH(aryl), unsubstituted or substituted CH(heterocycloalkyl), unsubstituted or substituted CH(heteroaryl), —CR7″R8″, C═O, O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), unsubstituted or substituted N(heteroaryl), and unsubstituted or substituted C-spirocycles;
the substituents R4″, R5″ and R6″ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, —S(═O)₂OH, sulfonyl, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9″R10″, and unsubstituted or substituted C-spirocycles;
R7″ and R8″ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl) and unsubstituted or substituted NR9″R10″;
amide″ is selected from the group consisting of NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9″R10″, unsubstituted or substituted —N(O-alkyl)(alkyl) (Weinreb amide), —N(OH)(H) (hydroxamate), and unsubstituted or substituted —N(OH)(alkyl) (alkylated hydroxamate);
R9″ und R10″ which may be identical or different, are independently from each other selected from the group consisting of H, OH, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, unsubstituted or substituted cycloalkyl, unsubstituted or substituted aryl, unsubstituted or substituted heterocylcoalkyl, and unsubstituted or substituted heteroaryl;
Y is selected from the group consisting of H and unsubstituted or substituted alkyl; and
R is unsubstituted or substituted alkyl;
or a tautomer or a stereoisomer or a salt of the afore-mentioned compounds;
or any mixture of the afore-mentioned compounds;
with the proviso, that the following compounds are disclaimed/excluded:

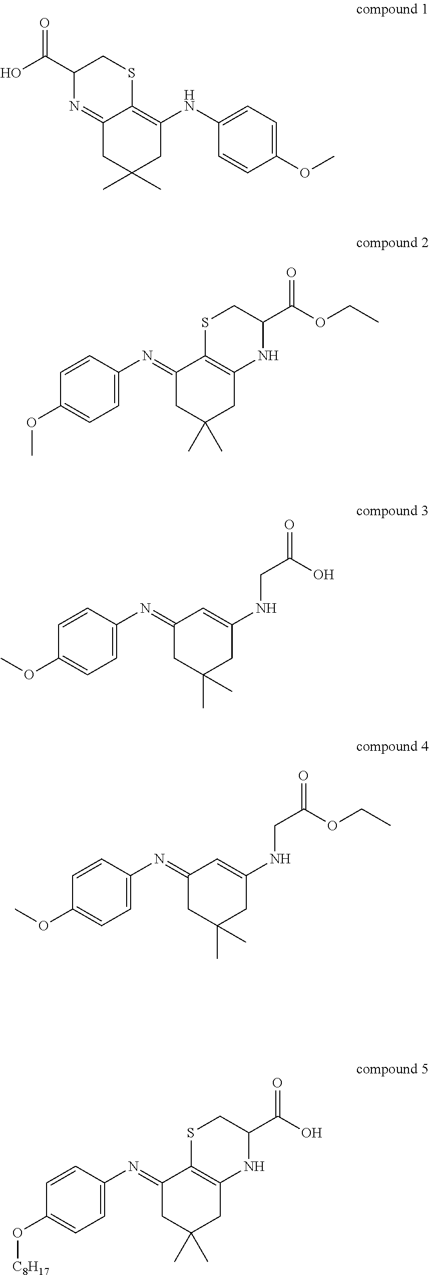

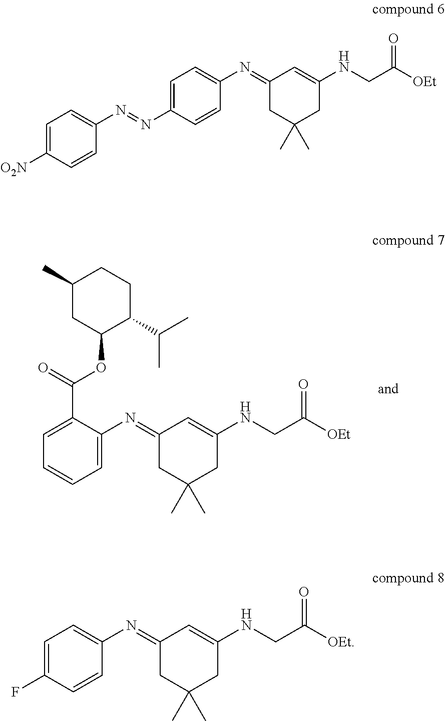 [SMILES: CCOC(=O)CNC1=C/C(=N/c2ccc(/N=N/c3ccc([N+](=O)[O-])cc3)cc2)CC(C)(C)C1 ; CCOC(=O)CNC1=C/C(=N/c2ccccc2C(=O)O[C@H]2C[C@@H](C)CC[C@@H]2C(C)C)CC(C)(C)C1 ; CCOC(=O)CNC1=C/C(=N/c2ccc(F)cc2)CC(C)(C)C1]

2. The sunscreen product, cosmetic or pharmaceutical preparation or homecare product according to claim 1, wherein the mycosporine-like amino acid compound is represented either by the general formula (VII) 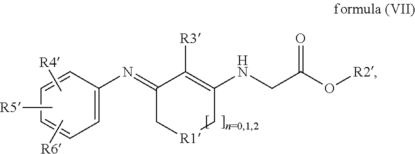
wherein R1′, R2′, R3′, R4′, R5′ and R6′ have the same meaning as defined for formula (V);
or by the general formula (VIII) 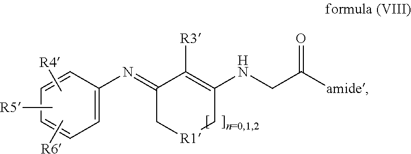
wherein R1′, amide′, R3′, R4′, R5′ and R6′ have the same meaning as defined for formula (V).

3. The sunscreen product, cosmetic or pharmaceutical preparation or homecare product according to claim 1, wherein in the general formula (VI) X is selected from the group consisting of CH₂, unsubstituted or substituted CH(alkyl), unsubstituted or substituted C(alkyl)₂, unsubstituted or substituted CH(alkenyl), unsubstituted or substituted CH(alkynyl), unsubstituted or substituted CH(alkoxy), unsubstituted or substituted CH(alkylthio), unsubstituted or substituted CH(cycloalkyl), unsubstituted or substituted CH(aryl), unsubstituted or substituted CH(heterocycloalkyl), unsubstituted or substituted CH(heteroaryl), —CR7″R8″, C═O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), unsubstituted or substituted N(heteroaryl), and unsubstituted or substituted C-spirocycles

4. The sunscreen product, cosmetic or pharmaceutical preparation or homecare product according to claim 1, wherein in the general formula (V)
Z is —O—R2′ or amide′; and/or
R1′ is selected from the group consisting of CH₂, C(alkyl)₂, O, S, SO, SO₂, NH and N(alkyl); and/or
R2′ is selected from the group consisting of H, methyl, ethyl, propyl, isopropyl, butyl, isobutyl, tert-butyl, 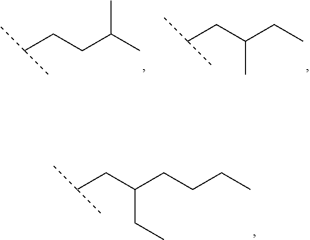
(2-ethylhexyl) and phenyl; or
amide′ is selected from the group consisting of NH₂, NH(alkyl) and N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl;
or wherein in the general formulae (VII) or (VIII)
R1′ is selected from the group consisting of CH₂, C(alkyl)₂, O, S, SO, SO₂, NH and N(alkyl); and/or
R2′ is selected from the group consisting of H, methyl, ethyl, propyl, isopropyl, butyl, isobutyl, tert-butyl, 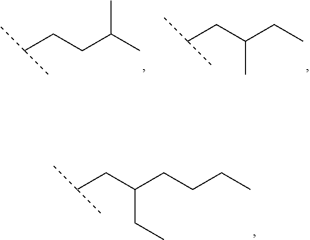
(2-ethylhexyl) and phenyl; or
amide′ is selected from the group consisting of NH₂, NH(alkyl) and N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formulae (VII) and (VIII);
wherein alkyl is selected from the group consisting of methyl, ethyl, propyl, isopropyl, butyl, isobutyl, and tert-butyl;
or wherein in the general formula (VI)
R1″ is selected from the group consisting of CH₂, C(alkyl)₂, O, S, SO, SO₂, NH and N(alkyl); and/or
R2″ is selected from the group consisting of —CH₂—OH, —C(═O)—O—Y, and —C(═O)-amide″; and/or
X is selected from the group consisting of CH₂, C═0, O, S, SO, SO₂, NH, and N(alkyl); and/or
Y is selected from the group consisting of H, methyl, ethyl, propyl, isopropyl, butyl, isobutyl, tert-butyl, 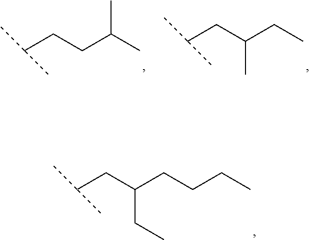
(2-ethylhexyl) and phenyl; and/or
amide″ is selected from the group consisting of NH₂, NH(alkyl) and N(alkyl)₂, —N(O-alkyl)(alkyl) (Weinreb amide) and —N(OH)(H) (hydroxamate); and/or
the substituents R4″, R5″ and R6″ have the same meaning as defined for formula (VI);
wherein alkyl is selected from the group consisting of methyl, ethyl, propyl, isopropyl, butyl, isobutyl, and tert-butyl.

5. The sunscreen product, cosmetic or pharmaceutical preparation or homecare product according to claim 1, wherein in the general formula (V)
Z is —O—R2′ or amide′; and/or
R1′ is selected from the group consisting of CH₂ and C(methyl)₂; and/or
R2′ is selected from the group consisting of H, methyl, ethyl, propyl, isopropyl, butyl, isobutyl, tert-butyl, 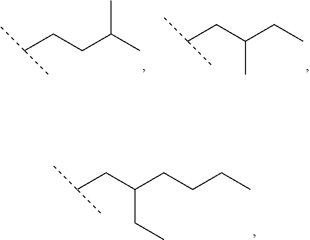
(2-ethylhexyl) and phenyl; or
amide′ is selected from the group consisting of NH₂, NH(alkyl) and N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl;
or wherein in the general formulae (VII) or (VIII)
R1′ is selected from the group consisting of CH₂, and C(methyl)₂;
R2′ is selected from the group consisting of H, methyl, ethyl, propyl, isopropyl, butyl, isobutyl, tert-butyl, 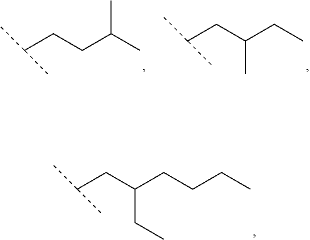
(2-ethylhexyl) and phenyl; or
amide′ is selected from the group consisting of NH₂, NH(alkyl) and N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formulae (VII) and (VIII);
wherein alkyl is selected from the group consisting of methyl, ethyl, propyl, isopropyl, butyl, isobutyl, and tert-butyl; and/or
or wherein in the general formula (VI)
R1″ is selected from the group consisting of CH₂ and C(methyl)₂; and/or
R2″ is selected from the group consisting of —CH₂—OH, —C(═O)—O—Y, and —C(═O)-amide″; and/or
X is selected from the group consisting of CH₂, C═O, O, S, SO, SO₂, NH, and N(alkyl); and/or
Y is selected from the group consisting of H, methyl, ethyl, propyl, isopropyl, butyl, isobutyl, tert-butyl, 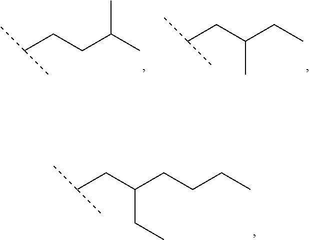
(2-ethylhexyl) and phenyl; and/or
amide″ is selected from the group consisting of NH₂, NH(alkyl) and N(alkyl)₂, —N(O-alkyl)(alkyl) (Weinreb amide) and —N(OH)(H) (hydroxamate); and/or
the substituents R4″, R5″ and R6″ have the same meaning as defined for formula (VI);
wherein alkyl is selected from the group consisting of methyl, ethyl, propyl, isopropyl, butyl, isobutyl, and tert-butyl.

6. The sunscreen product, cosmetic or pharmaceutical preparation or homecare product according to claim 1, wherein the mycosporine-like amino acid compound is selected from the group consisting of the following compounds I-1 to I-128 according to the general formula (V) and II-1 to II-192 according to the general formula (‘VI):

| 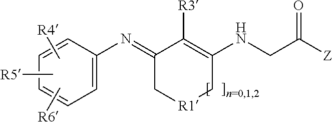 | R1′ | R3′ | Z |
|---|---|---|---|
| I-1 | CH2 | H | —O—R2′ |
| I-2 | CH2 | methyl | —O—R2′ |
| I-3 | CH2 | ethyl | —O—R2′ |
| I-4 | CH2 | —O-methyl | —O—R2′ |
| I-5 | CH2 | —C(═O)—OH | —O—R2′ |
| I-6 | CH2 | —C(═O)—CH3 | —O—R2′ |
| I-7 | CH2 | —C(═O)—C3H7 | —O—R2′ |
| I-8 | CH2 | —C(═O)—O-methyl | —O—R2′ |
| I-9 | CH2 | H | amide′ |
| I-10 | CH2 | methyl | amide′ |
| I-11 | CH2 | ethyl | amide′ |
| I-12 | CH2 | —O-methyl | amide′ |
| I-13 | CH2 | —C(═O)—OH | amide′ |
| I-14 | CH2 | —C(═O)—CH3 | amide′ |
| I-15 | CH2 | —C(═O)—C3H7 | amide′ |
| I-16 | CH2 | —C(═O)—O-methyl | amide′ |
| I-17 | C(methyl)2 | H | —O—R2′ |
| I-18 | C(methyl)2 | methyl | —O—R2′ |
| I-19 | C(methyl)2 | ethyl | —O—R2′ |
| I-20 | C(methyl)2 | —O-methyl | —O—R2′ |
| I-21 | C(methyl)2 | —C(═O)—OH | —O—R2′ |
| I-22 | C(methyl)2 | —C(═O)—CH3 | —O—R2′ |
| I-23 | C(methyl)2 | —C(═O)—C3H7 | —O—R2′ |
| I-24 | C(methyl)2 | —C(═O)—O-methyl | —O—R2′ |
| I-25 | C(methyl)2 | H | amide′ |
| I-26 | C(methyl)2 | methyl | amide′ |
| I-27 | C(methyl)2 | ethyl | amide′ |
| I-28 | C(methyl)2 | —O-methyl | amide′ |
| I-29 | C(methyl)2 | —C(═O)—OH | amide′ |
| I-30 | C(methyl)2 | —C(═O)—CH3 | amide′ |
| I-31 | C(methyl)2 | —C(═O)—C3H7 | amide′ |
| I-32 | C(methyl)2 | —C(═O)—O-methyl | amide′ |
| I-33 | O | H | —O—R2′ |
| I-34 | O | methyl | —O—R2′ |
| I-35 | O | ethyl | —O—R2′ |
| I-36 | O | —O-methyl | —O—R2′ |
| I-37 | O | —C(═O)—OH | —O—R2′ |
| I-38 | O | —C(═O)—CH3 | —O—R2′ |
| I-39 | O | —C(═O)—C3H7 | —O—R2′ |
| I-40 | O | —C(═O)—O-methyl | —O—R2′ |
| I-41 | O | H | amide′ |
| I-42 | O | methyl | amide′ |
| I-43 | O | ethyl | amide′ |
| I-44 | O | —O-methyl | amide′ |
| I-45 | O | —C(═O)—OH | amide′ |
| I-46 | O | —C(═O)—CH3 | amide′ |
| I-47 | O | —C(═O)—C3H7 | amide′ |
| I-48 | O | —C(═O)—O-methyl | amide′ |
| I-49 | S | H | —O—R2′ |
| I-50 | S | methyl | —O—R2′ |
| I-51 | S | ethyl | —O—R2′ |
| I-52 | S | —O-methyl | —O—R2′ |
| I-53 | S | —C(═O)—OH | —O—R2′ |
| I-54 | S | —C(═O)—CH3 | —O—R2′ |
| I-55 | S | —C(═O)—C3H7 | —O—R2′ |
| I-56 | S | —C(═O)—O-methyl | —O—R2′ |
| I-57 | S | H | amide′ |
| I-58 | S | methyl | amide′ |
| I-59 | S | ethyl | amide′ |
| I-60 | S | —O-methyl | amide′ |
| I-61 | S | —C(═O)—OH | amide′ |
| I-62 | S | —C(═O)—CH3 | amide′ |
| I-63 | S | —C(═O)—C3H7 | amide′ |
| I-64 | S | —C(═O)—O-methyl | amide′ |
| I-65 | SO | H | —O—R2′ |
| I-66 | SO | methyl | —O—R2′ |
| I-67 | SO | ethyl | —O—R2′ |
| I-68 | SO | —O-methyl | —O—R2′ |
| I-69 | SO | —C(═O)—OH | —O—R2′ |
| I-70 | SO | —C(═O)—CH3 | —O—R2′ |
| I-71 | SO | —C(═O)—C3H7 | —O—R2′ |
| I-72 | SO | —C(═O)—O-methyl | —O—R2′ |
| I-73 | SO | H | amide′ |
| I-74 | SO | methyl | amide′ |
| I-75 | SO | ethyl | amide′ |
| I-76 | SO | —O-methyl | amide′ |
| I-77 | SO | —C(═O)—OH | amide′ |
| I-78 | SO | —C(═O)—CH3 | amide′ |
| I-79 | SO | —C(═O)—C3H7 | amide′ |
| I-80 | SO | —C(═O)—O-methyl | amide′ |
| I-81 | SO2 | H | —O—R2′ |
| I-82 | SO2 | methyl | —O—R2′ |
| I-83 | SO2 | ethyl | —O—R2′ |
| I-84 | SO2 | —O-methyl | —O—R2′ |
| I-85 | SO2 | —C(═O)—OH | —O—R2′ |
| I-86 | SO2 | —C(═O)—CH3 | —O—R2′ |
| I-87 | SO2 | —C(═O)—C3H7 | —O—R2′ |
| I-88 | SO2 | —C(═O)—O-methyl | —O—R2′ |
| I-89 | SO2 | H | amide′ |
| I-90 | SO2 | methyl | amide′ |
| I-91 | SO2 | ethyl | amide′ |
| I-92 | SO2 | —O-methyl | amide′ |
| I-93 | SO2 | —C(═O)—OH | amide′ |
| I-94 | SO2 | —C(═O)—CH3 | amide′ |
| I-95 | SO2 | —C(═O)—C3H7 | amide′ |
| I-96 | SO2 | —C(═O)—O-methyl | amide′ |
| I-97 | NH | H | —O—R2′ |
| I-98 | NH | methyl | —O—R2′ |
| I-99 | NH | ethyl | —O—R2′ |
| I-100 | NH | —O-methyl | —O—R2′ |
| I-101 | NH | —C(═O)—OH | —O—R2′ |
| I-102 | NH | —C(═O)—CH3 | —O—R2′ |
| I-103 | NH | —C(═O)—C3H7 | —O—R2′ |
| I-104 | NH | —C(═O)—O-methyl | —O—R2′ |
| I-105 | NH | H | amide′ |
| I-106 | NH | methyl | amide′ |
| I-107 | NH | ethyl | amide′ |
| I-108 | NH | —O-methyl | amide′ |
| I-109 | NH | —C(═O)—OH | amide′ |
| I-110 | NH | —C(═O)—CH3 | amide′ |
| I-111 | NH | —C(═O)—C3H7 | amide′ |
| I-112 | NH | —C(═O)—O-methyl | amide′ |
| I-113 | N(alkyl) | H | —O—R2′ |
| I-114 | N(alkyl) | methyl | —O—R2′ |
| I-115 | N(alkyl) | ethyl | —O—R2′ |
| I-116 | N(alkyl) | —O-methyl | —O—R2′ |
| I-117 | N(alkyl) | —C(═O)—OH | —O—R2′ |
| I-118 | N(alkyl) | —C(═O)—CH3 | —O—R2′ |
| I-119 | N(alkyl) | —C(═O)—C3H7 | —O—R2′ |
| I-120 | N(alkyl) | —C(═O)—O-methyl | —O—R2′ |
| I-121 | N(alkyl) | H | amide′ |
| I-122 | N(alkyl) | methyl | amide′ |
| I-123 | N(alkyl) | ethyl | amide′ |
| I-124 | N(alkyl) | —O-methyl | amide′ |
| I-125 | N(alkyl) | —C(═O)—OH | amide′ |
| I-126 | N(alkyl) | —C(═O)—CH3 | amide′ |
| I-127 | N(alkyl) | —C(═O)—C3H7 | amide′ |
| I-128 | N(alkyl) | —C(═O)—O-methyl | amide′ |
| 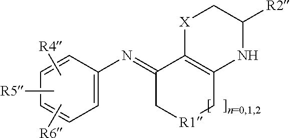 | R1″ | X | R2″ |
| II-1 | CH2 | CH2 | —CH2—OH |
| II-2 | CH2 | C═O | —CH2—OH |
| II-3 | CH2 | O | —CH2—OH |
| II-4 | CH2 | S | —CH2—OH |
| II-5 | CH2 | SO | —CH2—OH |
| II-6 | CH2 | SO2 | —CH2—OH |
| II-7 | CH2 | NH | —CH2—OH |
| II-8 | CH2 | N(alkyl) | —CH2—OH |
| II-9 | CH2 | CH2 | —C(═O)—O—Y |
| II-10 | CH2 | C═O | —C(═O)—O—Y |
| II-11 | CH2 | O | —C(═O)—O—Y |
| II-12 | CH2 | S | —C(═O)—O—Y |
| II-13 | CH2 | SO | —C(═O)—O—Y |
| II-14 | CH2 | SO2 | —C(═O)—O—Y |
| II-15 | CH2 | NH | —C(═O)—O—Y |
| II-16 | CH2 | N(alkyl) | —C(═O)—O—Y |
| II-17 | CH2 | CH2 | —C(═O)— |
| | | | amide″ |
| II-18 | CH2 | C═O | —C(═O)— |
| | | | amide″ |
| II-19 | CH2 | O | —C(═O)— |
| | | | amide″ |
| II-20 | CH2 | S | —C(═O)— |
| | | | amide″ |
| II-21 | CH2 | SC | —C(═O)— |
| | | | amide″ |
| II-22 | CH2 | SO2 | —C(═O)— |
| | | | amide″ |
| II-23 | CH2 | NH | —C(═O)— |
| | | | amide″ |
| II-24 | CH2 | N(alkyl) | —C(═O)— |
| | | | amide″ |
| II-25 | C(methyl)2 | CH2 | —CH2—OH |
| II-26 | C(methyl)2 | C═O | —CH2—OH |
| II-27 | C(methyl)2 | O | —CH2—OH |
| II-28 | C(methyl)2 | S | —CH2—OH |
| II-29 | C(methyl)2 | SO | —CH2—OH |
| II-30 | C(methyl)2 | SO2 | —CH2—OH |
| II-31 | C(methyl)2 | NH | —CH2—OH |
| II-32 | C(methyl)2 | N(alkyl) | —CH2—OH |
| II-33 | C(methyl)2 | CH2 | —C(═O)—O—Y |
| II-34 | C(methyl)2 | C═O | —C(═O)—O—Y |
| II-35 | C(methyl)2 | O | —C(═O)—O—Y |
| II-36 | C(methyl)2 | S | —C(═O)—O—Y |
| II-37 | C(methyl)2 | SC | —C(═O)—O—Y |
| II-38 | C(methyl)2 | SO2 | —C(═O)—O—Y |
| II-39 | C(methyl)2 | NH | —C(═O)—O—Y |
| II-40 | C(methyl)2 | N(alkyl) | —C(═O)—O—Y |
| II-41 | C(methyl)2 | CH2 | —C(═O)— |
| | | | amide″ |
| II-42 | C(methyl)2 | C═O | —C(═O)— |
| | | | amide″ |
| II-43 | C(methyl)2 | O | —C(═O)— |
| | | | amide″ |
| II-44 | C(methyl)2 | S | —C(═O)— |
| | | | amide″ |
| II-45 | C(methyl)2 | SC | —C(═O)— |
| | | | amide″ |
| II-46 | C(methyl)2 | SO2 | —C(═O)— |
| | | | amide″ |
| II-47 | C(methyl)2 | NH | —C(═O)— |
| | | | amide″ |
| II-48 | C(methyl)2 | N(alkyl) | —C(═O)— |
| | | | amide″ |
| II-49 | O | CH2 | —CH2—OH |
| II-50 | O | C═O | —CH2—OH |
| II-51 | O | O | —CH2—OH |
| II-52 | O | S | —CH2—OH |
| II-53 | O | SO | —CH2—OH |
| II-54 | O | SO2 | —CH2—OH |
| II-55 | O | NH | —CH2—OH |
| II-56 | O | N(alkyl) | —CH2—OH |
| II-57 | O | CH2 | —C(═O)—O—Y |
| II-58 | O | C═O | —C(═O)—O—Y |
| II-59 | O | O | —C(═O)—O—Y |
| II-60 | O | S | —C(═O)—O—Y |
| II-61 | O | SO | —C(═O)—O—Y |
| II-62 | O | SO2 | —C(═O)—O—Y |
| II-63 | O | NH | —C(═O)—O—Y |
| II-64 | O | N(alkyl) | —C(═O)—O—Y |
| II-65 | O | CH2 | —C(═O)— |
| | | | amide″ |
| II-66 | O | C═O | —C(═O)— |
| | | | amide″ |
| II-67 | O | O | —C(═O)— |
| | | | amide″ |
| II-68 | O | S | —C(═O)— |
| | | | amide″ |
| II-69 | O | SO | —C(═O)— |
| | | | amide″ |
| II-70 | O | SO2 | —C(═O)— |
| | | | amide″ |
| II-71 | O | NH | —C(═O)— |
| | | | amide″ |
| II-72 | O | N(alkyl) | —C(═O)— |
| | | | amide″ |
| II-73 | S | CH2 | —CH2—OH |
| II-74 | S | C═O | —CH2—OH |
| II-75 | S | O | —CH2—OH |
| II-76 | S | S | —CH2—OH |
| II-77 | S | SO | —CH2—OH |
| II-78 | S | SO2 | —CH2—OH |
| II-79 | S | NH | —CH2—OH |
| II-80 | S | N(alkyl) | —CH2—OH |
| II-81 | S | CH2 | —C(═O)—O—Y |
| II-82 | S | C═O | —C(═O)—O—Y |
| II-83 | S | O | —C(═O)—O—Y |
| II-84 | S | S | —C(═O)—O—Y |
| II-85 | S | SO | —C(═O)—O—Y |
| II-86 | S | SO2 | —C(═O)—O—Y |
| II-87 | S | NH | —C(═O)—O—Y |
| II-88 | S | N(alkyl) | —C(═O)—O—Y |
| II-89 | S | CH2 | —C(═O)— |
| | | | amide″ |
| II-90 | S | C═O | —C(═O)— |
| | | | amide″ |
| II-91 | S | O | —C(═O)— |
| | | | amide″ |
| II-92 | S | S | —C(═O)— |
| | | | amide″ |
| II-93 | S | SO | —C(═O)— |
| | | | amide″ |
| II-94 | S | SO2 | —C(═O)— |
| | | | amide″ |
| II-95 | S | NH | —C(═O)— |
| | | | amide″ |
| II-96 | S | N(alkyl) | —C(═O)— |
| | | | amide″ |
| II-97 | SO | CH2 | —CH2—OH |
| II-98 | SO | C═O | —CH2—OH |
| II-99 | SO | 0 | —CH2—OH |
| II-100 | SO | S | —CH2—OH |
| II-101 | SO | SO | —CH2—OH |
| II-102 | SO | SO2 | —CH2—OH |
| II-103 | SO | NH | —CH2—OH |
| II-104 | SO | N(alkyl) | —CH2—OH |
| II-105 | SO | CH2 | —C(═O)—O—Y |
| II-106 | SO | C═O | —C(═O)—O—Y |
| II-107 | SO | O | —C(═O)—O—Y |
| II-108 | SO | S | —C(═O)—O—Y |
| II-109 | SO | SO | —C(═O)—O—Y |
| II-110 | SO | SO2 | —C(═O)—O—Y |
| II-111 | SO | NH | —C(═O)—O—Y |
| II-112 | SO | N(alkyl) | —C(═O)—O—Y |
| II-113 | SO | CH2 | —C(═O)— |
| | | | amide″ |
| II-114 | SO | C═O | —C(═O)— |
| | | | amide″ |
| II-115 | SO | O | —C(═O)— |
| | | | amide″ |
| II-116 | SO | S | —C(═O)— |
| | | | amide″ |
| II-117 | SO | SO | —C(═O)— |
| | | | amide″ |
| II-118 | SO | SO2 | —C(═O)— |
| | | | amide″ |
| II-119 | SO | NH | —C(═O)— |
| | | | amide″ |
| II-120 | SO | N(alkyl) | —C(═O)— |
| | | | amide″ |
| II-121 | SO2 | CH2 | —CH2—OH |
| II-122 | SO2 | C═O | —CH2—OH |
| II-123 | SO2 | O | —CH2—OH |
| II-124 | SO2 | S | —CH2—OH |
| II-125 | SO2 | SO | —CH2—OH |
| II-126 | SO2 | SO2 | —CH2—OH |
| II-127 | SO2 | NH | —CH2—OH |
| II-128 | SO2 | N(alkyl) | —CH2—OH |
| II-129 | SO2 | CH2 | —C(═O)—O—Y |
| II-130 | SO2 | C═O | —C(═O)—O—Y |
| II-131 | SO2 | O | —C(═O)—O—Y |
| II-132 | SO2 | S | —C(═O)—O—Y |
| II-133 | SO2 | SO | —C(═O)—O—Y |
| II-134 | SO2 | SO2 | —C(═O)—O—Y |
| II-135 | SO2 | NH | —C(═O)—O—Y |
| II-136 | SO2 | N(alkyl) | —C(═O)—O—Y |
| II-137 | SO2 | CH2 | —C(═O)— |
| | | | amide″ |
| II-138 | SO2 | C═O | —C(═O)— |
| | | | amide″ |
| II-139 | SO2 | O | —C(═O)— |
| | | | amide″ |
| II-140 | SO2 | S | —C(═O)— |
| | | | amide″ |
| II-141 | SO2 | SO | —C(═O)— |
| | | | amide″ |
| II-142 | SO2 | SO2 | —C(═O)— |
| | | | amide″ |
| II-143 | SO2 | NH | —C(═O)— |
| | | | amide″ |
| II-144 | SO2 | N(alkyl) | —C(═O)— |
| | | | amide″ |
| II-145 | NH | CH2 | —CH2—OH |
| II-146 | NH | C═O | —CH2—OH |
| II-147 | NH | O | —CH2—OH |
| II-148 | NH | S | —CH2—OH |
| II-149 | NH | SO | —CH2—OH |
| II-150 | NH | SO2 | —CH2—OH |
| II-151 | NH | NH | —CH2—OH |
| II-152 | NH | N(alkyl) | —CH2—OH |
| II-153 | NH | CH2 | —C(═O)—O—Y |
| II-154 | NH | C═O | —C(═O)—O—Y |
| II-155 | NH | O | —C(═O)—O—Y |
| II-156 | NH | S | —C(═O)—O—Y |
| II-157 | NH | SO | —C(═O)—O—Y |
| II-158 | NH | SO2 | —C(═O)—O—Y |
| II-159 | NH | NH | —C(═O)—O—Y |
| II-160 | NH | N(alkyl) | —C(═O)—O—Y |
| II-161 | NH | CH2 | —C(═O)— |
| | | | amide″ |
| II-162 | NH | C═O | —C(═O)— |
| | | | amide″ |
| II-163 | NH | O | —C(═O)— |
| | | | amide″ |
| II-164 | NH | S | —C(═O)— |
| | | | amide″ |
| II-165 | NH | SO | —C(═O)— |
| | | | amide″ |
| II-166 | NH | SO2 | —C(═O)— |
| | | | amide″ |
| II-167 | NH | NH | —C(═O)— |
| | | | amide″ |
| II-168 | NH | N(alkyl) | —C(═O)— |
| | | | amide″ |
| II-169 | N(alkyl) | CH2 | —CH2—OH |
| II-170 | N(alkyl) | C═O | —CH2—OH |
| II-171 | N(alkyl) | O | —CH2—OH |
| II-172 | N(alkyl) | S | —CH2—OH |
| II-173 | N(alkyl) | SO | —CH2—OH |
| II-174 | N(alkyl) | SO2 | —CH2—OH |
| II-175 | N(alkyl) | NH | —CH2—OH |
| II-176 | N(alkyl) | N(alkyl) | —CH2—OH |
| II-177 | N(alkyl) | CH2 | —C(═O)—O—Y |
| II-178 | N(alkyl) | C═O | —C(═O)—O—Y |
| II-179 | N(alkyl) | O | —C(═O)—O—Y |
| II-180 | N(alkyl) | S | —C(═O)—O—Y |
| II-181 | N(alkyl) | SO | —C(═O)—O—Y |
| II-182 | N(alkyl) | SO2 | —C(═O)—O—Y |
| II-183 | N(alkyl) | NH | —C(═O)—O—Y |
| II-184 | N(alkyl) | N(alkyl) | —C(═O)—O—Y |
| II-185 | N(alkyl) | CH2 | —C(═O)— |
| | | | amide″ |
| II-186 | N(alkyl) | C═O | —C(═O)— |
| | | | amide″ |
| II-187 | N(alkyl) | O | —C(═O)— |
| | | | amide″ |
| II-188 | N(alkyl) | S | —C(═O)— |
| | | | amide″ |
| II-189 | N(alkyl) | SO | —C(═O)— |
| | | | amide″ |
| II-190 | N(alkyl) | SO2 | —C(═O)— |
| | | | amide″ |
| II-191 | N(alkyl) | NH | —C(═O)— |
| | | | amide″ |
| II-192 | N(alkyl) | N(alkyl) | —C(═O)— |
| | | | amide″ |

wherein in the above mycosporine-like amino acid compounds the phenyl ring may be unsubstituted or substituted, and R4′, R5′, R6′, R4″, R5″, R6″, amide′, Y, amide″ and alkyl have the same meaning as defined in any one of the preceding claims, or a tautomer or a stereoisomer or a salt of the afore-mentioned compounds;
or wherein the mycosporine-like amino acid is selected from the group consisting of 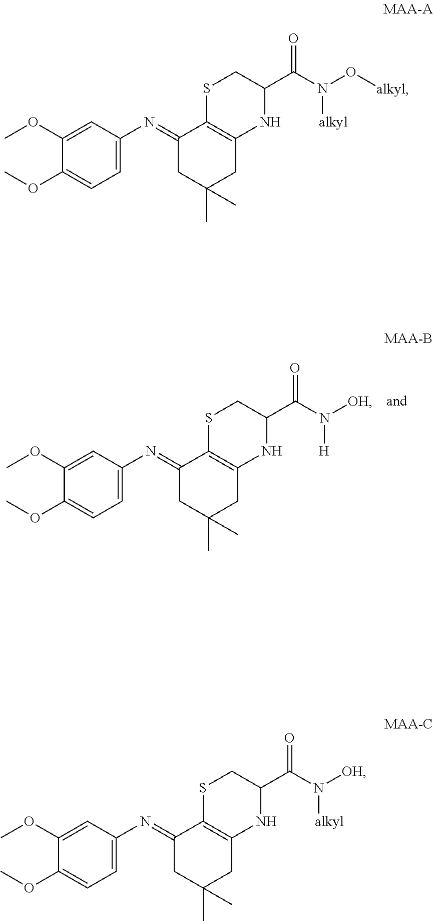
wherein alkyl is either methyl or ethyl or propyl or isopropyl or butyl or isobutyl or tert-butyl, or a tautomer or a stereoisomer or a salt of the afore-mentioned compounds;
or any mixture of the afore-mentioned compounds.

7. The sunscreen product, cosmetic or pharmaceutical preparation or homecare product according to claim 1, wherein in the general formulae (VI), X is S, SO or SO₂, and/or wherein in the general formulae (V) to (VIII) n=1.

8. The sunscreen product, cosmetic or pharmaceutical preparation or homecare product according to claim 1, wherein in the general formulae (V), (VII) and (VIII) the substituents R4′, R5′ and R6′ which may be identical or different, are independently from each other selected from the group consisting of H, OH, alkyl, alkoxy, —S(═O)₂OH, and sulfonyl;
or
wherein in the general formulae (VI), the substituents R4″, R5″ and R6″ which may be identical or different, are independently from each other selected from the group consisting of H, OH, alkyl, alkoxy, —S(═O)₂OH, and sulfonyl.

9. The sunscreen product, cosmetic or pharmaceutical preparation or homecare product according to claim 1, wherein in the general formulae (V) to (VIII) the substituted phenyl bonded to the imino functionality is selected from the group consisting of 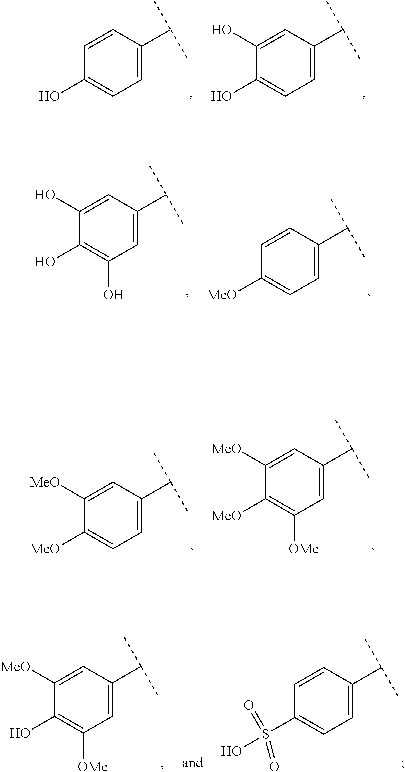
wherein the dotted line designates the binding site.

10. The sunscreen product, cosmetic or pharmaceutical preparation or homecare product according to claim 1, wherein in the general formulae (V), (VII) and (VIII), the substituents R4′, R5′, R6′, which may be identical or different, are independently from each other selected from the group consisting of H, OH, alkyl, alkoxy, —S(═O)₂OH, and sulfonyl; with the proviso, that if the substituted phenyl ring is monosubstituted, the substituent is either in the ortho or meta-position to the imino-functionality; or wherein in the general formula (VI), the substituents R4″, R5″ and R6″, which may be identical or different, are independently from each other selected from the group consisting of H, OH, alkyl, alkoxy, —S(═O)₂OH, and sulfonyl; with the proviso, that if the substituted phenyl ring is monosubstituted, the substituent is either in the ortho or meta-position to the imino-functionality of the general formulae (VI), but not in the para position to the imino-functionality.

11. The sunscreen product, cosmetic or pharmaceutical preparation or homecare product according to claim 1, wherein the mycosporine-like amino acid compound is selected from the group consisting of

| Compound | Structure | INCI/chemical name |
|---|---|---|
| MAA-1 | 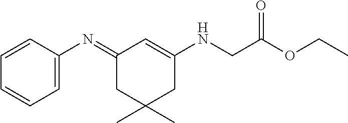 [SMILES: CCOC(=O)CNC1=C/C(=N/c2ccccc2)CC(C)(C)C1] | ethyl 2-[[5,5-dimethyl-3- phenylimino-cyclohexen-1- yl]amino]acetate |
| MAA-2 | 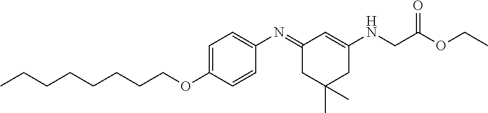 [SMILES: CCCCCCCCOc1ccc(/N=C2/C=C(NCC(=O)OCC)CC(C)(C)C2)cc1] | ethyl 2-((5,5-dimethyl-3-((4- octyoxy)phenyl)imino)cyclohex-1- en-1-yl)amino)acetate |
| MAA-3 | 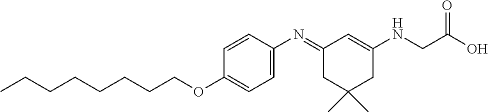 [SMILES: CCCCCCCCOc1ccc(/N=C2/C=C(NCC(=O)O)CC(C)(C)C2)cc1] | 5,5-dimethyl-3-((4- (octyloxy)phenyl)imino)cyclohex-1- en-1-yl-glycine |
| MAA-4 | 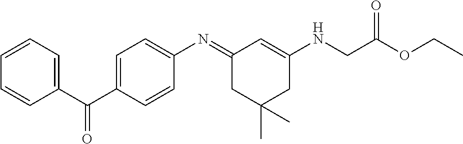 [SMILES: CCOC(=O)CNC1=C/C(=N/c2ccc(C(=O)c3ccccc3)cc2)CC(C)(C)C1] | ethyl 2-((3-((4- benzoylphenyl)imino)-5,5- dimethylcyclohex-1-en-1- yl)amino)acetate |
| MAA-5 | 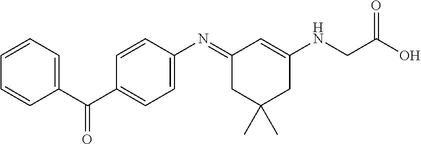 [SMILES: CC1(C)CC(NCC(=O)O)=C/C(=N/c2ccc(C(=O)c3ccccc3)cc2)C1] | 3-((4-benzoylphenyl)imino)-5,5- dimethylcyclohex-1-en-1-yl-glycine |
| MAA-6 | 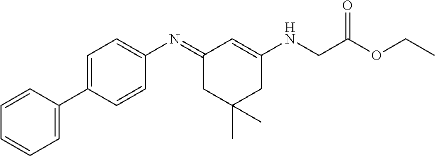 [SMILES: CCOC(=O)CNC1=C/C(=N/c2ccc(-c3ccccc3)cc2)CC(C)(C)C1] | ethyl 2-((3-([1,1′-biphenyl]-4- ylimino)-5,5-dimethylcyclohex-1-en- 1.yl)amino)acetate |
| MAA-7 | 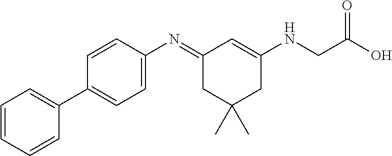 [SMILES: CC1(C)CC(NCC(=O)O)=C/C(=N/c2ccc(-c3ccccc3)cc2)C1] | 3-([1,1′-biphenyl]-4-ylimino)-5,5- dimethylcyclohex-1-en-1-yl-glycine |
| MAA-8 | 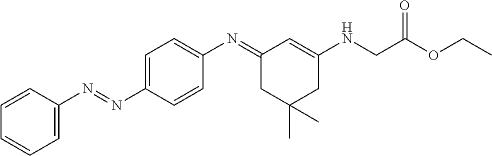 [SMILES: CCOC(=O)CNC1=C/C(=N/c2ccc(/N=N/c3ccccc3)cc2)CC(C)(C)C1] | ethyl 2-((5,5-dimethyl-3-((4- (phenyldiazenyl)phenyl)inino(cyclo- hex-1-en-1-yl)amino)acetate |
| MAA-9 | 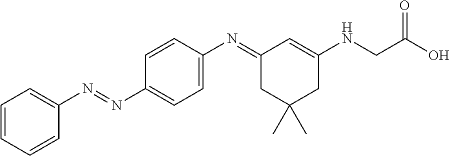 [SMILES: CC1(C)CC(NCC(=O)O)=C/C(=N/c2ccc(/N=N/c3ccccc3)cc2)C1] | 5,5-dimethyl-3-((4-((E)- phenyldiazenyl)phenyl)imino)cyclo- hex-1-en-1-yl-glycine |
| MAA-11 | 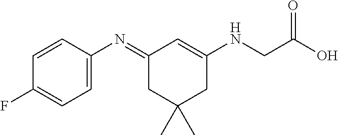 [SMILES: CC1(C)CC(NCC(=O)O)=C/C(=N/c2ccc(F)cc2)C1] | 3-((4-fluorophenyl)imino)-5,5- dimethylcyclohex-1-en-1-yl-glycine |
| MAA-12 | 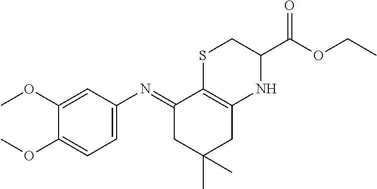 [SMILES: CCOC(=O)C1CSC2=C(CC(C)(C)C/C2=N\c2ccc(OC)c(OC)c2)N1] | ethyl 8-((3,4- dimethoyxphenyl)imino)-6,6- dimethyl-3,4,5,6,7,8-hexahydro-2-H- benzol[b][1,4]thiazine-3-carboxylate |
| MAA-13 | 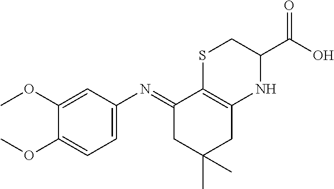 [SMILES: COc1ccc(/N=C2\CC(C)(C)CC3=C2SCC(C(=O)O)N3)cc1OC] | 8-((3,4-dimethoyxphenyl)imino)-6,6- dimethyl-3,4,5,6,7,8-hexahydro-2-H- benzol[b][1,4]thiazine-3-carboxylic acid |
| MAA-14 | 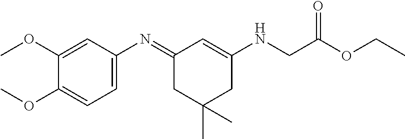 [SMILES: CCOC(=O)CNC1=C/C(=N/c2ccc(OC)c(OC)c2)CC(C)(C)C1] | ethyl 2-((3-((3,4- dimethoxyphenyl)imino)-5,5- dimethylcyclohex-1-en-1- yl)amino)acetate |
| MAA-16 | 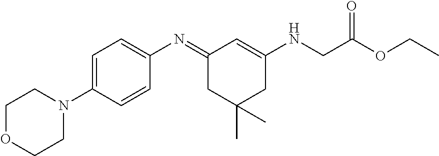 [SMILES: CCOC(=O)CNC1=C/C(=N/c2ccc(N3CCOCC3)cc2)CC(C)(C)C1] | ethyl 2-((5,5-dimethyl-3-((4- morpholinophenyl)imino)cyclohex- 1-en-1-yl)amino)acetate |
| MAA-17 | 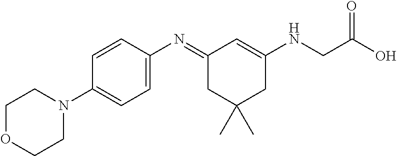 [SMILES: CC1(C)CC(NCC(=O)O)=C/C(=N/c2ccc(N3CCOCC3)cc2)C1] | 5,5-dimethyl-3-((4- morpholinophenyl)imino)cyclohex- 1-en-1-yl-glycine |
| MAA-20 | 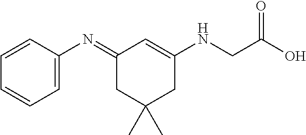 [SMILES: CC1(C)CC(NCC(=O)O)=C/C(=N/c2ccccc2)C1] | 2-[(5,5-dimethyl-3-phenylimino- cyclohexen-1-yl)amino]acetic acid |
| MAA-21 | 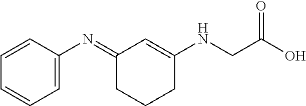 [SMILES: O=C(O)CNC1=C/C(=N/c2ccccc2)CCC1] | 2-[(3-phenyliminocyclohexen-1- yl)amino]acetic acid |
| MAA-22 | 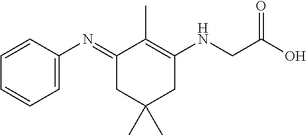 [SMILES: CC1=C(NCC(=O)O)CC(C)(C)C/C1=N\c1ccccc1] | 2,5,5-trimethyl-3- (phenylimino)cyclohex-1-en-1-yl- glycine |
| MAA-23 | 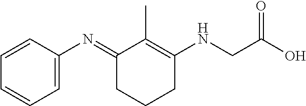 [SMILES: CC1=C(NCC(=O)O)CCC/C1=N\c1ccccc1] | 2-[(2-methyl-3-phenylimino- cyclohexen-1-yl)amino]acetic acid |
| MAA-24 | 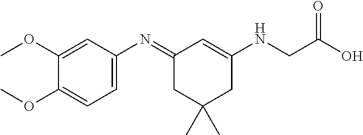 [SMILES: COc1ccc(/N=C2/C=C(NCC(=O)O)CC(C)(C)C2)cc1OC] | 3-((3,4-dimethoxyphenyl)imino)-5,5- dimethylcyclohex-1-en-1-yl-glycine |
| MAA-25 | 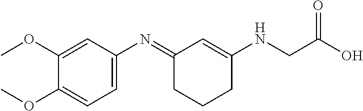 [SMILES: COc1ccc(/N=C2/C=C(NCC(=O)O)CCC2)cc1OC] | 3-((3,4- dimethoxyphenyl)imino)cyclohex-1- en-1-yl-glycine |
| MAA-26 | 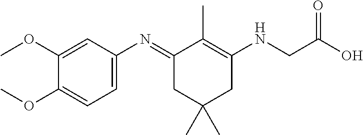 [SMILES: COc1ccc(/N=C2\CC(C)(C)CC(NCC(=O)O)=C2C)cc1OC] | 3-((3,4-dimethoxyphenyl)imino)- 2,5,5-trimethylcyclohex-1-en-1-yl- glycine |
| MAA-27 | 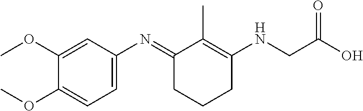 [SMILES: COc1ccc(/N=C2\CCCC(NCC(=O)O)=C2C)cc1OC] | 2-[(3-(3,4-dimethoxyphenyl)imino-2- methyl-cyclohexen-1-yl)amino]acetic acid |
| MAA-28 | 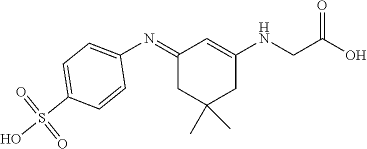 [SMILES: CC1(C)CC(NCC(=O)O)=C/C(=N/c2ccc(S(=O)(=O)O)cc2)C1] | 2-[(5,5-dimethyl-3-(4- sulfophenyl)imino-cyclohexen-1- yl)amino]acetic acid |
| MAA-29 | 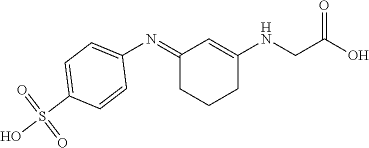 [SMILES: O=C(O)CNC1=C/C(=N/c2ccc(S(=O)(=O)O)cc2)CCC1] | 2-[(3-(4- sulfophenyl)iminocyclohexen-1- yl)amino]acetic acid |
| MAA-30 | 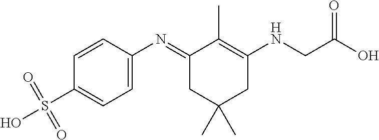 [SMILES: CC1=C(NCC(=O)O)CC(C)(C)C/C1=N\c1ccc(S(=O)(=O)O)cc1] | 2,5,5-trimethyl-3-((4- sulfophenyl)imino)cyclohex-1-en-1- yl-glycine |
| MAA-31 | 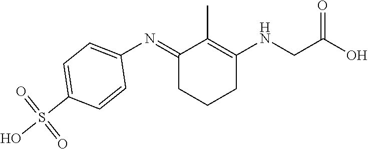 [SMILES: CC1=C(NCC(=O)O)CCC/C1=N\c1ccc(S(=O)(=O)O)cc1] | 2-methyl-3-((4- sulfophenyl)imino)cyclohex-1-en-1- yl-glycine |
| MAA-32 | 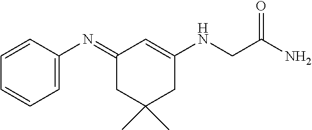 [SMILES: CC1(C)CC(NCC(N)=O)=C/C(=N/c2ccccc2)C1] | 2-[(5,5-dimethyl-3-phenylimino- cyclohexen-1-yl)amino]acetamide |
| MAA-33 | 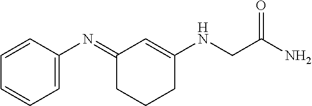 [SMILES: NC(=O)CNC1=C/C(=N/c2ccccc2)CCC1] | 2-[(3-phenyliminocyclohexen-1- yl)amino]acetamide |
| MAA-34 | 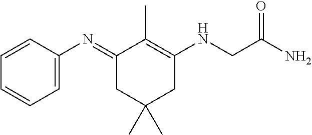 [SMILES: CC1=C(NCC(N)=O)CC(C)(C)C/C1=N\c1ccccc1] | 2-((2,5,5-trimethyl-3- (phenylimino)cyclohex-1-en-1- yl)amino)acetamide |
| MAA-35 | 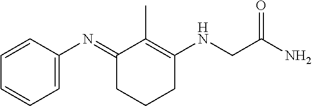 [SMILES: CC1=C(NCC(N)=O)CCC/C1=N\c1ccccc1] | 2-((2-methyl-3- (phenylimino)cyclohex-1-en-1- yl)amino)acetamide |
| MAA-36 | 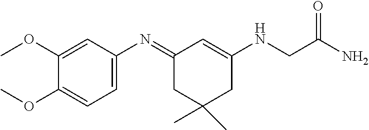 [SMILES: COc1ccc(/N=C2/C=C(NCC(N)=O)CC(C)(C)C2)cc1OC] | 2-((3-((3,4-dimethoxyphenyl)imino)- 5,5-dimethylcyclohex-1-en-1- yl)amino)acetamide |
| MAA-37 | 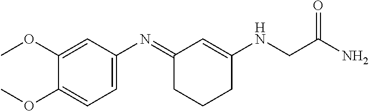 [SMILES: COc1ccc(/N=C2/C=C(NCC(N)=O)CCC2)cc1OC] | 2-[(3-(3,4- dimethoxyphenyl)iminocyclohexen- 1-yl)amino]acetamide |
| MAA-38 | 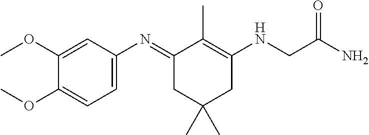 [SMILES: COc1ccc(/N=C2\CC(C)(C)CC(NCC(N)=O)=C2C)cc1OC] | 2-((3-((3,4-dimethoxyphenyl)imino)- 2,5,5-trimethylcyclohex-1-en-1- yl)amino)acetamide |
| MAA-39 | 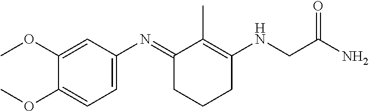 [SMILES: COc1ccc(/N=C2\CCCC(NCC(N)=O)=C2C)cc1OC] | 2-((3-((3,4-dimethoxyphenyl)imino)- 2-methylcyclohex-1-en-1- yl)amino)acetamide |

or a tautomer or a stereoisomer or a salt of the afore-mentioned compounds;
or any mixture of the afore-mentioned compounds.

12. The sunscreen product, cosmetic or pharmaceutical preparation or homecare product according to claim 1, wherein the at least one photo-unstable UV-filter is selected from the group consisting of Ethylhexyl-Methoxycinnamate (Neo Heliopan® AV), Butyl-Methoxydibenzoylmethane (Avobenzone; Neo Heliopan® 357), Isoamyl-p-Methoxycinnamate (Neo Heliopan® E1000), Diethylamino-Hydroxy-Benzoylhexylbenzoate (Uvinul A plus) and any mixture thereof.

13. The sunscreen product, cosmetic or pharmaceutical preparation or homecare product according to claim 1 further comprising at least one additional primary organic and/or inorganic UV-filter which is different from the at least one photo-unstable UV-filter, wherein the additional primary organic and/or inorganic UV-filter is selected from the group consisting of Camphor Benzalkonium Methosulfate, Homosalate, Benzophenone-3, Phenylbenzimidazole Sulfonic Acid, Terephthalylidene Dicamphor Sulfonic Acid, Benzylidene Camphor Sulfonic Acid, Octocrylene, Polyacrylamidomethyl Benzylidene Camphor, PEG-25 PABA, Ethylhexyl Triazone, Drometrizole Trisiloxane, Diethylhexyl Butamido Triazone, 4-Methylbenzylidene Camphor, Ethylhexyl Salicylate, Ethylhexyl Dimethyl PABA, Benzophenone-4, Benzophenone-5, Methylene Bis-Benzotriazolyl Tetramethylbutylphenol (nano), Disodium Phenyl Dibenzimidazole Tetrasulfonate, Bis-Ethylhexyloxyphenol Methoxyphenyl Triazine, Polysilicone-15, Titanium Dioxide, Titanium Dioxide (nano), Tris-biphenyl triazine (nano), Zinc Oxide, Zinc Oxide (nano), Phenylene Bis-Diphenyltriazine, Methoxypropylamino Cyclohexenylidene Ethoxyethylcyanoacetate, Bis-(Diethylaminohydroxybenzoyl Benzoyl) Piperazine, TEA-Salicylate, Benzophenone-8, PABA, Ethylhexyl Dimethyl PABA, Menthyl Anthranilate, and any mixture thereof.

14. The sunscreen product, cosmetic or pharmaceutical preparation or homecare product according to claim 1, comprising the photo-unstable UV-filter or a mixture thereof in an amount of 0.01 to 35.0% by weight, based on the total weight of the product or preparation.

15. The sunscreen product or cosmetic or pharmaceutical preparation according to claim 1, comprising the mycosporine-like amino acid compound or a mixture thereof in an amount of 0.001 to 15.0% by weight based on the total weight of the product or preparation.

16. The sunscreen product, cosmetic or pharmaceutical preparation or homecare product according to claim 1, further comprising at least one 1,2-alkanediol or one 2,3-alkanediol with 5 to 12 carbon atoms or a mixture thereof and/or further comprising one or more microbial agents or a mixture thereof and/or further comprising at least one excipient and/or active substance selected from the group consisting of UV-filters, carriers, oil components, rheology additives, hydrotropes solubilizing agents, powders, film formers, water resistance improving agents, skin moisturizing and/or me-retaining substances, lenitive substances, physiological cooling agents, alkanediols, antioxidants, agents against ageing of the skin, matrix-metalloproteinase inhibitors (MMPI), anti-inflammatory agents, TRPV1 antagonists, emulsifiers, antimicrobial agents, preservatives, antibacterial or antimycotic active substances, chelating agents, surfactants, fragrances/aroma or perfume oils, and any mixture of two or more of the afore-mentioned substances.

17-18. (canceled)

19. The sunscreen product or cosmetic or pharmaceutical preparation according claim 1 in the form of
(i) a dispersion;
(ii) a water free formulation; or
(iii) an aqueous, an aqueous/alcoholic, an aqueous/glycolic, or an alcoholic/glycolic solution.

20. (canceled)

21. The sunscreen product, cosmetic or pharmaceutical preparation or homecare product according to claim 1, wherein the degradation loss of the one photo-unstable UV-filter after irradiation at 5 MED determined as described in the application is reduced by at least 10%, compared to a composition comprising the at least one photo-unstable UV-filter without the mycosporine-like amino acid compound.

22-23. (canceled)

24. Method of stabilizing a photo-unstable UV-filter against the degradative effects of UV irradiation comprising mixing a photo-unstable UV-filter with an effective amount of at least one mycosporine-like amino acid compound or a mixture thereof.

25. The method of claim 24, wherein the degradation loss of the photo-unstable UV-filter after irradiation at 5 MED, determined as described in the present application, is reduced by at least 10%.
